(19) United States
(12) Patent Application Publication
(10) Pub. No.: US 2024/0360122 A1
(43) Pub. Date: Oct. 31, 2024

(54) POLYMORPHIC FORMS AND SALTS OF A GLP-1R AGONIST

(71) Applicants: Terns (Suzhou) Biotechnology Co., Ltd., Suzhou (CN); Terns Pharmaceuticals, Inc., Foster City, CA (US)

(72) Inventors: Jianwei BIAN, Foster, City, CA (US); Chunlong CAI, Foster City, CA (US); Lu A. CHOU, Foster City, CA (US)

(21) Appl. No.: 18/619,638

(22) Filed: Mar. 28, 2024

(30) Foreign Application Priority Data
Mar. 29, 2023 (WO) PCT/CN2023/084794

(51) Int. Cl. C07D 417/14; A61K 31/4545

(52) U.S. Cl. CPC C07D 417/14; A61K 31/4545

(57) ABSTRACT

The disclosure relates to solid forms of 2-((4-(6-((4-cyano-2-fluorobenzyl)oxy)pyridin-2-yl)piperidin-1-yl)methyl)-1-(thiazol-5-ylmethyl)-1H-benzo[d]imidazole-6-carboxylic acid (Compound 1), including solid forms of Compound 1, salts of Compound 1 and solid forms thereof, including crystalline forms of Compound 1 and salts thereof, as well as polymorphs of Compound 1 and salts of Compound 1.

### CROSS-REFERENCE TO RELATED APPLICATIONS

This application claims priority to, and the benefit of, International Application No. PCT/CN2023/084794, filed on Mar. 29, 2023, the entire contents of which are incorporated herein by reference.

### BACKGROUND

Diabetes is a major public health concern because of its increasing prevalence and associated health risks. The disease is characterized by high levels of blood glucose resulting from defects in insulin production, insulin action, or both. Two major forms of diabetes are recognized, Type 1 and Type 2. Type 1 diabetes (T1D) develops when the body's immune system destroys pancreatic beta cells, the only cells in the body that make the hormone insulin that regulates blood glucose. To survive, people with Type 1 diabetes must have insulin administered by injection or a pump. Type 2 diabetes mellitus (T2DM) usually begins with either insulin resistance or when there is insufficient production of insulin to maintain an acceptable glucose level.

Currently, various pharmacological approaches are available for treating hyperglycemia and subsequently, T2DM (Hampp, C. et al. Use of Antidiabetic Drugs in the U.S., 2003-2012, Diabetes Care 2014, 37, 1367-1374). One of them is glucagon-like peptide-1 receptor (GLP-1R) agonists (e.g., liraglutide, albiglutide, exenatide, lixisenatide, dulaglutide, semaglutide), which enhance secretion of insulin by acting on the pancreatic beta-cells. Marketed GLP-1R agonists are peptides administered by subcutaneous injection. Liraglutide is additionally approved for the treatment of obesity.

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. In non-clinical experiments GLP-1 promotes continued beta-cell competence by stimulating transcription of genes important for glucose-dependent insulin secretion and by promoting beta-cell neogenesis (Meier et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with T2DM, the normal post-prandial rise in GLP-1 is absent or reduced (Vilsboll T, et al. Diabetes. 2001. 50; 609-613).

Holst (Physiol. Rev. 2007, 87, 1409) and Meier (Nat. Rev. Endocrinol. 2012, 8, 728) describe that GLP-1 receptor agonists, such as liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

There remains a need of developing GLP-1 receptor agonists for an easily-administered prevention and/or treatment for cardiometabolic and associated diseases. The present disclosure provides solid forms of a GLP-1R agonist.

### SUMMARY

Disclosed herein solid forms of 2-((4-(6-((4-cyano-2-fluorobenzyl)oxy)pyridin-2-yl)piperidin-1-yl)methyl)-1-(thiazol-5-ylmethyl)-1H-benzo[d]imidazole-6-carboxylic acid (Compound 1), including solid forms of Compound 1, salts of Compound 1 and solid forms thereof, including crystalline forms of Compound 1 and salts thereof, as well as polymorphs of Compound 1 and salts of Compound 1.

Also disclosed are methods for making the salts and solid forms and methods for using the salts and solid forms of Compound 1. In some embodiments, the solid form of Compound 1 is a form of the free form of the compound, such as polymorph of the free form of the compound. In other embodiments, the solid form of Compound 1 is a salt, and may be a polymorph of the salt.

Also disclosed herein are methods for using the forms of Compound 1, such as a method for using form of Compound las a GLP-1R agonist, including, without limitation the use of Compound 1 as a therapy in a disease or disorder modulated by GLP-1R.

The foregoing and other objects, features, and advantages of the invention will become more apparent from the following detailed description, which proceeds with reference to the accompanying figures.

### BRIEF DESCRIPTION OF THE DRAWINGS

FIG. 1 provides an XRPD pattern of Meglumine salt Type A

FIG. 2 provides a TGA curve of Meglumine salt Type A

FIG. 3 provides a DSC curve of Meglumine salt Type A

FIG. 4 provides a DVS plot of Meglumine salt Type A

FIG. 5 provides an XRPD pattern of Fumarate Type A

FIG. 6 provides a TGA curve of Fumarate Type A

FIG. 7 provides a DSC curve of Fumarate Type A

FIG. 8 provides a DVS plot of Fumarate Type A

FIG. 9 provides an XRPD pattern of Potassium Salt Type A

FIG. 10 provides a TGA curve of Potassium Salt Type A

FIG. 11 provides a DSC curve of Potassium Salt Type A

FIG. 12 provides a DVS plot of Potassium Salt Type A

FIG. 13 provides an XRPD pattern of Tris salt Type C

FIG. 14 provides a TGA curve of Tris salt Type C

FIG. 15 provides a DSC curve of Tris salt Type C

FIG. 16 provides a DVS plot of Tris salt Type C

FIG. 17 provides an XRPD pattern of HCl salt Type A

FIG. 18 provides a TGA curve of HCl salt Type A

FIG. 19 provides a DSC curve of HCl salt Type A

FIG. 20 provides an XRPD pattern of Sulfate Type A

FIG. 21 provides a TGA curve of Sulfate Type A

FIG. 22 provides a DSC curve of Sulfate Type A

FIG. 23 provides an XRPD pattern of Sulfate Type B

FIG. 24 provides a TGA curve of Sulfate Type B

FIG. 25 provides a DSC curve of Sulfate Type B

FIG. 26 provides an XRPD pattern of Sulfate Type C

FIG. 27 provides a TGA curve of Sulfate Type C

FIG. 28 provides a DSC curve of Sulfate Type C

FIG. 29 provides an XRPD pattern of Phosphate Type A

FIG. 30 provides a TGA curve of Phosphate Type A

FIG. 31 provides a DSC curve of Phosphate Type A

FIG. 32 provides an XRPD pattern of Tartrate Type A

FIG. 33 provides a TGA curve of Tartrate Type A

FIG. 34 provides a DSC curve of Tartrate Type A

FIG. 35 provides an XRPD pattern of Mesylate Type A

FIG. 36 provides a TGA curve of Mesylate Type A

FIG. 37 provides a DSC curve of Mesylate Type A

FIG. 38 provides an XRPD pattern of Tosylate Type A

FIG. 39 provides a TGA curve of Tosylate Type A

FIG. 40 provides a DSC curve of Tosylate Type A

FIG. 41 provides an XRPD pattern of Tosylate Type B

FIG. 42 provides a TGA curve of Tosylate Type B

FIG. 43 provides a DSC curve of Tosylate Type B

FIG. 44 provides an XRPD pattern of Arginine salt Type A

FIG. 45 provides a TGA curve of Arginine salt Type A

FIG. 46 provides a DSC curve of Arginine salt Type A

FIG. 47 provides an XRPD pattern of Arginine salt Type B

FIG. 48 provides a TGA curve of Arginine salt Type B

FIG. 49 provides a DSC curve of Arginine salt Type B

FIG. 50 provides an XRPD pattern of Lysine salt Type A

FIG. 51 provides a TGA curve of Lysine salt Type A

FIG. 52 provides a DSC curve of Lysine salt Type A

FIG. 53 provides an XRPD pattern of Lysine salt Type B

FIG. 54 provides a TGA curve of Lysine salt Type B

FIG. 55 provides a DSC curve of Lysine salt Type B

FIG. 56 provides an XRPD pattern of Sodium salt Type A

FIG. 57 provides a TGA curve of Sodium salt Type A

FIG. 58 provides a DSC curve of Sodium salt Type A

FIG. 59 provides an XRPD pattern of Potassium salt Type B

FIG. 60 provides a TGA curve of Potassium salt Type B

FIG. 61 provides a DSC curve of Potassium salt Type B

FIG. 62 provides an XRPD pattern of Choline salt Type A

FIG. 63 provides a TGA curve of Choline salt Type A

FIG. 64 provides a DSC curve of Choline salt Type A

FIG. 65 provides an XRPD pattern of Ammonium salt Type A

FIG. 66 provides a TGA curve of Ammonium salt Type A

FIG. 67 provides a DSC curve of Ammonium salt Type A

FIG. 68 provides an XRPD pattern of Ammonium salt Type B

FIG. 69 provides a TGA curve of Ammonium salt Type B

FIG. 70 provides a DSC curve of Ammonium salt Type B

FIG. 71 provides an XRPD pattern of Tris salt Type A

FIG. 72 provides a TGA curve of Tris salt Type A

FIG. 73 provides a DSC curve of Tris salt Type A

FIG. 74 provides an XRPD pattern of Tris salt Type B

FIG. 75 provides a TGA curve of Tris salt Type B

FIG. 76 provides a DSC curve of Tris salt Type B

FIG. 77 provides an XRPD pattern of Meglumine salt Type B

FIG. 78 provides a TGA curve of Meglumine salt Type B

FIG. 79 provides a DSC curve of Meglumine salt Type B

FIG. 80 provides an XRPD pattern of Meglumine salt Type C

FIG. 81 provides a TGA curve of Meglumine salt Type C

FIG. 82 provides a DSC curve of Meglumine salt Type C

FIG. 83 provides an XRPD pattern of Freeform Type A

FIG. 84 provides a TGA curve of Freeform Type A

FIG. 85 provides a DSC curve of Freeform Type A

FIG. 86 provides an XRPD pattern of Freeform Type B

FIG. 87 provides a TGA curve of Freeform Type B

FIG. 88 provides a DSC curve of Freeform Type B

FIG. 89 provides an XRPD pattern of Freeform Type C

FIG. 90 provides a TGA curve of Freeform Type C

FIG. 91 provides a DSC curve of Freeform Type C

FIG. 92 provides a DVS plot of Freeform Type C

FIG. 93 provides an XRPD pattern of Freeform Type D

FIG. 94 provides a TGA curve of Freeform Type D

FIG. 95 provides a DSC curve of Freeform Type D

FIG. 96 provides an XRPD pattern of Freeform Type E

FIG. 97 provides a TGA curve of Freeform Type E

FIG. 98 provides a DSC curve of Freeform Type E

FIG. 99 provides an XRPD pattern of Freeform Type F

FIG. 100 provides an XRPD pattern of Freeform Type G

FIG. 101 provides a TGA curve of Freeform Type G

FIG. 102 provides a DSC curve of Freeform Type G

FIG. 103 provides an XRPD pattern of Freeform Type H

FIG. 104 provides a TGA curve of Freeform Type H

FIG. 105 provides a DSC curve of Freeform Type H

FIG. 106 provides an XRPD pattern of Freeform Type I

FIG. 107 provides a TGA curve of Freeform Type I

FIG. 108 provides a DSC curve of Freeform Type I

FIG. 109 provides an XRPD pattern of Freeform Type J

FIG. 110 provides a TGA curve of Freeform Type J

FIG. 111 provides a DSC curve of Freeform Type J

FIG. 112 provides an XRPD pattern of Freeform Type K

FIG. 113 provides a TGA curve of Freeform Type K

FIG. 114 provides a DSC curve of Freeform Type K

FIG. 115 provides an XRPD pattern of Freeform Type L

FIG. 116 provides a TGA curve of Freeform Type L

FIG. 117 provides a DSC curve of Freeform Type L

FIG. 118 provides an XRPD pattern of Freeform Type M

FIG. 119 provides a TGA curve of Freeform Type M

FIG. 120 provides a DSC curve of Freeform Type M

FIG. 121 provides an XRPD pattern of Freeform Type N

FIG. 122 provides a TGA curve of Freeform Type N

FIG. 123 provides a DSC curve of Freeform Type N

FIG. 124 provides an XRPD pattern of Freeform Type O

FIG. 125 provides an XRPD pattern of Freeform Type P

FIG. 126 provides a TGA curve of Freeform Type P

FIG. 127 provides a DSC curve of Freeform Type P

FIG. 128 Form conversion diagram of Compound 1 meglumine salt

FIG. 129 Form conversion diagram of Compound 1 freeform

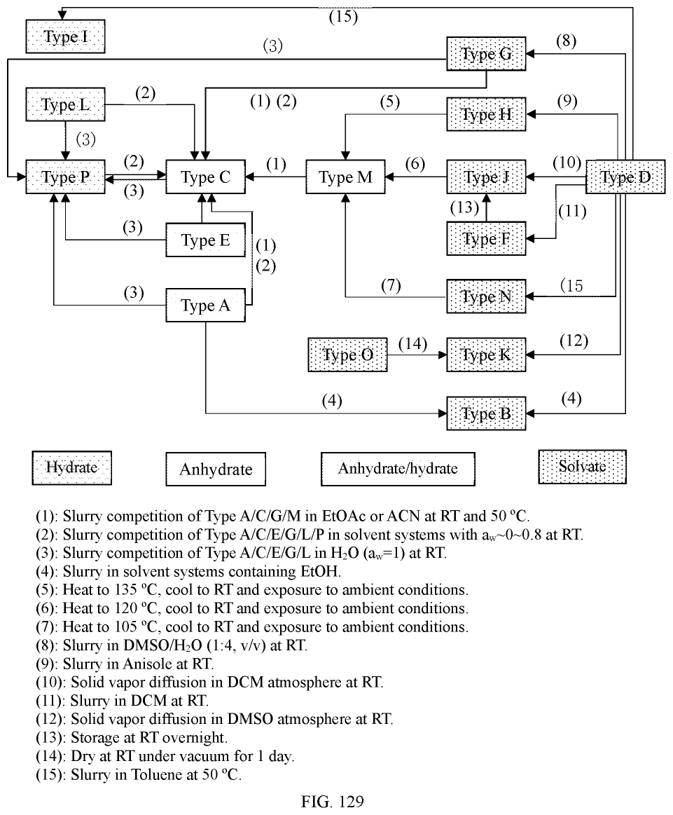

FIG. 130 provides a ¹H NMR spectrum of Meglumine salt Type A

FIG. 131 provides a ¹H NMR spectrum of Fumarate Type A

FIG. 132 provides a ¹H NMR spectrum of Potassium salt Type A

FIG. 133 provides a ¹H NMR spectrum of Tris salt Type C

FIG. 134 provides a ¹H NMR spectrum of HCl salt Type A

FIG. 135 provides a ¹H NMR spectrum of Sulfate Type A

FIG. 136 provides a ¹H NMR spectrum of Sulfate Type B

FIG. 137 provides a ¹H NMR spectrum of Sulfate Type C

FIG. 138 provides a ¹H NMR spectrum of Phosphate Type A

FIG. 139 provides a ¹H NMR spectrum of Tartrate Type A

FIG. 140 provides a ¹H NMR spectrum of Mesylate Type A

FIG. 141 provides a ¹H NMR spectrum of Tosylate Type A

FIG. 142 provides a ¹H NMR spectrum of Tosylate Type B

FIG. 143 provides a ¹H NMR spectrum of Arginine salt Type A

FIG. 144 provides a ¹H NMR spectrum of Arginine salt Type B

FIG. 145 provides a ¹H NMR spectrum of Lysine salt Type A

FIG. 146 provides a ¹H NMR spectrum of Lysine salt Type B

FIG. 147 provides a ¹H NMR spectrum of Na salt Type A

FIG. 148 provides a ¹H NMR spectrum of K salt Type A

FIG. 149 provides a ¹H NMR spectrum of Choline salt Type A

FIG. 150 provides a ¹H NMR spectrum of Ammonium salt Type A

FIG. 151 provides a ¹H NMR spectrum of Ammonium salt Type B

FIG. 152 provides a ¹H NMR spectrum of Tris salt Type A

FIG. 153 provides a ¹H NMR spectrum of Tris salt Type B

FIG. 154 provides a ¹H NMR spectrum of Meglumine salt Type B

FIG. 155 provides a ¹H NMR spectrum of Meglumine salt Type C

FIG. 156 provides a ¹H NMR spectrum of Freeform Type A

FIG. 157 provides a ¹H NMR spectrum of Freeform Type B

FIG. 158 provides a ¹H NMR spectrum of Freeform Type C

FIG. 159 provides a ¹H NMR spectrum of Freeform Type D

FIG. 160 provides a ¹H NMR spectrum of Freeform Type E

FIG. 161 provides a ¹H NMR spectrum of Freeform Type G

FIG. 162 provides a ¹H NMR spectrum of Freeform Type H

FIG. 163 provides a ¹H NMR spectrum of Freeform Type I

FIG. 164 provides a ¹H NMR spectrum of Freeform Type J

FIG. 165 provides a ¹H NMR spectrum of Freeform Type K

FIG. 166 provides a ¹H NMR spectrum of Freeform Type L

FIG. 167 provides a ¹H NMR spectrum of Freeform Type M

FIG. 168 provides a ¹H NMR spectrum of Freeform Type N

FIG. 169 provides a ¹H NMR spectrum of Freeform Type P

FIG. 170 VT-XRPD patterns of Meglumine salt Type A

FIG. 171 VH-XRPD patterns of Meglumine salt Type A

FIG. 172 VT-XRPD patterns of Meglumine salt Type B

FIG. 173: Overlay of the calculated XRPD of the Compound 1 meglumine salt Type A single crystal structure model and the experimental XRPD of the single crystal and the meglumine salt Type A reference

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by experimentation that is within the skill and judgment of the clinician.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As used herein, the term “about” represents a value that is in the range of t 10% of the value that follows the term “about.” Reference to “about” a value or parameter herein includes (and describes) variations that are directed to that value or parameter per se. For example, description referring to “about X” includes description of “X”. As it relates to any of the peaks of X-ray powder diffraction set throughout this application, “about” refers to ±0.2 of the 2θ value in degrees.

### II. Compounds

Disclosed herein are solid forms of 2-((4-(6-((4-cyano-2-fluorobenzyl)oxy)pyridin-2-yl)piperidin-1-yl)methyl)-1-(thiazol-5-ylmethyl)-1H-benzo[d]imidazole-6-carboxylic acid (Compound 1) and salt thereof. Also disclosed are methods for making the solid forms of Compound 1 and salts thereof and methods of administering solid forms of Compound 1 and salts thereof.

The structure of Compound 1 is shown below:

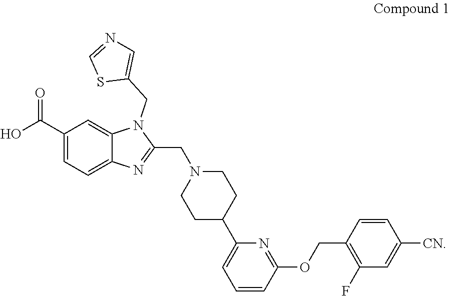 [SMILES: N#Cc1ccc(COc2cccc(C3CCN(Cc4nc5ccc(C(=O)O)cc5n4Cc4cncs4)CC3)n2)c(F)c1]

In some embodiments, a solid form of the compound is a crystalline form (e.g., of Compound 1) or a salt thereof.

In some embodiments, the solid form is a solid form of Compound 1. In some embodiments, the solid form is a crystalline form of Compound 1. In some embodiments, the solid form is a polymorph of Compound 1, such as a polymorph of the free form compound or a polymorph of the salt.

In some embodiments, the solid form of the compound is a salt of the compound. In some embodiments, the solid form is a crystalline form of a salt of Compound 1. In some embodiments, the solid form of the compound is a crystalline salt form of the compound, such as an acid or base addition salt form.

### III. Salts and Solid Forms

### Salts

In some embodiments, the solid form of Compound 1 comprises a salt of Compound 1. Suitable salts include pharmaceutically acceptable salts of Compound 1.

In some embodiments, the salt of Compound 1 may be formed from a suitable pharmaceutically acceptable acid, including, without limitation, inorganic acids such as hydrochloric acid, hydrobromic acid, sulfuric acid, nitric acid, phosphoric acid, and the like, as well as organic acids such as fumaric acid, formic acid, acetic acid, trifluoroacetic acid, propionic acid, glycolic acid, gentisic acid, lauric acid, stearic acid, pyruvic acid, oxalic acid, maleic acid, malonic acid, succinic acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like. The Compound 1 salt may be formed from a suitable alkali or alkaline earth metal such as sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine. The Compound 1 salt may be formed from an amino sugar such as meglumine, an amino acid such as lysine or arginine, or an amine such as choline, or tris(hydroxymethyl)aminomethane (Tris). Additional information concerning pharmaceutically acceptable salts can be found in, for example, S. M. Berge, et al., “Pharmaceutical Salts,” J. Pharm. Sci., 1977; 66:1-19 which is incorporated herein by reference.

The salts of Compound 1 disclosed herein can have any suitable stoichiometric ratio of the salt former (acid or base) to Compound 1. In one embodiment, the molar ratio of the salt former (acid or base) to Compound 1 is from about 0.5 to about 2.0, such as forms wherein the salt has a stoichiometric ratio of the salt former (acid or base) to Compound 1 of from about 0.5 to about 2, such as about 0.5, about 1 or about 2.

### Crystalline Forms of Compound 1 Salt and Free Form

### Fumarate Type A

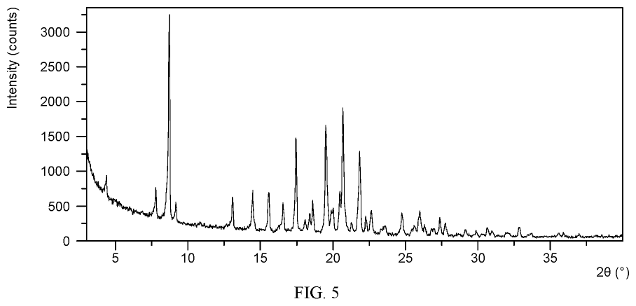

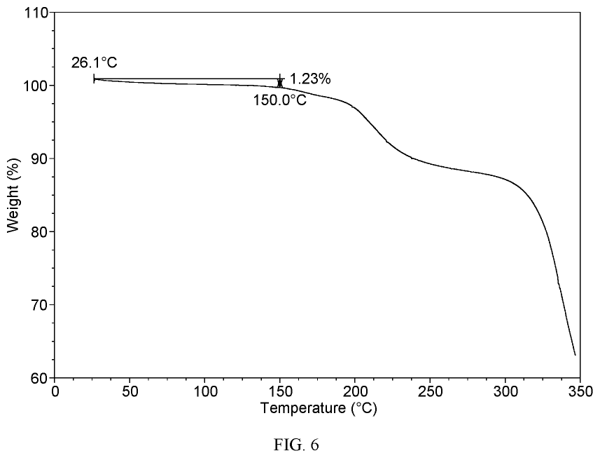

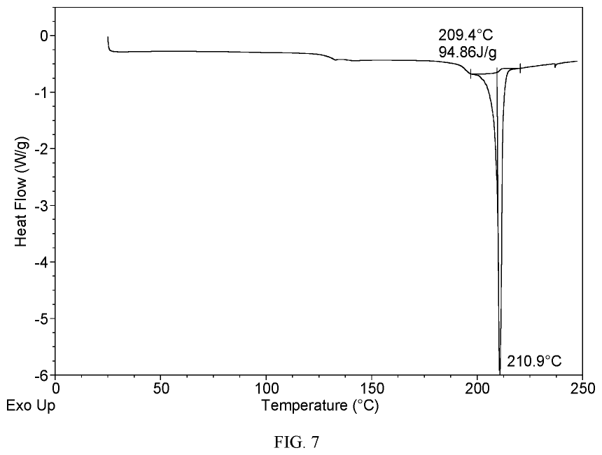

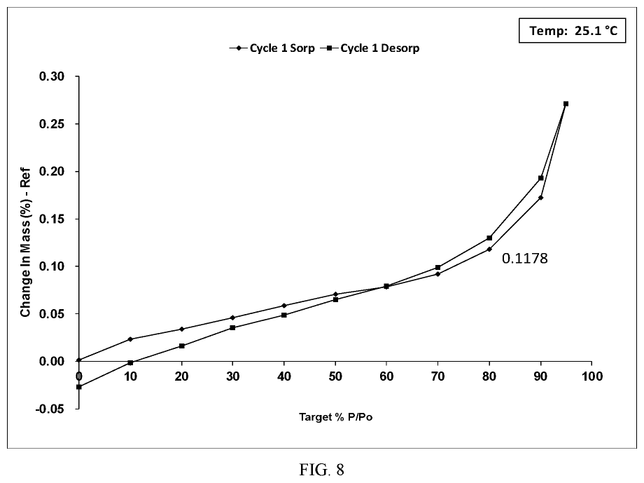

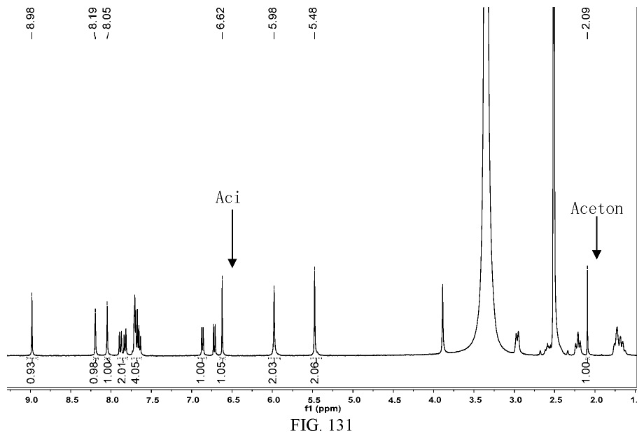

In some embodiments, the present disclosure provides solid forms of Fumarate Type A, e.g., crystalline forms of Fumarate Type A. In some embodiments, the Fumarate Type A XRPD profile is substantially similar to that shown in FIG. 5. In some embodiments, the Fumarate Type A TGA profile is substantially similar to that shown in FIG. 6. In some embodiments, the Fumarate Type A DSC curve is substantially similar to that shown in FIG. 7. In some embodiments, the Fumarate Type A DVS profile is substantially similar to that shown in FIG. 8. In some embodiments, the Fumarate Type A ¹H NMR profile is substantially similar to that shown in FIG. 131. In some embodiments, the Fumarate Type A is characterized by a DSC curve having an endotherm at 210.9° C.

In some embodiments, the solid form of Fumarate Type A is crystalline Fumarate Type A characterized by two or more, or three XRPD signals selected from the group consisting of 8.7 °2θ, 20.7 °2θ, and 19.5 °2θ (±0.2 °2θ; ±0.1 °2θ; or ±0.0 °2θ; Cu Kα1 radiation). In some embodiments, the solid form of Fumarate Type A is crystalline Fumarate Type A characterized by XRPD signals at 8.7 °2θ, 20.7 °2θ, and 19.5 °2θ (±0.2 °2θ; ±0.1 °2θ; or ±0.0 °2θ; Cu Kα1 radiation).

In some embodiments, the solid form of Fumarate Type A is crystalline Fumarate Type A characterized by two or more, or three or more XRPD signals selected from the group consisting of 8.7 °2θ, 20.7 °2θ, 19.5 °2θ, 17.5 °2θ, and 21.9 °2θ (±0.2 °2θ; ±0.1 °2θ; or ±0.0 °2θ; Cu Kα1 radiation). In some embodiments, the solid form of Fumarate Type A is crystalline Fumarate Type A characterized by XRPD signals at 8.7 °2θ, 20.7 °2θ, 19.5 °2θ, 17.5 °2θ, and 21.9 °2θ (±0.2 °2θ; ±0.1 °2θ; or ±0.0 °2θ; Cu Kα1 radiation).

In some embodiments, the solid form of Fumarate Type A is crystalline Fumarate Type A characterized by two or more, or three or more XRPD signals selected from the group consisting of 8.7 °2θ, 20.7 °2θ, 19.5 °2θ, 17.5 °2θ, 21.9 °2θ, 20.5 °2θ, and 15.6 °2θ (±0.2 °2θ; ±0.1 °2θ; or ±0.0 °2θ; Cu Kα1 radiation). In some embodiments, the solid form of Fumarate Type A is crystalline Fumarate Type A characterized by XRPD signals at 8.7 °2θ, 20.7 °2θ, 19.5 °2θ, 17.5 °2θ, 21.9 °2θ, 20.5 °2θ, and 15.6 °2θ (±0.2 °2θ; ±0.1 °2θ; or ±0.0 °2θ; Cu Kα1 radiation).

In some embodiments, the solid form of Fumarate Type A is crystalline Fumarate Type A characterized by two or more, or three or more XRPD signals selected from the group consisting of 8.7 °2θ, 20.7 °2θ, 19.5 °2θ, 17.5 °2θ, 21.9 °2θ, 20.5 °2θ, 15.6 °2θ, 14.5 °2θ, 13.1 °2θ, and 18.6 °2θ (±0.2 °2θ; ±0.1 °2θ; or ±0.0 °2θ; Cu Kα1 radiation). In some embodiments, the solid form of Fumarate Type A is crystalline Fumarate Type A characterized by XRPD signals at 8.7 °2θ, 20.7 °2θ, 19.5 °2θ, 17.5 °2θ, 21.9 °2θ, 20.5 °2θ, 15.6 °2θ, 14.5 °2θ, 13.1 °2θ, and 18.6 °2θ (±0.2 °2θ; ±0.1 °2θ; or ±0.0 °2θ; Cu Kα1 radiation).

In some embodiments, the crystalline Fumarate Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, or thirty-three XRPD signals selected from those set forth in Table 1.

TABLE 1
Fumarate Type A XRPD Signals

| Signal | Pos. | d-spacing | Rel. Int. |
|---|---|---|---|
| No. | [°2θ] | [Å] | [%] |
| 1 | 4.38 | 20.2 | 9.51 |
| 2 | 7.78 | 11.36 | 13.97 |
| 3 | 8.71 | 10.15 | 100 |
| 4 | 9.17 | 9.64 | 8.82 |
| 5 | 13.08 | 6.77 | 15.7 |
| 6 | 14.46 | 6.12 | 18.03 |
| 7 | 15.58 | 5.69 | 18.81 |
| 8 | 16.57 | 5.35 | 13.42 |
| 9 | 17.45 | 5.08 | 46.39 |
| 10 | 18.07 | 4.91 | 4.77 |
| 11 | 18.39 | 4.82 | 9.05 |
| 12 | 18.62 | 4.76 | 14.65 |
| 13 | 19.51 | 4.55 | 51.33 |
| 14 | 20.03 | 4.43 | 10.9 |
| 15 | 20.47 | 4.34 | 20.51 |
| 16 | 20.69 | 4.29 | 60.76 |
| 17 | 21.28 | 4.18 | 4.85 |
| 18 | 21.86 | 4.07 | 37.28 |
| 19 | 22.28 | 3.99 | 7.38 |
| 20 | 22.65 | 3.93 | 11.35 |
| 21 | 23.59 | 3.77 | 4.12 |
| 22 | 24.76 | 3.6 | 10.57 |
| 23 | 25.62 | 3.48 | 4.58 |
| 24 | 26.01 | 3.43 | 9.82 |
| 25 | 26.98 | 3.31 | 3.38 |
| 26 | 27.37 | 3.26 | 8.7 |
| 27 | 27.75 | 3.22 | 6.23 |
| 28 | 29.13 | 3.07 | 3.22 |
| 29 | 30.64 | 2.92 | 4.41 |
| 30 | 32.04 | 2.79 | 1.94 |
| 31 | 32.85 | 2.73 | 4.96 |
| 32 | 33.6 | 2.67 | 1.25 |
| 33 | 36.96 | 2.43 | 0.98 |

### Potassium Salt Type A

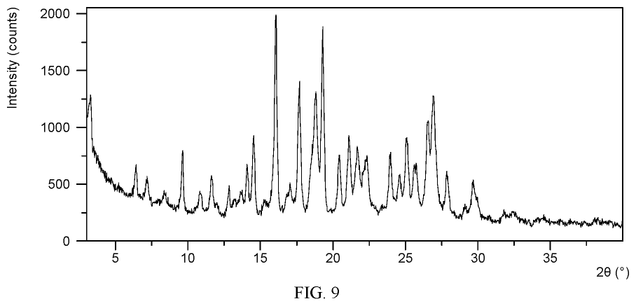

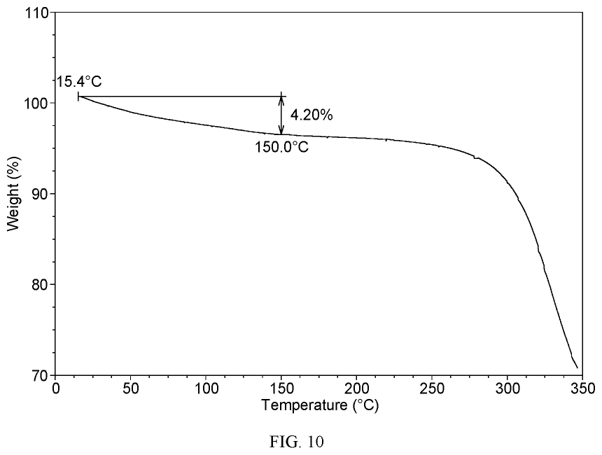

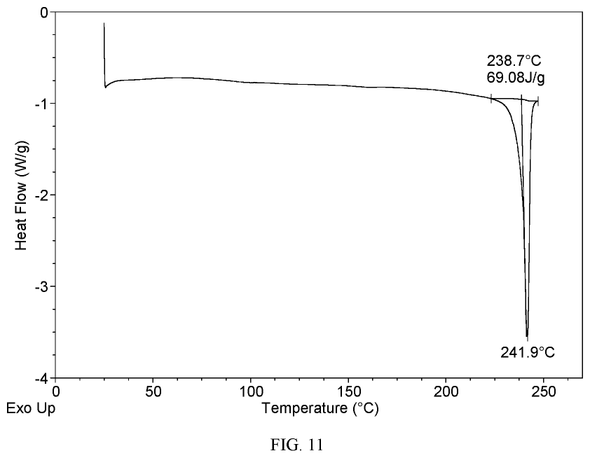

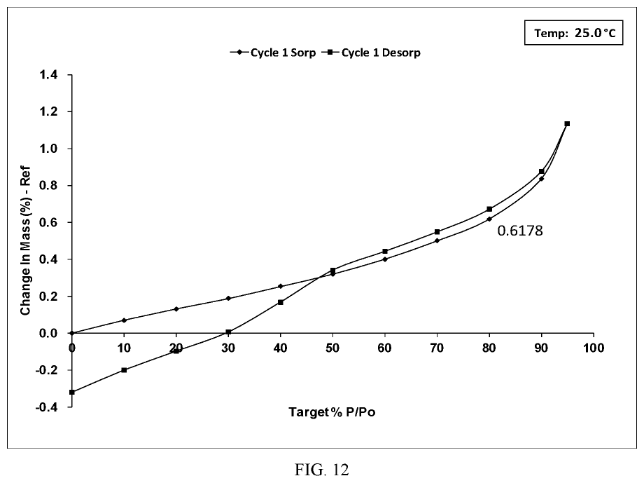

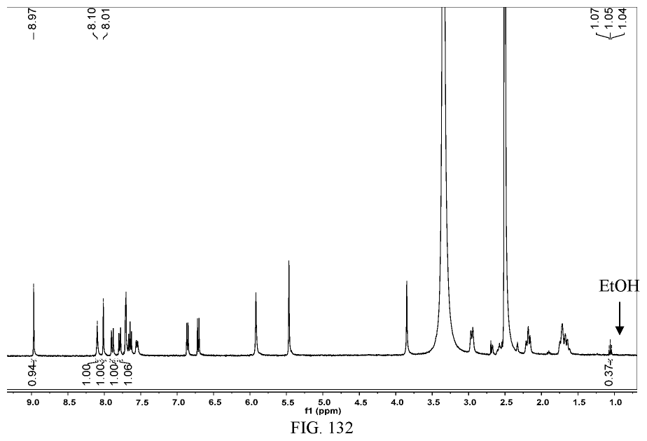

In some embodiments, the present disclosure provides solid forms of Potassium Type A, e.g., crystalline forms of Potassium Type A. In some embodiments, the Potassium Type A XRPD profile is substantially similar to that shown in FIG. 9. In some embodiments, the Potassium Type A TGA profile is substantially similar to that shown in FIG. 10. In some embodiments, the Potassium Type A DSC curve is substantially similar to that shown in FIG. 11. In some embodiments, the Potassium Type A DVS profile is substantially similar to that shown in FIG. 12. In some embodiments, the Potassium Type A ¹H NMR profile is substantially similar to that shown in FIG. 132. In some embodiments, the Potassium Type A is characterized by a DSC curve having an endotherm at 241.9° C.

In some embodiments, the solid form of Potassium Type A is crystalline Potassium Type A characterized by two or more, or three XRPD signals selected from the group consisting of 16.1 °2θ, 19.3 °2θ, and 17.7 °2θ (±0.2 °2θ; ±0.1 °2θ; or ±0.0 °2θ; Cu Kα1 radiation). In some embodiments, the solid form of Potassium Type A is crystalline Potassium Type A characterized by XRPD signals at 16.1 °2θ, 19.3 °2θ, and 17.7 °2θ (±0.2 °2θ; ±0.1 °2θ; or ±0.0 °2θ; Cu Kα1 radiation).

In some embodiments, the solid form of Potassium Type A is crystalline Potassium Type A characterized by two or more, or three or more XRPD signals selected from the group consisting of 16.1 °2θ, 19.3 °2θ, 17.7 °2θ, 26.9 °2θ, and 18.9 °2θ (±0.2 °2θ; ±0.1 °2θ; or ±0.0 °2θ; Cu Kα1 radiation). In some embodiments, the solid form of Potassium Type A is crystalline Potassium Type A characterized by XRPD signals at 16.1 °2θ, 19.3 °2θ, 17.7 °2θ, 26.9 °2θ, and 18.9 °2θ (±0.2 °2θ; ±0.1 °2θ; or ±0.0 °2θ; Cu Kα1 radiation).

In some embodiments, the solid form of Potassium Type A is crystalline Potassium Type A characterized by two or more, or three or more XRPD signals selected from the group consisting of 16.1 °2θ, 19.3 °2θ, 17.7 °2θ, 26.9 °2θ, 18.9 °2θ, 26.5 °2θ, and 14.5 °2θ (±0.2 °2θ; ±0.1 °2θ; or ±0.0 °2θ; Cu Kα1 radiation). In some embodiments, the solid form of Potassium Type A is crystalline Potassium Type A characterized by XRPD signals at 16.1 °2θ, 19.3 °2θ, 17.7 °2θ, 26.9 °2θ, 18.9 °2θ, 26.5 °2θ, and 14.5 °2θ (±0.2 °2θ; ±0.1 °2θ; or ±0.0 °2θ; Cu Kα1 radiation).

In some embodiments, the solid form of Potassium Type A is crystalline Potassium Type A characterized by two or more, or three or more XRPD signals selected from the group consisting of 16.1 °2θ, 19.3 °2θ, 17.7 °2θ, 26.9 °2θ, 18.9 °2θ, 26.5 °2θ, 14.5 °2θ, 21.1 °2θ, 25.1 °2θ, and 21.7 °2θ (±0.2 °2θ; ±0.1 °2θ; or ±0.0 °2θ; Cu Kα1 radiation). In some embodiments, the solid form of Potassium Type A is crystalline Potassium Type A characterized by XRPD signals at 16.1 °2θ, 19.3 °2θ, 17.7 °2θ, 26.9 °2θ, 18.9 °2θ, 26.5 °2θ, 14.5 °2θ, 21.1 °2θ, 25.1 °2θ, and 21.7 °2θ (±0.2 °2θ; ±0.1 °2θ; or ±0.0 °2θ; Cu Kα1 radiation).

In some embodiments, the crystalline Potassium Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, or twenty-six XRPD signals selected from those set forth in Table 2.

TABLE 2
Potassium Salt Type A XRPD Signals

| Signal | Pos. | d-spacing | Rel. Int. |
|---|---|---|---|
| No. | [°2θ] | [Å] | [%] |
| 1 | 3.3 | 26.81 | 21.82 |
| 2 | 6.44 | 13.73 | 14.5 |
| 3 | 7.17 | 12.32 | 10.59 |
| 4 | 8.4 | 10.53 | 6.21 |
| 5 | 9.63 | 9.18 | 29.76 |
| 6 | 10.86 | 8.15 | 10.08 |
| 7 | 11.64 | 7.6 | 18.18 |
| 8 | 12.85 | 6.89 | 13.92 |
| 9 | 14.07 | 6.3 | 24.77 |
| 10 | 14.52 | 6.1 | 38.22 |
| 11 | 16.06 | 5.52 | 100 |
| 12 | 17.68 | 5.02 | 63.73 |
| 13 | 18.85 | 4.71 | 55.91 |
| 14 | 19.29 | 4.6 | 91.79 |
| 15 | 20.42 | 4.35 | 26.67 |
| 16 | 21.1 | 4.21 | 37.74 |
| 17 | 21.7 | 4.1 | 31.49 |
| 18 | 22.38 | 3.97 | 25.47 |
| 19 | 23.93 | 3.72 | 27.9 |
| 20 | 24.59 | 3.62 | 19.18 |
| 21 | 25.06 | 3.55 | 36.55 |
| 22 | 25.67 | 3.47 | 22.52 |
| 23 | 26.48 | 3.37 | 42.54 |
| 24 | 26.94 | 3.31 | 59.96 |
| 25 | 27.84 | 3.2 | 21.45 |
| 26 | 29.67 | 3.01 | 18.14 |

### HCl Salt Type A

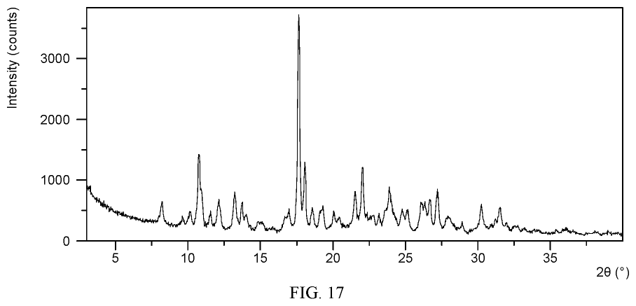

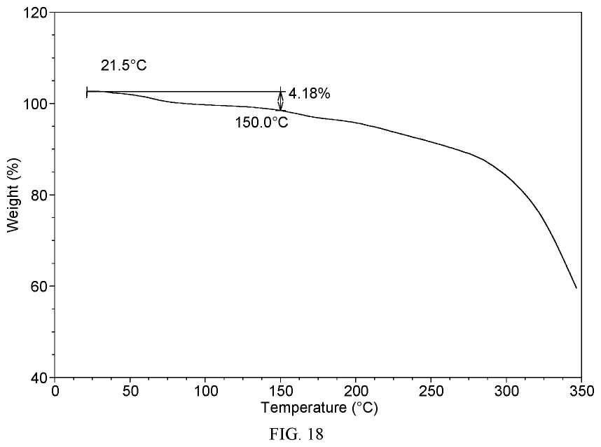

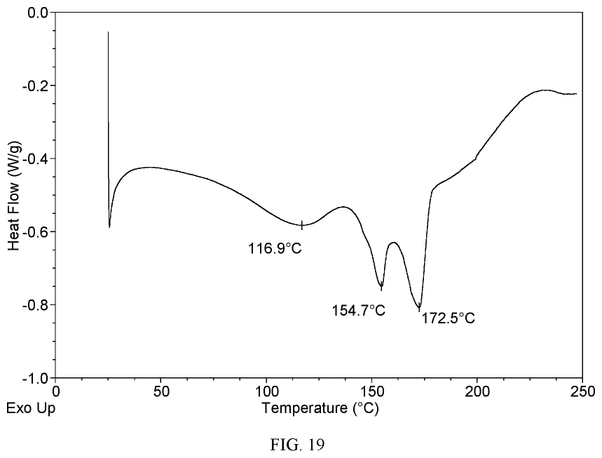

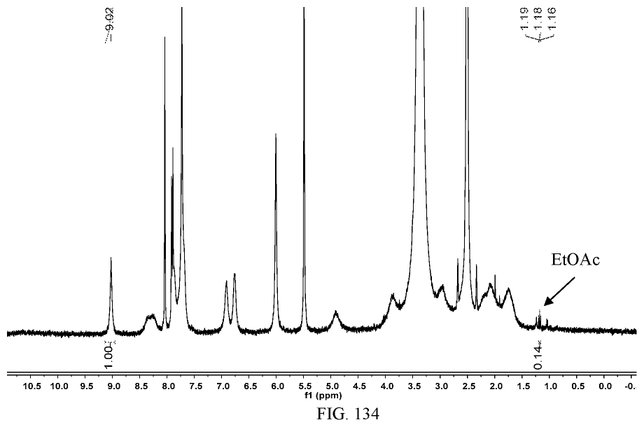

In some embodiments, the present disclosure provides solid forms of HCl Type A, e.g., crystalline forms of HCl Type A. In some embodiments, the HCl Type A XRPD profile is substantially similar to that shown in FIG. 17. In some embodiments, the HCl Type A TGA profile is substantially similar to that shown in FIG. 18. In some embodiments, the HCl Type A DSC curve is substantially similar to that shown in FIG. 19. In some embodiments, the HCl Type A ¹H NMR profile is substantially similar to that shown in FIG. 134. In some embodiments, the HCl Type A is characterized by a DSC curve having endotherms at about 116.9° C., about 154.7, and about 172.5° C.

In some embodiments, the solid form of HCl Type A is crystalline HCl Type A characterized by two or more, or three XRPD signals selected from the group consisting of 17.7 °2θ, 10.8 °2θ, and 22.1 °2θ (±0.2 °2θ; ±0.1 °2θ; or ±±0.0 °2θ; Cu Kα1 radiation). In some embodiments, the solid form of HCl Type A is crystalline HCl Type A characterized by XRPD signals at 17.7 °2θ, 10.8 °2θ, and 22.1 °2θ (±0.2 °2θ; A0.1 °2θ; or ±0.0 °2θ; Cu Kα1 radiation).

In some embodiments, the solid form of HCl Type A is crystalline HCl Type A characterized by two or more, or three or more XRPD signals selected from the group consisting of 17.7 °2θ, 10.8 °2θ, 22.1 °2θ, 18.1 °2θ, and 23.9 °2θ (±0.2 °2θ; ±0.1 °2θ; or ±0.0 °2θ; Cu Kα1 radiation). In some embodiments, the solid form of HCl Type A is crystalline HCl Type A characterized by XRPD signals at 17.7 °2θ, 10.8 °2θ, 22.1 °2θ, 18.1 °2θ, and 23.9 °2θ (±0.2 °2θ; ±0.1 °2θ; or ±0.0 °2θ; Cu Kα1 radiation).

In some embodiments, the solid form of HCl Type A is crystalline HCl Type A characterized by two or more, or three or more XRPD signals selected from the group consisting of 17.7 °2θ, 10.8 °2θ, 22.1 °2θ, 18.1 °2θ, 23.9 °2θ, 21.5 °2θ, and 13.2 °2θ (±0.2 °2θ; ±0.1 °2θ; or ±0.0 °2θ; Cu Kα1 radiation). In some embodiments, the solid form of HCl Type A is crystalline HCl Type A characterized by XRPD signals at 17.7 °2θ, 10.8 °2θ, 22.1 °2θ, 18.1 °2θ, 23.9 °2θ, 21.5 °2θ, and 13.2 °2θ (±0.2 °2θ; ±0.1 °2θ; or ±0.0 °2θ; Cu Kα1 radiation).

In some embodiments, the solid form of HCl Type A is crystalline HCl Type A characterized by two or more, or three or more XRPD signals selected from the group consisting of 17.7 °2θ, 10.8 °2θ, 22.1 °2θ, 18.1 °2θ, 23.9 °2θ, 21.5 °2θ, 13.2 °2θ, 27.2 °2θ, 26.7 °2θ, and 30.3 °2θ (±0.2 °2θ; ±0.1 °2θ; or ±0.0 °2θ; Cu Kα1 radiation). In some embodiments, the solid form of HCl Type A is crystalline HCl Type A characterized by XRPD signals at 17.7 °2θ, 10.8 °2θ, 22.1 °2θ, 18.1 °2θ, 23.9 °2θ, 21.5 °2θ, 13.2 °2θ, 27.2 °2θ, 26.7 °2θ, and 30.3 °2θ (±0.2 °2θ; ±0.1 °2θ; or ±0.0 °2θ; Cu Kα1 radiation).

In some embodiments, the crystalline HCl Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two XRPD signals selected from those set forth in Table 3.

TABLE 3
HCl Salt Type A XRPD Signals

| Signal | Pos. | d-spacing | Rel. Int. |
|---|---|---|---|
| No. | [°2θ] | [Å] | [%] |
| 1 | 8.21 | 10.77 | 10.12 |
| 2 | 9.64 | 9.18 | 3.83 |
| 3 | 10.16 | 8.71 | 6.91 |
| 4 | 10.75 | 8.23 | 34.86 |
| 5 | 11.57 | 7.65 | 7.42 |
| 6 | 12.13 | 7.3 | 13.29 |
| 7 | 13.23 | 6.69 | 17.2 |
| 8 | 13.72 | 6.45 | 12.58 |
| 9 | 14.99 | 5.91 | 3.38 |
| 10 | 16.97 | 5.22 | 9.59 |
| 11 | 17.65 | 5.02 | 100 |
| 12 | 18.09 | 4.91 | 29.42 |
| 13 | 18.57 | 4.78 | 10.48 |
| 14 | 19.32 | 4.59 | 11.22 |
| 15 | 20.06 | 4.43 | 8.12 |
| 16 | 20.43 | 4.35 | 5.51 |
| 17 | 21.52 | 4.13 | 18.03 |
| 18 | 22.05 | 4.03 | 29.47 |
| 19 | 22.79 | 3.9 | 6.63 |
| 20 | 23.17 | 3.84 | 6.75 |
| 21 | 23.91 | 3.72 | 18.39 |
| 22 | 24.8 | 3.59 | 9.1 |
| 23 | 25.18 | 3.54 | 9.5 |
| 24 | 26.09 | 3.42 | 12.99 |
| 25 | 26.71 | 3.34 | 15.03 |
| 26 | 27.23 | 3.28 | 17.07 |
| 27 | 27.86 | 3.2 | 6.35 |
| 28 | 28.93 | 3.09 | 4.08 |
| 29 | 30.25 | 2.95 | 13.34 |
| 30 | 30.89 | 2.89 | 2.75 |
| 31 | 31.53 | 2.84 | 12.09 |
| 32 | 36.01 | 2.49 | 2.01 |

### Sulfate Type A

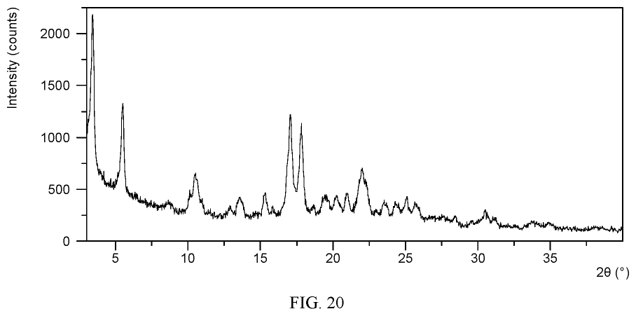

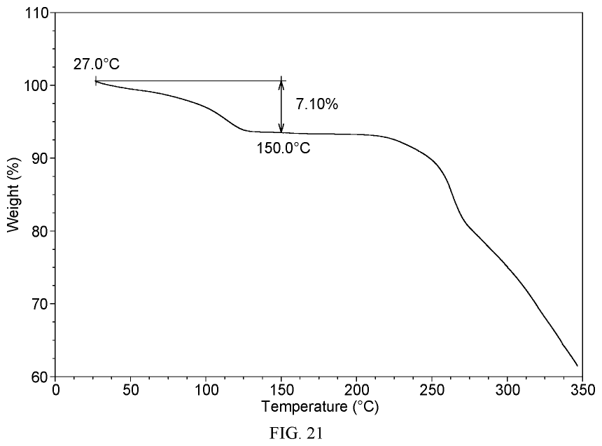

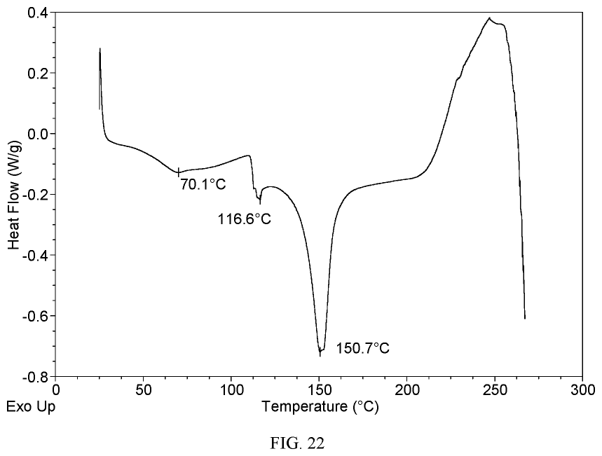

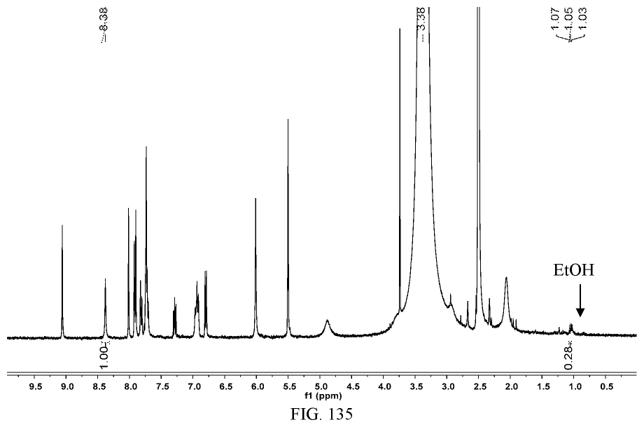

In some embodiments, the present disclosure provides solid forms of Sulfate Type A, e.g., crystalline forms of Sulfate Type A. In some embodiments, the Sulfate Type A XRPD profile is substantially similar to that shown in FIG. 20. In some embodiments, the Sulfate Type A TGA profile is substantially similar to that shown in FIG. 21. In some embodiments, the Sulfate Type A DSC curve is substantially similar to that shown in FIG. 22. In some embodiments, the Sulfate Type A ¹H NMR profile is substantially similar to that shown in FIG. 135. In some embodiments, the Sulfate Type A is characterized by a DSC curve having endotherms at about 70.1° C., about 116.6° C. and about 150.7° C.

In some embodiments, the solid form of Sulfate Type A is crystalline Sulfate Type A characterized by two or more, or three XRPD signals selected from the group consisting of 3.4 °2θ, 17.1 °2θ, and 17.8 °2θ (±0.2 °2θ; ±0.1 °2θ; or ±0.0 °2θ; Cu Kα1 radiation). In some embodiments, the solid form of Sulfate Type A is crystalline Sulfate Type A characterized by XRPD signals at 3.4 °2θ, 17.1 °2θ, and 17.8 °2θ (±0.2 °2θ; ±0.1 °2θ; or ±0.0 °2θ; Cu Kα1 radiation).

In some embodiments, the solid form of Sulfate Type A is crystalline Sulfate Type A characterized by two or more, or three or more XRPD signals selected from the group consisting of 3.4 °2θ, 17.1 °2θ, 17.8 °2θ, 5.5 °2θ, and 22.0 °2θ (±0.2 °2θ; ±0.1 °2θ; or ±0.0 °2θ; Cu Kα1 radiation). In some embodiments, the solid form of Sulfate Type A is crystalline Sulfate Type A characterized by XRPD signals at 3.4 °2θ, 17.1 °2θ, 17.8 °2θ, 5.5 °2θ, and 22.0 °2θ (±0.2 °2θ; ±0.1 °2θ; or ±0.0 °2θ; Cu Kα1 radiation).

In some embodiments, the solid form of Sulfate Type A is crystalline Sulfate Type A characterized by two or more, or three or more XRPD signals selected from the group consisting of 3.4 °2θ, 17.1 °2θ, 17.8 °2θ, 5.5 °2θ, 22.0 °2θ, 10.5 °2θ, and 15.3 °2θ (±0.2 °2θ; ±0.1 °2θ; or ±0.0 °2θ; Cu Kα1 radiation). In some embodiments, the solid form of Sulfate Type A is crystalline Sulfate Type A characterized by XRPD signals at 3.4 °2θ, 17.1 °2θ, 17.8 °2θ, 5.5 °2θ, 22.0 °2θ, 10.5 °2θ, and 15.3 °2θ (±0.2 °2θ; ±0.1 °2θ; or ±0.0 °2θ; Cu Kα1 radiation).

In some embodiments, the solid form of Sulfate Type A is crystalline Sulfate Type A characterized by two or more, or three or more XRPD signals selected from the group consisting of 3.4 °2θ, 17.1 °2θ, 17.8 °2θ, 5.5 °2θ, 22.0 °2θ, 10.5 °2θ, 15.3 °2θ, 21.0 °2θ, 25.1 °2θ, and 13.6 °2θ (±0.2 °2θ; ±0.1 °2θ; or ±0.0 °2θ; Cu Kα1 radiation). In some embodiments, the solid form of Sulfate Type A is crystalline Sulfate Type A characterized by XRPD signals at 3.4 °2θ, 17.1 °2θ, 17.8 °2θ, 5.5 °2θ, 22.0 °2θ, 10.5 °2θ, 15.3 °2θ, 21.0 °2θ, 25.1 °2θ, and 13.6 °2θ (±0.2 °2θ; ±0.1 °2θ; or ±0.0 °2θ; Cu Kα1 radiation).

In some embodiments, the crystalline Sulfate Type A is characterized by one, two, three, four, five, six, seven, eight, nine, ten, eleven, twelve, thirteen, fourteen, or fifteen XRPD signals selected from those set forth in Table 4.

TABLE 4
Sulfate Type A XRPD Signals

| Signal | Pos. | d-spacing | Rel. Int. |
|---|---|---|---|
| No. | [°2θ] | [Å] | [%] |
| 1 | 3.44 | 25.67 | 100 |
| 2 | 5.49 | 16.1 | 62.74 |
| 3 | 10.47 | 8.45 | 28.25 |
| 4 | 13.57 | 6.52 | 13.82 |
| 5 | 15.3 | 5.79 | 16.37 |
| 6 | 17.06 | 5.2 | 73.5 |
| 7 | 17.84 | 4.97 | 63.78 |
| 8 | 19.33 | 4.59 | 10.47 |
| 9 | 20.96 | 4.24 | 15.8 |
| 10 | 22.01 | 4.04 | 34.17 |
| 11 | 23.59 | 3.77 | 10.5 |
| 12 | 25.07 | 3.55 | 15.72 |
| 13 | 25.77 | 3.46 | 11.15 |
| 14 | 28.45 | 3.14 | 5.99 |
| 15 | 30.54 | 2.93 | 10.29 |

### Sulfate Type B

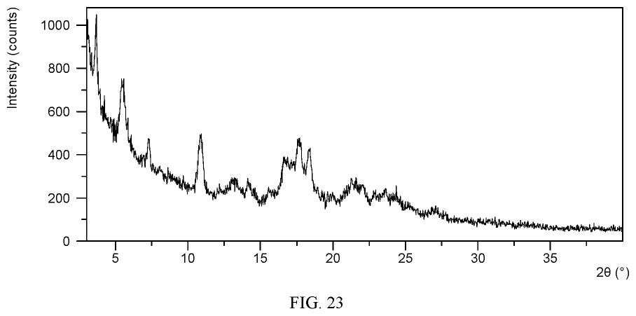

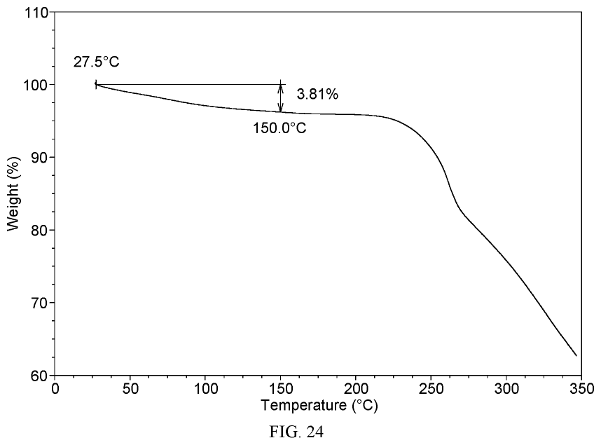

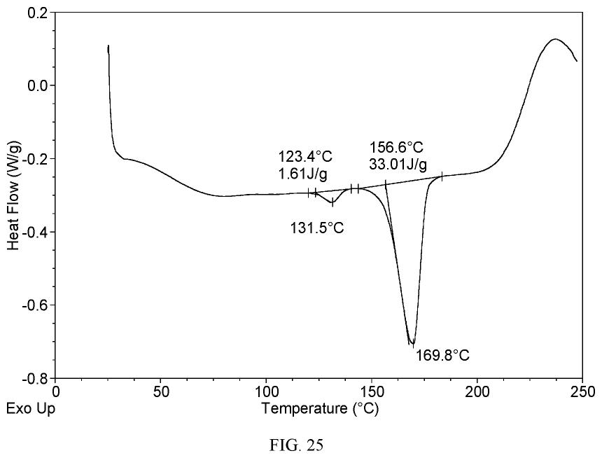

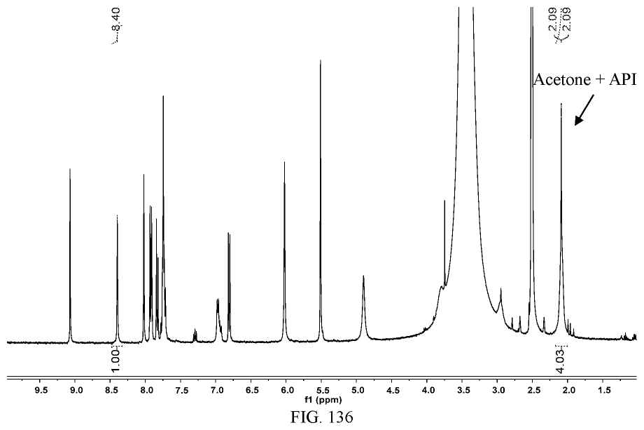

In some embodiments, the present disclosure provides solid forms of Sulfate Type B, e.g., crystalline forms of Sulfate Type B. In some embodiments, the Sulfate Type B XRPD profile is substantially similar to that shown in FIG. 23. In some embodiments, the Sulfate Type B TGA profile is substantially similar to that shown in FIG. 24. In some embodiments, the Sulfate Type B DSC curve is substantially similar to that shown in FIG. 25. In some embodiments, the Sulfate Type B ¹H NMR profile is substantially similar to that shown in FIG. 136. In some embodiments, the Sulfate Type B is characterized by a DSC curve having endotherms at about 131.5° C. and about 169.8° C.

In some embodiments, the solid form of Sulfate Type B is crystalline Sulfate Type B characterized by two or more, or three XRPD signals selected from the group consisting of 3.7 °2θ, 17.7 °2θ, and 5.5 °2θ (±0.2 °2θ; ±0.1 °2θ; or ±0.0 °2θ; Cu Kα1 radiation). In some embodiments, the solid form of Sulfate Type B is crystalline Sulfate Type B characterized by XRPD signals at 3.7 °2θ, 17.7 °2θ, and 5.5 °2θ (±0.2 °2θ; ±0.1 °2θ; or ±0.0 °2θ; Cu Kα1 radiation).

In some embodiments, the solid form of Sulfate Type B is crystalline Sulfate Type B characterized by two or more, or three or more XRPD signals selected from the group consisting of 3.7 °2θ, 17.7 °2θ, 5.5 °2θ, 10.9 °2θ, and 18.4 °2θ (±0.2 °2θ; ±0.1 °2θ; or ±0.0 °2θ; Cu Kα1 radiation). In some embodiments, the solid form of Sulfate Type B is crystalline Sulfate Type B characterized by XRPD signals at 3.7 °2θ, 17.7 °2θ, 5.5 °2θ, 10.9 °2θ, and 18.4 °2θ (±0.2 °2θ; ±0.1 °2θ; or ±0.0 °2θ; Cu Kα1 radiation).

In some embodiments, the solid form of Sulfate Type B is crystalline Sulfate Type B characterized by two or more, or three or more XRPD signals selected from the group consisting of 3.7 °2θ, 17.7 °2θ, 5.5 °2θ, 10.9 °2θ, 18.4 °2θ, 16.7 °2θ, and 7.3 °2θ (±0.2 °2θ; ±0.1 °2θ; or ±0.0 °2θ; Cu Kα1 radiation). In some embodiments, the solid form of Sulfate Type B is crystalline Sulfate Type B characterized by XRPD signals at 3.7 °2θ, 17.7 °2θ, 5.5 °2θ, 10.9 °2θ, 18.4 °2θ, 16.7 °2θ, and 7.3 °2θ (±0.2 °2θ; ±0.1 °2θ; or ±0.0 °2θ; Cu Kα1 radiation).

In some embodiments, the solid form of Sulfate Type B is crystalline Sulfate Type B characterized by two or more, or three or more XRPD signals selected from the group consisting of 3.7 °2θ, 17.7 °2θ, 5.5 °2θ, 10.9 °2θ, 18.4 °2θ, 16.7 °2θ, 7.3 °2θ, 13.2 °2θ, 0.0 °2θ, and 0.0 °2θ (±0.2 °2θ; ±0.1 °2θ; or ±0.0 °2θ; Cu Kα1 radiation). In some embodiments, the solid form of Sulfate Type B is crystalline Sulfate Type B characterized by XRPD signals at 3.7 °2θ, 17.7 °2θ, 5.5 °2θ, 10.9 °2θ, 18.4 °2θ, 16.7 °2θ, 7.3 °2θ, 13.2 °2θ, 0.0 °2θ, and 0.0 °2θ (±0.2 °2θ; ±0.1 °2θ; or ±0.0 °2θ; Cu Kα1 radiation).

In some embodiments, the crystalline Sulfate Type B is characterized by one, two, three, four, five, six, seven, or eight XRPD signals selected from those set forth in Table 5.

TABLE 5
Sulfate Type B XRPD Signals

| Signal | Pos. | d-spacing | Rel. Int. |
|---|---|---|---|
| No. | [°2θ] | [Å] | [%] |
| 1 | 3.67 | 24.1 | 100 |
| 2 | 5.46 | 16.18 | 81.58 |
| 3 | 7.31 | 12.09 | 40 |
| 4 | 10.9 | 8.12 | 80.17 |
| 5 | 13.16 | 6.73 | 25.58 |
| 6 | 16.65 | 5.32 | 61.58 |
| 7 | 17.73 | 5 | 84.23 |
| 8 | 18.43 | 4.81 | 77.82 |

### Sulfate Type C

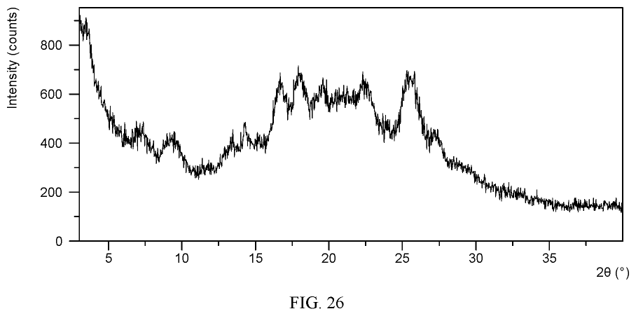

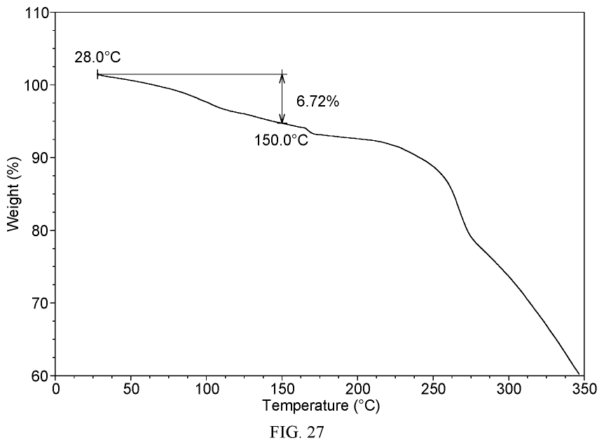

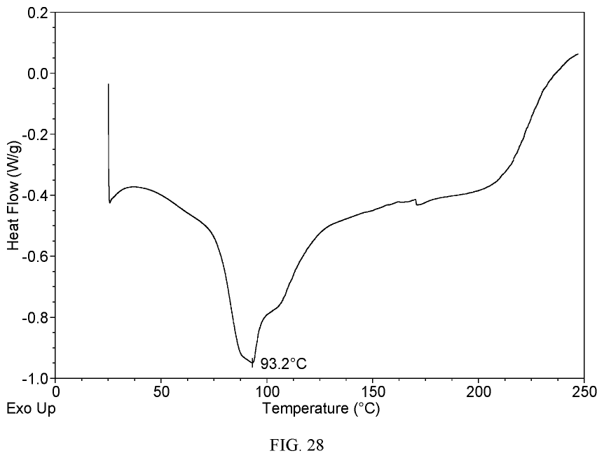

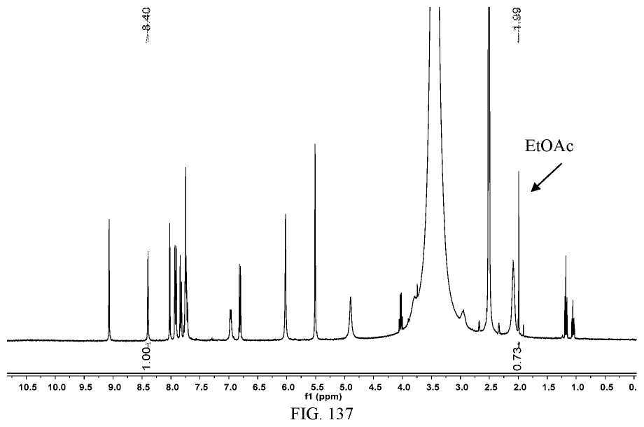

In some embodiments, the present disclosure provides solid forms of Sulfate Type C, e.g., crystalline forms of Sulfate Type C. In some embodiments, the Sulfate Type C XRPD profile is substantially similar to that shown in FIG. 26. In some embodiments, the Sulfate Type C 1H NMR spectrum is substantially similar to that shown in FIG. 137. In some embodiments, the Sulfate Type C TGA profile is substantially similar to that shown in FIG. 27. In some embodiments, the Sulfate Type C DSC curve is substantially similar to that shown in FIG. 28. In some embodiments, the Sulfate Type C ¹H NMR profile is substantially similar to that shown in FIG. 137. In some embodiments, the Sulfate Type C is characterized by a DSC curve having an endotherm at about 93.2° C.

In some embodiments, the solid form of Sulfate Type C is crystalline Sulfate Type C characterized by two or more, or three XRPD signals selected from the group consisting of 17.9 °2θ, 16.6 °2θ, and 22.5 °2θ (±0.2 °2θ; ±0.1 °2θ; or ±0.0 °2θ; Cu Kα1 radiation). In some embodiments, the solid form of Sulfate Type C is crystalline Sulfate Type C characterized by XRPD signals at 17.9 °2θ, 16.6 °2θ, and 22.5 °2θ (±0.2 °2θ; ±0.1 °2θ; or ±0.0 °2θ; Cu Kα1 radiation).

In some embodiments, the solid form of Sulfate Type C is crystalline Sulfate Type C characterized by two or more, or three or more XRPD signals selected from the group consisting of 17.9 °2θ, 16.6 °2θ, 22.5 °2θ, 19.5 °2θ, and 9.5 °2θ (±0.2 °2θ; ±0.1 °2θ; or ±0.0 °2θ; Cu Kα1 radiation). In some embodiments, the solid form of Sulfate Type C is crystalline Sulfate Type C characterized by XRPD signals at 17.9 °2θ, 16.6 °2θ, 22.5 °2θ, 19.5 °2θ, and 9.5 °2θ (±0.2 °2θ; ±0.1 °2θ; or ±0.0 °2θ; Cu Kα1 radiation).

In some embodiments, the solid form of Sulfate Type C is crystalline Sulfate Type C characterized by two or more, or three or more XRPD signals selected from the group consisting of 17.9 °2θ, 16.6 °2θ, 22.5 °2θ, 19.5 °2θ, 9.5 °2θ, 3.5 °2θ, and 7.2 °2θ (±0.2 °2θ; ±0.1 °2θ; or ±0.0 °2θ; Cu Kα1 radiation). In some embodiments, the solid form of Sulfate Type C is crystalline Sulfate Type C characterized by XRPD signals at 17.9 °2θ, 16.6 °2θ, 22.5 °2θ, 19.5 °2θ, 9.5 °2θ, 3.5 °2θ, and 7.2 °2θ (±0.2 °2θ; ±0.1 °2θ; or ±0.0 °2θ; Cu Kα1 radiation).

In some embodiments, the solid form of Sulfate Type C is crystalline Sulfate Type C characterized by two or more, or three or more XRPD signals selected from the group consisting of 17.9 °2θ, 16.6 °2θ, 22.5 °2θ, 19.5 °2θ, 9.5 °2θ, 3.5 °2θ, 7.2 °2θ, 0.0 °2θ, 0.0 °2θ, and 0.0 °2θ (±0.2 °2θ; ±0.1 °2θ; or ±0.0 °2θ; Cu Kα1 radiation). In some embodiments, the solid form of Sulfate Type C is crystalline Sulfate Type C characterized by XRPD signals at 17.9 °2θ, 16.6 °2θ, 22.5 °2θ, 19.5 °2θ, 9.5 °2θ, 3.5 °2θ, 7.2 °2θ, 0.0 °2θ, 0.0 °2θ, and 0.0 °2θ (±0.2 °2θ; ±0.1 °2θ; or ±0.0 °2θ; Cu Kα1 radiation).

In some embodiments, the crystalline Sulfate Type C is characterized by one, two, three, four, five, six, or seven XRPD signals selected from those set forth in Table 6.

TABLE 6
Sulfate Type C XRPD Signals

| Signal | Pos. | d-spacing | Rel. Int. |
|---|---|---|---|
| No. | [°2θ] | [Å] | [%] |
| 1 | 3.52 | 25.11 | 34.45 |
| 2 | 7.22 | 12.24 | 30.29 |
| 3 | 9.47 | 9.34 | 40.96 |
| 4 | 16.63 | 5.33 | 95.75 |
| 5 | 17.88 | 4.96 | 100 |
| 6 | 19.47 | 4.56 | 74.22 |
| 7 | 22.54 | 3.95 | 82.89 |

### Phosphate Type A

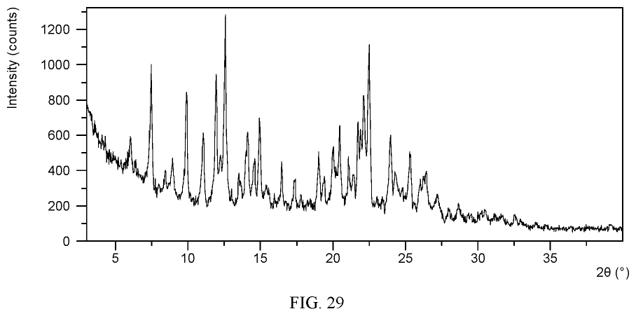

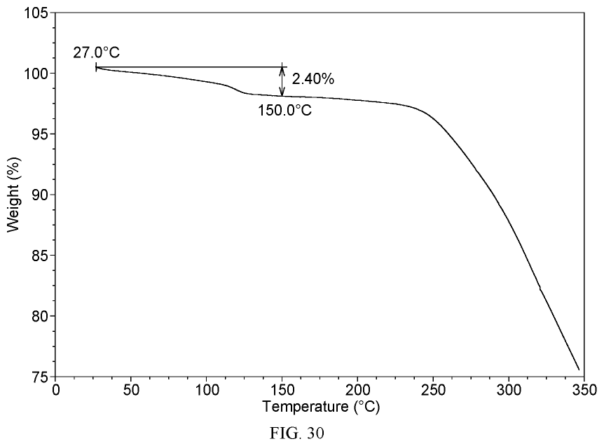

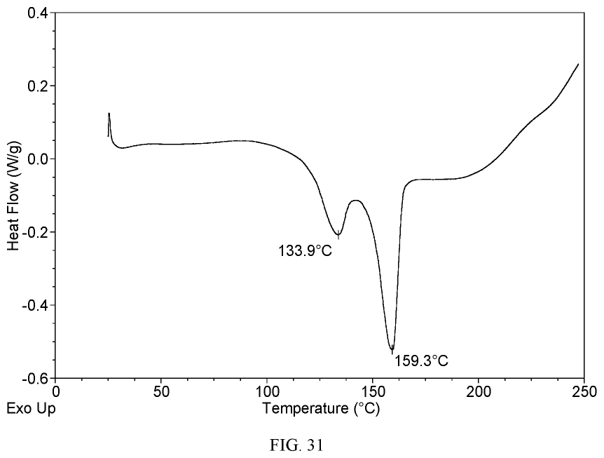

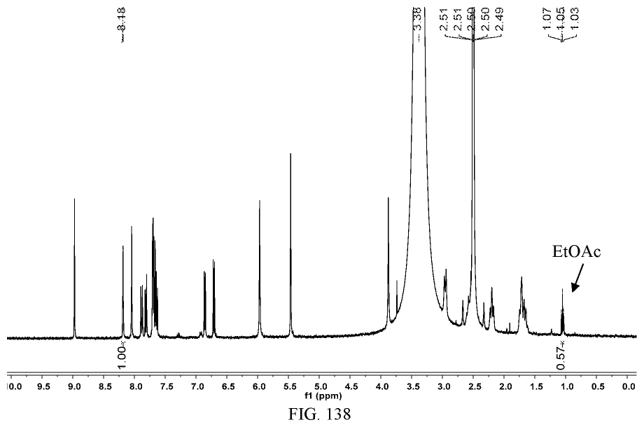

In some embodiments, the present disclosure provides solid forms of Phosphate Type A, e.g., crystalline forms of Phosphate Type A. In some embodiments, the Phosphate Type A XRPD profile is substantially similar to that shown in FIG. 29. In some embodiments, the Phosphate Type A TGA profile is substantially similar to that shown in FIG. 30. In some embodiments, the Phosphate Type A DSC curve is substantially similar to that shown in FIG. 31. In some embodiments, the Phosphate Type A ¹H NMR profile is substantially similar to that shown in FIG. 138. In some embodiments, the Phosphate Type A is characterized by a DSC curve having endotherms at about 133.9° C. and about 159.3° C.

In some embodiments, the solid form of Phosphate Type A is crystalline Phosphate Type A characterized by two or more, or three XRPD signals selected from the group consisting of 12.6 °2θ, 22.5 °2θ, and 12.0 °2θ (±0.2 °2θ; ±0.1 °2θ; or ±0.0 °2θ; Cu Kα1 radiation). In some embodiments, the solid form of Phosphate Type A is crystalline Phosphate Type A characterized by XRPD signals at 12.6 °2θ, 22.5 °2θ, and 12.0 °2θ (±0.2 °2θ; ±0.1 °2θ; or ±0.0 °2θ; Cu Kα1 radiation).

In some embodiments, the solid form of Phosphate Type A is crystalline Phosphate Type A characterized by two or more, or three or more XRPD signals selected from the group consisting of 12.6 °2θ, 22.5 °2θ, 12.0 °2θ, 7.5 °2θ, and 22.1 °2θ (±0.2 °2θ; ±0.1 °2θ; or ±0.0 °2θ; Cu Kα1 radiation). In some embodiments, the solid form of Phosphate Type A is crystalline Phosphate Type A characterized by XRPD signals at 12.6 °2θ, 22.5 °2θ, 12.0 °2θ, 7.5 °2θ, and 22.1 °2θ (±0.2 °2θ; ±0.1 °2θ; or ±0.0 °2θ; Cu Kα1 radiation).

In some embodiments, the solid form of Phosphate Type A is crystalline Phosphate Type A characterized by two or more, or three or more XRPD signals selected from the group consisting of 12.6 °2θ, 22.5 °2θ, 12.0 °2θ, 7.5 °2θ, 22.1 °2θ, 9.9 °2θ, and 21.7 °2θ (±0.2 °2θ; ±0.1 °2θ; or ±0.0 °2θ; Cu Kα1 radiation). In some embodiments, the solid form of Phosphate Type A is crystalline Phosphate Type A characterized by XRPD signals at 12.6 °2θ, 22.5 °2θ, 12.0 °2θ, 7.5 °2θ, 22.1 °2θ, 9.9 °2θ, and 21.7 °2θ (±0.2 °2θ; ±0.1 °2θ; or ±0.0 °2θ; Cu Kα1 radiation).

In some embodiments, the solid form of Phosphate Type A is crystalline Phosphate Type A characterized by two or more, or three or more XRPD signals selected from the group consisting of 12.6 °2θ, 22.5 °2θ, 12.0 °2θ, 7.5 °2θ, 22.1 °2θ, 9.9 °2θ, 21.7 °2θ, 15.0 °2θ, 20.5 °2θ, and 24.0 °2θ (±0.2 °2θ; ±0.1 °2θ; or ±0.0 °2θ; Cu Kα1 radiation). In some embodiments, the solid form of Phosphate Type A is crystalline Phosphate Type A characterized by XRPD signals at 12.6 °2θ, 22.5 °2θ, 12.0 °2θ, 7.5 °2θ, 22.1 °2θ, 9.9 °2θ, 21.7 °2θ, 15.0 °2θ, 20.5 °2θ, and 24.0 °2θ (±0.2 °2θ; ±0.1 °2θ; or ±0.0 °2θ; Cu Kα1 radiation).

In some embodiments, the crystalline Phosphate Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, or twenty-six XRPD signals selected from those set forth in Table 7.

TABLE 7
Phosphate Type A XRPD Signals

| Signal | Pos. | d-spacing | Rel. Int. |
|---|---|---|---|
| No. | [°2θ] | [Å] | [%] |
| 1 | 6.04 | 14.63 | 18.86 |
| 2 | 7.47 | 11.84 | 63.22 |
| 3 | 8.93 | 9.9 | 17.99 |
| 4 | 9.91 | 8.92 | 56.7 |
| 5 | 11.06 | 8 | 37.18 |
| 6 | 11.95 | 7.41 | 66.75 |
| 7 | 12.58 | 7.04 | 100 |
| 8 | 13.55 | 6.54 | 13.15 |
| 9 | 14.13 | 6.27 | 38.25 |
| 10 | 14.59 | 6.07 | 24.15 |
| 11 | 14.95 | 5.93 | 46.37 |
| 12 | 16.47 | 5.38 | 21.72 |
| 13 | 17.36 | 5.11 | 13.71 |
| 14 | 19.02 | 4.67 | 27.85 |
| 15 | 20 | 4.44 | 31.69 |
| 16 | 20.47 | 4.34 | 44.18 |
| 17 | 21.08 | 4.21 | 23.68 |
| 18 | 21.73 | 4.09 | 46.77 |
| 19 | 22.12 | 4.02 | 60.85 |
| 20 | 22.51 | 3.95 | 87.44 |
| 21 | 23.97 | 3.71 | 41.1 |
| 22 | 25.32 | 3.52 | 31.94 |
| 23 | 26.44 | 3.37 | 22.72 |
| 24 | 27.22 | 3.28 | 11.37 |
| 25 | 28.65 | 3.12 | 8.72 |
| 26 | 30.45 | 2.94 | 6.12 |

### Tartrate Type A

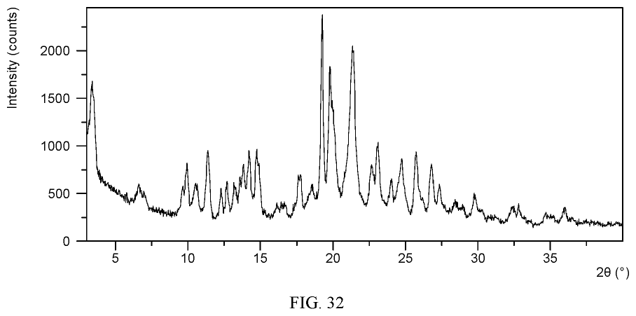

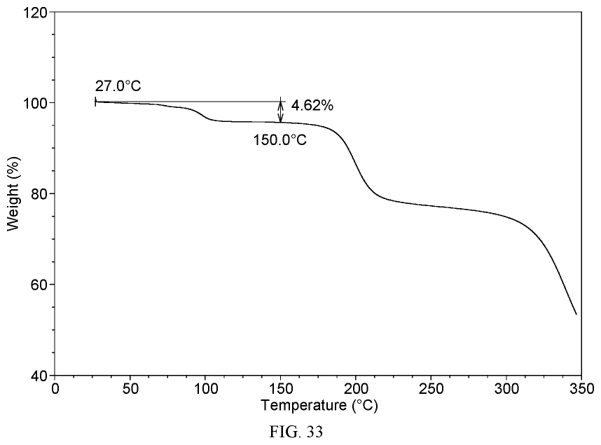

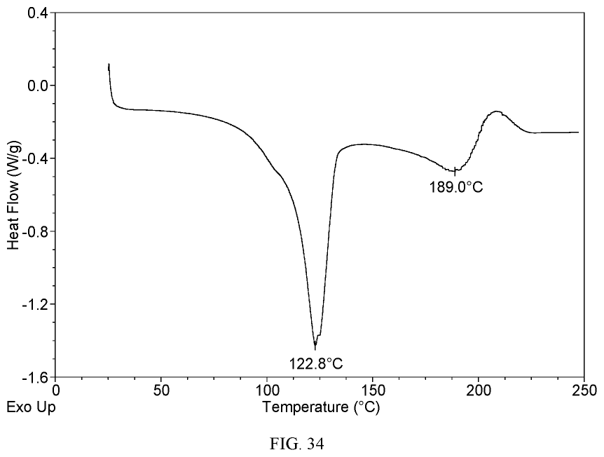

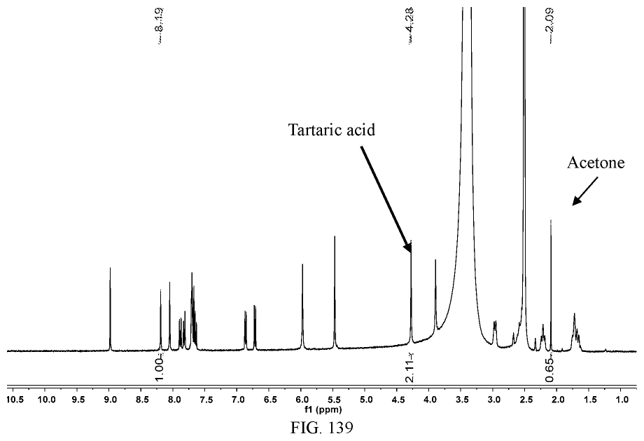

In some embodiments, the present disclosure provides solid forms of Tartrate Type A, e.g., crystalline forms of Tartrate Type A. In some embodiments, the Tartrate Type A XRPD profile is substantially similar to that shown in FIG. 32. In some embodiments, the Tartrate Type A TGA profile is substantially similar to that shown in FIG. 33. In some embodiments, the Tartrate Type A DSC curve is substantially similar to that shown in FIG. 34. In some embodiments, the Tartrate Type A ¹H NMR profile is substantially similar to that shown in FIG. 139. In some embodiments, the Tartrate Type A is characterized by a DSC curve having endotherms at about 122.8° C. and about 189.0° C.

In some embodiments, the solid form of Tartrate Type A is crystalline Tartrate Type A characterized by two or more, or three XRPD signals selected from the group consisting of 19.3 °2θ, 19.8 °2θ, and 21.5 °2θ (±0.2 °2θ; ±0.1 °2θ; or ±0.0 °2θ; Cu Kα1 radiation). In some embodiments, the solid form of Tartrate Type A is crystalline Tartrate Type A characterized by XRPD signals at 19.3 °2θ, 19.8 °2θ, and 21.5 °2θ (±0.2 °2θ; ±0.1 °2θ; or ±0.0 °2θ; Cu Kα1 radiation).

In some embodiments, the solid form of Tartrate Type A is crystalline Tartrate Type A characterized by two or more, or three or more XRPD signals selected from the group consisting of 19.3 °2θ, 19.8 °2θ, 21.5 °2θ, 3.4 °2θ, and 14.2 °2θ (±0.2 °2θ; ±0.1 °2θ; or ±0.0 °2θ; Cu Kα1 radiation). In some embodiments, the solid form of Tartrate Type A is crystalline Tartrate Type A characterized by XRPD signals at 19.3 °2θ, 19.8 °2θ, 21.5 °2θ, 3.4 °2θ, and 14.2 °2θ (±0.2 °2θ; ±0.1 °2θ; or ±0.0 °2θ; Cu Kα1 radiation).

In some embodiments, the solid form of Tartrate Type A is crystalline Tartrate Type A characterized by two or more, or three or more XRPD signals selected from the group consisting of 19.3 °2θ, 19.8 °2θ, 21.5 °2θ, 3.4 °2θ, 14.2 °2θ, 11.3 °2θ, and 23.1 °2θ (±0.2 °2θ; ±0.1 °2θ; or ±0.0 °2θ; Cu Kα1 radiation). In some embodiments, the solid form of Tartrate Type A is crystalline Tartrate Type A characterized by XRPD signals at 19.3 °2θ, 19.8 °2θ, 21.5 °2θ, 3.4 °2θ, 14.2 °2θ, 11.3 °2θ, and 23.1 °2θ (±0.2 °2θ; ±0.1 °2θ; or ±0.0 °2θ; Cu Kα1 radiation).

In some embodiments, the solid form of Tartrate Type A is crystalline Tartrate Type A characterized by two or more, or three or more XRPD signals selected from the group consisting of 19.3 °2θ, 19.8 °2θ, 21.5 °2θ, 3.4 °2θ, 14.2 °2θ, 11.3 °2θ, 23.1 °2θ, 14.7 °2θ, 25.7 °2θ, and 24.8 °2θ (±0.2 °2θ; ±0.1 °2θ; or ±±0.0 °2θ; Cu Kα1 radiation). In some embodiments, the solid form of Tartrate Type A is crystalline Tartrate Type A characterized by XRPD signals at 19.3 °2θ, 19.8 °2θ, 21.5 °2θ, 3.4 °2θ, 14.2 °2θ, 11.3 °2θ, 23.1 °2θ, 14.7 °2θ, 25.7 °2θ, and 24.8 °2θ (±0.2 °2θ; ±0.1 °2θ; or ±0.0 °2θ; Cu Kα1 radiation).

In some embodiments, the crystalline Tartrate Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty XRPD signals selected from those set forth in Table 8.

TABLE 8
Tartrate Type A XRPD Signals

| Signal | | d-spacing | Rel. Int. |
|---|---|---|---|
| No. | Pos. [°2θ] | [Å] | [%] |
| 1 | 3.4 | 25.97 | 36.91 |
| 2 | 6.63 | 13.34 | 10.22 |
| 3 | 9.95 | 8.89 | 26.26 |
| 4 | 10.58 | 8.36 | 15 |
| 5 | 11.34 | 7.8 | 32.8 |
| 6 | 12.28 | 7.21 | 14.8 |
| 7 | 12.69 | 6.97 | 18.34 |
| 8 | 13.23 | 6.69 | 15.46 |
| 9 | 13.72 | 6.45 | 20.39 |
| 10 | 14.22 | 6.23 | 33.22 |
| 11 | 14.72 | 6.02 | 32.13 |
| 12 | 16.37 | 5.41 | 3.77 |
| 13 | 17.79 | 4.99 | 18.49 |
| 14 | 18.57 | 4.78 | 12.56 |
| 15 | 19.26 | 4.61 | 100 |
| 16 | 19.79 | 4.49 | 72.61 |
| 17 | 21.45 | 4.14 | 72.02 |
| 18 | 22.68 | 3.92 | 21.98 |
| 19 | 23.07 | 3.85 | 32.2 |
| 20 | 24.04 | 3.7 | 16.05 |
| 21 | 24.75 | 3.6 | 26.51 |
| 22 | 25.72 | 3.46 | 29.19 |
| 23 | 26.81 | 3.33 | 25.25 |
| 24 | 27.37 | 3.26 | 14.69 |
| 25 | 28.44 | 3.14 | 6.63 |
| 26 | 29.79 | 3 | 11.34 |
| 27 | 32.38 | 2.77 | 8.2 |
| 28 | 32.82 | 2.73 | 8.27 |
| 29 | 34.68 | 2.59 | 5.06 |
| 30 | 35.99 | 2.5 | 8.03 |

### Mesylate Type A

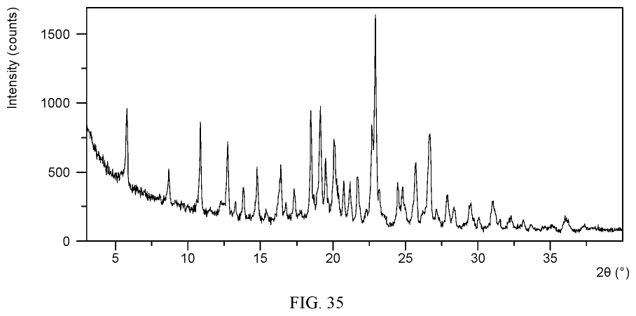

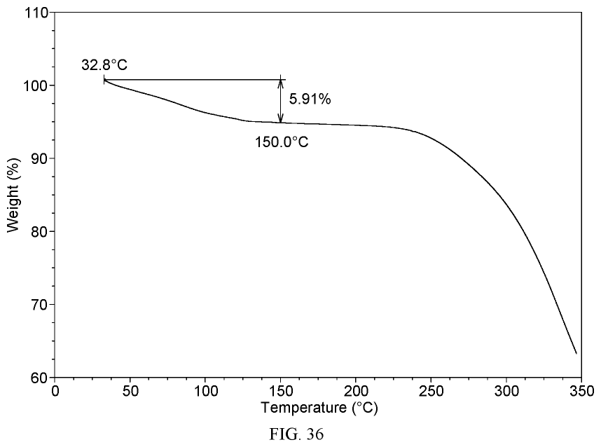

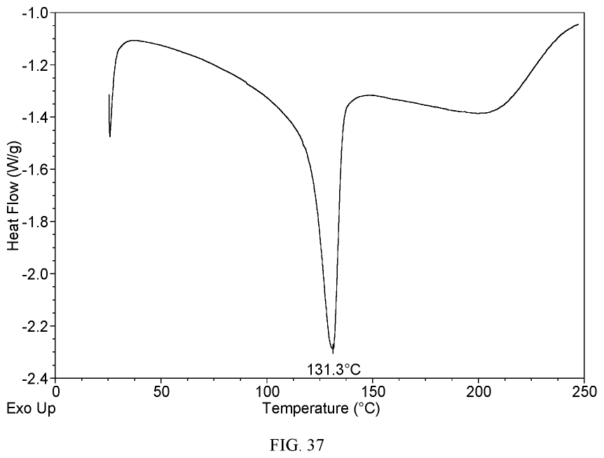

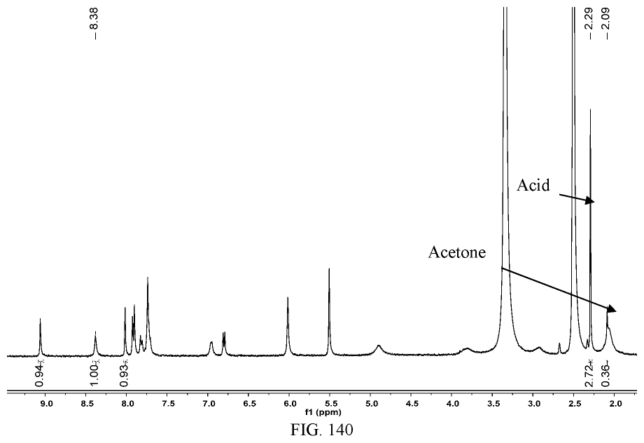

In some embodiments, the present disclosure provides solid forms of Mesylate Type A, e.g., crystalline forms of Mesylate Type A. In some embodiments, the Mesylate Type A XRPD profile is substantially similar to that shown in FIG. 35. In some embodiments, the Mesylate Type A TGA profile is substantially similar to that shown in FIG. 36. In some embodiments, the Mesylate Type A DSC curve is substantially similar to that shown in FIG. 37. In some embodiments, the Mesylate Type A ¹H NMR profile is substantially similar to that shown in FIG. 140. In some embodiments, the Mesylate Type A is characterized by a DSC curve having an endotherm at about 131.3° C.

In some embodiments, the solid form of Mesylate Type A is crystalline Mesylate Type A characterized by two or more, or three XRPD signals selected from the group consisting of 22.9 °2θ, 19.1 °2θ, and 18.5 °2θ (±0.2 °2θ; ±0.1 °2θ; or ±0.0 °2θ; Cu Kα1 radiation). In some embodiments, the solid form of Mesylate Type A is crystalline Mesylate Type A characterized by XRPD signals at 22.9 °2θ, 19.1 °2θ, and 18.5 °2θ (±0.2 °2θ; ±0.1 °2θ; or ±0.0 °2θ; Cu Kα1 radiation).

In some embodiments, the solid form of Mesylate Type A is crystalline Mesylate Type A characterized by two or more, or three or more XRPD signals selected from the group consisting of 22.9 °2θ, 19.1 °2θ, 18.5 °2θ, 22.7 °2θ, and 26.7 °2θ (±0.2 °2θ; ±0.1 °2θ; or ±0.0 °2θ; Cu Kα1 radiation). In some embodiments, the solid form of Mesylate Type A is crystalline Mesylate Type A characterized by XRPD signals at 22.9 °2θ, 19.1 °2θ, 18.5 °2θ, 22.7 °2θ, and 26.7 °2θ (±0.2 °2θ; ±0.1 °2θ; or ±0.0 °2θ; Cu Kα1 radiation).

In some embodiments, the solid form of Mesylate Type A is crystalline Mesylate Type A characterized by two or more, or three or more XRPD signals selected from the group consisting of 22.9 °2θ, 19.1 °2θ, 18.5 °2θ, 22.7 °2θ, 26.7 °2θ, 10.9 °2θ, and 20.1 °2θ (±0.2 °2θ; ±0.1 °2θ; or ±0.0 °2θ; Cu Kα1 radiation). In some embodiments, the solid form of Mesylate Type A is crystalline Mesylate Type A characterized by XRPD signals at 22.9 °2θ, 19.1 °2θ, 18.5 °2θ, 22.7 °2θ, 26.7 °2θ, 10.9 °2θ, and 20.1 °2θ (±0.2 °2θ; ±0.1 °2θ; or ±0.0 °2θ; Cu Kα1 radiation).

In some embodiments, the solid form of Mesylate Type A is crystalline Mesylate Type A characterized by two or more, or three or more XRPD signals selected from the group consisting of 22.9 °2θ, 19.1 °2θ, 18.5 °2θ, 22.7 °2θ, 26.7 °2θ, 10.9 °2θ, 20.1 °2θ, 5.8 °2θ, 12.7 °2θ, and 25.7 °2θ (±0.2 °2θ; ±0.1 °2θ; or ±0.0 °2θ; Cu Kα1 radiation). In some embodiments, the solid form of Mesylate Type A is crystalline Mesylate Type A characterized by XRPD signals at 22.9 °2θ, 19.1 °2θ, 18.5 °2θ, 22.7 °2θ, 26.7 °2θ, 10.9 °2θ, 20.1 °2θ, 5.8 °2θ, 12.7 °2θ, and 25.7 °2θ (±0.2 °2θ; ±0.1 °2θ; or ±0.0 °2θ; Cu Kα1 radiation).

In some embodiments, the crystalline Mesylate Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty XRPD signals selected from those set forth in Table 9.

TABLE 9
Mesylate Type A XRPD Signals

| Signal | | d-spacing | Rel. Int. |
|---|---|---|---|
| No. | Pos. [°2θ] | [Å] | [%] |
| 1 | 5.79 | 15.28 | 37.79 |
| 2 | 8.67 | 10.2 | 14.86 |
| 3 | 10.86 | 8.15 | 43.76 |
| 4 | 12.74 | 6.95 | 35.69 |
| 5 | 13.28 | 6.67 | 6.13 |
| 6 | 13.84 | 6.4 | 13.7 |
| 7 | 14.78 | 5.99 | 21.74 |
| 8 | 16.42 | 5.4 | 25.52 |
| 9 | 17.34 | 5.11 | 13.64 |
| 10 | 18.48 | 4.8 | 52.78 |
| 11 | 19.14 | 4.64 | 55.34 |
| 12 | 19.5 | 4.55 | 29.75 |
| 13 | 20.09 | 4.42 | 38.75 |
| 14 | 20.75 | 4.28 | 19.43 |
| 15 | 21.18 | 4.2 | 19.47 |
| 16 | 21.68 | 4.1 | 22.25 |
| 17 | 22.69 | 3.92 | 46.52 |
| 18 | 22.94 | 3.88 | 100 |
| 19 | 24.49 | 3.64 | 19.08 |
| 20 | 24.82 | 3.59 | 19.06 |
| 21 | 25.72 | 3.46 | 30.87 |
| 22 | 26.7 | 3.34 | 45.66 |
| 23 | 27.87 | 3.2 | 15.56 |
| 24 | 28.42 | 3.14 | 8.27 |
| 25 | 29.47 | 3.03 | 11.29 |
| 26 | 30.08 | 2.97 | 5.04 |
| 27 | 31 | 2.88 | 12.86 |
| 28 | 32.29 | 2.77 | 6.6 |
| 29 | 33.11 | 2.71 | 3.51 |
| 30 | 36.02 | 2.49 | 5.7 |

### Tosylate Type A

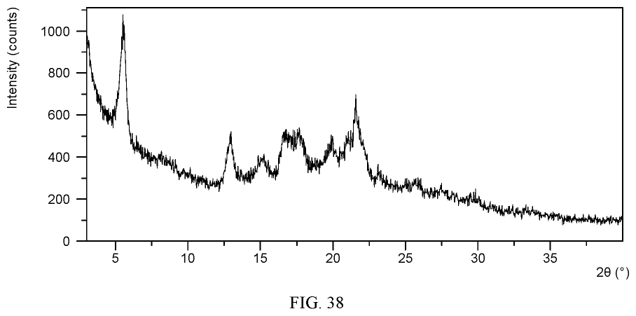

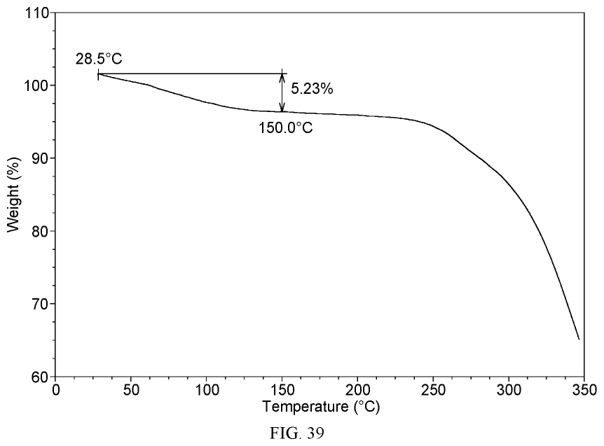

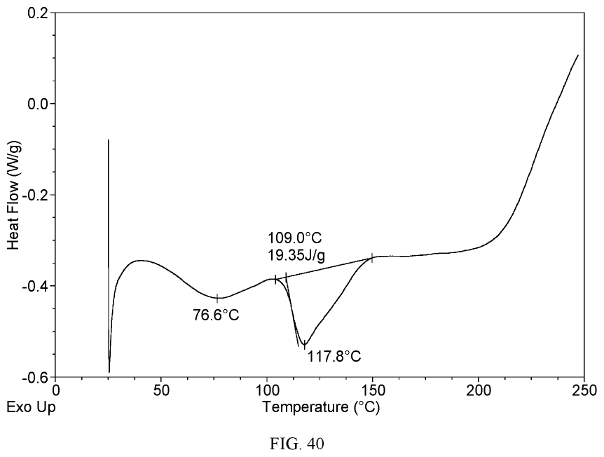

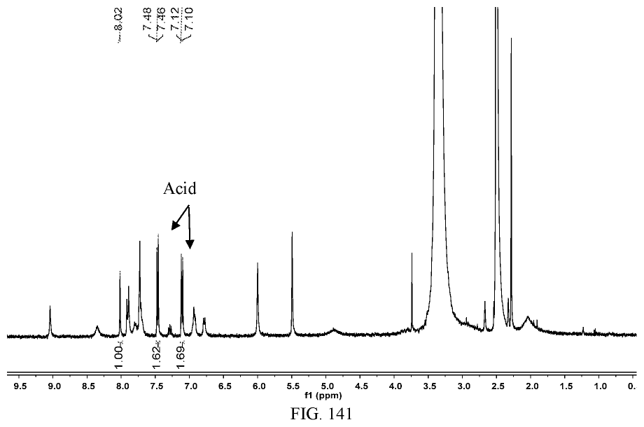

In some embodiments, the present disclosure provides solid forms of Tosylate Type A, e.g., crystalline forms of Tosylate Type A. In some embodiments, the Tosylate Type A XRPD profile is substantially similar to that shown in FIG. 38. In some embodiments, the Tosylate Type A TGA profile is substantially similar to that shown in FIG. 39. In some embodiments, the Tosylate Type A DSC curve is substantially similar to that shown in FIG. 40. In some embodiments, the Tosylate Type A ¹H NMR profile is substantially similar to that shown in FIG. 141. In some embodiments, the Tosylate Type A is characterized by a DSC curve having endotherms at about 76.6° C. and about 117.8° C.

In some embodiments, the solid form of Tosylate Type A is crystalline Tosylate Type A characterized by two or more, or three XRPD signals selected from the group consisting of 5.6 °2θ, 21.5 °2θ, and 12.9 °2θ (±0.2 °2θ; ±0.1 °2θ; or ±0.0 °2θ; Cu Kα1 radiation). In some embodiments, the solid form of Tosylate Type A is crystalline Tosylate Type A characterized by XRPD signals at 5.6 °2θ, 21.5 °2θ, and 12.9 °2θ (±0.2 °2θ; ±0.1 °2θ; or ±0.0 °2θ; Cu Kα1 radiation).

In some embodiments, the solid form of Tosylate Type A is crystalline Tosylate Type A characterized by two or more, or three or more XRPD signals selected from the group consisting of 5.6 °2θ, 21.5 °2θ, 12.9 °2θ, 17.7 °2θ, and 16.6 °2θ (±0.2 °2θ; ±0.1 °2θ; or ±0.0 °2θ; Cu Kα1 radiation). In some embodiments, the solid form of Tosylate Type A is crystalline Tosylate Type A characterized by XRPD signals at 5.6 °2θ, 21.5 °2θ, 12.9 °2θ, 17.7 °2θ, and 16.6 °2θ (±0.2 020; ±0.1 °2θ; or ±0.0 °2θ; Cu Kα1 radiation).

In some embodiments, the solid form of Tosylate Type A is crystalline Tosylate Type A characterized by two or more, or three or more XRPD signals selected from the group consisting of 5.6 °2θ, 21.5 °2θ, 12.9 °2θ, 17.7 °2θ, 16.6 °2θ, 19.9 °2θ, and 15.1 °2θ (±0.2 °2θ; ±0.1 °2θ; or ±0.0 °2θ; Cu Kα1 radiation). In some embodiments, the solid form of Tosylate Type A is crystalline Tosylate Type A characterized by XRPD signals at 5.6 °2θ, 21.5 °2θ, 12.9 °2θ, 17.7 °2θ, 16.6 °2θ, 19.9 °2θ, and 15.1 °2θ (±0.2 °2θ; ±0.1 °2θ; or ±0.0 °2θ; Cu Kα1 radiation).

In some embodiments, the crystalline Tosylate Type A is characterized by one, two, three, four, five, six, or seven XRPD signals selected from those set forth in Table 10.

TABLE 10
Tosylate Type A XRPD Signals

| | | d-spacing | Rel. Int. |
|---|---|---|---|
| Signal No. | Pos. [°2θ] | [Å] | [%] |
| 1 | 5.57 | 15.86 | 100 |
| 2 | 12.94 | 6.84 | 41.75 |
| 3 | 15.12 | 5.86 | 17.6 |
| 4 | 16.58 | 5.35 | 36.67 |
| 5 | 17.73 | 5 | 38.3 |
| 6 | 19.9 | 4.46 | 31.5 |
| 7 | 21.54 | 4.13 | 64.72 |

### Tosylate Type B

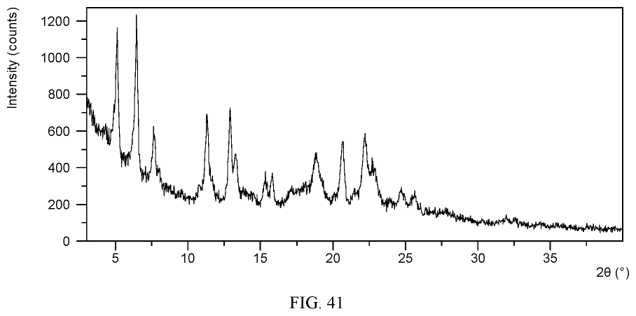

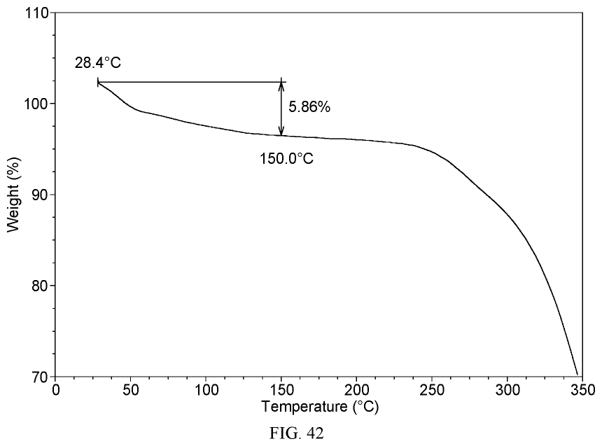

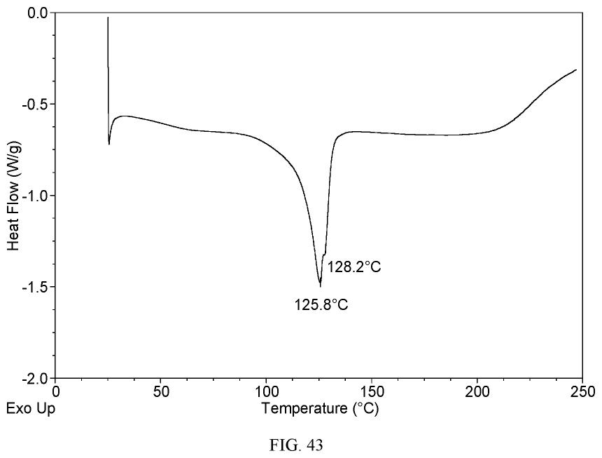

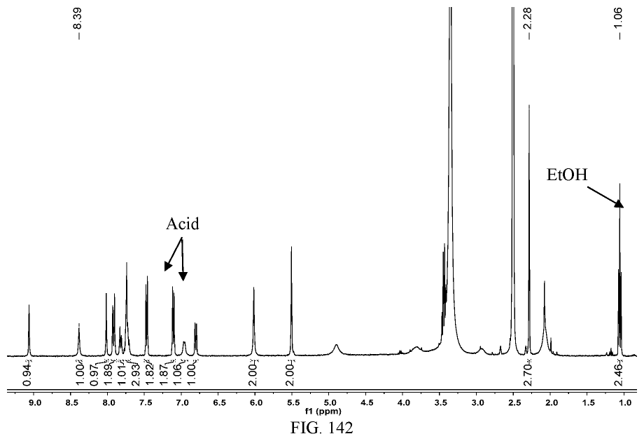

In some embodiments, the present disclosure provides solid forms of Tosylate Type B, e.g., crystalline forms of Tosylate Type B. In some embodiments, the Tosylate Type B XRPD profile is substantially similar to that shown in FIG. 41. In some embodiments, the Tosylate Type B TGA profile is substantially similar to that shown in FIG. 42. In some embodiments, the Tosylate Type B DSC curve is substantially similar to that shown in FIG. 43. In some embodiments, the Tosylate Type B ¹H NMR profile is substantially similar to that shown in FIG. 142. In some embodiments, the Tosylate Type B is characterized by a DSC curve having endotherms at about 125.8° C. and about 128.2° C.

In some embodiments, the solid form of Tosylate Type B is crystalline Tosylate Type B characterized by two or more, or three XRPD signals selected from the group consisting of 6.5 °2θ, 5.1 °2θ, and 12.9 °2θ (±0.2 °2θ; ±0.1 °2θ; or ±0.0 °2θ; Cu Kα1 radiation). In some embodiments, the solid form of Tosylate Type B is crystalline Tosylate Type B characterized by XRPD signals at 6.5 °2θ, 5.1 °2θ, and 12.9 °2θ (±0.2 °2θ; ±0.1 °2θ; or ±0.0 °2θ; Cu Kα1 radiation).

In some embodiments, the solid form of Tosylate Type B is crystalline Tosylate Type B characterized by two or more, or three or more XRPD signals selected from the group consisting of 6.5 °2θ, 5.1 °2θ, 12.9 °2θ, 11.3 °2θ, and 22.2 °2θ (±0.2 °2θ; ±0.1 °2θ; or ±0.0 °2θ; Cu Kα1 radiation). In some embodiments, the solid form of Tosylate Type B is crystalline Tosylate Type B characterized by XRPD signals at 6.5 °2θ, 5.1 °2θ, 12.9 °2θ, 11.3 °2θ, and 22.2 °2θ (±0.2 °2θ; ±0.1 °2θ; or ±0.0 °2θ; Cu Kα1 radiation).

In some embodiments, the solid form of Tosylate Type B is crystalline Tosylate Type B characterized by two or more, or three or more XRPD signals selected from the group consisting of 6.5 °2θ, 5.1 °2θ, 12.9 °2θ, 11.3 °2θ, 22.2 °2θ, 20.7 °2θ, and 18.9 °2θ (±0.2 °2θ; ±0.1 °2θ; or ±0.0 °2θ; Cu Kα1 radiation). In some embodiments, the solid form of Tosylate Type B is crystalline Tosylate Type B characterized by XRPD signals at 6.5 °2θ, 5.1 °2θ, 12.9 °2θ, 11.3 °2θ, 22.2 °2θ, 20.7 °2θ, and 18.9 °2θ (±0.2 °2θ; ±0.1 °2θ; or ±0.0 °2θ; Cu Kα1 radiation).

In some embodiments, the solid form of Tosylate Type B is crystalline Tosylate Type B characterized by two or more, or three or more XRPD signals selected from the group consisting of 6.5 °2θ, 5.1 °2θ, 12.9 °2θ, 11.3 °2θ, 22.2 °2θ, 20.7 °2θ, 18.9 °2θ, 13.3 °2θ, 7.7 °2θ, and 22.9 °2θ (±0.2 °2θ; ±0.1 °2θ; or ±0.0 °2θ; Cu Kα1 radiation). In some embodiments, the solid form of Tosylate Type B is crystalline Tosylate Type B characterized by XRPD signals at 6.5 °2θ, 5.1 °2θ, 12.9 °2θ, 11.3 °2θ, 22.2 °2θ, 20.7 °2θ, 18.9 °2θ, 13.3 °2θ, 7.7 °2θ, and 22.9 °2θ (±0.2 °2θ; ±0.1 °2θ; or ±0.0 °2θ; Cu Kα1 radiation).

In some embodiments, the crystalline Tosylate Type B is characterized by one, two, three, four, five, six, seven, eight, nine, ten, eleven, twelve, thirteen, or fourteen XRPD signals selected from those set forth in Table 11.

TABLE 11
Tosylate Type B XRPD Signals

| Signal | | d-spacing | Rel. Int. |
|---|---|---|---|
| No. | Pos. [°2θ] | [Å] | [%] |
| 1 | 5.12 | 17.25 | 85.83 |
| 2 | 6.46 | 13.68 | 100 |
| 3 | 7.67 | 11.53 | 30.16 |
| 4 | 11.32 | 7.82 | 59.58 |
| 5 | 12.9 | 6.86 | 61.69 |
| 6 | 13.31 | 6.65 | 31.89 |
| 7 | 15.34 | 5.78 | 16.88 |
| 8 | 15.81 | 5.61 | 19.29 |
| 9 | 18.85 | 4.71 | 33.53 |
| 10 | 20.67 | 4.3 | 44.46 |
| 11 | 22.19 | 4.01 | 49.45 |
| 12 | 22.86 | 3.89 | 26.2 |
| 13 | 24.74 | 3.6 | 14.03 |
| 14 | 25.59 | 3.48 | 11.92 |

### Arginine Salt Type A

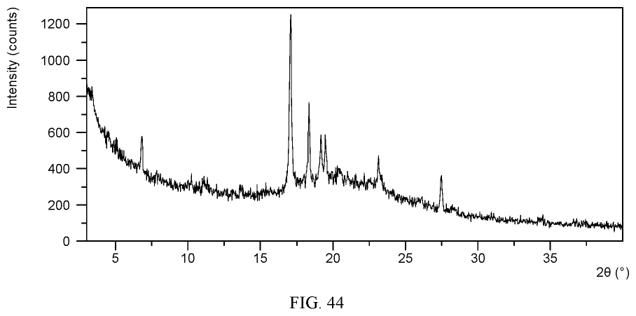

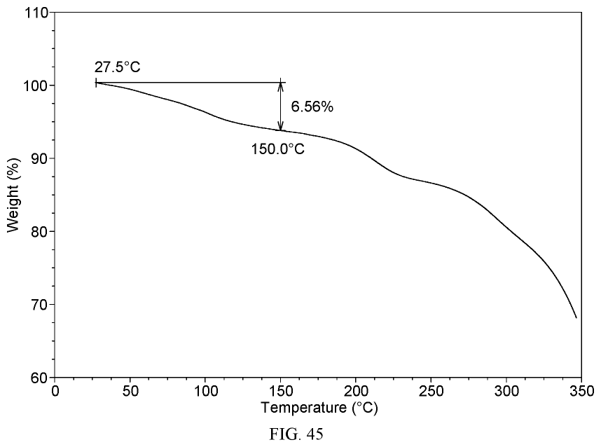

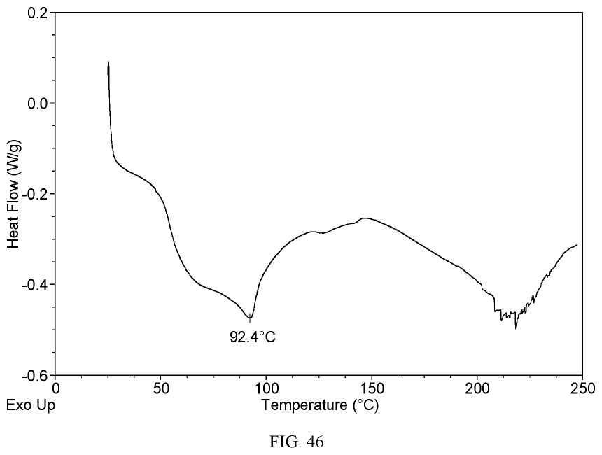

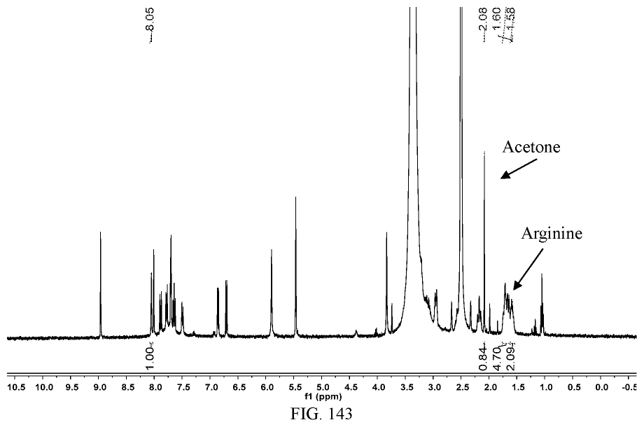

In some embodiments, the present disclosure provides solid forms of Arginine Type A, e.g., crystalline forms of Arginine Type A. In some embodiments, the Arginine Type A XRPD profile is substantially similar to that shown in FIG. 44. In some embodiments, the Arginine Type A TGA profile is substantially similar to that shown in FIG. 45. In some embodiments, the Arginine Type A DSC curve is substantially similar to that shown in FIG. 46. In some embodiments, the Arginine Type A ¹H NMR profile is substantially similar to that shown in FIG. 143. In some embodiments, the Arginine Type A is characterized by a DSC curve having endotherms at about 92.4° C.

In some embodiments, the solid form of Arginine Type A is crystalline Arginine Type A characterized by two or more, or three XRPD signals selected from the group consisting of 17.1 °2θ, 18.4 °2θ, and 19.5 °2θ (±0.2 °2θ; ±0.1 °2θ; or ±0.0 °2θ; Cu Kα1 radiation). In some embodiments, the solid form of Arginine Type A is crystalline Arginine Type A characterized by XRPD signals at 17.1 °2θ, 18.4 °2θ, and 19.5 °2θ (±0.2 °2θ; ±0.1 °2θ; or ±0.0 °2θ; Cu Kα1 radiation).

In some embodiments, the solid form of Arginine Type A is crystalline Arginine Type A characterized by two or more, or three or more XRPD signals selected from the group consisting of 17.1 °2θ, 18.4 °2θ, 19.5 °2θ, 19.2 °2θ, and 23.1 °2θ (±0.2 °2θ; ±0.1 °2θ; or ±0.0 °2θ; Cu Kα1 radiation). In some embodiments, the solid form of Arginine Type A is crystalline Arginine Type A characterized by XRPD signals at 17.1 °2θ, 18.4 °2θ, 19.5 °2θ, 19.2 °2θ, and 23.1 °2θ (±0.2 °2θ; ±0.1 °2θ; or ±0.0 °2θ; Cu Kα1 radiation).

In some embodiments, the solid form of Arginine Type A is crystalline Arginine Type A characterized by two or more, or three or more XRPD signals selected from the group consisting of 17.1 °2θ, 18.4 °2θ, 19.5 °2θ, 19.2 °2θ, 23.1 °2θ, 6.8 °2θ, and 27.5 °2θ (±0.2 °2θ; ±0.1 °2θ; or ±0.0 °2θ; Cu Kα1 radiation). In some embodiments, the solid form of Arginine Type A is crystalline Arginine Type A characterized by XRPD signals at 17.1 °2θ, 18.4 °2θ, 19.5 °2θ, 19.2 °2θ, 23.1 °2θ, 6.8 °2θ, and 27.5 °2θ (±0.2 °2θ; ±0.1 °2θ; or ±0.0 °2θ; Cu Kα1 radiation).

In some embodiments, the solid form of Arginine Type A is crystalline Arginine Type A characterized by two or more, or three or more XRPD signals selected from the group consisting of 17.1 °2θ, 18.4 °2θ, 19.5 °2θ, 19.2 °2θ, 23.1 °2θ, 6.8 °2θ, 27.5 °2θ, 0.0 °2θ, 0.0 °2θ, and 0.0 °2θ (±0.2 °2θ; ±0.1 °2θ; or ±0.0 °2θ; Cu Kα1 radiation). In some embodiments, the solid form of Arginine Type A is crystalline Arginine Type A characterized by XRPD signals at 17.1 °2θ, 18.4 °2θ, 19.5 °2θ, 19.2 °2θ, 23.1 °2θ, 6.8 °2θ, 27.5 °2θ, 0.0 °2θ, 0.0 °2θ, and 0.0 °2θ (±0.2 °2θ; ±0.1 °2θ; or ±0.0 °2θ; Cu Kα1 radiation).

In some embodiments, the crystalline Arginine Type A is characterized by one, two, three, four, five, six, or seven XRPD signals selected from those set forth in Table 12.

TABLE 12
Arginine Type A XRPD Signals

| Signal | | d-spacing | Rel. Int. |
|---|---|---|---|
| No. | Pos. [°2θ] | [Å] | [%] |
| 1 | 6.82 | 12.96 | 21.13 |
| 2 | 17.08 | 5.19 | 100 |
| 3 | 18.35 | 4.83 | 49.83 |
| 4 | 19.17 | 4.63 | 30.01 |
| 5 | 19.48 | 4.56 | 31.54 |
| 6 | 23.14 | 3.84 | 22.31 |
| 7 | 27.47 | 3.25 | 20.19 |

### Arginine Type B

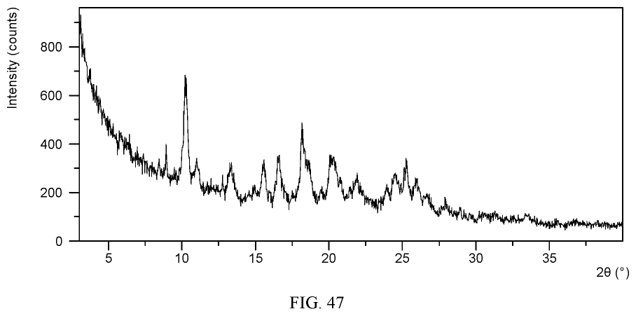

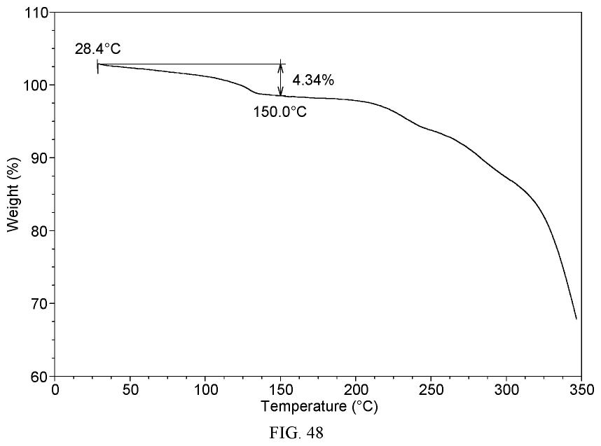

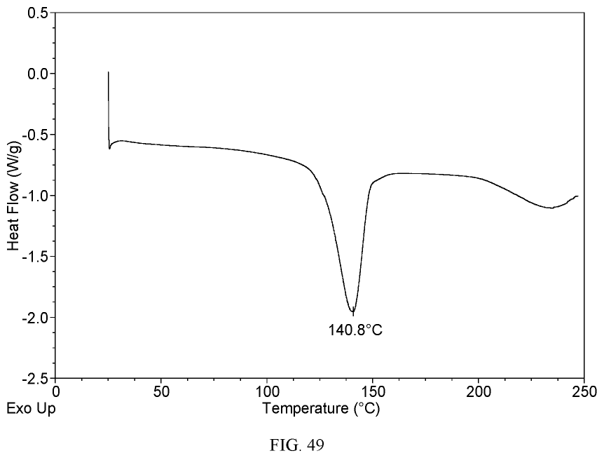

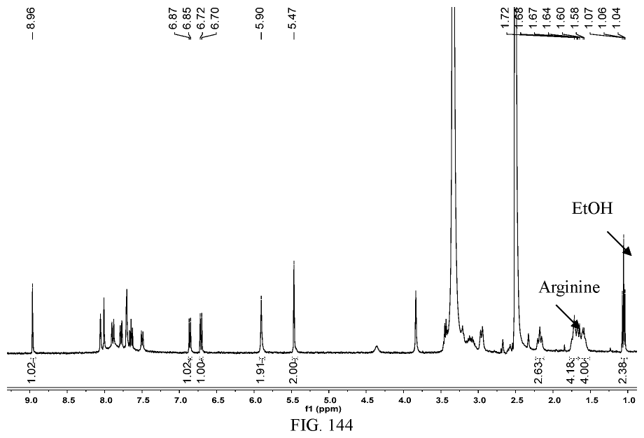

In some embodiments, the present disclosure provides solid forms of Arginine Type B, e.g., crystalline forms of Arginine Type B. In some embodiments, the Arginine Type B XRPD profile is substantially similar to that shown in FIG. 47. In some embodiments, the Arginine Type B TGA profile is substantially similar to that shown in FIG. 48. In some embodiments, the Arginine Type B DSC curve is substantially similar to that shown in FIG. 49. In some embodiments, the Arginine Type B ¹H NMR profile is substantially similar to that shown in FIG. 144. In some embodiments, the Arginine Type B is characterized by a DSC curve having an endotherm at about 140.8° C.

In some embodiments, the solid form of Arginine Type B is crystalline Arginine Type B characterized by two or more, or three XRPD signals selected from the group consisting of 10.3 °2θ, 18.2 °2θ, and 16.6 °2θ (±0.2 °2θ; ±0.1 °2θ; or ±0.0 °2θ; Cu Kα1 radiation). In some embodiments, the solid form of Arginine Type B is crystalline Arginine Type B characterized by XRPD signals at 10.3 °2θ, 18.2 °2θ, and 16.6 °2θ (±0.2 °2θ; ±0.1 °2θ; or ±0.0 °2θ; Cu Kα1 radiation).

In some embodiments, the solid form of Arginine Type B is crystalline Arginine Type B characterized by two or more, or three or more XRPD signals selected from the group consisting of 10.3 °2θ, 18.2 °2θ, 16.6 °2θ, 20.2 °2θ, and 25.2 °2θ (±0.2 °2θ; ±0.1 °2θ; or ±0.0 °2θ; Cu Kα1 radiation). In some embodiments, the solid form of Arginine Type B is crystalline Arginine Type B characterized by XRPD signals at 10.3 °2θ, 18.2 °2θ, 16.6 °2θ, 20.2 °2θ, and 25.2 °2θ (±0.2 °2θ; ±0.1 °2θ; or ±0.0 °2θ; Cu Kα1 radiation).

In some embodiments, the solid form of Arginine Type B is crystalline Arginine Type B characterized by two or more, or three or more XRPD signals selected from the group consisting of 10.3 °2θ, 18.2 °2θ, 16.6 °2θ, 20.2 °2θ, 25.2 °2θ, 15.6 °2θ, and 24.5 °2θ (±0.2 °2θ; ±0.1 °2θ; or ±0.0 °2θ; Cu Kα1 radiation). In some embodiments, the solid form of Arginine Type B is crystalline Arginine Type B characterized by XRPD signals at 10.3 °2θ, 18.2 °2θ, 16.6 °2θ, 20.2 °2θ, 25.2 °2θ, 15.6 °2θ, and 24.5 °2θ (±0.2 °2θ; ±0.1 °2θ; or ±0.0 °2θ; Cu Kα1 radiation).

In some embodiments, the solid form of Arginine Type B is crystalline Arginine Type B characterized by two or more, or three or more XRPD signals selected from the group consisting of 10.3 °2θ, 18.2 °2θ, 16.6 °2θ, 20.2 °2θ, 25.2 °2θ, 15.6 °2θ, 24.5 °2θ, 26.0 °2θ, 13.3 °2θ, and 21.9 °2θ (±0.2 °2θ; ±0.1 °2θ; or ±0.0 °2θ; Cu Kα1 radiation). In some embodiments, the solid form of Arginine Type B is crystalline Arginine Type B characterized by XRPD signals at 10.3 °2θ, 18.2 °2θ, 16.6 °2θ, 20.2 °2θ, 25.2 °2θ, 15.6 °2θ, 24.5 °2θ, 26.0 °2θ, 13.3 °2θ, and 21.9 °2θ (±0.2 °2θ; ±0.1 °2θ; or ±0.0 °2θ; Cu Kα1 radiation).

In some embodiments, the crystalline Arginine Type B is characterized by one, two, three, four, five, six, seven, eight, nine, ten, or eleven XRPD signals selected from those set forth in Table 13.

TABLE 13
Arginine Type B XRPD Signals

| Signal | | d-spacing | Rel. Int. |
|---|---|---|---|
| No. | Pos. [°2θ] | [Å] | [%] |
| 1 | 10.26 | 8.63 | 100 |
| 2 | 11.02 | 8.03 | 23.35 |
| 3 | 13.3 | 6.66 | 28.97 |
| 4 | 15.58 | 5.69 | 40.53 |
| 5 | 16.58 | 5.35 | 43.83 |
| 6 | 18.16 | 4.88 | 69.24 |
| 7 | 20.19 | 4.4 | 42.61 |
| 8 | 21.88 | 4.06 | 25.15 |
| 9 | 24.48 | 3.64 | 33.16 |
| 10 | 25.24 | 3.53 | 41.45 |
| 11 | 25.98 | 3.43 | 29.41 |

### Lysine Salt Type A

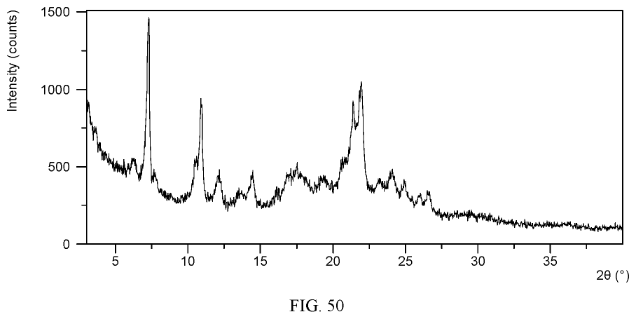

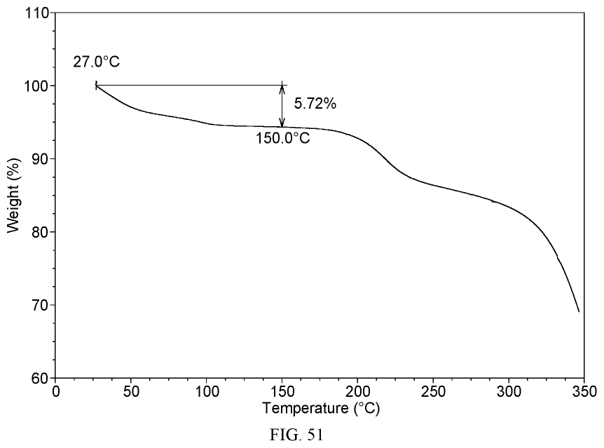

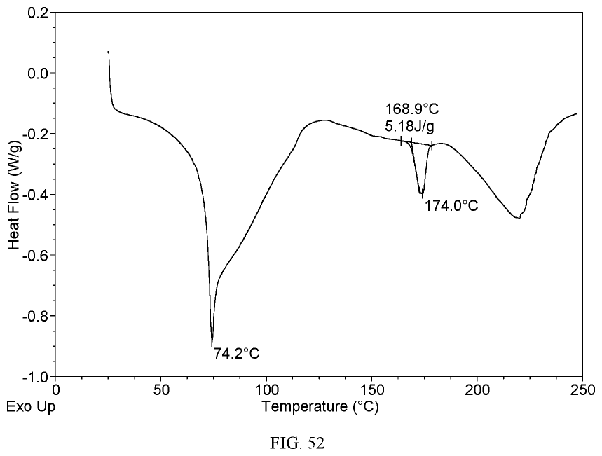

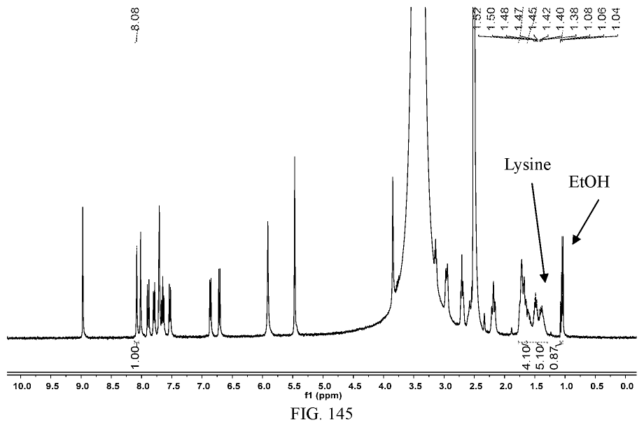

In some embodiments, the present disclosure provides solid forms of Lysine Type A, e.g., crystalline forms of Lysine Type A. In some embodiments, the Lysine Type A XRPD profile is substantially similar to that shown in FIG. 50. In some embodiments, the Lysine Type A TGA profile is substantially similar to that shown in FIG. 51. In some embodiments, the Lysine Type A DSC curve is substantially similar to that shown in FIG. 52. In some embodiments, the Lysine Type A ¹H NMR profile is substantially similar to that shown in FIG. 145. In some embodiments, the Lysine Type A is characterized by a DSC curve having endotherms at about 74.2° C. and about 174.0° C.

In some embodiments, the solid form of Lysine Type A is crystalline Lysine Type A characterized by two or more, or three XRPD signals selected from the group consisting of 7.3 °2θ, 22.0 °2θ, and 10.9 °2θ (±0.2 °2θ; ±0.1 °2θ; or ±0.0 °2θ; Cu Kα1 radiation). In some embodiments, the solid form of Lysine Type A is crystalline Lysine Type A characterized by XRPD signals at 7.3 °2θ, 22.0 °2θ, and 10.9 °2θ (±0.2 °2θ; ±0.1 °2θ; or ±0.0 °2θ; Cu Kα1 radiation).

In some embodiments, the solid form of Lysine Type A is crystalline Lysine Type A characterized by two or more, or three or more XRPD signals selected from the group consisting of 7.3 °2θ, 22.0 °2θ, 10.9 °2θ, 21.4 °2θ, and 14.5 °2θ (±0.2 °2θ; ±0.1 °2θ; or ±0.0 °2θ; Cu Kα1 radiation). In some embodiments, the solid form of Lysine Type A is crystalline Lysine Type A characterized by XRPD signals at 7.3 °2θ, 22.0 °2θ, 10.9 °2θ, 21.4 °2θ, and 14.5 °2θ (±0.2 °2θ; ±0.1 °2θ; or ±0.0 °2θ; Cu Kα1 radiation).

In some embodiments, the solid form of Lysine Type A is crystalline Lysine Type A characterized by two or more, or three or more XRPD signals selected from the group consisting of 7.3 °2θ, 22.0 °2θ, 10.9 °2θ, 21.4 °2θ, 14.5 °2θ, 24.2 °2θ, and 12.1 °2θ (±0.2 °2θ; ±0.1 °2θ; or ±0.0 °2θ; Cu Kα1 radiation). In some embodiments, the solid form of Lysine Type A is crystalline Lysine Type A characterized by XRPD signals at 7.3 °2θ, 22.0 °2θ, 10.9 °2θ, 21.4 °2θ, 14.5 °2θ, 24.2 °2θ, and 12.1 °2θ (±0.2 °2θ; ±0.1 °2θ; or ±0.0 °2θ; Cu Kα1 radiation).

In some embodiments, the solid form of Lysine Type A is crystalline Lysine Type A characterized by two or more, or three or more XRPD signals selected from the group consisting of 7.3 °2θ, 22.0 °2θ, 10.9 °2θ, 21.4 °2θ, 14.5 °2θ, 24.2 °2θ, 12.1 °2θ, 24.9 °2θ, 26.6 °2θ, and 0.0 °2θ (±0.2 °2θ; ±0.1 °2θ; or ±0.0 °2θ; Cu Kα1 radiation). In some embodiments, the solid form of Lysine Type A is crystalline Lysine Type A characterized by XRPD signals at 7.3 20, 22.0 20, 10.9 °2θ, 21.4 °2θ, 14.5 °2θ, 24.2 °2θ, 12.1 °2θ, 24.9 °2θ, 26.6 °2θ, and 0.0 °2θ (±0.2 °2θ; ±0.1 °2θ; or ±0.0 °2θ; Cu Kα1 radiation).

In some embodiments, the crystalline Lysine Type A is characterized by one, two, three, four, five, six, seven, eight, or nine XRPD signals selected from those set forth in Table 14.

TABLE 14
Lysine Type A XRPD Signals

| Signal | | d-spacing | Rel. Int. |
|---|---|---|---|
| No. | Pos. [°2θ] | [Å] | [%] |
| 1 | 7.29 | 12.13 | 100 |
| 2 | 10.94 | 8.09 | 59.75 |
| 3 | 12.1 | 7.31 | 16.04 |
| 4 | 14.45 | 6.13 | 18.14 |
| 5 | 21.38 | 4.16 | 55.6 |
| 6 | 21.97 | 4.05 | 70.7 |
| 7 | 24.17 | 3.68 | 16.57 |
| 8 | 24.94 | 3.57 | 13.95 |
| 9 | 26.59 | 3.35 | 11.38 |

### Lysine Salt Type B

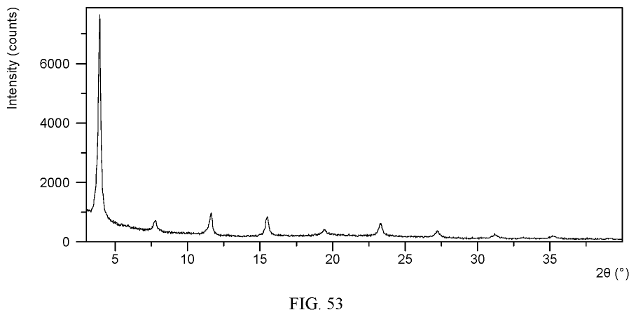

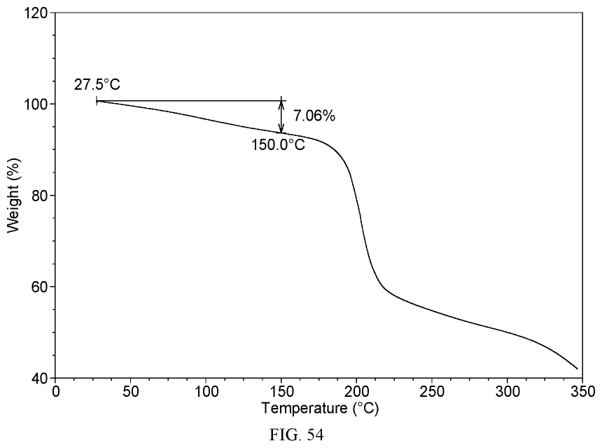

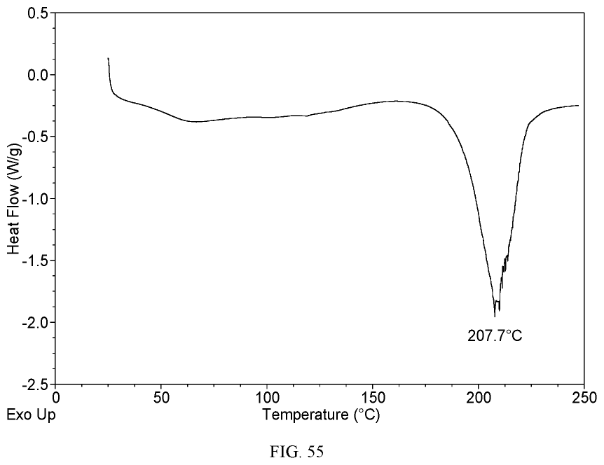

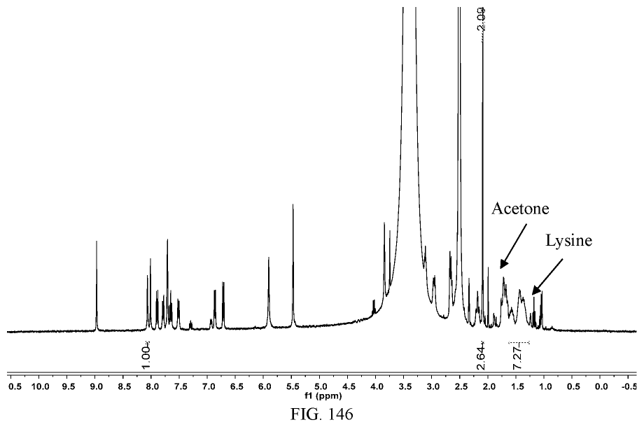

In some embodiments, the present disclosure provides solid forms of Lysine Type B, e.g., crystalline forms of Lysine Type B. In some embodiments, the Lysine Type B XRPD profile is substantially similar to that shown in FIG. 53. In some embodiments, the Lysine Type B TGA profile is substantially similar to that shown in FIG. 54. In some embodiments, the Lysine Type B DSC curve is substantially similar to that shown in FIG. 55. In some embodiments, the Lysine Type B ¹H NMR profile is substantially similar to that shown in FIG. 146. In some embodiments, the Lysine Type B is characterized by a DSC curve having an endotherm at about 207.7° C.

In some embodiments, the solid form of Lysine Type B is crystalline Lysine Type B characterized by two or more, or three XRPD signals selected from the group consisting of 3.9 °2θ, 11.6 °2θ, and 15.5 °2θ (±0.2 °2θ; ±0.1 °2θ; or ±0.0 °2θ; Cu Kα1 radiation). In some embodiments, the solid form of Lysine Type B is crystalline Lysine Type B characterized by XRPD signals at 3.9 °2θ, 11.6 °2θ, and 15.5 °2θ (±0.2 °2θ; ±0.1 °2θ; or ±0.0 °2θ; Cu Kα1 radiation).

In some embodiments, the solid form of Lysine Type B is crystalline Lysine Type B characterized by two or more, or three or more XRPD signals selected from the group consisting of 3.9 °2θ, 11.6 °2θ, 15.5 °2θ, 23.3 °2θ, and 7.8 °2θ (±0.2 °2θ; ±0.1 °2θ; or ±0.0 °2θ; Cu Kα1 radiation). In some embodiments, the solid form of Lysine Type B is crystalline Lysine Type B characterized by XRPD signals at 3.9 °2θ, 11.6 °2θ, 15.5 °2θ, 23.3 °2θ, and 7.8 °2θ (±0.2 °2θ; ±0.1 °2θ; or ±0.0 °2θ; Cu Kα1 radiation).

In some embodiments, the solid form of Lysine Type B is crystalline Lysine Type B characterized by two or more, or three or more XRPD signals selected from the group consisting of 3.9 °2θ, 11.6 °2θ, 15.5 °2θ, 23.3 °2θ, 7.8 °2θ, 27.2 °2θ, and 19.4 °2θ (±0.2 °2θ; ±0.1 °2θ; or ±0.0 °2θ; Cu Kα1 radiation). In some embodiments, the solid form of Lysine Type B is crystalline Lysine Type B characterized by XRPD signals at 3.9 °2θ, 11.6 °2θ, 15.5 °2θ, 23.3 °2θ, 7.8 °2θ, 27.2 °2θ, and 19.4 °2θ (±0.2 °2θ; ±0.1 °2θ; or ±0.0 °2θ; Cu Kα1 radiation).

In some embodiments, the solid form of Lysine Type B is crystalline Lysine Type B characterized by two or more, or three or more XRPD signals selected from the group consisting of 3.9 °2θ, 11.6 °2θ, 15.5 °2θ, 23.3 °2θ, 7.8 °2θ, 27.2 °2θ, 19.4 °2θ, 31.2 °2θ, 35.2 °2θ, and 0.0 °2θ (±0.2 °2θ; ±0.1 °2θ; or ±0.0 °2θ; Cu Kα1 radiation). In some embodiments, the solid form of Lysine Type B is crystalline Lysine Type B characterized by XRPD signals at 3.9 °2θ, 11.6 °2θ, 15.5 °2θ, 23.3 °2θ, 7.8 °2θ, 27.2 °2θ, 19.4 °2θ, 31.2 °2θ, 35.2 °2θ, and 0.0 °2θ (±0.2 °2θ; ±0.1 °2θ; or ±0.0 °2θ; Cu Kα1 radiation).

In some embodiments, the crystalline Lysine Type B is characterized by one, two, three, four, five, six, seven, eight, or nine XRPD signals selected from those set forth in Table 15.

TABLE 15
Lysine Type B XRPD Signals

| Signal | | d-spacing | Rel. Int. |
|---|---|---|---|
| No. | Pos. [°2θ] | [Å] | [%] |
| 1 | 3.94 | 22.43 | 100 |
| 2 | 7.79 | 11.34 | 5.43 |
| 3 | 11.63 | 7.61 | 10.79 |
| 4 | 15.52 | 5.71 | 9.72 |
| 5 | 19.44 | 4.57 | 3.26 |
| 6 | 23.3 | 3.82 | 6.58 |
| 7 | 27.24 | 3.27 | 3.55 |
| 8 | 31.23 | 2.86 | 1.91 |
| 9 | 35.23 | 2.55 | 1.41 |

### Sodium Type A

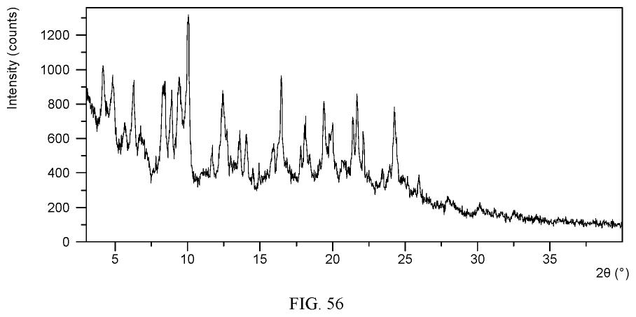

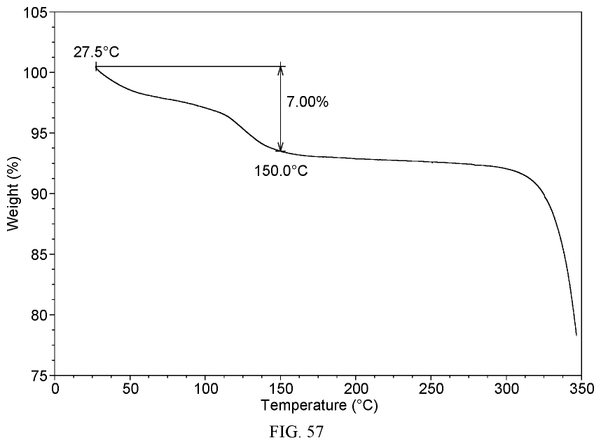

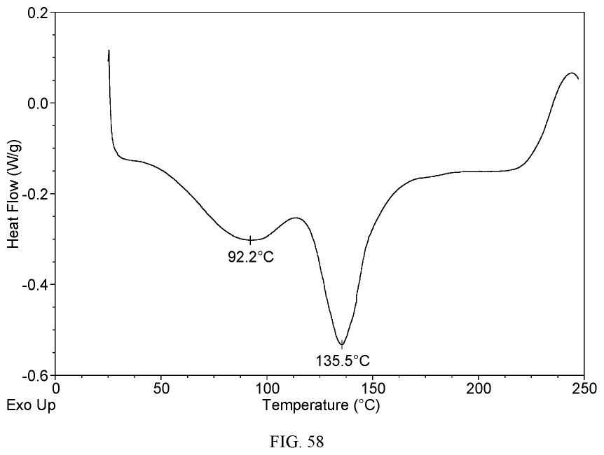

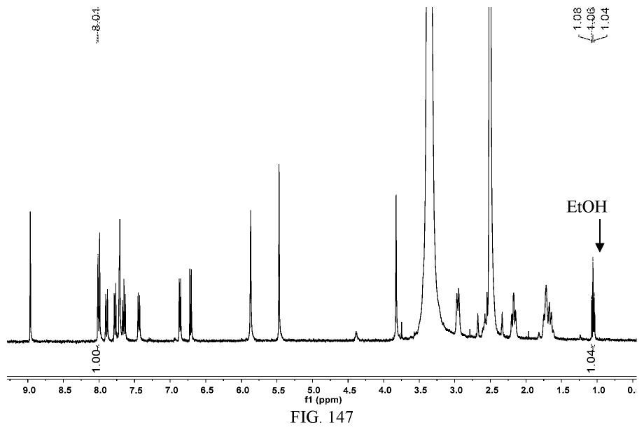

In some embodiments, the present disclosure provides solid forms of Sodium Type A, e.g., crystalline forms of Sodium Type A. In some embodiments, the Sodium Type A XRPD profile is substantially similar to that shown in FIG. 56. In some embodiments, the Sodium Type A TGA profile is substantially similar to that shown in FIG. 57. In some embodiments, the Sodium Type A DSC profile is substantially similar to that shown in FIG. 58. In some embodiments, the Sodium Type A ¹H NMR profile is substantially similar to that shown in FIG. 147. In some embodiments, the Sodium Type A is characterized by a DSC curve having endotherms at about 92.2° C. and about 135.5° C.

In some embodiments, the solid form of Sodium Type A is crystalline Sodium Type A characterized by two or more, or three XRPD signals selected from the group consisting of 10.0 °2θ, 16.5 °2θ, and 21.7 °2θ (±0.2 °2θ; ±0.1 °2θ; or ±0.0 °2θ; Cu Kα1 radiation). In some embodiments, the solid form of Sodium Type A is crystalline Sodium Type A characterized by XRPD signals at 10.0 °2θ, 16.5 °2θ, and 21.7 °2θ (±0.2 °2θ; ±0.1 °2θ; or ±0.0 °2θ; Cu Kα1 radiation).

In some embodiments, the solid form of Sodium Type A is crystalline Sodium Type A characterized by two or more, or three or more XRPD signals selected from the group consisting of 10.0 °2θ, 16.5 °2θ, 21.7 °2θ, 9.4 °2θ, and 12.4 °2θ (±0.2 °2θ; ±0.1 °2θ; or ±0.0 °2θ; Cu Kα1 radiation). In some embodiments, the solid form of Sodium Type A is crystalline Sodium Type A characterized by XRPD signals at 10.0 °2θ, 16.5 °2θ, 21.7 °2θ, 9.4 °2θ, and 12.4 °2θ (±0.2 °2θ; ±0.1 °2θ; or ±0.0 °2θ; Cu Kα1 radiation).

In some embodiments, the solid form of Sodium Type A is crystalline Sodium Type A characterized by two or more, or three or more XRPD signals selected from the group consisting of 10.0 °2θ, 16.5 °2θ, 21.7 °2θ, 9.4 °2θ, 12.4 °2θ, 8.4 °2θ, and 24.3 °2θ (±0.2 °2θ; ±0.1 °2θ; or ±0.0 °2θ; Cu Kα1 radiation). In some embodiments, the solid form of Sodium Type A is crystalline Sodium Type A characterized by XRPD signals at 10.0 °2θ, 16.5 °2θ, 21.7 °2θ, 9.4 °2θ, 12.4 °2θ, 8.4 °2θ, and 24.3 °2θ (±0.2 °2θ; ±0.1 °2θ; or ±0.0 °2θ; Cu Kα1 radiation).

In some embodiments, the solid form of Sodium Type A is crystalline Sodium Type A characterized by two or more, or three or more XRPD signals selected from the group consisting of 10.0 °2θ, 16.5 °2θ, 21.7 °2θ, 9.4 °2θ, 12.4 °2θ, 8.4 °2θ, 24.3 °2θ, 19.4 °2θ, 8.9 °2θ, and 6.3 °2θ; ±0.1 °2θ; or ±0.0 °2θ; Cu Kαl radiation). In some embodiments, the solid form of Sodium Type A is crystalline Sodium Type A characterized by XRPD signals at 10.0 °2θ, 16.5 °2θ, 21.7 °2θ, 9.4 °2θ, 12.4 °2θ, 8.4 °2θ, 24.3 °2θ, 19.4 °2θ, 8.9 °2θ, and 6.3 °2θ (±0.2 °2θ; ±0.1 °2θ; or ±0.0 °2θ; Cu Kα1 radiation).

In some embodiments, the crystalline Sodium Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, or twenty-three XRPD signals selected from those set forth in Table 16.

TABLE 16
Sodium Type A XRPD Signals

| Signal | | d-spacing | Rel. Int. |
|---|---|---|---|
| No. | Pos. [°2θ] | [Å] | [%] |
| 1 | 4.17 | 21.2 | 40.55 |
| 2 | 4.85 | 18.21 | 38.68 |
| 3 | 6.27 | 14.09 | 45.92 |
| 4 | 8.37 | 10.56 | 52.65 |
| 5 | 8.91 | 9.92 | 46.59 |
| 6 | 9.35 | 9.46 | 55.71 |
| 7 | 10.04 | 8.81 | 100 |
| 8 | 11.69 | 7.57 | 18.98 |
| 9 | 12.44 | 7.11 | 53.24 |
| 10 | 13.62 | 6.5 | 30.72 |
| 11 | 14.05 | 6.3 | 30.17 |
| 12 | 15.9 | 5.57 | 24.53 |
| 13 | 16.47 | 5.38 | 68.95 |
| 14 | 18.16 | 4.88 | 32.38 |
| 15 | 19.41 | 4.57 | 51.23 |
| 16 | 19.97 | 4.45 | 36.68 |
| 17 | 21.41 | 4.15 | 45.34 |
| 18 | 21.69 | 4.1 | 58.86 |
| 19 | 22.13 | 4.02 | 35.37 |
| 20 | 24.27 | 3.67 | 51.7 |
| 21 | 25.96 | 3.43 | 14.49 |
| 22 | 28.14 | 3.17 | 4.87 |
| 23 | 30.23 | 2.96 | 5.4 |

### Potassium Type B

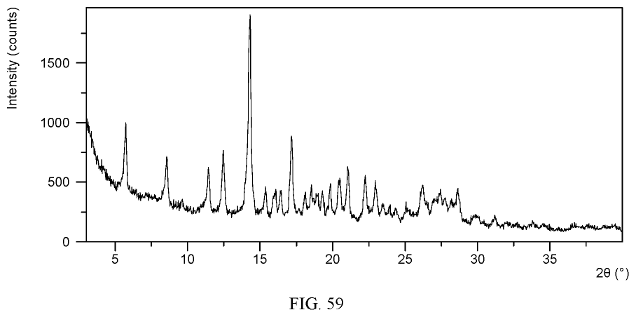

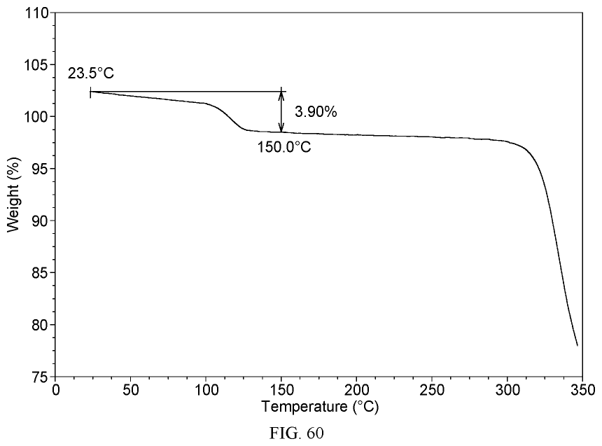

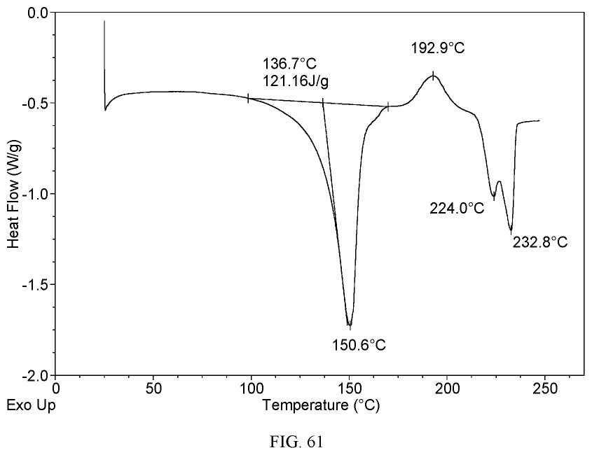

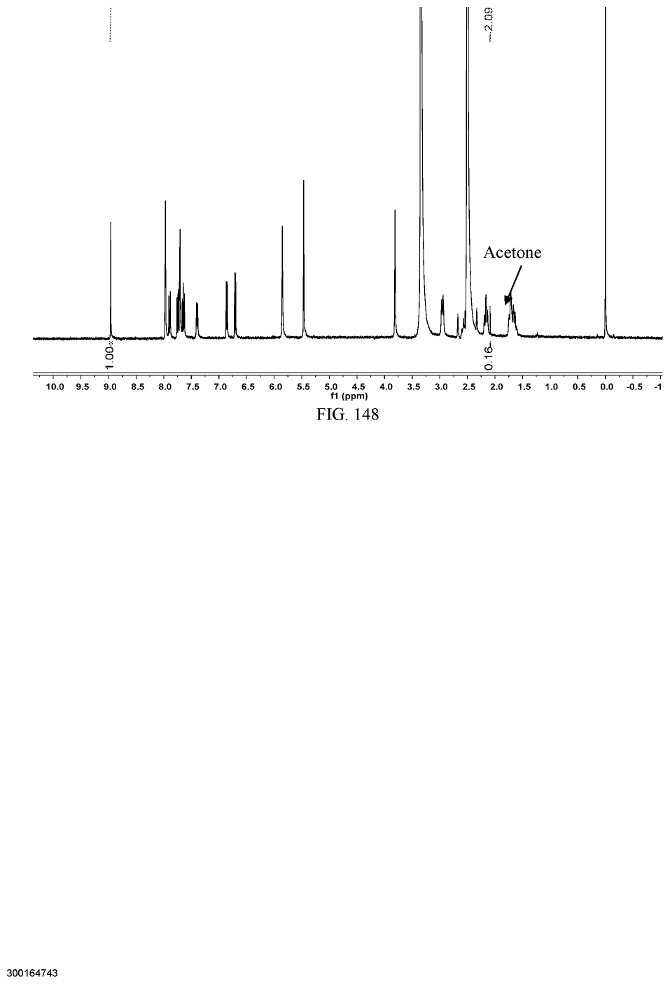

In some embodiments, the present disclosure provides solid forms of Potassium Type B, e.g., crystalline forms of Potassium Type B. In some embodiments, the Potassium Type B XRPD profile is substantially similar to that shown in FIG. 59. In some embodiments, the Potassium Type B TGA profile is substantially similar to that shown in FIG. 60. In some embodiments, the Potassium Type B DSC profile is substantially similar to that shown in FIG. 61. In some embodiments, the Potassium Type B ¹H NMR profile is substantially similar to that shown in FIG. 148. In some embodiments, the Potassium Type B is characterized by a DSC curve having endotherms at about 150.6° C., about 224.0° C., and about 232.8° C.

In some embodiments, the solid form of Potassium Type B is crystalline Potassium Type B characterized by two or more, or three XRPD signals selected from the group consisting of 14.3 °2θ, 17.2 °2θ, and 5.8 °2θ (±0.2 °2θ; ±0.1 °2θ; or ±0.0 °2θ; Cu Kα1 radiation). In some embodiments, the solid form of Potassium Type B is crystalline Potassium Type B characterized by XRPD signals at 14.3 °2θ, 17.2 °2θ, and 5.8 °2θ (±0.2 °2θ; ±0.1 °2θ; or ±0.0 °2θ; Cu Kα1 radiation).

In some embodiments, the solid form of Potassium Type B is crystalline Potassium Type B characterized by two or more, or three or more XRPD signals selected from the group consisting of 14.3 °2θ, 17.2 °2θ, 5.8 °2θ, 12.5 °2θ, and 8.6 °2θ (±0.2 °2θ; ±0.1 °2θ; or ±0.0 °2θ; Cu Kα1 radiation). In some embodiments, the solid form of Potassium Type B is crystalline Potassium Type B characterized by XRPD signals at 14.3 °2θ, 17.2 °2θ, 5.8 °2θ, 12.5 °2θ, and 8.6 °2θ (±0.2 °2θ; ±0.1 °2θ; or ±0.0 °2θ; Cu Kα1 radiation).

In some embodiments, the solid form of Potassium Type B is crystalline Potassium Type B characterized by two or more, or three or more XRPD signals selected from the group consisting of 14.3 °2θ, 17.2 °2θ, 5.8 °2θ, 12.5 °2θ, 8.6 °2θ, 21.1 °2θ, and 11.4 °2θ (±0.2 °2θ; ±0.1 °2θ; or ±0.0 °2θ; Cu Kα1 radiation). In some embodiments, the solid form of Potassium Type B is crystalline Potassium Type B characterized by XRPD signals at 14.3 °2θ, 17.2 °2θ, 5.8 °2θ, 12.5 °2θ, 8.6 °2θ, 21.1 °2θ, and 11.4 °2θ (±0.2 °2θ; ±0.1 °2θ; or ±0.0 °2θ; Cu Kα1 radiation).

In some embodiments, the solid form of Potassium Type B is crystalline Potassium Type B characterized by two or more, or three or more XRPD signals selected from the group consisting of 14.3 °2θ, 17.2 °2θ, 5.8 °2θ, 12.5 °2θ, 8.6 °2θ, 21.1 °2θ, 11.4 °2θ, 22.3 °2θ, 20.5 °2θ, and 23.0 °2θ (±0.2 °2θ; ±0.1 °2θ; or ±0.0 °2θ; Cu Kα1 radiation). In some embodiments, the solid form of Potassium Type B is crystalline Potassium Type B characterized by XRPD signals at 14.3 °2θ, 17.2 °2θ, 5.8 °2θ, 12.5 °2θ, 8.6 °2θ, 21.1 °2θ, 11.4 °2θ, 22.3 °2θ, 20.5 °2θ, and 23.0 °2θ (±0.2 °2θ; ±0.1 °2θ; or ±0.0 °2θ; Cu Kα1 radiation).

In some embodiments, the crystalline Potassium Type B is characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 17.

TABLE 17
Potassium Type B XRPD Signals

| Signal | | d-spacing | Rel. Int. |
|---|---|---|---|
| No. | Pos. [°2θ] | [Å] | [%] |
| 1 | 5.75 | 15.38 | 34.45 |
| 2 | 8.56 | 10.33 | 25.25 |
| 3 | 11.44 | 7.73 | 22.87 |
| 4 | 12.47 | 7.1 | 31.45 |
| 5 | 14.31 | 6.19 | 100 |
| 6 | 15.4 | 5.75 | 13.09 |
| 7 | 16.03 | 5.53 | 10.32 |
| 8 | 16.42 | 5.4 | 11.04 |
| 9 | 17.17 | 5.17 | 39.97 |
| 10 | 18.09 | 4.9 | 10.61 |
| 11 | 18.53 | 4.79 | 13.82 |
| 12 | 19.31 | 4.6 | 12.1 |
| 13 | 19.85 | 4.47 | 16.91 |
| 14 | 20.52 | 4.33 | 19.15 |
| 15 | 21.07 | 4.22 | 24.23 |
| 16 | 22.27 | 3.99 | 20.29 |
| 17 | 22.95 | 3.87 | 18.71 |
| 18 | 25.11 | 3.55 | 5.81 |
| 19 | 26.2 | 3.4 | 17.83 |
| 20 | 28.66 | 3.12 | 16.37 |
| 21 | 29.89 | 2.99 | 4.02 |
| 22 | 31.18 | 2.87 | 4.05 |

### Choline Salt Type A

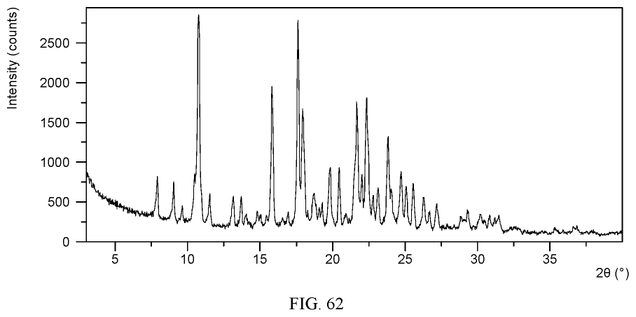

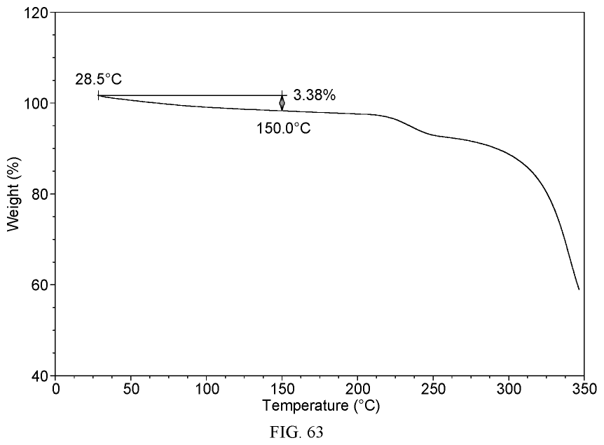

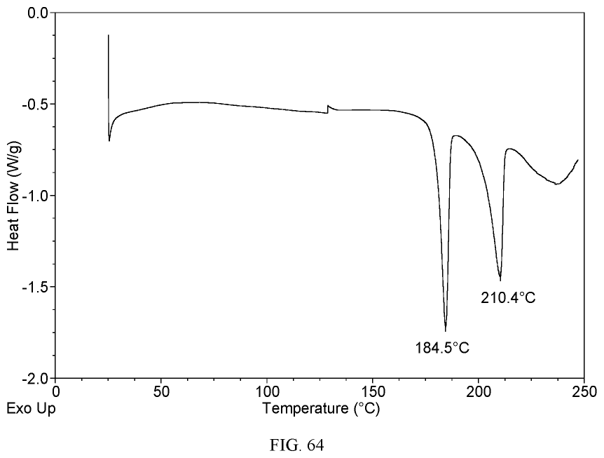

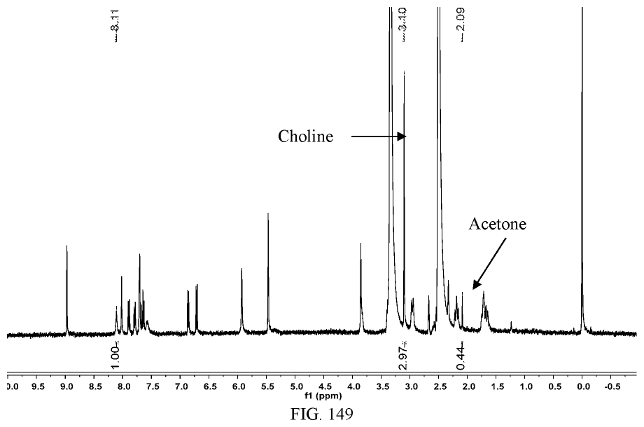

In some embodiments, the present disclosure provides solid forms of Choline Type A, e.g., crystalline forms of Choline Type A. In some embodiments, the Choline Type A XRPD profile is substantially similar to that shown in FIG. 62. In some embodiments, the Choline Type A TGA profile is substantially similar to that shown in FIG. 63. In some embodiments, the Choline Type A DSC profile is substantially similar to that shown in FIG. 64. In some embodiments, the Choline Type A ¹H NMR profile is substantially similar to that shown in FIG. 149. In some embodiments, the Choline Type A is characterized by a DSC curve having endotherms at about 184.5° C. and about 210.4° C.

In some embodiments, the solid form of Choline Type A is crystalline Choline Type A characterized by two or more, or three XRPD signals selected from the group consisting of 10.8 °2θ, 17.6 °2θ, and 15.8 °2θ (±0.2 °2θ; ±0.1 °2θ; or ±0.0 °2θ; Cu Kα1 radiation). In some embodiments, the solid form of Choline Type A is crystalline Choline Type A characterized by XRPD signals at 10.8 °2θ, 17.6 °2θ, and 15.8 °2θ (±0.2 °2θ; ±0.1 °2θ; or ±0.0 °2θ; Cu Kα1 radiation).

In some embodiments, the solid form of Choline Type A is crystalline Choline Type A characterized by two or more, or three or more XRPD signals selected from the group consisting of 10.8 °2θ, 17.6 °2θ, 15.8 °2θ, 22.3 °2θ, and 21.7 °2θ (±0.2 °2θ; ±0.1 °2θ; or ±0.0 °2θ; Cu Kα1 radiation). In some embodiments, the solid form of Choline Type A is crystalline Choline Type A characterized by XRPD signals at 10.8 °2θ, 17.6 °2θ, 15.8 °2θ, 22.3 °2θ, and 21.7 °2θ (±0.2 °2θ; ±0.1 °2θ; or ±0.0 °2θ; Cu Kα1 radiation).

In some embodiments, the solid form of Choline Type A is crystalline Choline Type A characterized by two or more, or three or more XRPD signals selected from the group consisting of 10.8 °2θ, 17.6 °2θ, 15.8 °2θ, 22.3 °2θ, 21.7 °2θ, 17.9 °2θ, and 23.8 °2θ (±0.2 °2θ; ±0.1 °2θ; or ±0.0 °2θ; Cu Kα1 radiation). In some embodiments, the solid form of Choline Type A is crystalline Choline Type A characterized by XRPD signals at 10.8 °2θ, 17.6 °2θ, 15.8 °2θ, 22.3 °2θ, 21.7 °2θ, 17.9 °2θ, and 23.8 °2θ (±0.2 °2θ; ±0.1 °2θ; or ±0.0 °2θ; Cu Kα1 radiation).

In some embodiments, the solid form of Choline Type A is crystalline Choline Type A characterized by two or more, or three or more XRPD signals selected from the group consisting of 10.8 °2θ, 17.6 °2θ, 15.8 °2θ, 22.3 °2θ, 21.7 °2θ, 17.9 °2θ, 23.8 °2θ, 19.8 °2θ, 20.5 °2θ, and 24.7 °2θ (±0.2 °2θ; ±0.1 °2θ; or ±0.0 °2θ; Cu Kα1 radiation). In some embodiments, the solid form of Choline Type A is crystalline Choline Type A characterized by XRPD signals at 10.8 °2θ, 17.6 °2θ, 15.8 °2θ, 22.3 °2θ, 21.7 °2θ, 17.9 °2θ, 23.8 °2θ, 19.8 °2θ, 20.5 °2θ, and 24.7 °2θ (±0.2 °2θ; ±0.1 °2θ; or ±0.0 °2θ; Cu Kα1 radiation).

In some embodiments, the crystalline Choline Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, or thirty-seven XRPD signals selected from those set forth in Table 18.

TABLE 18
Choline Type A XRPD Signals

| Signal | | d-spacing | Rel. Int. |
|---|---|---|---|
| No. | Pos. [°2θ] | [Å] | [%] |
| 1 | 7.92 | 11.17 | 20.36 |
| 2 | 9.05 | 9.78 | 17.95 |
| 3 | 9.63 | 9.19 | 7.04 |
| 4 | 10.78 | 8.2 | 100 |
| 5 | 11.53 | 7.67 | 15.32 |
| 6 | 13.17 | 6.72 | 12.95 |
| 7 | 13.7 | 6.46 | 14.27 |
| 8 | 14.8 | 5.99 | 6.07 |
| 9 | 15.83 | 5.6 | 67.67 |
| 10 | 16.93 | 5.24 | 5.37 |
| 11 | 17.62 | 5.03 | 97.31 |
| 12 | 17.94 | 4.94 | 56.22 |
| 13 | 18.7 | 4.75 | 15.06 |
| 14 | 19.29 | 4.6 | 10.69 |
| 15 | 19.84 | 4.47 | 27.56 |
| 16 | 20.45 | 4.34 | 27.5 |
| 17 | 20.9 | 4.25 | 4.99 |
| 18 | 21.69 | 4.1 | 58.68 |
| 19 | 22.03 | 4.03 | 23.12 |
| 20 | 22.33 | 3.98 | 61.38 |
| 21 | 22.79 | 3.9 | 14.24 |
| 22 | 23.13 | 3.84 | 18.47 |
| 23 | 23.83 | 3.73 | 44.3 |
| 24 | 24.07 | 3.7 | 17.18 |
| 25 | 24.72 | 3.6 | 26.87 |
| 26 | 25.08 | 3.55 | 19.73 |
| 27 | 25.57 | 3.48 | 20.66 |
| 28 | 26.29 | 3.39 | 14.99 |
| 29 | 26.68 | 3.34 | 6.74 |
| 30 | 27.18 | 3.28 | 11.94 |
| 31 | 28.84 | 3.1 | 6.14 |
| 32 | 29.32 | 3.05 | 8.36 |
| 33 | 30.2 | 2.96 | 7.86 |
| 34 | 30.85 | 2.9 | 7.87 |
| 35 | 31.47 | 2.84 | 7.45 |
| 36 | 32.74 | 2.74 | 2.06 |
| 37 | 36.77 | 2.44 | 3.24 |

### Ammonium Type A

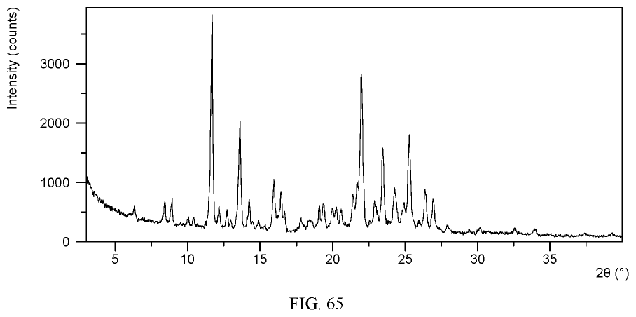

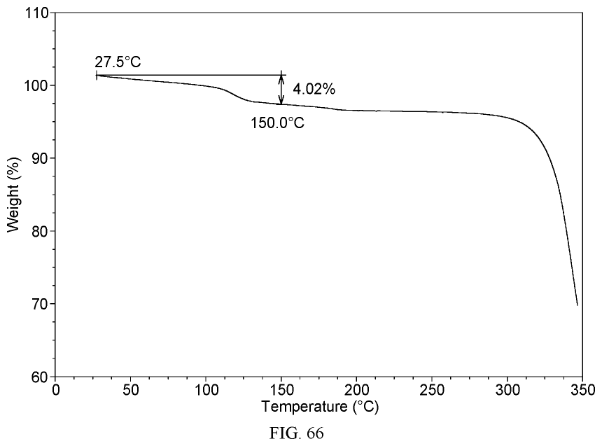

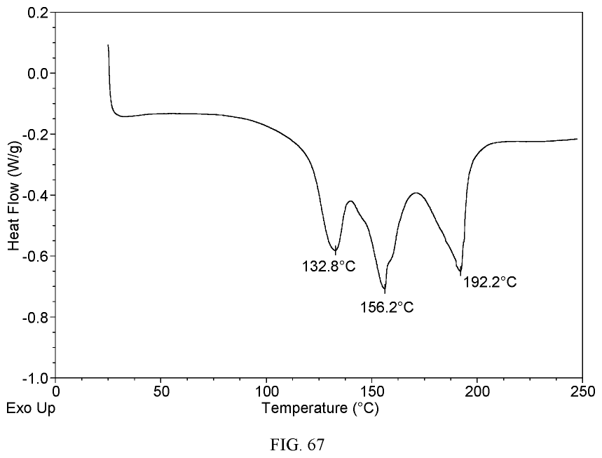

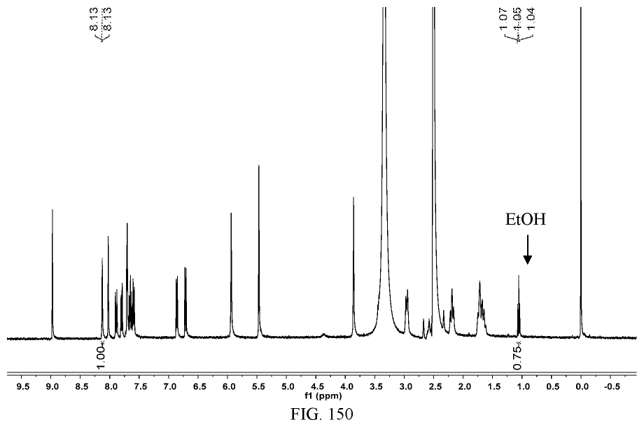

In some embodiments, the present disclosure provides solid forms of Ammonium Type A, e.g., crystalline forms of Ammonium Type A. In some embodiments, the Ammonium Type A XRPD profile is substantially similar to that shown in FIG. 65. In some embodiments, the Ammonium Type A TGA profile is substantially similar to that shown in FIG. 66. In some embodiments, the Ammonium Type A DSC profile is substantially similar to that shown in FIG. 67. In some embodiments, the Ammonium Type A ¹H NMR profile is substantially similar to that shown in FIG. 150. In some embodiments, the Ammonium Type A is characterized by a DSC curve having endotherms at about 132.8° C., about 156.2° C., and about 192.2° C.

In some embodiments, the solid form of Ammonium Type A is crystalline Ammonium Type A characterized by two or more, or three XRPD signals selected from the group consisting of 11.7 °2θ, 22.0 °2θ, and 13.6 °2θ (±0.2 °2θ; ±0.1 °2θ; or ±0.0 °2θ; Cu Kα1 radiation). In some embodiments, the solid form of Ammonium Type A is crystalline Ammonium Type A characterized by XRPD signals at 11.7 °2θ, 22.0 °2θ, and 13.6 °2θ (±0.2 °2θ; ±0.1 °2θ; or ±0.0 °2θ; Cu Kα1 radiation).

In some embodiments, the solid form of Ammonium Type A is crystalline Ammonium Type A characterized by two or more, or three or more XRPD signals selected from the group consisting of 11.7 °2θ, 22.0 °2θ, 13.6 °2θ, 25.3 °2θ, and 23.5 °2θ (±0.2 °2θ; ±0.1 °2θ; or ±0.0 °2θ; Cu Kα1 radiation). In some embodiments, the solid form of Ammonium Type A is crystalline Ammonium Type A characterized by XRPD signals at 11.7 °2θ, 22.0 °2θ, 13.6 °2θ, 25.3 °2θ, and 23.5 °2θ (±0.2 °2θ; ±0.1 °2θ; or ±0.0 °2θ; Cu Kα1 radiation).

In some embodiments, the solid form of Ammonium Type A is crystalline Ammonium Type A characterized by two or more, or three or more XRPD signals selected from the group consisting of 11.7 °2θ, 22.0 °2θ, 13.6 °2θ, 25.3 °2θ, 23.5 °2θ, 16.0 °2θ, and 21.7 °2θ (±0.2 °2θ; ±0.1 °2θ; or ±0.0 °2θ; Cu Kα1 radiation). In some embodiments, the solid form of Ammonium Type A is crystalline Ammonium Type A characterized by XRPD signals at 11.7 °2θ, 22.0 °2θ, 13.6 °2θ, 25.3 °2θ, 23.5 °2θ, 16.0 °2θ, and 21.7 °2θ (±0.2 °2θ; ±0.1 °2θ; or ±0.0 °2θ; Cu Kα1 radiation).

In some embodiments, the solid form of Ammonium Type A is crystalline Ammonium Type A characterized by two or more, or three or more XRPD signals selected from the group consisting of 11.7 °2θ, 22.0 °2θ, 13.6 °2θ, 25.3 °2θ, 23.5 °2θ, 16.0 °2θ, 21.7 °2θ, 26.4 °2θ, 24.3 °2θ, and 16.5 °2θ (±0.2 °2θ; ±0.1 °2θ; or ±0.0 °2θ; Cu Kα1 radiation). In some embodiments, the solid form of Ammonium Type A is crystalline Ammonium Type A characterized by XRPD signals at 11.7 °2θ, 22.0 °2θ, 13.6 °2θ, 25.3 °2θ, 23.5 °2θ, 16.0 °2θ, 21.7 °2θ, 26.4 °2θ, 24.3 °2θ, and 16.5 °2θ (±0.2 °2θ; ±0.1 °2θ; or ±0.0 °2θ; Cu Kα1 radiation).

In some embodiments, the crystalline Ammonium Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, or thirty-five XRPD signals selected from those set forth in Table 19.

TABLE 19
Ammonium Type A XRPD Signals

| Signal | | d-spacing | Rel. Int. |
|---|---|---|---|
| No. | Pos. [°2θ] | [Å] | [%] |
| 1 | 6.33 | 13.95 | 4.7 |
| 2 | 8.43 | 10.49 | 10.65 |
| 3 | 8.92 | 9.92 | 12.14 |
| 4 | 10.02 | 8.82 | 3.57 |
| 5 | 10.41 | 8.5 | 3.9 |
| 6 | 11.69 | 7.57 | 100 |
| 7 | 12.18 | 7.27 | 9.28 |
| 8 | 12.72 | 6.96 | 8.69 |
| 9 | 13.61 | 6.5 | 53.36 |
| 10 | 14.25 | 6.21 | 13.65 |
| 11 | 14.88 | 5.95 | 3.43 |
| 12 | 15.96 | 5.55 | 24.63 |
| 13 | 16.45 | 5.39 | 18.65 |
| 14 | 16.69 | 5.31 | 9.28 |
| 15 | 17.82 | 4.98 | 5.42 |
| 16 | 19.08 | 4.65 | 11.22 |
| 17 | 19.39 | 4.58 | 12.6 |
| 18 | 19.97 | 4.45 | 9.44 |
| 19 | 20.26 | 4.38 | 9.4 |
| 20 | 20.6 | 4.31 | 8.71 |
| 21 | 21.4 | 4.15 | 16.48 |
| 22 | 21.67 | 4.1 | 19.97 |
| 23 | 21.98 | 4.04 | 74.5 |
| 24 | 22.9 | 3.88 | 13.27 |
| 25 | 23.45 | 3.79 | 37.93 |
| 26 | 24.27 | 3.67 | 18.76 |
| 27 | 24.89 | 3.58 | 10.97 |
| 28 | 25.3 | 3.52 | 45.45 |
| 29 | 26.39 | 3.38 | 19.8 |
| 30 | 26.95 | 3.31 | 15.22 |
| 31 | 27.92 | 3.2 | 3.32 |
| 32 | 30.18 | 2.96 | 2.91 |
| 33 | 32.58 | 2.75 | 2.86 |
| 34 | 33.95 | 2.64 | 2.72 |
| 35 | 37.41 | 2.4 | 1.05 |

### Ammonium Type B

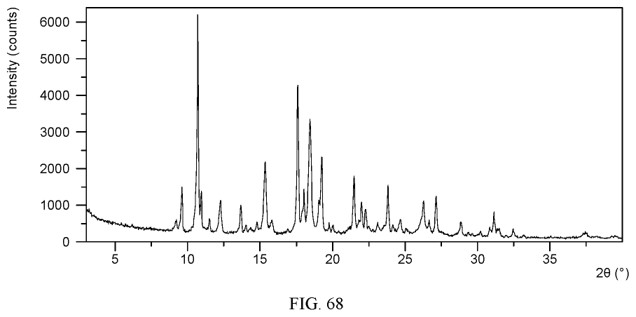

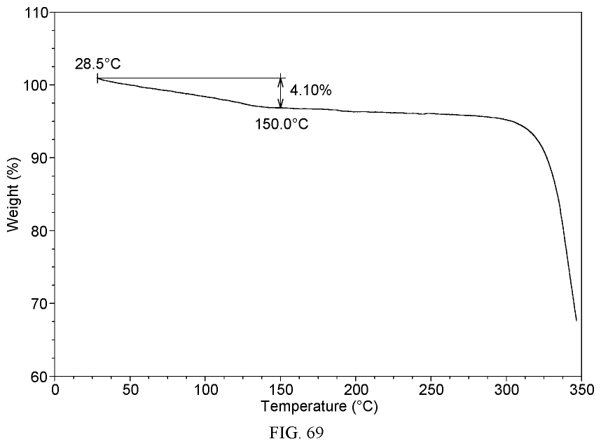

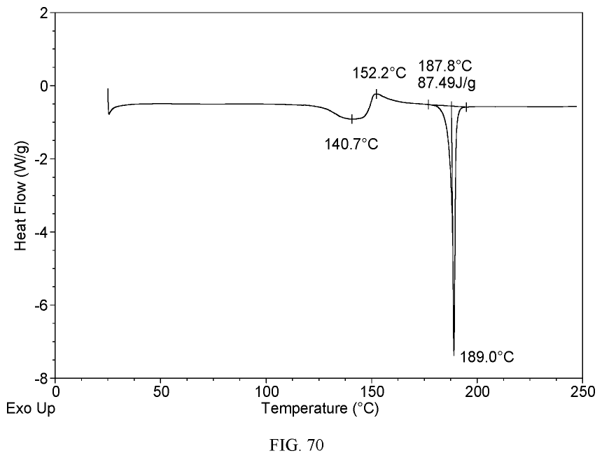

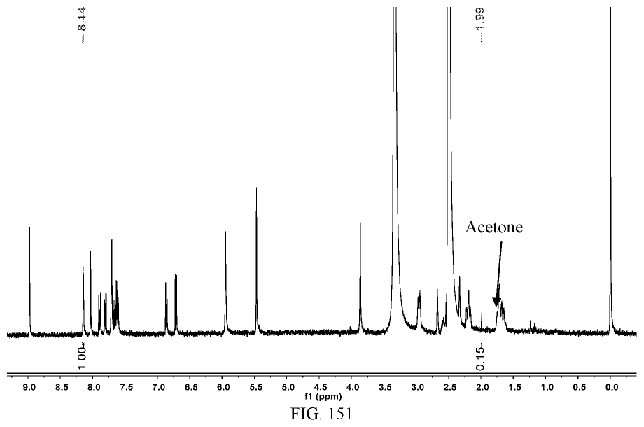

In some embodiments, the present disclosure provides solid forms of Ammonium Type B, e.g., crystalline forms of Ammonium Type B. In some embodiments, the Ammonium Type B XRPD profile is substantially similar to that shown in FIG. 68. In some embodiments, the Ammonium Type B TGA profile is substantially similar to that shown in FIG. 69. In some embodiments, the Ammonium Type B DSC profile is substantially similar to that shown in FIG. 70. In some embodiments, the Ammonium Type B ¹H NMR profile is substantially similar to that shown in FIG. 151. In some embodiments, the Ammonium Type B is characterized by a DSC curve having endotherms at about 140.7° C. and about 189.0° C.

In some embodiments, the solid form of Ammonium Type B is crystalline Ammonium Type B characterized by two or more, or three XRPD signals selected from the group consisting of 10.7 °2θ, 17.6 °2θ, and 18.4 °2θ (±0.2 °2θ; ±0.1 °2θ; or ±0.0 °2θ; Cu Kα1 radiation). In some embodiments, the solid form of Ammonium Type B is crystalline Ammonium Type B characterized by XRPD signals at 10.7 °2θ, 17.6 °2θ, and 18.4 °2θ (±0.2 °2θ; ±0.1 °2θ; or ±0.0 °2θ; Cu Kα1 radiation).

In some embodiments, the solid form of Ammonium Type B is crystalline Ammonium Type B characterized by two or more, or three or more XRPD signals selected from the group consisting of 10.7 °2θ, 17.6 °2θ, 18.4 °2θ, 19.3 °2θ, and 15.4 °2θ (±0.2 °2θ; ±0.1 °2θ; or ±0.0 °2θ; Cu Kα1 radiation). In some embodiments, the solid form of Ammonium Type B is crystalline Ammonium Type B characterized by XRPD signals at 10.7 °2θ, 17.6 °2θ, 18.4 °2θ, 19.3 °2θ, and 15.4 °2θ (±0.2 °2θ; ±0.1 °2θ; or ±0.0 °2θ; Cu Kα1 radiation).

In some embodiments, the solid form of Ammonium Type B is crystalline Ammonium Type B characterized by two or more, or three or more XRPD signals selected from the group consisting of 10.7 °2θ, 17.6 °2θ, 18.4 °2θ, 19.3 °2θ, 15.4 °2θ, 21.5 °2θ, and 23.8 °2θ (±0.2 °2θ; ±0.1 °2θ; or ±0.0 °2θ; Cu Kα1 radiation). In some embodiments, the solid form of Ammonium Type B is crystalline Ammonium Type B characterized by XRPD signals at 10.7 °2θ, 17.6 °2θ, 18.4 °2θ, 19.3 °2θ, 15.4 °2θ, 21.5 °2θ, and 23.8 °2θ (±0.2 °2θ; ±0.1 °2θ; or ±0.0 °2θ; Cu Kα1 radiation).

In some embodiments, the solid form of Ammonium Type B is crystalline Ammonium Type B characterized by two or more, or three or more XRPD signals selected from the group consisting of 10.7 °2θ, 17.6 °2θ, 18.4 °2θ, 19.3 °2θ, 15.4 °2θ, 21.5 °2θ, 23.8 °2θ, 18.0 °2θ, 9.6 °2θ, and 10.9 °2θ (±0.2 °2θ; ±0.1 °2θ; or ±0.0 °2θ; Cu Kα1 radiation). In some embodiments, the solid form of Ammonium Type B is crystalline Ammonium Type B characterized by XRPD signals at 10.7 °2θ, 17.6 °2θ, 18.4 °2θ, 19.3 °2θ, 15.4 °2θ, 21.5 °2θ, 23.8 °2θ, 18.0 °2θ, 9.6 °2θ, and 10.9 °2θ (±0.2 °2θ; ±0.1 °2θ; or ±0.0 °2θ; Cu Kα1 radiation).

In some embodiments, the crystalline Ammonium Type B is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, or thirty-seven XRPD signals selected from those set forth in Table 20.

TABLE 20
Ammonium Type B XRPD Signals

| Signal | | d-spacing | Rel. Int. |
|---|---|---|---|
| No. | Pos. [°2θ] | [Å] | [%] |
| 1 | 9.21 | 9.6 | 4.91 |
| 2 | 9.61 | 9.21 | 19.14 |
| 3 | 10.71 | 8.26 | 100 |
| 4 | 10.94 | 8.09 | 19.06 |
| 5 | 11.52 | 7.68 | 6.1 |
| 6 | 12.27 | 7.21 | 14.97 |
| 7 | 13.68 | 6.47 | 12.92 |
| 8 | 14.02 | 6.32 | 3.65 |
| 9 | 14.79 | 5.99 | 5.13 |
| 10 | 15.36 | 5.77 | 32.48 |
| 11 | 15.84 | 5.59 | 5.8 |
| 12 | 17.6 | 5.04 | 68.34 |
| 13 | 18.03 | 4.92 | 19.93 |
| 14 | 18.44 | 4.81 | 53.03 |
| 15 | 19.05 | 4.66 | 14.56 |
| 16 | 19.26 | 4.61 | 35.79 |
| 17 | 19.77 | 4.49 | 4.28 |
| 18 | 20.02 | 4.43 | 3.56 |
| 19 | 21.48 | 4.14 | 26.33 |
| 20 | 22 | 4.04 | 14.27 |
| 21 | 22.28 | 3.99 | 11.11 |
| 22 | 23.1 | 3.85 | 4.56 |
| 23 | 23.82 | 3.74 | 22.4 |
| 24 | 24.14 | 3.69 | 4.06 |
| 25 | 24.69 | 3.61 | 6.48 |
| 26 | 25.11 | 3.55 | 2.25 |
| 27 | 26.28 | 3.39 | 15.45 |
| 28 | 26.66 | 3.34 | 6.86 |
| 29 | 27.14 | 3.29 | 18 |
| 30 | 28.86 | 3.09 | 6.58 |
| 31 | 30.2 | 2.96 | 2.08 |
| 32 | 30.83 | 2.9 | 4.39 |
| 33 | 31.13 | 2.87 | 11.47 |
| 34 | 31.47 | 2.84 | 3.63 |
| 35 | 32.45 | 2.76 | 3.84 |
| 36 | 33.2 | 2.7 | 1.12 |
| 37 | 37.49 | 2.4 | 2.5 |

### Tris Type A

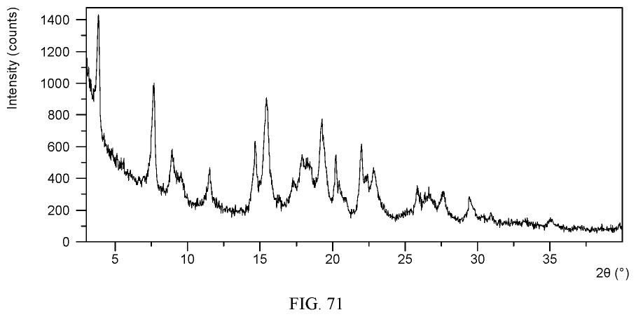

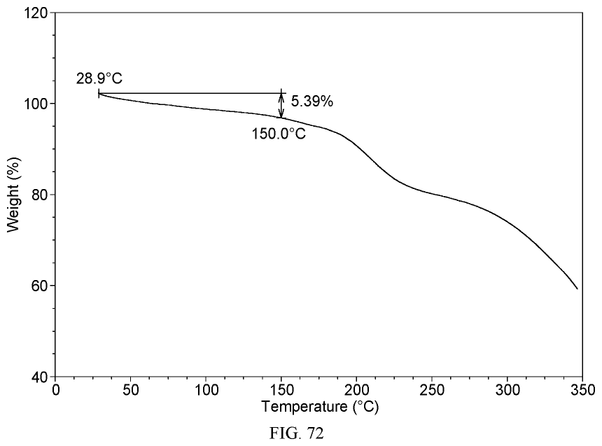

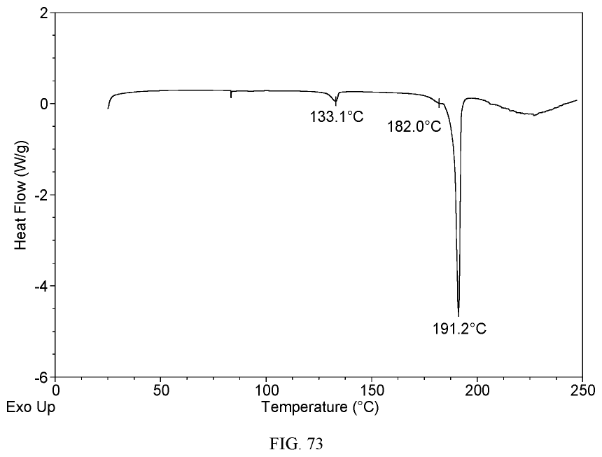

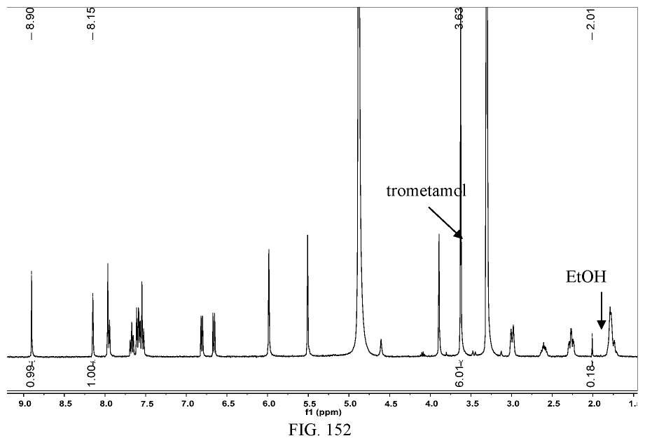

In some embodiments, the present disclosure provides solid forms of Tris Type A, e.g., crystalline forms of Tris Type A. In some embodiments, the Tris Type A XRPD profile is substantially similar to that shown in FIG. 71. In some embodiments, the Tris Type A TGA profile is substantially similar to that shown in FIG. 72. In some embodiments, the Tris Type A DSC profile is substantially similar to that shown in FIG. 73. In some embodiments, the Tris Type A ¹H NMR profile is substantially similar to that shown in FIG. 152. In some embodiments, the Tris Type A is characterized by a DSC curve having endotherms at about 133.1° C., about 182.0, and about 191.2° C.

In some embodiments, the solid form of Tris Type A is crystalline Tris Type A characterized by two or more, or three XRPD signals selected from the group consisting of 3.9 °2θ, 15.5 °2θ, and 7.7 °2θ (±0.2 °2θ; ±0.1 °2θ; or ±0.0 °2θ; Cu Kα1 radiation). In some embodiments, the solid form of Tris Type A is crystalline Tris Type A characterized by XRPD signals at 3.9 °2θ, 15.5 °2θ, and 7.7 °2θ (±0.2 °2θ; ±0.1 °2θ; or ±0.0 °2θ; Cu Kα1 radiation).

In some embodiments, the solid form of Tris Type A is crystalline Tris Type A characterized by two or more, or three or more XRPD signals selected from the group consisting of 3.9 °2θ, 15.5 °2θ, 7.7 °2θ, 19.2 °2θ, and 14.7 °2θ (±0.2 °2θ; ±0.1 °2θ; or ±0.0 °2θ; Cu Kα1 radiation). In some embodiments, the solid form of Tris Type A is crystalline Tris Type A characterized by XRPD signals at 3.9 °2θ, 15.5 °2θ, 7.7 °2θ, 19.2 °2θ, and 14.7 °2θ (±0.2 °2θ; ±0.1 °2θ; or ±0.0 °2θ; Cu Kα1 radiation).

In some embodiments, the solid form of Tris Type A is crystalline Tris Type A characterized by two or more, or three or more XRPD signals selected from the group consisting of 3.9 °2θ, 15.5 °2θ, 7.7 °2θ, 19.2 °2θ, 14.7 °2θ, 22.0 °2θ, and 20.2 °2θ (±0.2 °2θ; ±0.1 °2θ; or ±0.0 °2θ; Cu Kα1 radiation). In some embodiments, the solid form of Tris Type A is crystalline Tris Type A characterized by XRPD signals at 3.9 °2θ, 15.5 °2θ, 7.7 °2θ, 19.2 °2θ, 14.7 °2θ, 22.0 °2θ, and 20.2 °2θ (±0.2 °2θ; ±0.1 °2θ; or ±0.0 °2θ; Cu Kα1 radiation).

In some embodiments, the solid form of Tris Type A is crystalline Tris Type A characterized by two or more, or three or more XRPD signals selected from the group consisting of 3.9 °2θ, 15.5 °2θ, 7.7 °2θ, 19.2 °2θ, 14.7 °2θ, 22.0 °2θ, 20.2 °2θ, 17.9 °2θ, 22.9 °2θ, and 8.9 °2θ (±0.2 °2θ; ±0.1 °2θ; or ±0.0 °2θ; Cu Kα1 radiation). In some embodiments, the solid form of Tris Type A is crystalline Tris Type A characterized by XRPD signals at 3.9 °2θ, 15.5 °2θ, 7.7 °2θ, 19.2 °2θ, 14.7 °2θ, 22.0 °2θ, 20.2 °2θ, 17.9 °2θ, 22.9 °2θ, and 8.9 °2θ (±0.2 °2θ; ±0.1 °2θ; or ±0.0 °2θ; Cu Kα1 radiation).

In some embodiments, the crystalline Tris Type A is characterized by one, two, three, four, five, six, seven, eight, nine, ten, eleven, twelve, thirteen, fourteen, or fifteen XRPD signals selected from those set forth in Table 21.

TABLE 21
Tris Type A XRPD Signals

| | | d-spacing | Rel. Int. |
|---|---|---|---|
| Signal No. | Pos. [°2θ] | [Å] | [%] |
| 1 | 3.85 | 22.95 | 100 |
| 2 | 7.67 | 11.52 | 89.38 |
| 3 | 8.92 | 9.91 | 38.57 |
| 4 | 11.52 | 7.68 | 32.03 |
| 5 | 14.66 | 6.04 | 64.39 |
| 6 | 15.5 | 5.72 | 93.86 |
| 7 | 17.88 | 4.96 | 47.76 |
| 8 | 19.24 | 4.61 | 80.3 |
| 9 | 20.22 | 4.39 | 52.13 |
| 10 | 22 | 4.04 | 63.89 |
| 11 | 22.85 | 3.89 | 40.7 |
| 12 | 25.84 | 3.45 | 25.85 |
| 13 | 27.66 | 3.23 | 24.58 |
| 14 | 29.52 | 3.03 | 19.16 |
| 15 | 35.15 | 2.55 | 5.43 |

### Tris Type B

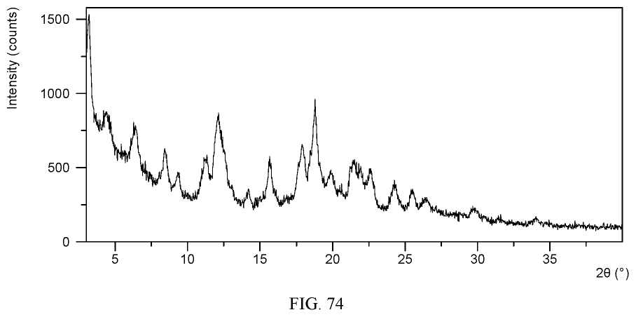

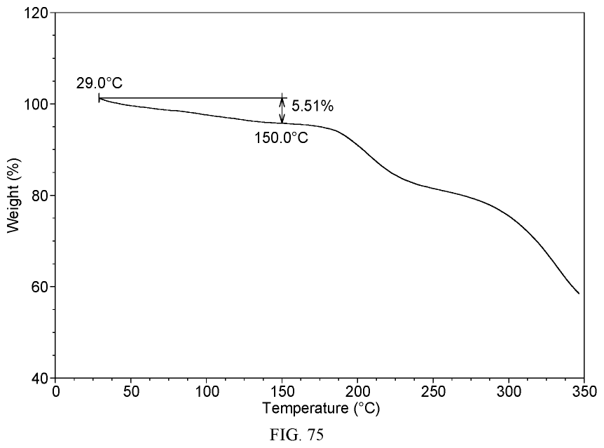

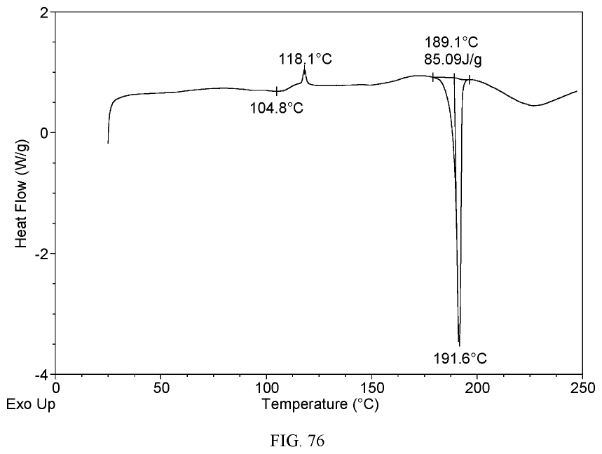

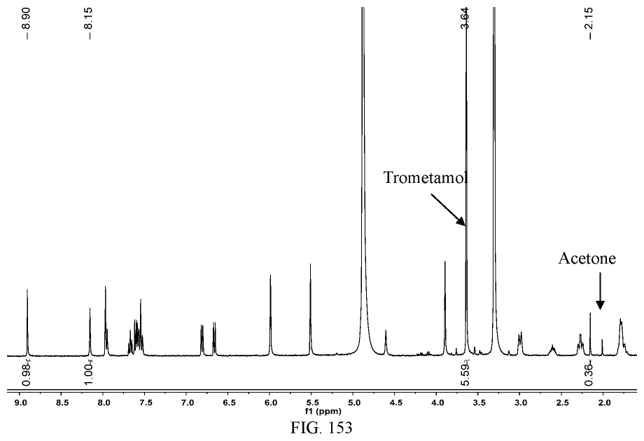

In some embodiments, the present disclosure provides solid forms of Tris Type B, e.g., crystalline forms of Tris Type B. In some embodiments, the Tris Type B XRPD profile is substantially similar to that shown in FIG. 74. In some embodiments, the Tris Type B TGA profile is substantially similar to that shown in FIG. 75. In some embodiments, the Tris Type B DSC profile is substantially similar to that shown in FIG. 76. In some embodiments, the Tris Type B ¹H NMR profile is substantially similar to that shown in FIG. 153. In some embodiments, the Tris Type B is characterized by a DSC curve having endotherms at about 104.8° C. and about 191.6° C.

In some embodiments, the solid form of Tris Type B is crystalline Tris Type B characterized by two or more, or three XRPD signals selected from the group consisting of 18.8 °2θ, 12.1 °2θ, and 17.9 °2θ (±0.2 °2θ; ±0.1 °2θ; or ±0.0 °2θ; Cu Kα1 radiation). In some embodiments, the solid form of Tris Type B is crystalline Tris Type B characterized by XRPD signals at 18.8 °2θ, 12.1 °2θ, and 17.9 °2θ (±0.2 °2θ; ±0.1 °2θ; or ±0.0 °2θ; Cu Kα1 radiation).

In some embodiments, the solid form of Tris Type B is crystalline Tris Type B characterized by two or more, or three or more XRPD signals selected from the group consisting of 18.8 °2θ, 12.1 °2θ, 17.9 °2θ, 15.7 °2θ, and 21.4 °2θ (±0.2 °2θ; ±0.1 °2θ; or ±0.0 °2θ; Cu Kα1 radiation). In some embodiments, the solid form of Tris Type B is crystalline Tris Type B characterized by XRPD signals at 18.8 °2θ, 12.1 °2θ, 17.9 °2θ, 15.7 °2θ, and 21.4 °2θ (±0.2 °2θ; ±0.1 °2θ; or ±0.0 °2θ; Cu Kα1 radiation).

In some embodiments, the solid form of Tris Type B is crystalline Tris Type B characterized by two or more, or three or more XRPD signals selected from the group consisting of 18.8 °2θ, 12.1 °2θ, 17.9 °2θ, 15.7 °2θ, 21.4 °2θ, 11.3 °2θ, and 6.5 °2θ (±0.2 °2θ; ±0.1 °2θ; or ±0.0 °2θ; Cu Kα1 radiation). In some embodiments, the solid form of Tris Type B is crystalline Tris Type B characterized by XRPD signals at 18.8 °2θ, 12.1 °2θ, 17.9 °2θ, 15.7 °2θ, 21.4 °2θ, 11.3 °2θ, and 6.5 °2θ (±0.2 °2θ; ±0.1 °2θ; or ±0.0 °2θ; Cu Kα1 radiation).

In some embodiments, the solid form of Tris Type B is crystalline Tris Type B characterized by two or more, or three or more XRPD signals selected from the group consisting of 18.8 °2θ, 12.1 °2θ, 17.9 °2θ, 15.7 °2θ, 21.4 °2θ, 11.3 °2θ, 6.5 °2θ, 22.6 °2θ, 8.4 °2θ, and 19.9 °2θ (±0.2 °2θ; ±0.1 °2θ; or ±0.0 °2θ; Cu Kα1 radiation). In some embodiments, the solid form of Tris Type B is crystalline Tris Type B characterized by XRPD signals at 18.8 °2θ, 12.1 °2θ, 17.9 °2θ, 15.7 °2θ, 21.4 °2θ, 11.3 °2θ, 6.5 °2θ, °2θ, 8.4 °2θ, and 19.9 °2θ (±0.2 °2θ; ±0.1 °2θ; or ±0.0 °2θ; Cu Kα1 radiation).

In some embodiments, the crystalline Tris Type B is characterized by one, two, three, four, five, six, seven, eight, nine, ten, eleven, twelve, thirteen, fourteen, fifteen, sixteen, or seventeen XRPD signals selected from those set forth in Table 22.

TABLE 22
Tris Type B XRPD Signals

| | | d-spacing | Rel. Int. |
|---|---|---|---|
| Signal No. | Pos. [°2θ] | [Å] | [%] |
| 1 | 4.45 | 19.84 | 25.65 |
| 2 | 6.45 | 13.7 | 41.01 |
| 3 | 8.44 | 10.47 | 37.98 |
| 4 | 9.35 | 9.45 | 19.89 |
| 5 | 11.25 | 7.87 | 42.35 |
| 6 | 12.1 | 7.31 | 88.88 |
| 7 | 14.18 | 6.25 | 10.55 |
| 8 | 15.67 | 5.66 | 46.03 |
| 9 | 17.93 | 4.95 | 59.84 |
| 10 | 18.79 | 4.72 | 100 |
| 11 | 19.91 | 4.46 | 32.87 |
| 12 | 21.35 | 4.16 | 42.78 |
| 13 | 22.63 | 3.93 | 38.7 |
| 14 | 24.28 | 3.67 | 24.75 |
| 15 | 25.44 | 3.5 | 20.85 |
| 16 | 26.44 | 3.37 | 14.47 |
| 17 | 29.74 | 3 | 11.02 |

### Tris Salt Type C

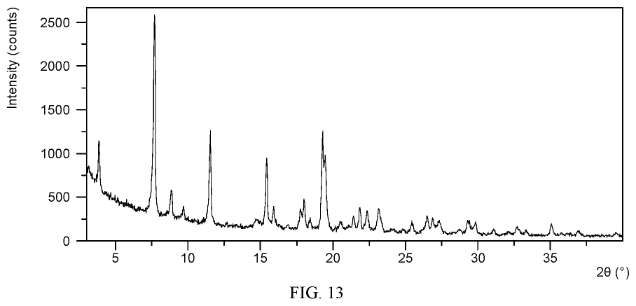

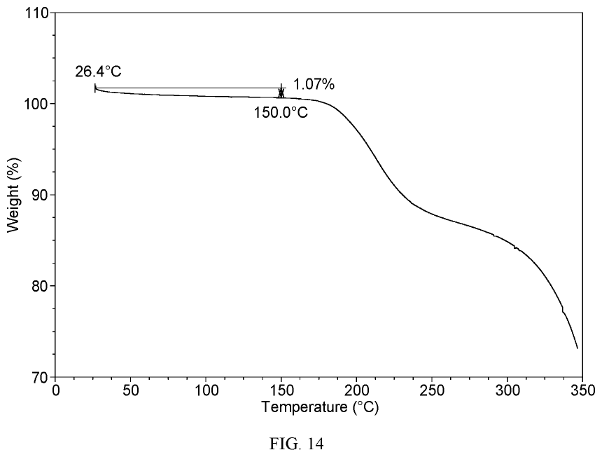

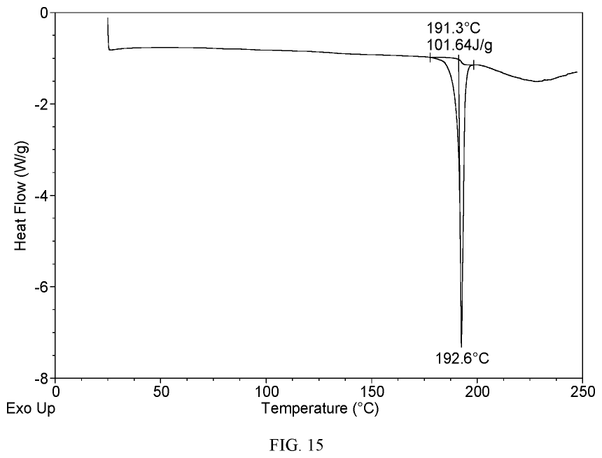

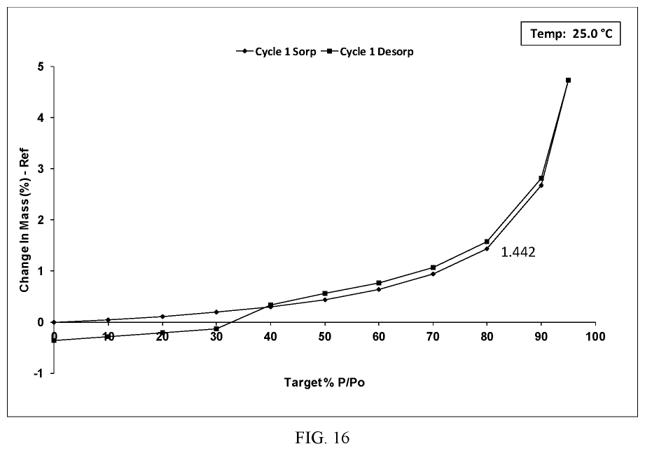

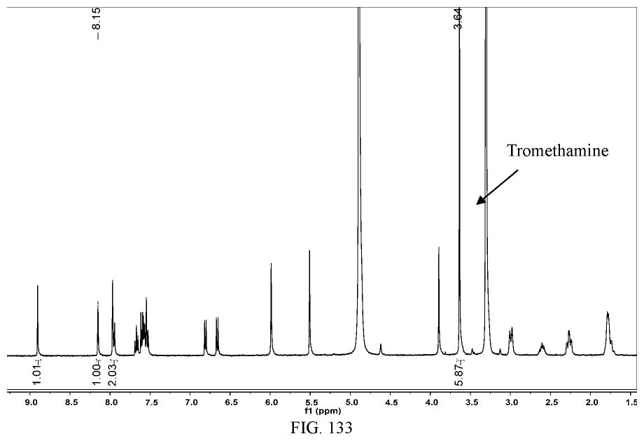

In some embodiments, the present disclosure provides solid forms of Tris Type C, e.g., crystalline forms of Tris Type C. In some embodiments, the Tris Type C XRPD profile is substantially similar to that shown in FIG. 13. In some embodiments, the Tris Type C TGA profile is substantially similar to that shown in FIG. 14. In some embodiments, the Tris Type C DSC profile is substantially similar to that shown in FIG. 15. In some embodiments, the Tris Type C DVS profile is substantially similar to that shown in FIG. 16. In some embodiments, the Tris Type C ¹H NMR profile is substantially similar to that shown in FIG. 133. In some embodiments, the Tris Type C is characterized by a DSC curve having an endotherm at about 192.6° C.

In some embodiments, the solid form of Tris Type C is crystalline Tris Type C characterized by two or more, or three XRPD signals selected from the group consisting of 7.7 °2θ, 19.3 °2θ, and 11.6 °2θ (±0.2 °2θ; ±0.1 °2θ; or ±0.0 °2θ; Cu Kα1 radiation). In some embodiments, the solid form of Tris Type C is crystalline Tris Type C characterized by XRPD signals at 7.7 °2θ, 19.3 °2θ, and 11.6 °2θ (±0.2 °2θ; ±0.1 °2θ; or ±0.0 °2θ; Cu Kα1 radiation).

In some embodiments, the solid form of Tris Type C is crystalline Tris Type C characterized by two or more, or three or more XRPD signals selected from the group consisting of 7.7 °2θ, 19.3 °2θ, 11.6 °2θ, 19.5 °2θ, and 15.4 °2θ (±0.2 °2θ; ±0.1 °2θ; or ±0.0 °2θ; Cu Kα1 radiation). In some embodiments, the solid form of Tris Type C is crystalline Tris Type C characterized by XRPD signals at 7.7 °2θ, 19.3 °2θ, 11.6 °2θ, 19.5 °2θ, and 15.4 °2θ (±0.2 °2θ; ±0.1 °2θ; or ±0.0 °2θ; Cu Kα1 radiation).

In some embodiments, the solid form of Tris Type C is crystalline Tris Type C characterized by two or more, or three or more XRPD signals selected from the group consisting of 7.7 °2θ, 19.3 °2θ, 11.6 °2θ, 19.5 °2θ, 15.4 °2θ, 3.9 °2θ, and 18.0 °2θ (±0.2 °2θ; ±0.1 °2θ; or ±0.0 °2θ; Cu Kα1 radiation). In some embodiments, the solid form of Tris Type C is crystalline Tris Type C characterized by XRPD signals at 7.7 °2θ, 19.3 °2θ, 11.6 °2θ, 19.5 °2θ, 15.4 °2θ, 3.9 °2θ, and 18.0 °2θ (±0.2 °2θ; ±0.1 °2θ; or ±0.0 °2θ; Cu Kα1 radiation).

In some embodiments, the solid form of Tris Type C is crystalline Tris Type C characterized by two or more, or three or more XRPD signals selected from the group consisting of 27.7 °2θ, 19.3 °2θ, 11.6 °2θ, 19.5 °2θ, 15.4 °2θ, 3.9 °2θ, 18.0 °2θ, 8.9 °2θ, 21.9 °2θ, and 23.1 °2θ (±0.2 °2θ; ±0.1 °2θ; or ±0.0 °2θ; Cu Kα1 radiation). In some embodiments, the solid form of Tris Type C is crystalline Tris Type C characterized by XRPD signals at 7.7 °2θ, 19.3 °2θ, 11.6 °2θ, 19.5 °2θ, 15.4 °2θ, 3.9 °2θ, 18.0 °2θ, 8.9 °2θ, 21.9 °2θ, and 23.1 °2θ (±0.2 °2θ; ±0.1 °2θ; or ±0.0 °2θ; Cu Kα1 radiation).

In some embodiments, the crystalline Tris Type C is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five XRPD signals selected from those set forth in Table 23.

TABLE 23
Tris Salt Type C XRPD Signals

| Signal No. | Pos. [°2θ] | d-spacing [Å] | Rel. Int. [%] |
|---|---|---|---|
| 1 | 3.86 | 22.88 | 24.17 |
| 2 | 7.7 | 11.49 | 100 |
| 3 | 8.86 | 9.98 | 13.65 |
| 4 | 9.68 | 9.14 | 5.98 |
| 5 | 11.55 | 7.66 | 46 |
| 6 | 14.75 | 6.01 | 4.04 |
| 7 | 15.44 | 5.74 | 34.45 |
| 8 | 15.9 | 5.57 | 10.92 |
| 9 | 18.02 | 4.92 | 14.64 |
| 10 | 18.41 | 4.82 | 5.08 |
| 11 | 19.28 | 4.6 | 48.95 |
| 12 | 19.48 | 4.56 | 35.27 |
| 13 | 20.54 | 4.32 | 3.88 |
| 14 | 21.86 | 4.07 | 11.96 |
| 15 | 22.37 | 3.97 | 10.52 |
| 16 | 23.14 | 3.84 | 11.72 |
| 17 | 25.46 | 3.5 | 5.3 |
| 18 | 26.5 | 3.36 | 8.97 |
| 19 | 27.35 | 3.26 | 6.15 |
| 20 | 29.33 | 3.05 | 6.41 |
| 21 | 29.83 | 2.99 | 6.15 |
| 22 | 31.1 | 2.88 | 2.43 |
| 23 | 32.69 | 2.74 | 4.07 |
| 24 | 35.05 | 2.56 | 5.53 |
| 25 | 36.96 | 2.43 | 2.28 |

### Meglumine Salt Type A

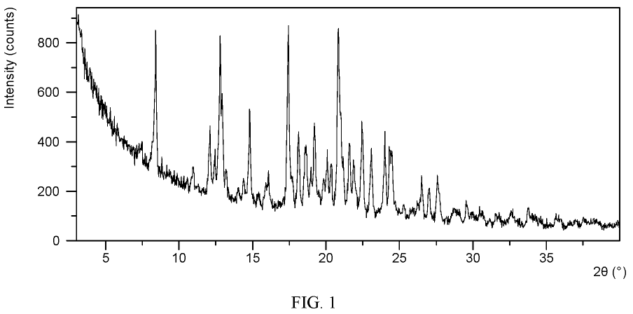

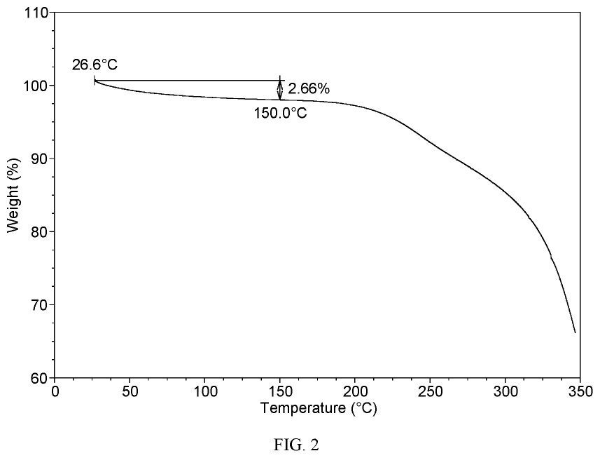

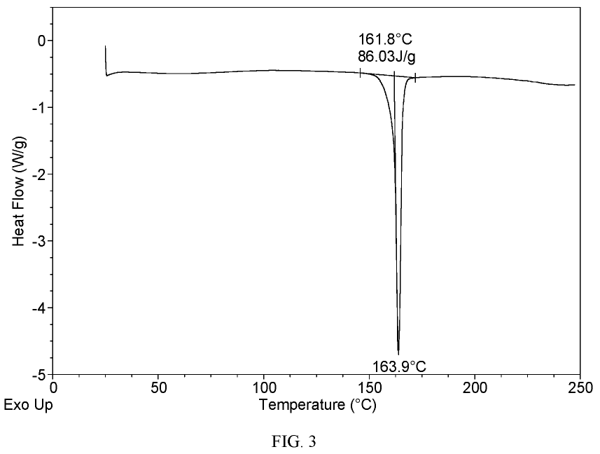

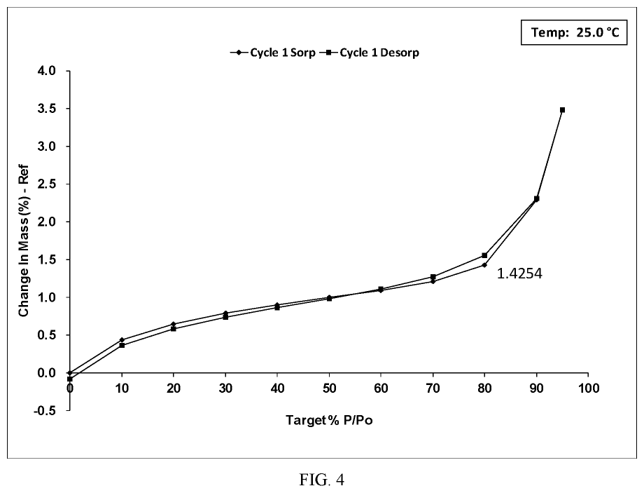

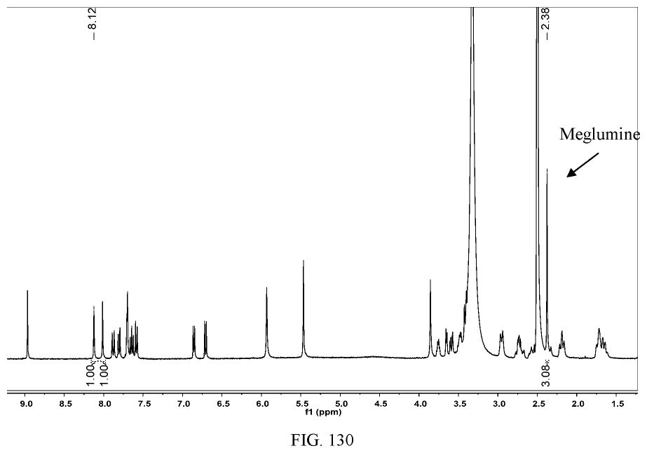

In some embodiments, the present disclosure provides solid forms of Meglumine Type A, e.g., crystalline forms of Meglumine Type A. In some embodiments, the Meglumine Type A XRPD profile is substantially similar to that shown in FIG. 1. In some embodiments, the Meglumine Type A TGA profile is substantially similar to that shown in FIG. 2. In some embodiments, the Meglumine Type A DSC profile is substantially similar to that shown in FIG. 3. In some embodiments, the Meglumine Type A DVS profile is substantially similar to that shown in FIG. 4. In some embodiments, the Meglumine Type A ¹H NMR profile is substantially similar to that shown in FIG. 130. In some embodiments, the Meglumine Type A is characterized by a DSC curve having an endotherm at about 163.9° C.

In some embodiments, the solid form of Meglumine Type A is crystalline Meglumine Type A characterized by two or more, or three XRPD signals selected from the group consisting of 20.8 °2θ, 17.4 °2θ, and 12.8 °2θ (±0.2 °2θ; ±0.1 °2θ; or ±0.0 °2θ; Cu Kα1 radiation). In some embodiments, the solid form of Meglumine Type A is crystalline Meglumine Type A characterized by XRPD signals at 20.8 °2θ, 17.4 °2θ, and 12.8 °2θ (±0.2 °2θ; ±0.1 °2θ; or ±0.0 °2θ; Cu Kα1 radiation).

In some embodiments, the solid form of Meglumine Type A is crystalline Meglumine Type A characterized by two or more, or three or more XRPD signals selected from the group consisting of 20.8 °2θ, 17.4 °2θ, 12.8 °2θ, 8.4 °2θ, and 13.0 °2θ (±0.2 °2θ; ±0.1 °2θ; or ±0.0 °2θ; Cu Kα1 radiation). In some embodiments, the solid form of Meglumine Type A is crystalline Meglumine Type A characterized by XRPD signals at 20.8 °2θ, 17.4 °2θ, 12.8 °2θ, 8.4 °2θ, and 13.0 °2θ (±0.2 °2θ; ±0.1 °2θ; or ±0.0 °2θ; Cu Kα1 radiation).

In some embodiments, the solid form of Meglumine Type A is crystalline Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, and 22.5 °2θ (±0.2 °2θ; ±0.1 °2θ; or ±0.0 °2θ; Cu Kα1 radiation). In some embodiments, the solid form of Meglumine Type A is crystalline Meglumine Type A characterized by XRPD signals at 20.8 °2θ, 17.4 °2θ, 12.8 °2θ, 8.4 °2θ, 13.0 °2θ, 14.8 °2θ, and 22.5 °2θ (±0.2 °2θ; ±0.1 °2θ; or 10.0 °2θ; Cu Kα1 radiation).

In some embodiments, the solid form of Meglumine Type A is crystalline Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, 22.5 °2θ, 19.2 °2θ, 18.1 °2θ, and 24.0 °2θ (±0.2 °2θ; 0.1 °2θ; or 0.0 °2θ; Cu Kα1 radiation). In some embodiments, the solid form of Meglumine Type A is crystalline Meglumine Type A characterized by XRPD signals at 20.8 °2θ, 17.4 °2θ, 12.8 °2θ, 8.4 °2θ, 13.0 °2θ, 14.8 °2θ, 22.5 °2θ, 19.2 °2θ, 18.1 °2θ, and 24.0 °2θ (±0.2 °2θ; ±0.1 °2θ; or ±0.0 °2θ; Cu Kα1 radiation).

In some embodiments, the crystalline Meglumine Type A is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, or twenty-six XRPD signals selected from those set forth in Table 24.

TABLE 24
Meglumine Type A Table XRPD Signals

| Signal No. | Pos. [°2θ] | d-spacing [Å] | Rel. Int. [%] |
|---|---|---|---|
| 1 | 8.42 | 10.51 | 78.83 |
| 2 | 12.1 | 7.31 | 31.5 |
| 3 | 12.79 | 6.92 | 87.89 |
| 4 | 12.95 | 6.84 | 53.29 |
| 5 | 14.82 | 5.98 | 50.04 |
| 6 | 16 | 5.54 | 11.96 |
| 7 | 17.43 | 5.09 | 98.22 |
| 8 | 18.14 | 4.89 | 39.86 |
| 9 | 18.64 | 4.76 | 34.3 |
| 10 | 19.22 | 4.62 | 45.67 |
| 11 | 20.21 | 4.39 | 15.52 |
| 12 | 20.36 | 4.36 | 25.11 |
| 13 | 20.82 | 4.27 | 100 |
| 14 | 21.62 | 4.11 | 37.16 |
| 15 | 22.47 | 3.96 | 47.92 |
| 16 | 23.08 | 3.85 | 35.21 |
| 17 | 24.01 | 3.71 | 37.74 |
| 18 | 24.32 | 3.66 | 36.52 |
| 19 | 26.52 | 3.36 | 23.09 |
| 20 | 27.03 | 3.3 | 16.88 |
| 21 | 27.58 | 3.23 | 23.42 |
| 22 | 28.74 | 3.11 | 5.92 |
| 23 | 29.58 | 3.02 | 9.47 |
| 24 | 32.63 | 2.74 | 6.14 |
| 25 | 33.77 | 2.65 | 9.17 |
| 26 | 35.79 | 2.51 | 3.87 |

### Meglumine Type B

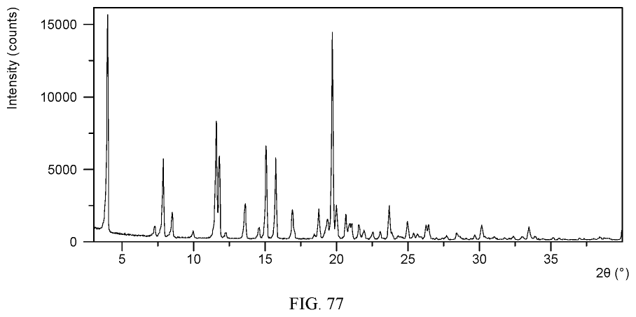

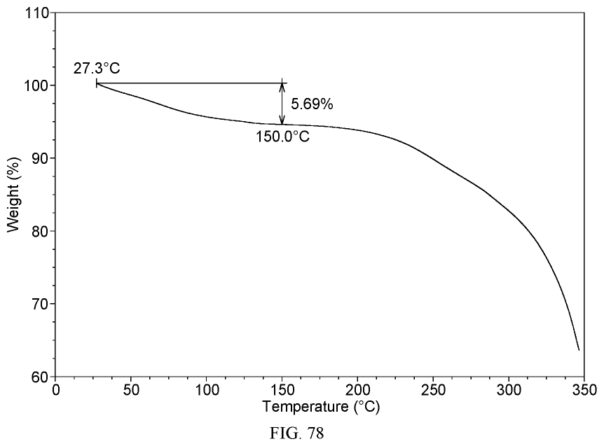

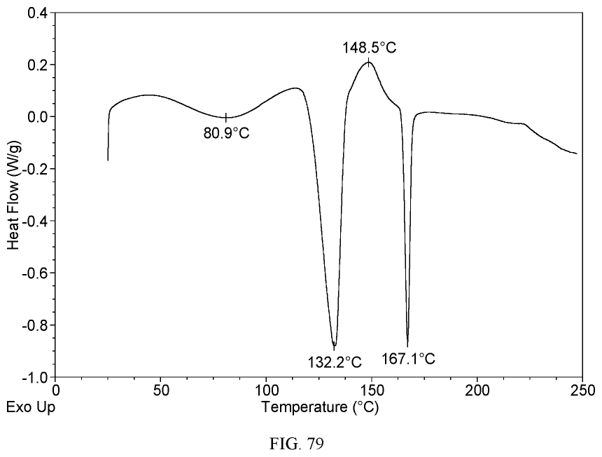

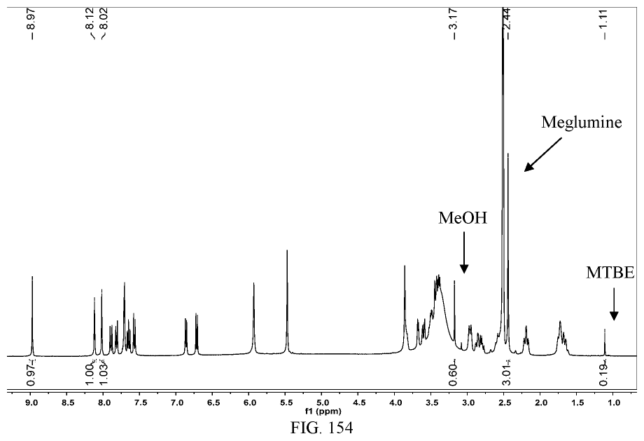

In some embodiments, the present disclosure provides solid forms of Meglumine Type B, e.g., crystalline forms of Meglumine Type B. In some embodiments, the Meglumine Type B XRPD profile is substantially similar to that shown in FIG. 77. In some embodiments, the Meglumine Type B TGA profile is substantially similar to that shown in FIG. 78. In some embodiments, the Meglumine Type B DSC profile is substantially similar to that shown in FIG. 79. In some embodiments, the Meglumine Type B ¹H NMR profile is substantially similar to that shown in FIG. 154. In some embodiments, the Meglumine Type B is characterized by a DSC curve having endotherms at about 80.9° C., about 132.2° and about 167.1° C.

In some embodiments, the solid form of Meglumine Type B is crystalline Meglumine Type B characterized by two or more, or three XRPD signals selected from the group consisting of 4.0 °2θ, 19.7 °2θ, and 11.6 °2θ (±0.2 °2θ; ±0.1 °2θ; or ±0.0 °2θ; Cu Kα1 radiation). In some embodiments, the solid form of Meglumine Type B is crystalline Meglumine Type B characterized by XRPD signals at 4.0 °2θ, 19.7 °2θ, and 11.6 °2θ (±0.2 °2θ; ±0.1 °2θ; or ±0.0 °2θ; Cu Kα1 radiation).

In some embodiments, the solid form of Meglumine Type B is crystalline Meglumine Type B characterized by two or more, or three or more XRPD signals selected from the group consisting of 4.0 °2θ, 19.7 °2θ, 11.6 °2θ, 15.1 °2θ, and 11.8 °2θ (±0.2 °2θ; ±0.1 °2θ; or ±0.0 °2θ; Cu Kα1 radiation). In some embodiments, the solid form of Meglumine Type B is crystalline Meglumine Type B characterized by XRPD signals at 4.0 °2θ, 19.7 °2θ, 11.6 °2θ, 15.1 °2θ, and 11.8 °2θ (±0.2 °2θ; ±0.1 °2θ; or ±0.0 °2θ; Cu Kα1 radiation).

In some embodiments, the solid form of Meglumine Type B is crystalline Meglumine Type B characterized by two or more, or three or more XRPD signals selected from the group consisting of 4.0 °2θ, 19.7 °2θ, 11.6 °2θ, 15.1 °2θ, 11.8 °2θ, 15.8 °2θ, and 7.9 °2θ (±0.2 °2θ; ±0.1 °2θ; or ±0.0 °2θ; Cu Kα1 radiation). In some embodiments, the solid form of Meglumine Type B is crystalline Meglumine Type B characterized by XRPD signals at 4.0 °2θ, 19.7 °2θ, 11.6 °2θ, 15.1 °2θ, 11.8 °2θ, 15.8 °2θ, and 7.9 °2θ (±0.2 °2θ; ±0.1 °2θ; or ±0.0 °2θ; Cu Kα1 radiation).

In some embodiments, the solid form of Meglumine Type B is crystalline Meglumine Type B characterized by two or more, or three or more XRPD signals selected from the group consisting of 4.0 °2θ, 19.7 °2θ, 11.6 °2θ, 15.1 °2θ, 11.8 °2θ, 15.8 °2θ, 7.9 °2θ, 13.6 °2θ, 20.0 °2θ, and 23.7 °2θ (±0.2 °2θ; ±0.1 °2θ; or ±0.0 °2θ; Cu Kα1 radiation). In some embodiments, the solid form of Meglumine Type B is crystalline Meglumine Type B characterized by XRPD signals at 4.0 °2θ, 19.7 °2θ, 11.6 °2θ, 15.1 °2θ, 11.8 °2θ, 15.8 °2θ, 7.9 °2θ, 13.6 °2θ, 20.0 °2θ, and 23.7 °2θ (±0.2 °2θ; ±0.1 °2θ; or ±0.0 °2θ; Cu Kα1 radiation).

In some embodiments, the crystalline Meglumine Type B is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, or forty-two XRPD signals selected from those set forth in Table 25.

TABLE 25
Meglumine Type B XRPD Signals

| Signal No. | Pos. [°2θ] | d-spacing [Å] | Rel. Int. [%] |
|---|---|---|---|
| 1 | 3.98 | 22.2 | 100 |
| 2 | 7.29 | 12.13 | 4.98 |
| 3 | 7.88 | 11.22 | 36.66 |
| 4 | 8.51 | 10.39 | 11.59 |
| 5 | 9.97 | 8.87 | 3.36 |
| 6 | 11.59 | 7.64 | 55.32 |
| 7 | 11.8 | 7.5 | 38.67 |
| 8 | 12.25 | 7.22 | 2.63 |
| 9 | 13.62 | 6.5 | 16.63 |
| 10 | 14.57 | 6.08 | 5.35 |
| 11 | 15.08 | 5.88 | 43.04 |
| 12 | 15.75 | 5.63 | 37.77 |
| 13 | 16.9 | 5.25 | 13.42 |
| 14 | 18.43 | 4.81 | 2.18 |
| 15 | 18.76 | 4.73 | 14.41 |
| 16 | 19.37 | 4.58 | 9.38 |
| 17 | 19.71 | 4.5 | 97.59 |
| 18 | 19.99 | 4.44 | 15.91 |
| 19 | 20.65 | 4.3 | 11.61 |
| 20 | 20.91 | 4.25 | 7.36 |
| 21 | 21.08 | 4.22 | 7.18 |
| 22 | 21.56 | 4.12 | 6.8 |
| 23 | 21.94 | 4.05 | 3.97 |
| 24 | 22.55 | 3.94 | 3.29 |
| 25 | 23.05 | 3.86 | 3.46 |
| 26 | 23.69 | 3.76 | 15.73 |
| 27 | 24.29 | 3.66 | 1.37 |
| 28 | 24.96 | 3.57 | 8.17 |
| 29 | 25.4 | 3.51 | 2.81 |
| 30 | 25.66 | 3.47 | 2.47 |
| 31 | 26.25 | 3.39 | 6.59 |
| 32 | 26.44 | 3.37 | 6.73 |
| 33 | 27.71 | 3.22 | 1.72 |
| 34 | 28.39 | 3.14 | 2.97 |
| 35 | 29.66 | 3.01 | 2.15 |
| 36 | 30.15 | 2.96 | 6.77 |
| 37 | 31.01 | 2.88 | 1.24 |
| 38 | 31.76 | 2.82 | 0.78 |
| 39 | 32.37 | 2.77 | 1.55 |
| 40 | 32.99 | 2.71 | 1.28 |
| 41 | 33.45 | 2.68 | 6.01 |
| 42 | 33.86 | 2.65 | 1.7 |

### Meglumine Type C

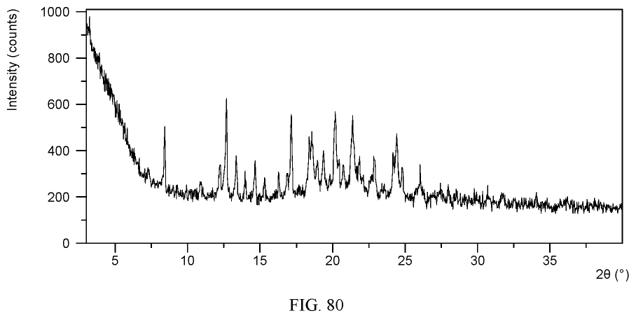

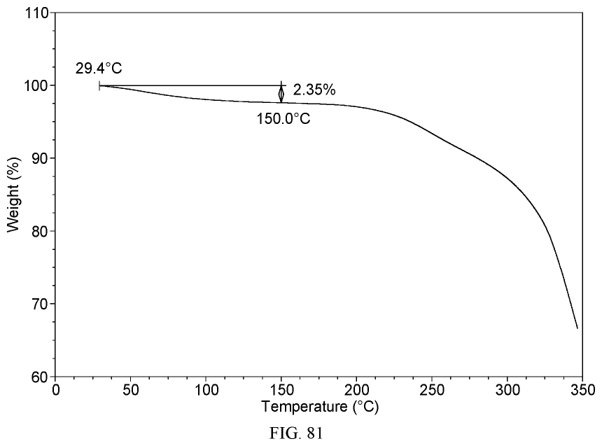

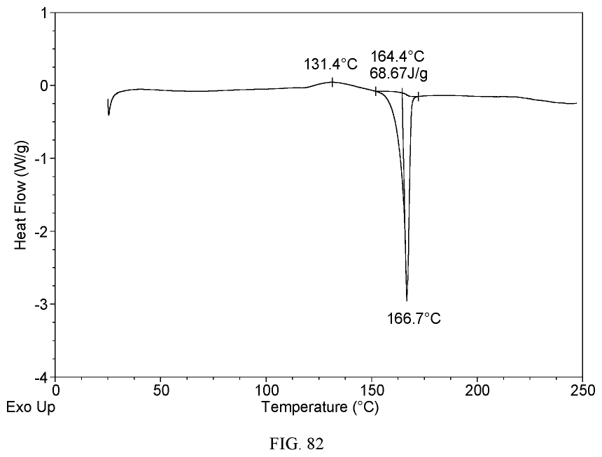

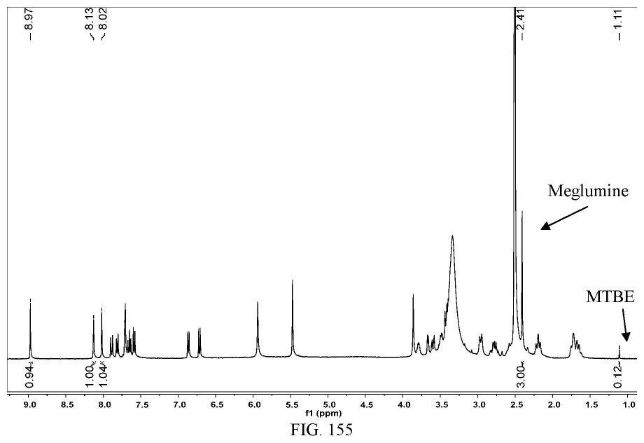

In some embodiments, the present disclosure provides solid forms of Meglumine Type C, e.g., crystalline forms of Meglumine Type C. In some embodiments, the Meglumine Type C XRPD profile is substantially similar to that shown in FIG. 80. In some embodiments, the Meglumine Type C TGA profile is substantially similar to that shown in FIG. 81. In some embodiments, the Meglumine Type C DSC profile is substantially similar to that shown in FIG. 82. In some embodiments, the Meglumine Type C ¹H NMR profile is substantially similar to that shown in FIG. 155. In some embodiments, the Meglumine Type C is characterized by a DSC curve having an endotherm at about 166.7° C.

In some embodiments, the solid form of Meglumine Type C is crystalline Meglumine Type C characterized by two or more, or three XRPD signals selected from the group consisting of 12.7 °2θ, 20.2 °2θ, and 17.1 °2θ (±0.2 °2θ; ±0.1 °2θ; or ±0.0 °2θ; Cu Kα1 radiation). In some embodiments, the solid form of Meglumine Type C is crystalline Meglumine Type C characterized by XRPD signals at 12.7 °2θ, 20.2 °2θ, and 17.1 °2θ (±0.2 °2θ; ±0.1 °2θ; or ±0.0 °2θ; Cu Kα1 radiation).

In some embodiments, the solid form of Meglumine Type C is crystalline Meglumine Type C characterized by two or more, or three or more XRPD signals selected from the group consisting of 12.7 °2θ, 20.2 °2θ, 17.1 °2θ, 21.4 °2θ, and 24.4 °2θ (±0.2 °2θ; ±0.1 °2θ; or ±0.0 °2θ; Cu Kα1 radiation). In some embodiments, the solid form of Meglumine Type C is crystalline Meglumine Type C characterized by XRPD signals at 12.7 °2θ, 20.2 °2θ, 17.1 °2θ, 21.4 °2θ, and 24.4 °2θ (±0.2 °2θ; ±0.1 °2θ; or ±0.0 °2θ; Cu Kα1 radiation).

In some embodiments, the solid form of Meglumine Type C is crystalline Meglumine Type C characterized by two or more, or three or more XRPD signals selected from the group consisting of 12.7 °2θ, 20.2 °2θ, 17.1 °2θ, 21.4 °2θ, 24.4 °2θ, 8.4 °2θ, and 18.6 °2θ (±0.2 °2θ; ±0.1 °2θ; or ±0.0 °2θ; Cu Kα1 radiation). In some embodiments, the solid form of Meglumine Type C is crystalline Meglumine Type C characterized by XRPD signals at 12.7 °2θ, 20.2 °2θ, 17.1 °2θ, 21.4 °2θ, 24.4 °2θ, 8.4 °2θ, and 18.6 °2θ (±0.2 °2θ; ±0.1 °2θ; or ±0.0 °2θ; Cu Kα1 radiation).

In some embodiments, the solid form of Meglumine Type C is crystalline Meglumine Type C characterized by two or more, or three or more XRPD signals selected from the group consisting of 12.7 °2θ, 20.2 °2θ, 17.1 °2θ, 21.4 °2θ, 24.4 °2θ, 8.4 °2θ, 18.6 °2θ, 13.4 °2θ, 22.9 °2θ, and 19.4 °2θ (±0.2 °2θ; ±0.1 °2θ; or ±0.0 °2θ; Cu Kα1 radiation). In some embodiments, the solid form of Meglumine Type C is crystalline Meglumine Type C characterized by XRPD signals at 12.7 °2θ, 20.2 °2θ, 17.1 °2θ, 21.4 °2θ, 24.4 °2θ, 8.4 °2θ, 18.6 °2θ, 13.4 °2θ, 22.9 °2θ, and 19.4 °2θ (±0.2 °2θ; ±0.1 °2θ; or ±0.0 °2θ; Cu Kα1 radiation).

In some embodiments, the crystalline Meglumine Type C is characterized by one, two, three, four, five, six, seven, eight, nine, ten, eleven, twelve, thirteen, or fourteen XRPD signals selected from those set forth in Table 26.

TABLE 26
Meglumine Type C XRPD Signals

| Signal No. | Pos. [°2θ] | d-spacing [Å] | Rel. Int. [%] |
|---|---|---|---|
| 1 | 8.41 | 10.51 | 64.52 |
| 2 | 12.68 | 6.98 | 100 |
| 3 | 13.36 | 6.63 | 44.44 |
| 4 | 13.96 | 6.35 | 18.62 |
| 5 | 14.67 | 6.04 | 37.48 |
| 6 | 17.14 | 5.17 | 85.35 |
| 7 | 18.6 | 4.77 | 60.05 |
| 8 | 19.36 | 4.59 | 41.78 |
| 9 | 20.19 | 4.4 | 87.59 |
| 10 | 21.38 | 4.16 | 72.64 |
| 11 | 22.89 | 3.88 | 41.84 |
| 12 | 24.44 | 3.64 | 68.14 |
| 13 | 24.82 | 3.59 | 32.08 |
| 14 | 26.09 | 3.42 | 20.98 |

### Freeform Type A

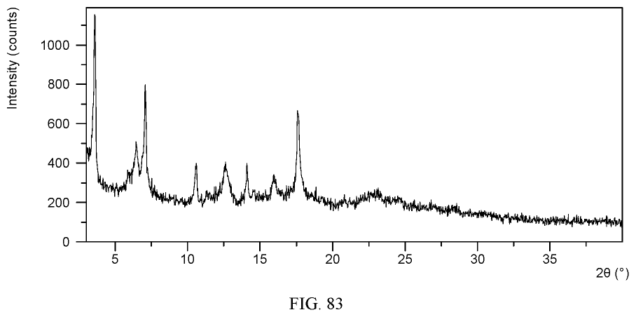

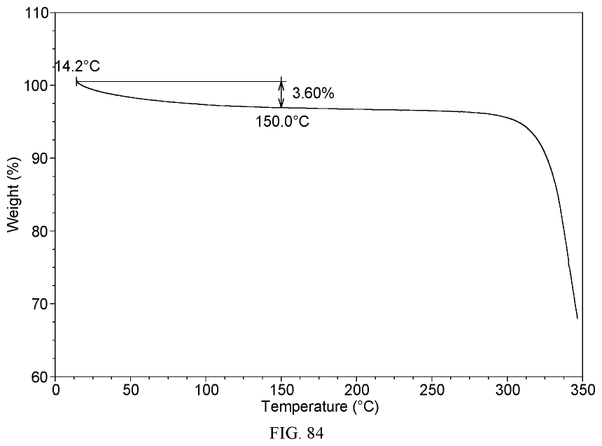

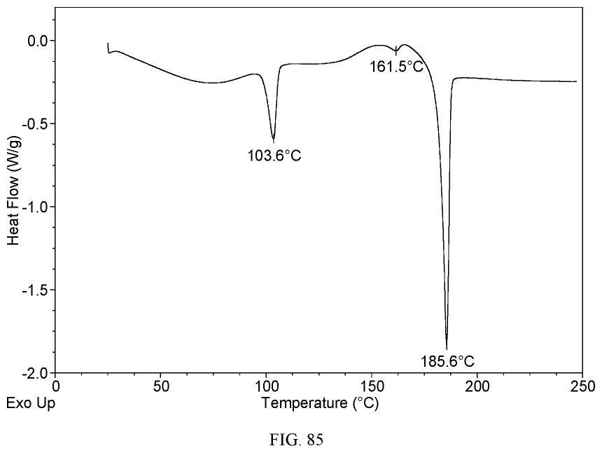

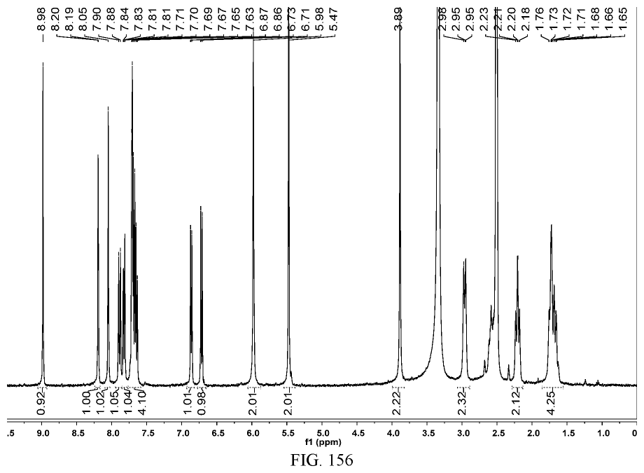

In some embodiments, the present disclosure provides solid forms of Freeform Type A, e.g., crystalline forms of Freeform Type A. In some embodiments, the Freeform Type A XRPD profile is substantially similar to that shown in FIG. 83. In some embodiments, the Freeform Type A TGA profile is substantially similar to that shown in FIG. 84. In some embodiments, the Freeform Type A DSC profile is substantially similar to that shown in FIG. 85. In some embodiments, the Freeform Type A ¹H NMR profile is substantially similar to that shown in FIG. 156. In some embodiments, the Freeform Type A is characterized by a DSC curve having endotherms at about 103.6° C., about 161.5° C., and about 185.6° C.

In some embodiments, the solid form of Freeform Type A is crystalline Freeform Type A characterized by two or more, or three XRPD signals selected from the group consisting of 3.6 °2θ, 7.1 °2θ, and 17.6 °2θ (±0.2 °2θ; ±0.1 °2θ; or ±0.0 °2θ; Cu Kα1 radiation). In some embodiments, the solid form of Freeform Type A is crystalline Freeform Type A characterized by XRPD signals at 3.6 °2θ, 7.1 °2θ, and 17.6 °2θ (±0.2 °2θ; ±0.1 °2θ; or ±0.0 °2θ; Cu Kα1 radiation).

In some embodiments, the solid form of Freeform Type A is crystalline Freeform Type A characterized by two or more, or three or more XRPD signals selected from the group consisting of 3.6 °2θ, 7.1 °2θ, 17.6 °2θ, 6.5 °2θ, and 12.6 °2θ (±0.2 °2θ; ±0.1 °2θ; or ±0.0 °2θ; Cu Kα1 radiation). In some embodiments, the solid form of Freeform Type A is crystalline Freeform Type A characterized by XRPD signals at 3.6 °2θ, 7.1 °2θ, 17.6 °2θ, 6.5 °2θ, and 12.6 °2θ (±0.2 °2θ; ±0.1 °2θ; or ±0.0 °2θ; Cu Kα1 radiation).

In some embodiments, the solid form of Freeform Type A is crystalline Freeform Type A characterized by two or more, or three or more XRPD signals selected from the group consisting of 3.6 °2θ, 7.1 °2θ, 17.6 °2θ, 6.5 °2θ, 12.6 °2θ, 10.6 °2θ, and 14.1 °2θ (±0.2 °2θ; ±0.1 °2θ; or ±0.0 °2θ; Cu Kα1 radiation). In some embodiments, the solid form of Freeform Type A is crystalline Freeform Type A characterized by XRPD signals at 3.6 °2θ, 7.1 °2θ, 17.6 °2θ, 6.5 °2θ, 12.6 °2θ, 10.6 °2θ, and 14.1 °2θ (±0.2 °2θ; ±0.1 °2θ; or ±0.0 °2θ; Cu Kα1 radiation).

In some embodiments, the crystalline Freeform Type A is characterized by one, two, three, four, five, six, seven, or eight XRPD signals selected from those set forth in Table 27.

TABLE 27
Freeform Type A XRPD Signals

| Signal No. | Pos. [°2θ] | d-spacing [Å] | Rel. Int. [%] |
|---|---|---|---|
| 1 | 3.6 | 24.53 | 100 |
| 2 | 6.46 | 13.67 | 32.04 |
| 3 | 7.09 | 12.47 | 68.78 |
| 4 | 10.59 | 8.36 | 22.77 |
| 5 | 12.6 | 7.03 | 22.94 |
| 6 | 14.09 | 6.29 | 21.51 |
| 7 | 15.94 | 5.56 | 15.55 |
| 8 | 17.61 | 5.04 | 57.92 |

### Freeform Type B

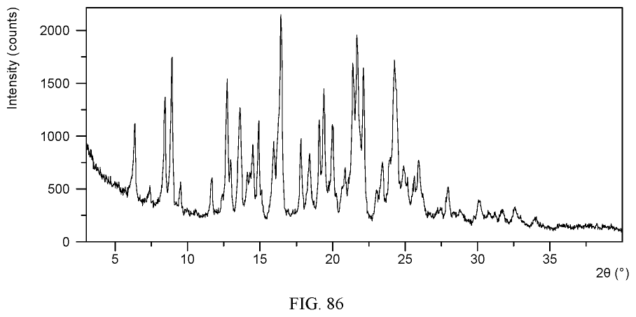

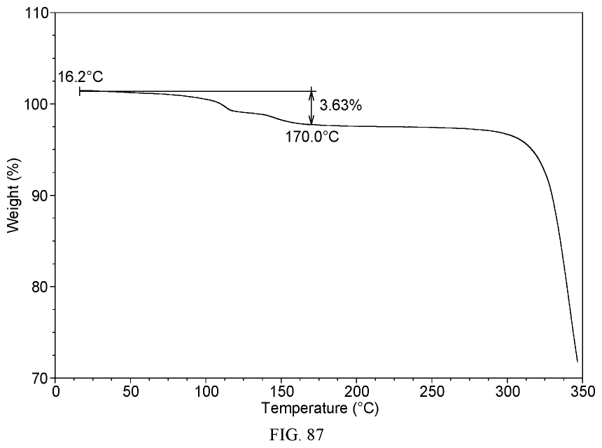

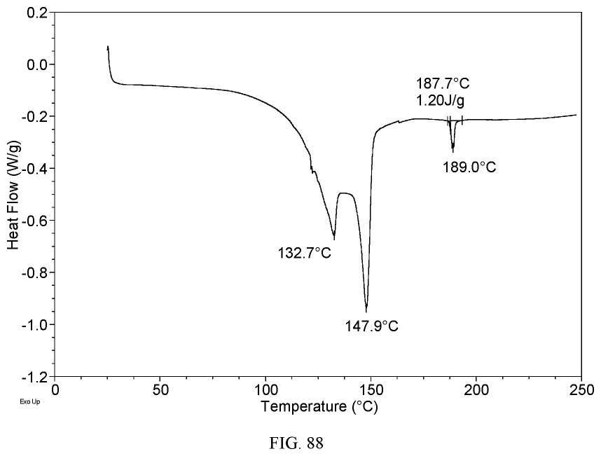

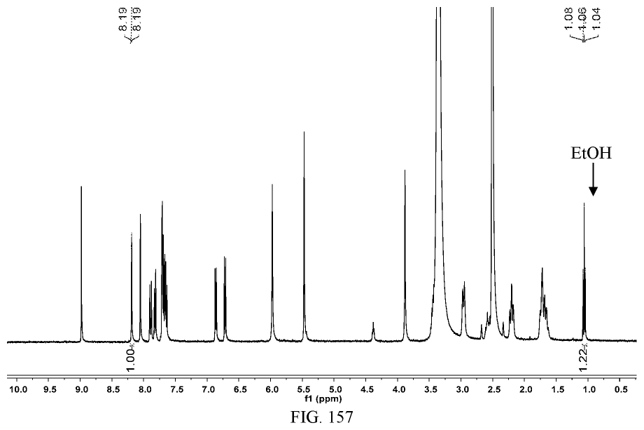

In some embodiments, the present disclosure provides solid forms of Freeform Type B, e.g., crystalline forms of Freeform Type B. In some embodiments, the Freeform Type B XRPD profile is substantially similar to that shown in FIG. 86. In some embodiments, the Freeform Type B TGA profile is substantially similar to that shown in FIG. 87. In some embodiments, the Freeform Type B DSC profile is substantially similar to that shown in FIG. 88. In some embodiments, the Freeform Type B ¹H NMR profile is substantially similar to that shown in FIG. 157. In some embodiments, the Freeform Type B is characterized by a DSC curve having endotherms at about 132.7° C., about 147.9° C., and about 189.0° C.

In some embodiments, the solid form of Freeform Type B is crystalline Freeform Type B characterized by two or more, or three XRPD signals selected from the group consisting of 16.5 °2θ, 21.7 °2θ, and 21.4 °2θ (±0.2 °2θ; ±0.1 °2θ; or ±0.0 °2θ; Cu Kα1 radiation). In some embodiments, the solid form of Freeform Type B is crystalline Freeform Type B characterized by XRPD signals at 16.5 °2θ, 21.7 °2θ, and 21.4 °2θ (±0.2 °2θ; ±0.1 °2θ; or ±0.0 °2θ; Cu Kα1 radiation).

In some embodiments, the solid form of Freeform Type B is crystalline Freeform Type B characterized by two or more, or three or more XRPD signals selected from the group consisting of 16.5 °2θ, 21.7 °2θ, 21.4 °2θ, 8.9 °2θ, and 24.2 °2θ (±0.2 °2θ; ±0.1 °2θ; or ±0.0 °2θ; Cu Kα1 radiation). In some embodiments, the solid form of Freeform Type B is crystalline Freeform Type B characterized by XRPD signals at 16.5 °2θ, 21.7 °2θ, 21.4 °2θ, 8.9 °2θ, and 24.2 °2θ (±0.2 °2θ; ±0.1 °2θ; or ±0.0 °2θ; Cu Kα1 radiation).

In some embodiments, the solid form of Freeform Type B is crystalline Freeform Type B characterized by two or more, or three or more XRPD signals selected from the group consisting of 16.5 °2θ, 21.7 °2θ, 21.4 °2θ, 8.9 °2θ, 24.2 °2θ, 22.1 °2θ, and 12.7 °2θ (±0.2 °2θ; ±0.1 °2θ; or ±0.0 °2θ; Cu Kα1 radiation). In some embodiments, the solid form of Freeform Type B is crystalline Freeform Type B characterized by XRPD signals at 16.5 °2θ, 21.7 °2θ, 21.4 °2θ, 8.9 °2θ, 24.2 °2θ, 22.1 °2θ, and 12.7 °2θ (±0.2 °2θ; ±0.1 °2θ; or ±0.0 °2θ; Cu Kα1 radiation).

In some embodiments, the solid form of Freeform Type B is crystalline Freeform Type B characterized by two or more, or three or more XRPD signals selected from the group consisting of 16.5 °2θ, 21.7 °2θ, 21.4 °2θ, 8.9 °2θ, 24.2 °2θ, 22.1 °2θ, 12.7 °2θ, 19.4 °2θ, 8.4 °2θ, and 13.6 °2θ (±0.2 °2θ; ±0.1 °2θ; or ±0.0 °2θ; Cu Kα1 radiation). In some embodiments, the solid form of Freeform Type B is crystalline Freeform Type B characterized by XRPD signals at 16.5 °2θ, 21.7 °2θ, 21.4 °2θ, 8.9 °2θ, 24.2 °2θ, 22.1 °2θ, 12.7 °2θ, 19.4 °2θ, 8.4 °2θ, and 13.6 °2θ (±0.2 °2θ; ±0.1 °2θ; or ±0.0 °2θ; Cu Kα1 radiation).

In some embodiments, the crystalline Freeform Type B is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, or thirty-three XRPD signals selected from those set forth in Table 28.

TABLE 28
Freeform Type B XRPD Signals

| Signal No. | Pos. [°2θ] | d-spacing [Å] | Rel. Int. [%] |
|---|---|---|---|
| 1 | 6.36 | 13.9 | 38.96 |
| 2 | 7.36 | 12.02 | 6.65 |
| 3 | 8.43 | 10.48 | 57.91 |
| 4 | 8.92 | 9.92 | 78.2 |
| 5 | 9.51 | 9.3 | 14.95 |
| 6 | 11.66 | 7.59 | 19.5 |
| 7 | 12.73 | 6.95 | 70.64 |
| 8 | 12.99 | 6.82 | 27.24 |
| 9 | 13.62 | 6.5 | 56.55 |
| 10 | 14.51 | 6.1 | 37.25 |
| 11 | 14.9 | 5.94 | 49.09 |
| 12 | 15.92 | 5.57 | 37.21 |
| 13 | 16.46 | 5.39 | 100 |
| 14 | 17.81 | 4.98 | 39.18 |
| 15 | 18.43 | 4.81 | 32.03 |
| 16 | 19.08 | 4.65 | 49.61 |
| 17 | 19.4 | 4.57 | 63 |
| 18 | 20 | 4.44 | 47.27 |
| 19 | 20.85 | 4.26 | 24.79 |
| 20 | 21.41 | 4.15 | 78.9 |
| 21 | 21.69 | 4.1 | 93.51 |
| 22 | 22.13 | 4.02 | 75.47 |
| 23 | 23.01 | 3.86 | 12.89 |
| 24 | 23.47 | 3.79 | 26.82 |
| 25 | 24.24 | 3.67 | 77.16 |
| 26 | 25.6 | 3.48 | 18.9 |
| 27 | 25.94 | 3.43 | 30.4 |
| 28 | 27.95 | 3.19 | 16.67 |
| 29 | 28.87 | 3.09 | 5.27 |
| 30 | 30.11 | 2.97 | 11.5 |
| 31 | 31.73 | 2.82 | 7.44 |
| 32 | 32.52 | 2.75 | 8.76 |
| 33 | 33.98 | 2.64 | 4.73 |

### Freeform Type C

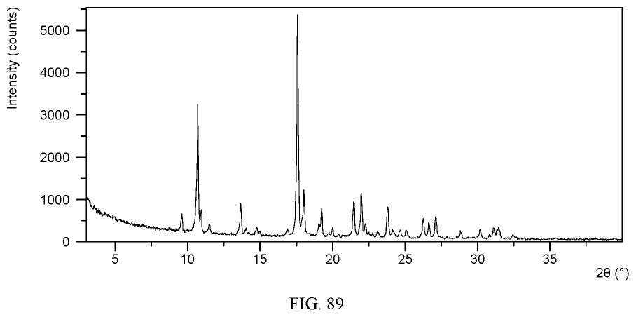

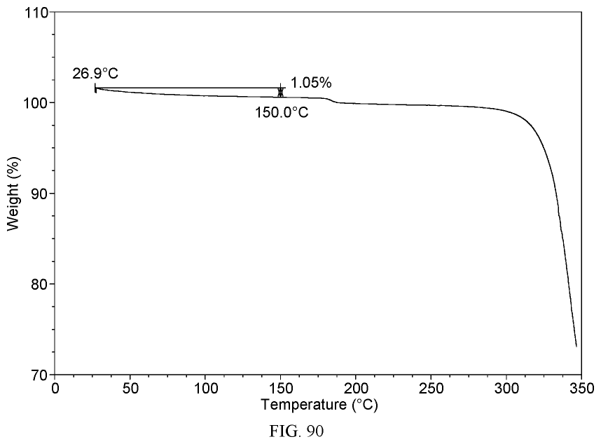

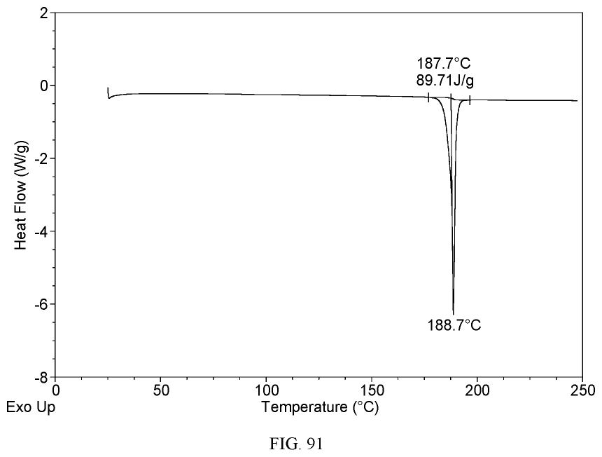

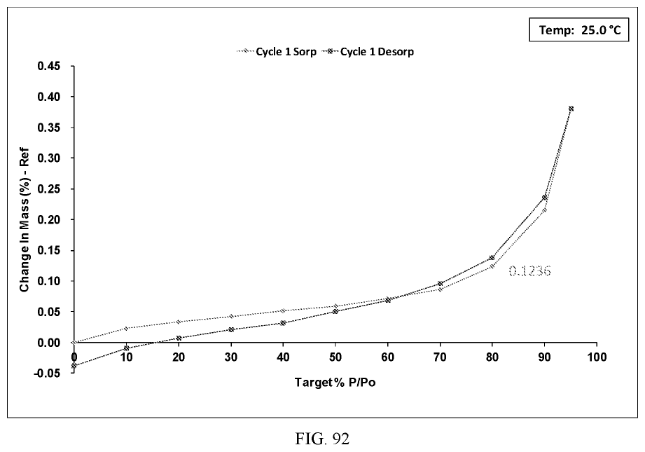

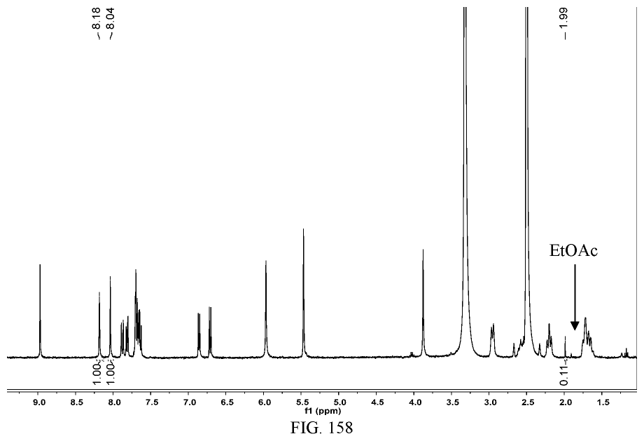

In some embodiments, the present disclosure provides solid forms of Freeform Type C, e.g., crystalline forms of Freeform Type C. In some embodiments, the Freeform Type C XRPD profile is substantially similar to that shown in FIG. 89. In some embodiments, the Freeform Type C TGA profile is substantially similar to that shown in FIG. 90. In some embodiments, the Freeform Type C DSC profile is substantially similar to that shown in FIG. 91. In some embodiments, the Freeform Type C DVS profile is substantially similar to that shown in FIG. 92. In some embodiments, the Freeform Type C ¹H NMR profile is substantially similar to that shown in FIG. 158. In some embodiments, the Freeform Type C is characterized by a DSC curve having an endotherm at about 188.7° C.

In some embodiments, the solid form of Freeform Type C is crystalline Freeform Type C characterized by two or more, or three XRPD signals selected from the group consisting of 17.6 °2θ, 10.7 °2θ, and 18.0 °2θ (±0.2 °2θ; ±0.1 °2θ; or ±0.0 °2θ; Cu Kα1 radiation). In some embodiments, the solid form of Freeform Type C is crystalline Freeform Type C characterized by XRPD signals at 17.6 °2θ, 10.7 °2θ, and 18.0 °2θ (±0.2 °2θ; ±0.1 °2θ; or ±0.0 °2θ; Cu Kα1 radiation).

In some embodiments, the solid form of Freeform Type C is crystalline Freeform Type C characterized by two or more, or three or more XRPD signals selected from the group consisting of 17.6 °2θ, 10.7 °2θ, 18.0 °2θ, 22.0 °2θ, and 21.5 °2θ (±0.2 °2θ; ±0.1 °2θ; or ±0.0 °2θ; Cu Kα1 radiation). In some embodiments, the solid form of Freeform Type C is crystalline Freeform Type C characterized by XRPD signals at 17.6 °2θ, 10.7 °2θ, 18.0 °2θ, 22.0 °2θ, and 21.5 °2θ (±0.2 °2θ; ±0.1 °2θ; or ±0.0 °2θ; Cu Kα1 radiation).

In some embodiments, the solid form of Freeform Type C is crystalline Freeform Type C characterized by two or more, or three or more XRPD signals selected from the group consisting of 17.6 °2θ, 10.7 °2θ, 18.0 °2θ, 22.0 °2θ, 21.5 °2θ, 13.7 °2θ, and 23.8 °2θ (±0.2 °2θ; ±0.1 °2θ; or ±0.0 °2θ; Cu Kα1 radiation). In some embodiments, the solid form of Freeform Type C is crystalline Freeform Type C characterized by XRPD signals at 17.6 °2θ, 10.7 °2θ, 18.0 °2θ, 22.0 °2θ, 21.5 °2θ, 13.7 °2θ, and 23.8 °2θ (±0.2 °2θ; ±0.1 °2θ; or ±0.0 °2θ; Cu Kα1 radiation).

In some embodiments, the solid form of Freeform Type C is crystalline Freeform Type C characterized by two or more, or three or more XRPD signals selected from the group consisting of 17.6 °2θ, 10.7 °2θ, 18.0 °2θ, 22.0 °2θ, 21.5 °2θ, 13.7 °2θ, 23.8 °2θ, 19.3 °2θ, 10.9 °2θ, and 27.1 °2θ (±0.2 °2θ; ±0.1 °2θ; or ±0.0 °2θ; Cu Kα1 radiation). In some embodiments, the solid form of Freeform Type C is crystalline Freeform Type C characterized by XRPD signals at 17.6 °2θ, 10.7 °2θ, 18.0 °2θ, 22.0 °2θ, 21.5 °2θ, 13.7 °2θ, 23.8 °2θ, 19.3 °2θ, 10.9 °2θ, and 27.1 °2θ (±0.2 °2θ; ±0.1 °2θ; or ±0.0 °2θ; Cu Kα1 radiation).

In some embodiments, the crystalline Freeform Type C is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, or twenty-seven XRPD signals selected from those set forth in Table 29.

TABLE 29
Freeform Type C XRPD Signals

| Signal No. | Pos. [°2θ] | d-spacing [Å] | Rel. Int. [%] |
|---|---|---|---|
| 1 | 9.5985 | 9.21 | 7.48 |
| 2 | 10.7018 | 8.27 | 61.62 |
| 3 | 10.9353 | 8.09 | 11.23 |
| 4 | 11.5114 | 7.69 | 4.26 |
| 5 | 13.6661 | 6.48 | 15.1 |
| 6 | 14.0147 | 6.32 | 3.19 |
| 7 | 14.7798 | 5.99 | 4.02 |
| 8 | 16.9046 | 5.24 | 2.77 |
| 9 | 17.5905 | 5.04 | 100 |
| 10 | 18.0325 | 4.92 | 21.55 |
| 11 | 19.2556 | 4.61 | 12.04 |
| 12 | 20.001 | 4.44 | 4.16 |
| 13 | 21.4649 | 4.14 | 17.6 |
| 14 | 21.9861 | 4.04 | 20.7 |
| 15 | 22.2747 | 3.99 | 6.27 |
| 16 | 23.122 | 3.85 | 2.59 |
| 17 | 23.8041 | 3.74 | 14.67 |
| 18 | 24.6489 | 3.61 | 3.49 |
| 19 | 25.079 | 3.55 | 3.7 |
| 20 | 26.2668 | 3.39 | 8.75 |
| 21 | 26.6518 | 3.34 | 7.67 |
| 22 | 27.1174 | 3.29 | 10.39 |
| 23 | 28.8328 | 3.1 | 3.64 |
| 24 | 30.1707 | 2.96 | 4.39 |
| 25 | 31.1121 | 2.87 | 5.14 |
| 26 | 31.4773 | 2.84 | 5.77 |
| 27 | 32.4419 | 2.76 | 1.97 |

### Freeform Type D

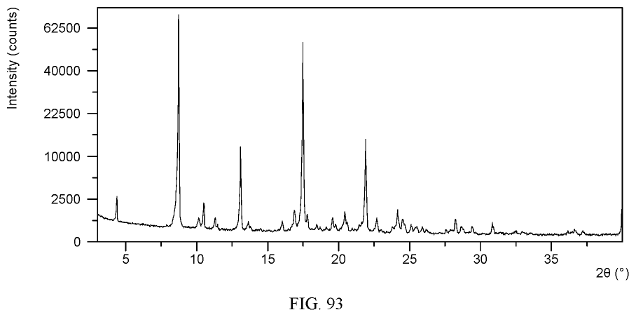

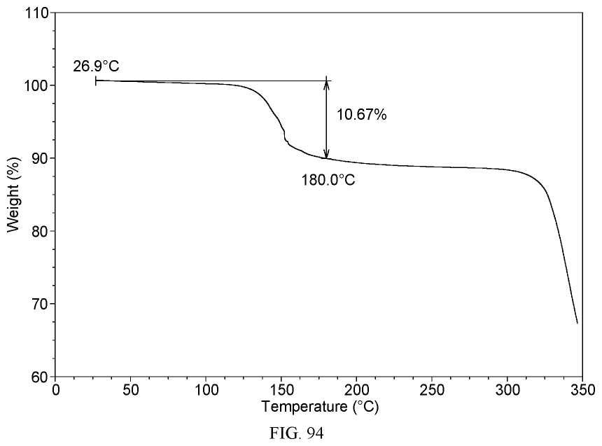

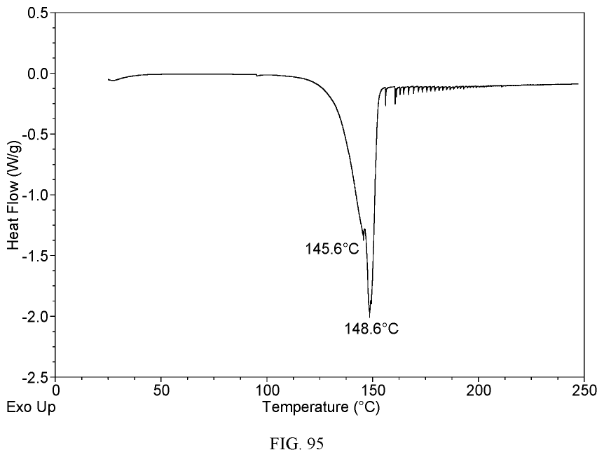

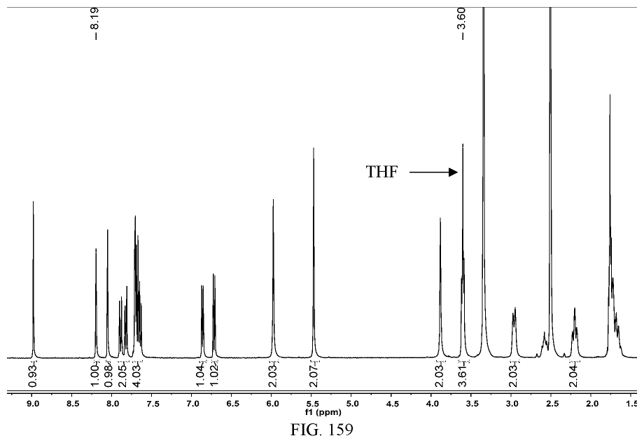

In some embodiments, the present disclosure provides solid forms of Freeform Type D, e.g., crystalline forms of Freeform Type D. In some embodiments, the Freeform Type D XRPD profile is substantially similar to that shown in FIG. 93. In some embodiments, the Freeform Type D TGA profile is substantially similar to that shown in FIG. 94. In some embodiments, the Freeform Type D DSC profile is substantially similar to that shown in FIG. 95. In some embodiments, the Freeform Type D ¹H NMR profile is substantially similar to that shown in FIG. 159. In some embodiments, the Freeform Type D is characterized by a DSC curve having endotherms at about 145.6° C. and about 148.6° C.

In some embodiments, the solid form of Freeform Type D is crystalline Freeform Type D characterized by two or more, or three XRPD signals selected from the group consisting of 8.7 °2θ, 17.5 °2θ, and 13.1 °2θ (±0.2 °2θ; ±0.1 °2θ; or ±0.0 °2θ; Cu Kα1 radiation). In some embodiments, the solid form of Freeform Type D is crystalline Freeform Type D characterized by XRPD signals at 8.7 °2θ, 17.5 °2θ, and 13.1 °2θ (±0.2 °2θ; ±0.1 °2θ; or ±0.0 °2θ; Cu Kα1 radiation).

In some embodiments, the solid form of Freeform Type D is crystalline Freeform Type D characterized by two or more, or three or more XRPD signals selected from the group consisting of 8.7 °2θ, 17.5 °2θ, 13.1 °2θ, 21.9 °2θ, and 4.4 °2θ (±0.2 °2θ; ±0.1 °2θ; or ±0.0 °2θ; Cu Kα1 radiation). In some embodiments, the solid form of Freeform Type D is crystalline Freeform Type D characterized by XRPD signals at 8.7 °2θ, 17.5 °2θ, 13.1 °2θ, 21.9 °2θ, and 4.4 °2θ (±0.2 °2θ; ±0.1 °2θ; or ±0.0 °2θ; Cu Kα1 radiation).

In some embodiments, the solid form of Freeform Type D is crystalline Freeform Type D characterized by two or more, or three or more XRPD signals selected from the group consisting of 8.7 °2θ, 17.5 °2θ, 13.1 °2θ, 21.9 °2θ, 4.4 °2θ, 10.5 °2θ, and 24.2 °2θ (±0.2 °2θ; ±0.1 °2θ; or ±0.0 °2θ; Cu Kα1 radiation). In some embodiments, the solid form of Freeform Type D is crystalline Freeform Type D characterized by XRPD signals at 8.7 °2θ, 17.5 °2θ, 13.1 °2θ, 21.9 °2θ, 4.4 °2θ, 10.5 °2θ, and 24.2 °2θ (±0.2 °2θ; ±0.1 °2θ; or ±0.0 °2θ; Cu Kα1 radiation).

In some embodiments, the solid form of Freeform Type D is crystalline Freeform Type D characterized by two or more, or three or more XRPD signals selected from the group consisting of 8.7 °2θ, 17.5 °2θ, 13.1 °2θ, 21.9 °2θ, 4.4 °2θ, 10.5 °2θ, 24.2 °2θ, 16.9 °2θ, 20.4 °2θ, and 17.8 °2θ (±0.2 °2θ; ±0.1 °2θ; or ±0.0 °2θ; Cu Kα1 radiation). In some embodiments, the solid form of Freeform Type D is crystalline Freeform Type D characterized by XRPD signals at 8.7 °2θ, 17.5 °2θ, 13.1 °2θ, 21.9 °2θ, 4.4 °2θ, 10.5 °2θ, 24.2 °2θ, 16.9 °2θ, 20.4 °2θ, and 17.8 °2θ (±0.2 °2θ; ±0.1 °2θ; or ±0.0 °2θ; Cu Kα1 radiation).

In some embodiments, the crystalline Freeform Type D is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, or twenty-seven XRPD signals selected from those set forth in Table 30.

TABLE 30
Freeform Type D XRPD Signals

| Signal No. | Pos. [°2θ] | d-spacing [Å] | Rel. Int. [%] |
|---|---|---|---|
| 1 | 4.37 | 20.21 | 2.72 |
| 2 | 8.73 | 10.13 | 100 |
| 3 | 10.15 | 8.71 | 0.66 |
| 4 | 10.51 | 8.42 | 2.59 |
| 5 | 11.3 | 7.83 | 0.81 |
| 6 | 13.1 | 6.76 | 16.39 |
| 7 | 13.64 | 6.49 | 0.55 |
| 8 | 16.04 | 5.52 | 0.61 |
| 9 | 16.89 | 5.25 | 1.65 |
| 10 | 17.49 | 5.07 | 65.84 |
| 11 | 17.8 | 4.98 | 1.17 |
| 12 | 18.46 | 4.81 | 0.37 |
| 13 | 19.57 | 4.54 | 0.88 |
| 14 | 19.79 | 4.49 | 0.38 |
| 15 | 20.44 | 4.34 | 1.56 |
| 16 | 21.91 | 4.06 | 15.52 |
| 17 | 22.7 | 3.92 | 0.85 |
| 18 | 23.79 | 3.74 | 0.29 |
| 19 | 24.16 | 3.68 | 1.74 |
| 20 | 24.51 | 3.63 | 0.84 |
| 21 | 25.12 | 3.55 | 0.37 |
| 22 | 25.54 | 3.49 | 0.25 |
| 23 | 25.91 | 3.44 | 0.26 |
| 24 | 28.24 | 3.16 | 0.84 |
| 25 | 28.65 | 3.12 | 0.29 |
| 26 | 29.42 | 3.04 | 0.32 |
| 27 | 30.83 | 2.9 | 0.56 |

### Freeform Type E

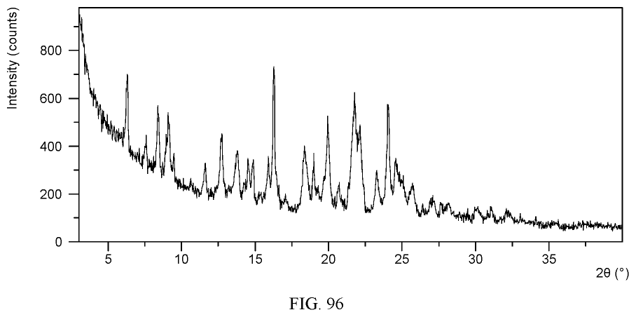

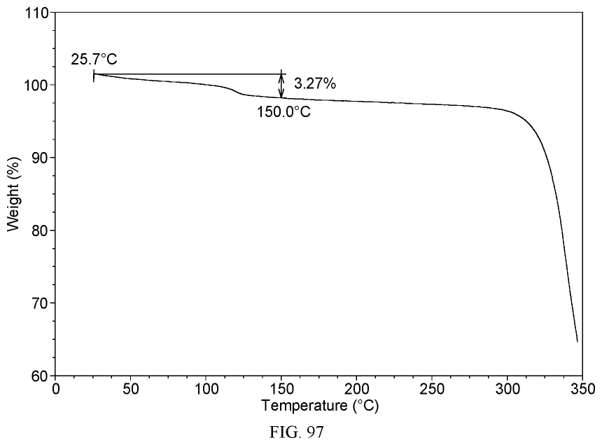

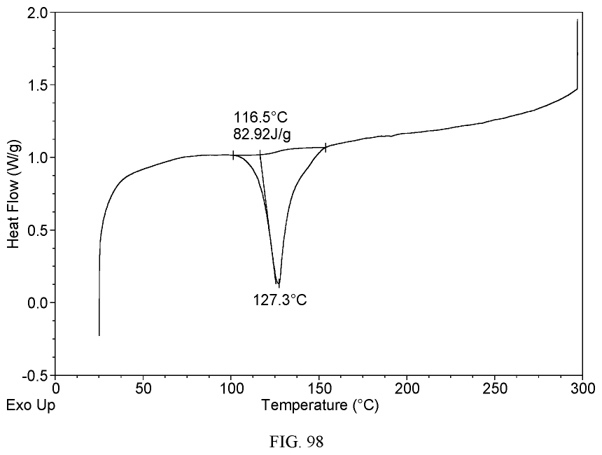

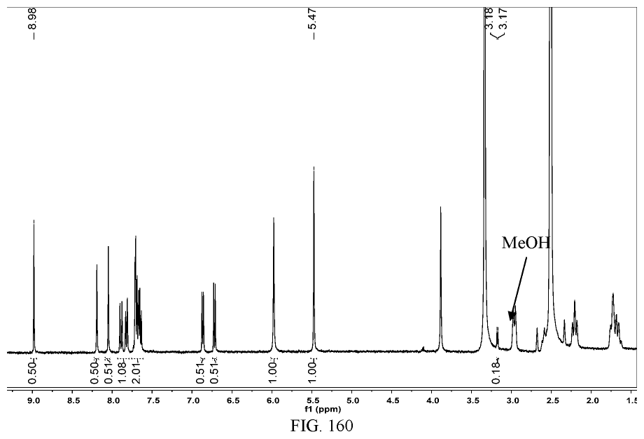

In some embodiments, the present disclosure provides solid forms of Freeform Type E, e.g., crystalline forms of Freeform Type E. In some embodiments, the Freeform Type E XRPD profile is substantially similar to that shown in FIG. 96. In some embodiments, the Freeform Type E TGA profile is substantially similar to that shown in FIG. 97. In some embodiments, the Freeform Type E DSC profile is substantially similar to that shown in FIG. 98. In some embodiments, the Freeform Type E ¹H NMR profile is substantially similar to that shown in FIG. 160. In some embodiments, the Freeform Type E is characterized by a DSC curve having endotherms at about 127.3° C.

In some embodiments, the solid form of Freeform Type E is crystalline Freeform Type E characterized by two or more, or three XRPD signals selected from the group consisting of 16.3 °2θ, 21.8 °2θ, and 24.1 °2θ (±0.2 °2θ; ±0.1 °2θ; or ±0.0 °2θ; Cu Kα1 radiation). In some embodiments, the solid form of Freeform Type E is crystalline Freeform Type E characterized by XRPD signals at 16.3 °2θ, 21.8 °2θ, and 24.1 °2θ (±0.2 °2θ; ±0.1 °2θ; or ±0.0 °2θ; Cu Kα1 radiation).

In some embodiments, the solid form of Freeform Type E is crystalline Freeform Type E characterized by two or more, or three or more XRPD signals selected from the group consisting of 16.3 °2θ, 21.8 °2θ, 24.1 °2θ, 20.0 °2θ, and 22.2 °2θ (±0.2 °2θ; ±0.1 °2θ; or ±0.0 °2θ; Cu Kα1 radiation). In some embodiments, the solid form of Freeform Type E is crystalline Freeform Type E characterized by XRPD signals at 16.3 °2θ, 21.8 °2θ, 24.1 °2θ, 20.0 °2θ, and 22.2 °2θ (±0.2 °2θ; ±0.1 °2θ; or ±0.0 °2θ; Cu Kα1 radiation).

In some embodiments, the solid form of Freeform Type E is crystalline Freeform Type E characterized by two or more, or three or more XRPD signals selected from the group consisting of 16.3 °2θ, 21.8 °2θ, 24.1 °2θ, 20.0 °2θ, 22.2 °2θ, 6.3 °2θ, and 12.7 °2θ (±0.2 °2θ; ±0.1 °2θ; or ±0.0 °2θ; Cu Kα1 radiation). In some embodiments, the solid form of Freeform Type E is crystalline Freeform Type E characterized by XRPD signals at 16.3 °2θ, 21.8 °2θ, 24.1 °2θ, 20.0 °2θ, 22.2 °2θ, 6.3 °2θ, and 12.7 °2θ (±0.2 °2θ; ±0.1 °2θ; or ±0.0 °2θ; Cu Kα1 radiation).

In some embodiments, the solid form of Freeform Type E is crystalline Freeform Type E characterized by two or more, or three or more XRPD signals selected from the group consisting of 16.3 °2θ, 21.8 °2θ, 24.1 °2θ, 20.0 °2θ, 22.2 °2θ, 6.3 °2θ, 12.7 °2θ, 8.4 °2θ, 18.4 °2θ, and 9.1 °2θ (±0.2 °2θ; ±0.1 °2θ; or ±0.0 °2θ; Cu Kα1 radiation). In some embodiments, the solid form of Freeform Type E is crystalline Freeform Type E characterized by XRPD signals at 16.3 °2θ, 21.8 °2θ, 24.1 °2θ, 20.0 °2θ, 22.2 °2θ, 6.3 °2θ, 12.7 °2θ, 8.4 °2θ, 18.4 °2θ, and 9.1 °2θ (±0.2 °2θ; ±0.1 °2θ; or ±0.0 °2θ; Cu Kα1 radiation).

In some embodiments, the crystalline Freeform Type E is characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 31.

TABLE 31
Freeform Type E XRPD Signals

| Signal No. | Pos. [°2θ] | d-spacing [Å] | Rel. Int. [%] |
|---|---|---|---|
| 1 | 6.3 | 14.04 | 53.11 |
| 2 | 8.4 | 10.53 | 41.67 |
| 3 | 9.13 | 9.69 | 40.47 |
| 4 | 11.59 | 7.63 | 19.84 |
| 5 | 12.73 | 6.95 | 45.1 |
| 6 | 13.82 | 6.41 | 34.43 |
| 7 | 14.54 | 6.09 | 27.51 |
| 8 | 14.87 | 5.96 | 29.07 |
| 9 | 15.89 | 5.58 | 31.23 |
| 10 | 16.28 | 5.44 | 100 |
| 11 | 18.36 | 4.83 | 41.35 |
| 12 | 18.99 | 4.67 | 33.24 |
| 13 | 19.95 | 4.45 | 63.89 |
| 14 | 20.72 | 4.29 | 16.03 |
| 15 | 21.76 | 4.08 | 78.5 |
| 16 | 22.15 | 4.01 | 59.37 |
| 17 | 23.28 | 3.82 | 26.97 |
| 18 | 24.06 | 3.7 | 74.17 |
| 19 | 24.58 | 3.62 | 35.82 |
| 20 | 25.73 | 3.46 | 20.93 |
| 21 | 30.11 | 2.97 | 7.67 |
| 22 | 32.19 | 2.78 | 7.2 |

### Freeform Type F

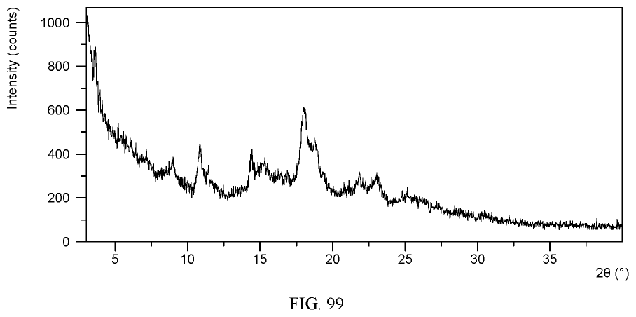

In some embodiments, the present disclosure provides solid forms of Freeform Type F, e.g., crystalline forms of Freeform Type F. In some embodiments, the Freeform Type F XRPD profile is substantially similar to that shown in FIG. 99.

In some embodiments, the solid form of Freeform Type F is crystalline Freeform Type F characterized by two or more, or three XRPD signals selected from the group consisting of 18.0 °2θ, 10.8 °2θ, and 14.4 °2θ (±0.2 °2θ; ±0.1 °2θ; or ±0.0 °2θ; Cu Kα1 radiation). In some embodiments, the solid form of Freeform Type F is crystalline Freeform Type F characterized by XRPD signals at 18.0 °2θ, 10.8 °2θ, and 14.4 °2θ (±0.2 °2θ; ±0.1 °2θ; or ±0.0 °2θ; Cu Kα1 radiation).

In some embodiments, the solid form of Freeform Type F is crystalline Freeform Type F characterized by two or more, or three or more XRPD signals selected from the group consisting of 18.0 °2θ, 10.8 °2θ, 14.4 °2θ, 23.1 °2θ, and 8.9 °2θ (±0.2 °2θ; ±0.1 °2θ; or ±0.0 °2θ; Cu Kα1 radiation). In some embodiments, the solid form of Freeform Type F is crystalline Freeform Type F characterized by XRPD signals at 18.0 °2θ, 10.8 °2θ, 14.4 °2θ, 23.1 °2θ, and 8.9 °2θ (±0.2 °2θ; ±0.1 °2θ; or ±0.0 °2θ; Cu Kα1 radiation).

In some embodiments, the crystalline Freeform Type F is characterized by one, two, three, four, or five XRPD signals selected from those set forth in Table 32.

TABLE 32
Freeform Type F XRPD Signals

| Signal No. | Pos. [°2θ] | d-spacing [Å] | \|Rel. Int. [%] |
|---|---|---|---|
| 1 | 8.93 | 9.9 | 18.98 |
| 2 | 10.84 | 8.16 | 52.45 |
| 3 | 14.36 | 6.17 | 40.47 |
| 4 | 18 | 4.93 | 100 |
| 5 | 23.07 | 3.85 | 27.28 |

### Freeform Type G

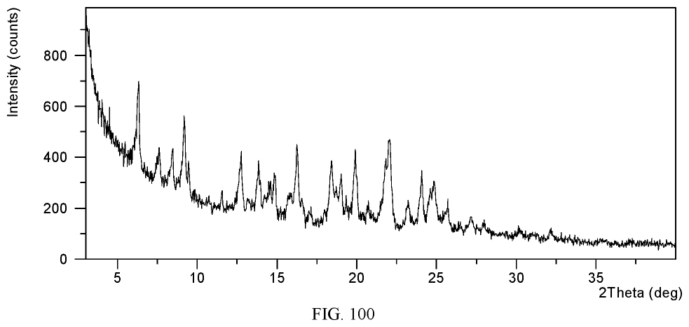

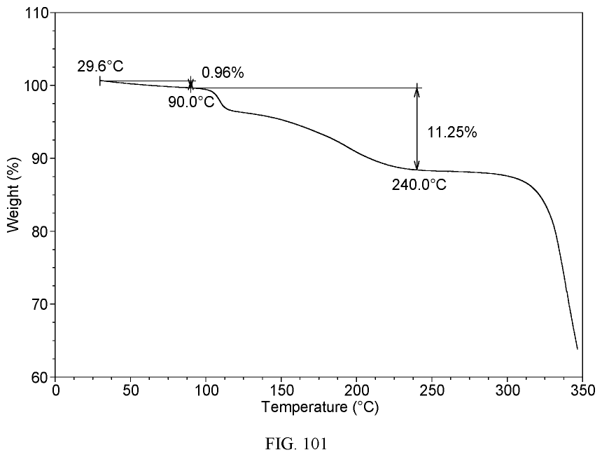

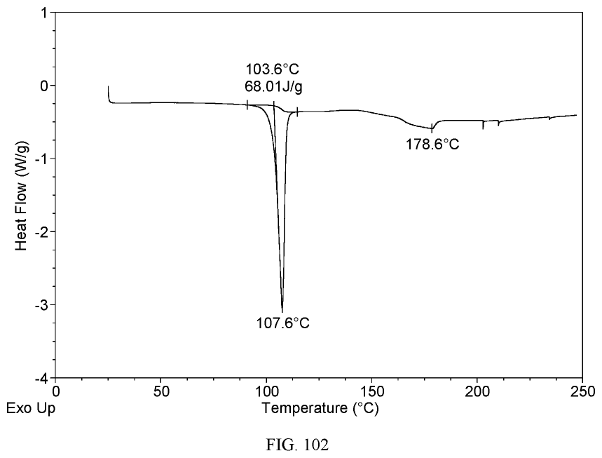

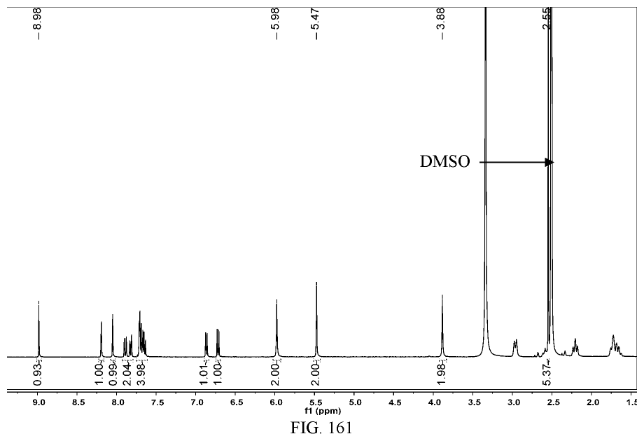

In some embodiments, the present disclosure provides solid forms of Freeform Type G, e.g., crystalline forms of Freeform Type G. In some embodiments, the Freeform Type G XRPD profile is substantially similar to that shown in FIG. 100. In some embodiments, the Freeform Type G TGA profile is substantially similar to that shown in FIG. 101. In some embodiments, the Freeform Type G DSC profile is substantially similar to that shown in FIG. 102. In some embodiments, the Freeform Type G ¹H NMR profile is substantially similar to that shown in FIG. 161. In some embodiments, the Freeform Type G is characterized by a DSC curve having endotherms at about 107.6° C. and about 178.6° C.

In some embodiments, the solid form of Freeform Type G is crystalline Freeform Type G characterized by two or more, or three XRPD signals selected from the group consisting of 6.3 °2θ, 22.1 °2θ, and 9.2 °2θ (±0.2 °2θ; ±0.1 °2θ; or ±0.0 °2θ; Cu Kα1 radiation). In some embodiments, the solid form of Freeform Type G is crystalline Freeform Type G characterized by XRPD signals at 6.3 °2θ, 22.1 °2θ, and 9.2 °2θ (±0.2 °2θ; ±0.1 °2θ; or ±0.0 °2θ; Cu Kα1 radiation).

In some embodiments, the solid form of Freeform Type G is crystalline Freeform Type G characterized by two or more, or three or more XRPD signals selected from the group consisting of 6.3 °2θ, 22.1 °2θ, 9.2 °2θ, 16.3 °2θ, and 19.9 °2θ (±0.2 °2θ; ±0.1 °2θ; or ±0.0 °2θ; Cu Kα1 radiation). In some embodiments, the solid form of Freeform Type G is crystalline Freeform Type G characterized by XRPD signals at 6.3 °2θ, 22.1 °2θ, 9.2 °2θ, 16.3 °2θ, and 19.9 °2θ (±0.2 °2θ; ±0.1 °2θ; or ±0.0 °2θ; Cu Kα1 radiation).

In some embodiments, the solid form of Freeform Type G is crystalline Freeform Type G characterized by two or more, or three or more XRPD signals selected from the group consisting of 6.3 °2θ, 22.1 °2θ, 9.2 °2θ, 16.3 °2θ, 19.9 °2θ, 18.4 °2θ, and 12.7 °2θ (±0.2 °2θ; ±0.1 °2θ; or ±0.0 °2θ; Cu Kα1 radiation). In some embodiments, the solid form of Freeform Type G is crystalline Freeform Type G characterized by XRPD signals at 6.3 °2θ, 22.1 °2θ, 9.2 °2θ, 16.3 °2θ, 19.9 °2θ, 18.4 °2θ, and 12.7 °2θ (±0.2 °2θ; ±0.1 °2θ; or ±0.0 °2θ; Cu Kα1 radiation).

In some embodiments, the solid form of Freeform Type G is crystalline Freeform Type G characterized by two or more, or three or more XRPD signals selected from the group consisting of 6.3 °2θ, 22.1 °2θ, 9.2 °2θ, 16.3 °2θ, 19.9 °2θ, 18.4 °2θ, 12.7 °2θ, 24.1 °2θ, 13.9 °2θ, and 19.0 °2θ (±0.2 °2θ; ±0.1 °2θ; or ±0.0 °2θ; Cu Kα1 radiation). In some embodiments, the solid form of Freeform Type G is crystalline Freeform Type G characterized by XRPD signals at 6.3 °2θ, 22.1 °2θ, 9.2 °2θ, 16.3 °2θ, 19.9 °2θ, 18.4 °2θ, 12.7 °2θ, 24.1 °2θ, 13.9 °2θ, and 19.0 °2θ (±0.2 °2θ; ±0.1 °2θ; or ±0.0 °2θ; Cu Kα1 radiation).

In some embodiments, the crystalline Freeform Type G is characterized by one, two, three, four, five, six, seven, eight, nine, ten, eleven, twelve, thirteen, fourteen, fifteen, or sixteen XRPD signals selected from those set forth in Table 33.

TABLE 33
Freeform Type G XRPD Signals

| Signal No. | Pos. [°2θ] | d-spacing [Å] | Rel. Int. [%] |
|---|---|---|---|
| 1 | 6.32 | 13.98 | 100 |
| 2 | 7.6 | 11.63 | 38.41 |
| 3 | 8.44 | 10.48 | 43.74 |
| 4 | 9.19 | 9.62 | 90.17 |
| 5 | 12.74 | 6.95 | 64.96 |
| 6 | 13.86 | 6.39 | 55.57 |
| 7 | 14.77 | 6 | 32.74 |
| 8 | 16.26 | 5.45 | 87.04 |
| 9 | 18.42 | 4.82 | 72.84 |
| 10 | 19.01 | 4.67 | 53.46 |
| 11 | 19.91 | 4.46 | 85.2 |
| 12 | 22.1 | 4.02 | 95.94 |
| 13 | 23.2 | 3.83 | 27.77 |
| 14 | 24.08 | 3.7 | 60.25 |
| 15 | 24.77 | 3.6 | 47.64 |
| 16 | 25.65 | 3.47 | 26.62 |

### Freeform Type H

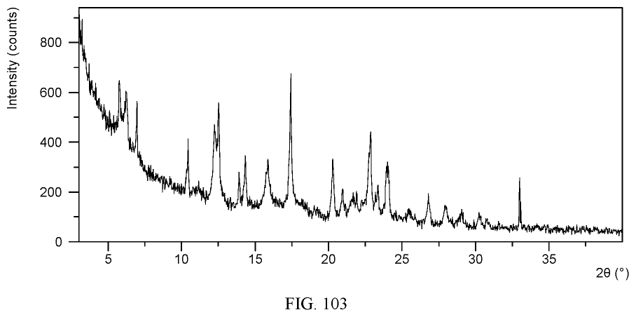

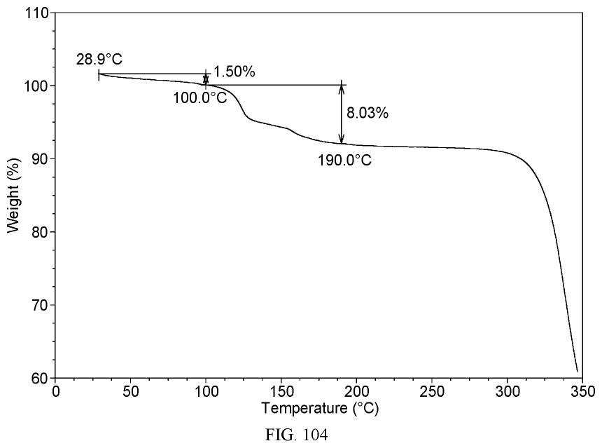

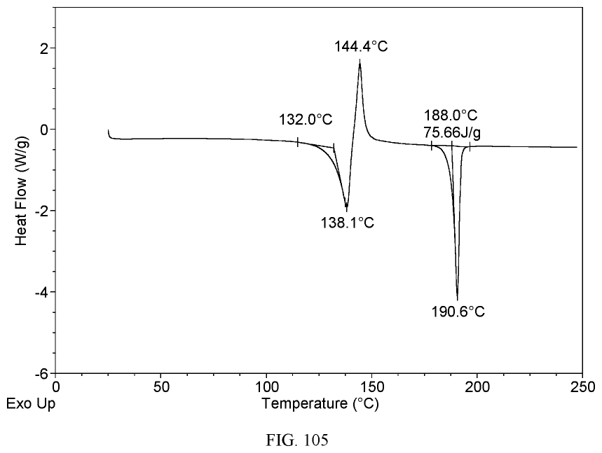

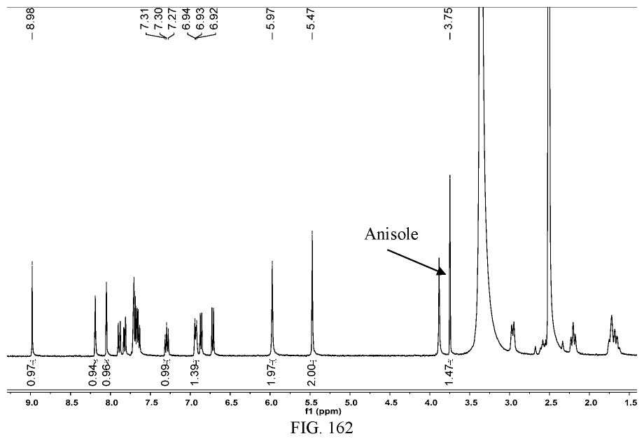

In some embodiments, the present disclosure provides solid forms of Freeform Type H, e.g., crystalline forms of Freeform Type H. In some embodiments, the Freeform Type H XRPD profile is substantially similar to that shown in FIG. 103. In some embodiments, the Freeform Type H TGA profile is substantially similar to that shown in FIG. 104. In some embodiments, the Freeform Type H DSC profile is substantially similar to that shown in FIG. 105. In some embodiments, the Freeform Type H ¹H NMR profile is substantially similar to that shown in FIG. 162. In some embodiments, the Freeform Type H is characterized by a DSC curve having endotherms at about 138.1° C. and about 190.6° C.

In some embodiments, the solid form of Freeform Type H is crystalline Freeform Type H characterized by two or more, or three XRPD signals selected from the group consisting of 17.4 °2θ, 12.5 °2θ, and 22.9 °2θ (±0.2 °2θ; ±0.1 °2θ; or ±0.0 °2θ; Cu Kα1 radiation). In some embodiments, the solid form of Freeform Type H is crystalline Freeform Type H characterized by XRPD signals at 17.4 °2θ, 12.5 °2θ, and 22.9 °2θ (±0.2 °2θ; ±0.1 °2θ; or ±0.0 °2θ; Cu Kα1 radiation).

In some embodiments, the solid form of Freeform Type H is crystalline Freeform Type H characterized by two or more, or three or more XRPD signals selected from the group consisting of 17.4 °2θ, 12.5 °2θ, 22.9 °2θ, 12.2 °2θ, and 20.3 °2θ (±0.2 °2θ; ±0.1 °2θ; or ±0.0 °2θ; Cu Kα1 radiation). In some embodiments, the solid form of Freeform Type H is crystalline Freeform Type H characterized by XRPD signals at 17.4 °2θ, 12.5 °2θ, 22.9 °2θ, 12.2 °2θ, and 20.3 °2θ (±0.2 °2θ; ±0.1 °2θ; or ±0.0 °2θ; Cu Kα1 radiation).

In some embodiments, the solid form of Freeform Type H is crystalline Freeform Type H characterized by two or more, or three or more XRPD signals selected from the group consisting of 17.4 °2θ, 12.5 °2θ, 22.9 °2θ, 12.2 °2θ, 20.3 °2θ, 7.0 °2θ, and 6.2 °2θ (±0.2 °2θ; ±0.1 °2θ; or ±0.0 °2θ; Cu Kα1 radiation). In some embodiments, the solid form of Freeform Type H is crystalline Freeform Type H characterized by XRPD signals at 17.4 °2θ, 12.5 °2θ, 22.9 °2θ, 12.2 °2θ, 20.3 °2θ, 7.0 °2θ, and 6.2 °2θ (±0.2 °2θ; ±0.1 °2θ; or ±0.0 °2θ; Cu Kα1 radiation).

In some embodiments, the solid form of Freeform Type H is crystalline Freeform Type H characterized by two or more, or three or more XRPD signals selected from the group consisting of 17.4 °2θ, 12.5 °2θ, 22.9 °2θ, 12.2 °2θ, 20.3 °2θ, 7.0 °2θ, 6.2 °2θ, 14.3 °2θ, 10.4 °2θ, and 24.0 °2θ (±0.2 °2θ; ±0.1 °2θ; or ±0.0 °2θ; Cu Kα1 radiation). In some embodiments, the solid form of Freeform Type H is crystalline Freeform Type H characterized by XRPD signals at 17.4 20, 12.5 °2θ, 22.9 °2θ, 12.2 °2θ, 20.3 °2θ, 7.0 °2θ, 6.2 °2θ, 14.3 °2θ, 10.4 °2θ, and 24.0 °2θ (±0.2 °2θ; ±0.1 °2θ; or ±0.0 °2θ; Cu Kα1 radiation).

In some embodiments, the crystalline Freeform Type H is characterized by one, two, three, four, five, six, seven, eight, nine, ten, eleven, twelve, thirteen, fourteen, fifteen, sixteen, seventeen, eighteen, or nineteen XRPD signals selected from those set forth in Table 34.

TABLE 34
Freeform Type H XRPD Signals

| Signal No. | Pos. [°2θ] | d-spacing [Å] | Rel. Int. [%] |
|---|---|---|---|
| 1 | 6.24 | 14.17 | 42.53 |
| 2 | 6.96 | 12.7 | 43.2 |
| 3 | 10.44 | 8.47 | 40.53 |
| 4 | 12.24 | 7.23 | 54.36 |
| 5 | 12.53 | 7.06 | 73.59 |
| 6 | 13.92 | 6.36 | 25.1 |
| 7 | 14.34 | 6.18 | 40.95 |
| 8 | 15.83 | 5.6 | 33.73 |
| 9 | 17.43 | 5.09 | 100 |
| 10 | 20.29 | 4.38 | 44.13 |
| 11 | 20.92 | 4.25 | 17.94 |
| 12 | 22.87 | 3.89 | 65.59 |
| 13 | 23.37 | 3.81 | 25.16 |
| 14 | 24.01 | 3.71 | 40.41 |
| 15 | 26.82 | 3.32 | 18.52 |
| 16 | 27.96 | 3.19 | 14.06 |
| 17 | 29.02 | 3.08 | 8.3 |
| 18 | 30.45 | 2.94 | 3.35 |
| 19 | 32.99 | 2.72 | 34.6 |

### Freeform Type I

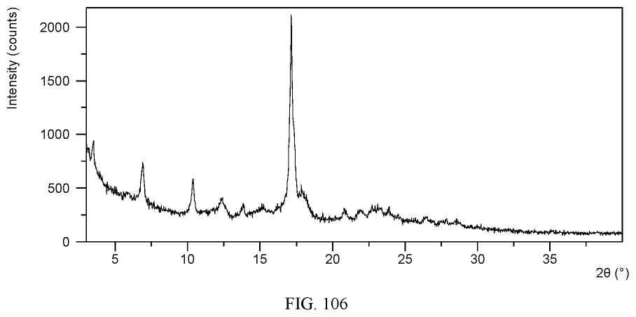

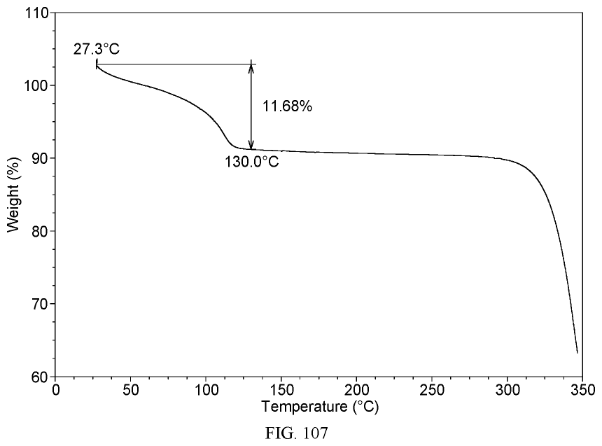

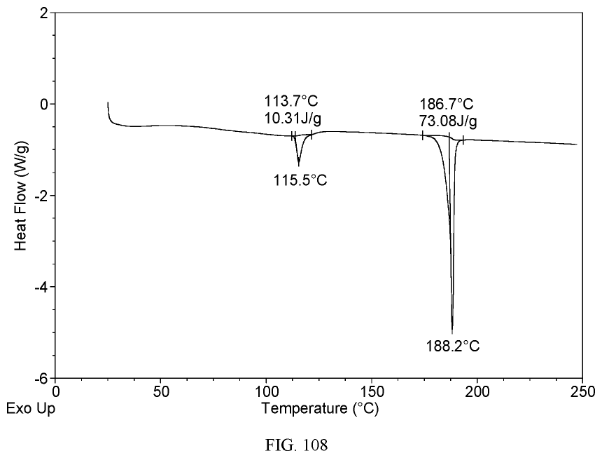

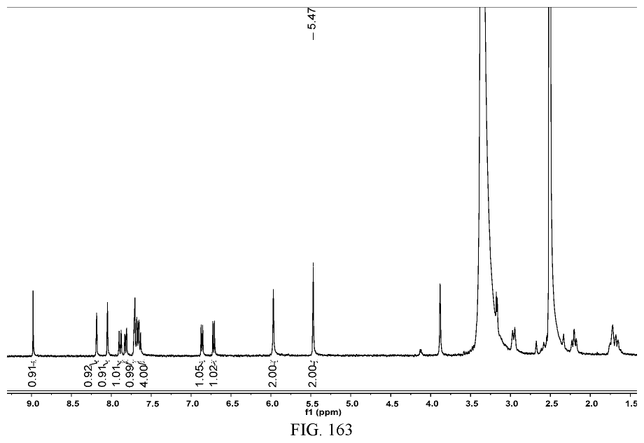

In some embodiments, the present disclosure provides solid forms of Freeform Type I, e.g., crystalline forms of Freeform Type I. In some embodiments, the Freeform Type I XRPD profile is substantially similar to that shown in FIG. 106. In some embodiments, the Freeform Type I TGA profile is substantially similar to that shown in FIG. 107. In some embodiments, the Freeform Type I DSC profile is substantially similar to that shown in FIG. 108. In some embodiments, the Freeform Type I ¹H NMR profile is substantially similar to that shown in FIG. 163. In some embodiments, the Freeform Type I is characterized by a DSC curve having endotherms at about 115.5° C. and about 188.2° C.

In some embodiments, the solid form of Freeform Type I is crystalline Freeform Type I characterized by two or more, or three XRPD signals selected from the group consisting of 17.2 °2θ, 6.9 °2θ, and 10.4 °2θ (±0.2 °2θ; ±0.1 °2θ; or ±0.0 °2θ; Cu Kα1 radiation). In some embodiments, the solid form of Freeform Type I is crystalline Freeform Type I characterized by XRPD signals at 17.2 °2θ, 6.9 °2θ, and 10.4 °2θ (±0.2 °2θ; ±0.1 °2θ; or ±0.0 °2θ; Cu Kα1 radiation).

In some embodiments, the solid form of Freeform Type I is crystalline Freeform Type I characterized by two or more, or three or more XRPD signals selected from the group consisting of 17.2 °2θ, 6.9 °2θ, 10.4 °2θ, 18.0 °2θ, and 3.5 °2θ (±0.2 °2θ; ±0.1 °2θ; or ±0.0 °2θ; Cu Kα1 radiation). In some embodiments, the solid form of Freeform Type I is crystalline Freeform Type I characterized by XRPD signals at 17.2 °2θ, 6.9 °2θ, 10.4 °2θ, 18.0 °2θ, and 3.5 °2θ (±0.2 °2θ; ±0.1 °2θ; or ±0.0 °2θ; Cu Kα1 radiation).

In some embodiments, the solid form of Freeform Type I is crystalline Freeform Type I characterized by two or more, or three or more XRPD signals selected from the group consisting of 17.2 °2θ, 6.9 °2θ, 10.4 °2θ, 18.0 °2θ, 3.5 °2θ, 12.4 °2θ, and 23.0 °2θ (±0.2 °2θ; ±0.1 °2θ; or ±0.0 °2θ; Cu Kα1 radiation). In some embodiments, the solid form of Freeform Type I is crystalline Freeform Type I characterized by XRPD signals at 17.2 °2θ, 6.9 °2θ, 10.4 °2θ, 18.0 °2θ, 3.5 °2θ, 12.4 °2θ, and 23.0 °2θ (±0.2 °2θ; ±0.1 °2θ; or ±0.0 °2θ; Cu Kα1 radiation).

In some embodiments, the solid form of Freeform Type I is crystalline Freeform Type I characterized by two or more, or three or more XRPD signals selected from the group consisting of 17.2 °2θ, 6.9 °2θ, 10.4 °2θ, 18.0 °2θ, 3.5 °2θ, 12.4 °2θ, 23.0 °2θ, 13.8 °2θ, 20.8 °2θ, and 0.0 °2θ (±0.2 °2θ; ±0.1 °2θ; or ±0.0 °2θ; Cu Kα1 radiation). In some embodiments, the solid form of Freeform Type I is crystalline Freeform Type I characterized by XRPD signals at 17.2 20, 6.9 °2θ, 10.4 °2θ, 18.0 °2θ, 3.5 °2θ, 12.4 °2θ, 23.0 °2θ, 13.8 °2θ, 20.8 °2θ, and 0.0 °2θ (±0.2 °2θ; ±0.1 °2θ; or ±0.0 °2θ; Cu Kα1 radiation).

In some embodiments, the crystalline Freeform Type I is characterized by one, two, three, four, five, six, seven, eight, or nine XRPD signals selected from those set forth in Table 35.

TABLE 35
Freeform Type I XRPD Signals

| Signal No. | Pos. [°2θ] | d-spacing [Å] | Rel. Int. [%] |
|---|---|---|---|
| 1 | 3.48 | 25.37 | 10.52 |
| 2 | 6.92 | 12.77 | 20.1 |
| 3 | 10.37 | 8.53 | 17.05 |
| 4 | 12.38 | 7.15 | 8.23 |
| 5 | 13.8 | 6.42 | 5.28 |
| 6 | 17.15 | 5.17 | 100 |
| 7 | 18.02 | 4.92 | 11.71 |
| 8 | 20.84 | 4.26 | 4.89 |
| 9 | 22.99 | 3.87 | 5.91 |

### Freeform Type J

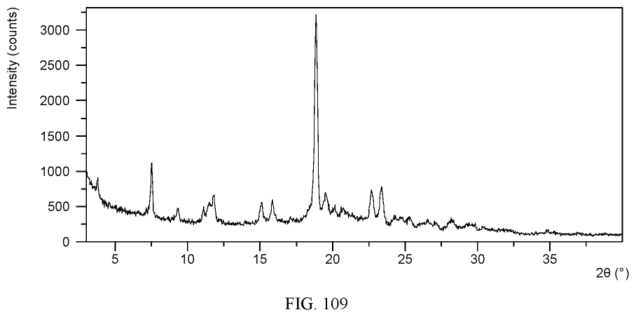

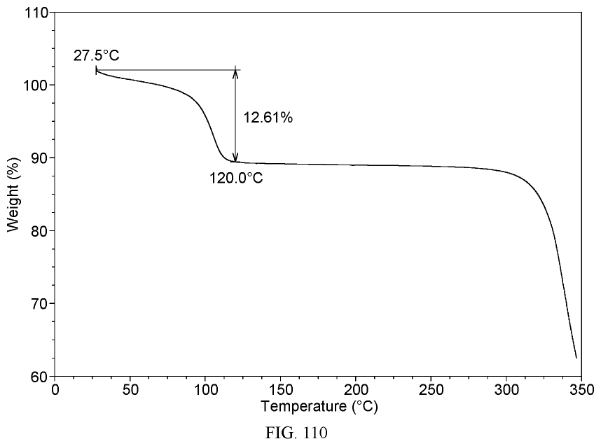

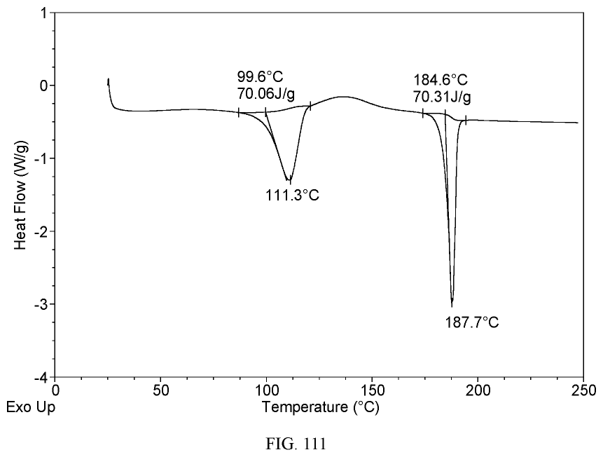

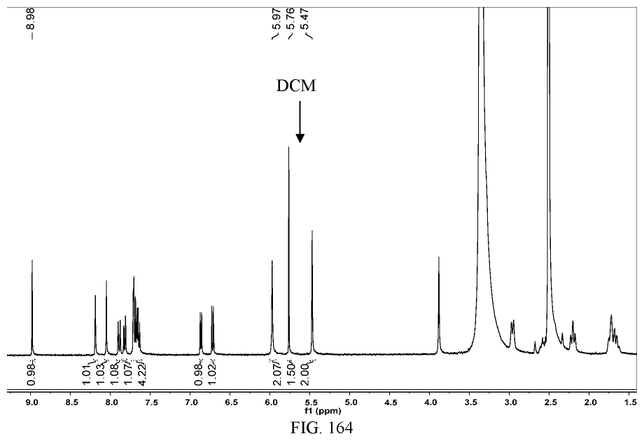

In some embodiments, the present disclosure provides solid forms of Freeform Type J, e.g., crystalline forms of Freeform Type J. In some embodiments, the Freeform Type J XRPD profile is substantially similar to that shown in FIG. 109. In some embodiments, the Freeform Type J TGA profile is substantially similar to that shown in FIG. 110. In some embodiments, the Freeform Type J DSC profile is substantially similar to that shown in FIG. 111. In some embodiments, the Freeform Type J ¹H NMR profile is substantially similar to that shown in FIG. 164. In some embodiments, the Freeform Type J is characterized by a DSC curve having endotherms at about 111.3° C. and about 187.7° C.

In some embodiments, the solid form of Freeform Type J is crystalline Freeform Type J characterized by two or more, or three XRPD signals selected from the group consisting of 18.9 °2θ, 7.5 °2θ, and 23.4 °2θ (±0.2 °2θ; ±0.1 °2θ; or ±0.0 °2θ; Cu Kα1 radiation). In some embodiments, the solid form of Freeform Type J is crystalline Freeform Type J characterized by XRPD signals at 18.9 °2θ, 7.5 °2θ, and 23.4 °2θ (±0.2 °2θ; ±0.1 °2θ; or ±0.0 °2θ; Cu Kα1 radiation).

In some embodiments, the solid form of Freeform Type J is crystalline Freeform Type J characterized by two or more, or three or more XRPD signals selected from the group consisting of 18.9 °2θ, 7.5 °2θ, 23.4 °2θ, 22.7 °2θ, and 19.6 °2θ (±0.2 °2θ; ±0.1 °2θ; or ±0.0 °2θ; Cu Kα1 radiation). In some embodiments, the solid form of Freeform Type J is crystalline Freeform Type J characterized by XRPD signals at 18.9 °2θ, 7.5 °2θ, 23.4 °2θ, 22.7 °2θ, and 19.6 °2θ (±0.2 °2θ; ±0.1 °2θ; or ±0.0 °2θ; Cu Kα1 radiation).

In some embodiments, the solid form of Freeform Type J is crystalline Freeform Type J characterized by two or more, or three or more XRPD signals selected from the group consisting of 18.9 °2θ, 7.5 °2θ, 23.4 °2θ, 22.7 °2θ, 19.6 °2θ, 11.8 °2θ, and 15.8 °2θ (±0.2 °2θ; ±0.1 °2θ; or ±0.0 °2θ; Cu Kα1 radiation). In some embodiments, the solid form of Freeform Type J is crystalline Freeform Type J characterized by XRPD signals at 18.9 °2θ, 7.5 °2θ, 23.4 °2θ, 22.7 °2θ, 19.6 °2θ, 11.8 °2θ, and 15.8 °2θ (±0.2 °2θ; ±0.1 °2θ; or ±0.0 °2θ; Cu Kα1 radiation).

In some embodiments, the solid form of Freeform Type J is crystalline Freeform Type J characterized by two or more, or three or more XRPD signals selected from the group consisting of 18.9 °2θ, 7.5 °2θ, 23.4 °2θ, 22.7 °2θ, 19.6 °2θ, 11.8 °2θ, 15.8 °2θ, 15.1 °2θ, 3.8 °2θ, and 20.7 °2θ (±0.2 °2θ; ±0.1 °2θ; or ±0.0 °2θ; Cu Kα1 radiation). In some embodiments, the solid form of Freeform Type J is crystalline Freeform Type J characterized by XRPD signals at 18.9 20, 7.5 °2θ, 23.4 °2θ, 22.7 °2θ, 19.6 °2θ, 11.8 °2θ, 15.8 °2θ, 15.1 °2θ, 3.8 °2θ, and 20.7 °2θ (±0.2 °2θ; ±0.1 °2θ; or ±0.0 °2θ; Cu Kα1 radiation).

In some embodiments, the crystalline Freeform Type J is characterized by one, two, three, four, five, six, seven, eight, nine, ten, eleven, twelve, thirteen, or fourteen XRPD signals selected from those set forth in Table 36.

TABLE 36
Freeform Type J XRPD Signals

| Signal No. | Pos. [°2θ] | d-spacing [Å] | Rel. Int. [%] |
|---|---|---|---|
| 1 | 3.79 | 23.33 | 6.98 |
| 2 | 7.53 | 11.74 | 25.04 |
| 3 | 9.34 | 9.47 | 5.9 |
| 4 | 11.79 | 7.51 | 12.89 |
| 5 | 15.12 | 5.86 | 9.83 |
| 6 | 15.84 | 5.6 | 10.02 |
| 7 | 18.87 | 4.7 | 100 |
| 8 | 19.55 | 4.54 | 13.37 |
| 9 | 20.74 | 4.28 | 6.23 |
| 10 | 22.67 | 3.92 | 16.01 |
| 11 | 23.41 | 3.8 | 16.96 |
| 12 | 25.33 | 3.52 | 3.69 |
| 13 | 28.3 | 3.15 | 3.99 |
| 14 | 34.96 | 2.57 | 1.43 |

### Freeform Type K

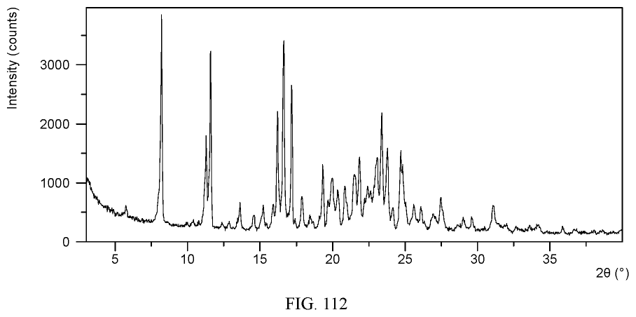

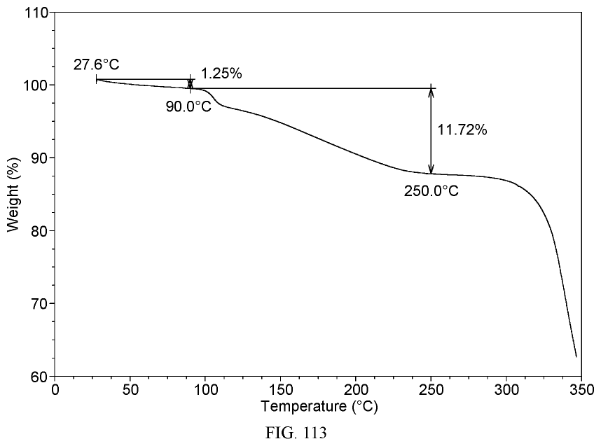

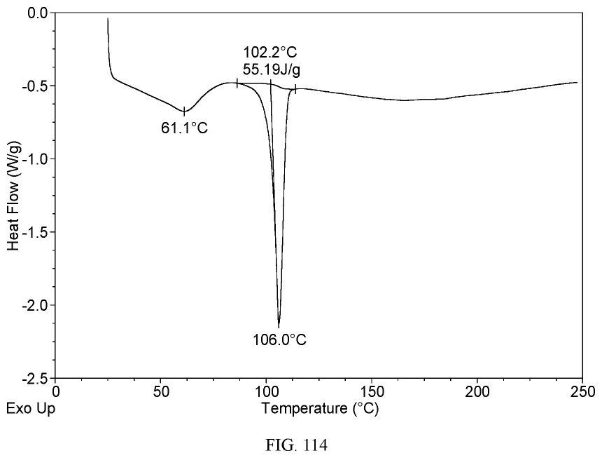

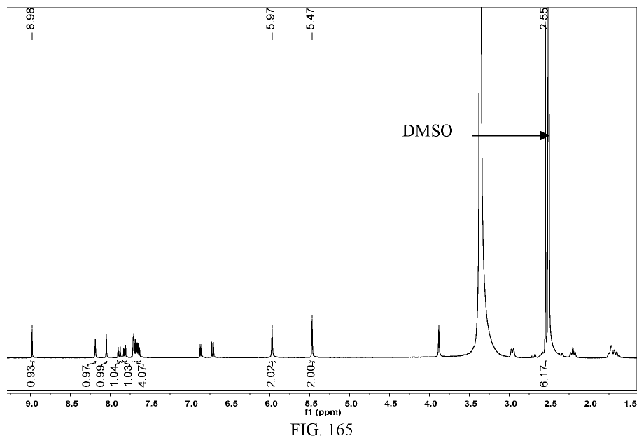

In some embodiments, the present disclosure provides solid forms of Freeform Type K, e.g., crystalline forms of Freeform Type K. In some embodiments, the Freeform Type K XRPD profile is substantially similar to that shown in FIG. 112. In some embodiments, the Freeform Type K TGA profile is substantially similar to that shown in FIG. 113. In some embodiments, the Freeform Type K DSC profile is substantially similar to that shown in FIG. 114. In some embodiments, the Freeform Type K ¹H NMR profile is substantially similar to that shown in FIG. 165. In some embodiments, the Freeform Type K is characterized by a DSC curve having endotherms at about 61.1° C. and about 106.0° C.

In some embodiments, the solid form of Freeform Type K is crystalline Freeform Type K characterized by two or more, or three XRPD signals selected from the group consisting of 8.2 °2θ, 16.6 °2θ, and 11.6 °2θ (±0.2 °2θ; ±0.1 °2θ; or ±0.0 °2θ; Cu Kα1 radiation). In some embodiments, the solid form of Freeform Type K is crystalline Freeform Type K characterized by XRPD signals at 8.2 °2θ, 16.6 °2θ, and 11.6 °2θ (±0.2 °2θ; ±0.1 °2θ; or ±0.0 °2θ; Cu Kα1 radiation).

In some embodiments, the solid form of Freeform Type K is crystalline Freeform Type K characterized by two or more, or three or more XRPD signals selected from the group consisting of 8.2 °2θ, 16.6 °2θ, 11.6 °2θ, 17.2 °2θ, and 16.2 °2θ (±0.2 °2θ; ±0.1 °2θ; or ±0.0 °2θ; Cu Kα1 radiation). In some embodiments, the solid form of Freeform Type K is crystalline Freeform Type K characterized by XRPD signals at 8.2 °2θ, 16.6 °2θ, 11.6 °2θ, 17.2 °2θ, and 16.2 °2θ (±0.2 °2θ; ±0.1 °2θ; or ±0.0 °2θ; Cu Kα1 radiation).

In some embodiments, the solid form of Freeform Type K is crystalline Freeform Type K characterized by two or more, or three or more XRPD signals selected from the group consisting of 8.2 °2θ, 16.6 °2θ, 11.6 °2θ, 17.2 °2θ, 16.2 °2θ, 23.4 °2θ, and 11.3 °2θ (±0.2 °2θ; ±0.1 °2θ; or ±0.0 °2θ; Cu Kα1 radiation). In some embodiments, the solid form of Freeform Type K is crystalline Freeform Type K characterized by XRPD signals at 8.2 °2θ, 16.6 °2θ, 11.6 °2θ, 17.2 °2θ, 16.2 °2θ, 23.4 °2θ, and 11.3 °2θ (±0.2 °2θ; ±0.1 °2θ; or ±0.0 °2θ; Cu Kα1 radiation).

In some embodiments, the solid form of Freeform Type K is crystalline Freeform Type K characterized by two or more, or three or more XRPD signals selected from the group consisting of 8.2 °2θ, 16.6 °2θ, 11.6 °2θ, 17.2 °2θ, 16.2 °2θ, 23.4 °2θ, 11.3 °2θ, 23.8 °2θ, 24.7 °2θ, and 21.9 °2θ (±0.2 °2θ; ±0.1 °2θ; or ±0.0 °2θ; Cu Kα1 radiation). In some embodiments, the solid form of Freeform Type K is crystalline Freeform Type K characterized by XRPD signals at 8.2 20, 16.6 °2θ, 11.6 °2θ, 17.2 °2θ, 16.2 °2θ, 23.4 °2θ, 11.3 °2θ, 23.8 °2θ, 24.7 °2θ, and 21.9 °2θ (±0.2 °2θ; ±0.1 °2θ; or ±0.0 °2θ; Cu Kα1 radiation).

In some embodiments, the crystalline Freeform Type K is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or forty XRPD signals selected from those set forth in Table 37.

TABLE 37
Freeform Type K XRPD Signals

| Signal No. | Pos. [°2θ] | d-spacing [Å] | Rel. Int. [%] |
|---|---|---|---|
| 1 | 5.75 | 15.36 | 4.55 |
| 2 | 8.2 | 10.78 | 100 |
| 3 | 11.28 | 7.84 | 43.06 |
| 4 | 11.59 | 7.64 | 83.96 |
| 5 | 12.85 | 6.89 | 3.01 |
| 6 | 13.62 | 6.5 | 12.55 |
| 7 | 14.58 | 6.08 | 6.51 |
| 8 | 15.22 | 5.82 | 10.75 |
| 9 | 15.91 | 5.57 | 11.4 |
| 10 | 16.2 | 5.47 | 55.02 |
| 11 | 16.63 | 5.33 | 89.35 |
| 12 | 17.18 | 5.16 | 65.85 |
| 13 | 17.89 | 4.96 | 14.49 |
| 14 | 18.44 | 4.81 | 5.09 |
| 15 | 19.34 | 4.59 | 27.75 |
| 16 | 19.67 | 4.51 | 11.67 |
| 17 | 19.98 | 4.44 | 22.41 |
| 18 | 20.39 | 4.36 | 15.81 |
| 19 | 20.83 | 4.26 | 17.56 |
| 20 | 21.45 | 4.14 | 22.65 |
| 21 | 21.85 | 4.07 | 32.15 |
| 22 | 22.42 | 3.97 | 17.93 |
| 23 | 23.08 | 3.85 | 31.79 |
| 24 | 23.39 | 3.8 | 52.45 |
| 25 | 23.79 | 3.74 | 36.02 |
| 26 | 24.17 | 3.68 | 8.63 |
| 27 | 24.69 | 3.61 | 33.98 |
| 28 | 24.86 | 3.58 | 26.89 |
| 29 | 25.61 | 3.48 | 10.43 |
| 30 | 26.1 | 3.41 | 9.39 |
| 31 | 26.87 | 3.32 | 5.19 |
| 32 | 27.46 | 3.25 | 14.44 |
| 33 | 29.03 | 3.08 | 4.83 |
| 34 | 29.6 | 3.02 | 5.63 |
| 35 | 31.09 | 2.88 | 11.71 |
| 36 | 32 | 2.8 | 2.68 |
| 37 | 32.72 | 2.74 | 1.8 |
| 38 | 34.2 | 2.62 | 3.57 |
| 39 | 35.88 | 2.5 | 3 |
| 40 | 36.71 | 2.45 | 1.81 |

### Freeform Type L

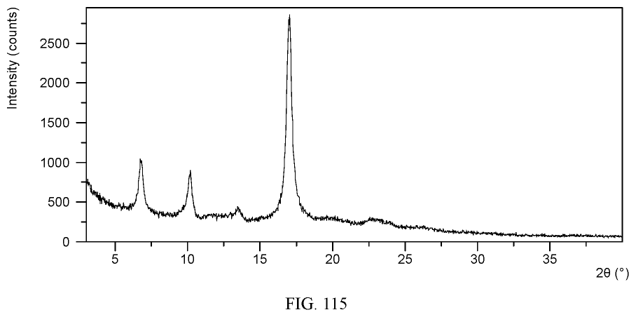

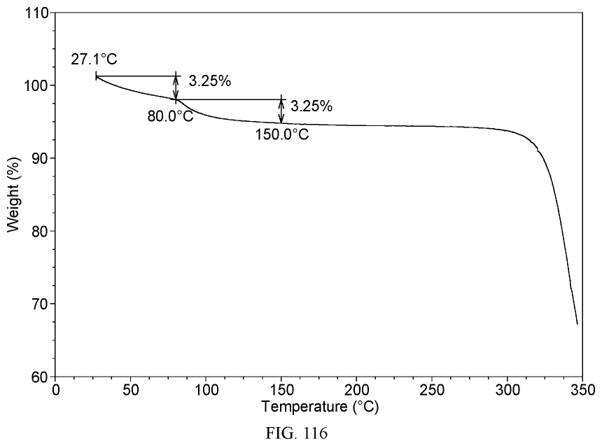

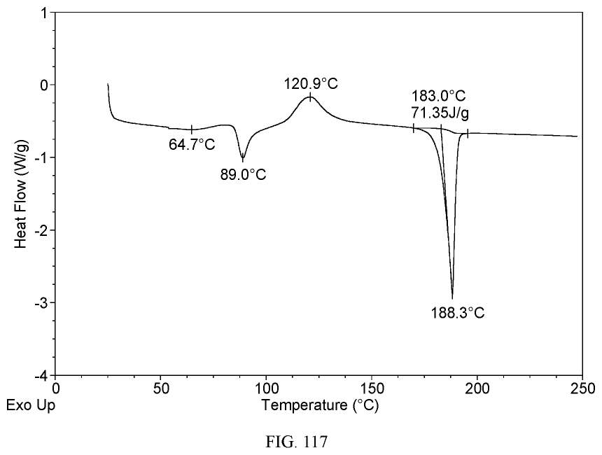

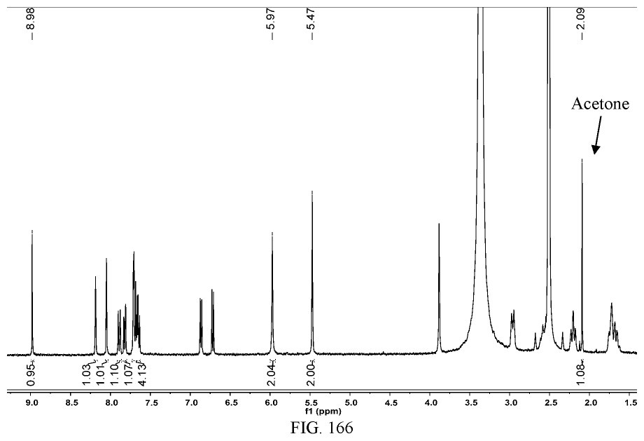

In some embodiments, the present disclosure provides solid forms of Freeform Type L, e.g., crystalline forms of Freeform Type L. In some embodiments, the Freeform Type L XRPD profile is substantially similar to that shown in FIG. 115. In some embodiments, the Freeform Type L TGA profile is substantially similar to that shown in FIG. 116. In some embodiments, the Freeform Type L DSC profile is substantially similar to that shown in FIG. 117. In some embodiments, the Freeform Type L ¹H NMR profile is substantially similar to that shown in FIG. 166. In some embodiments, the Freeform Type L is characterized by a DSC curve having endotherms at about 64.7° C., about 89.0° C. and about 188.3° C.

In some embodiments, the solid form of Freeform Type L is crystalline Freeform Type L characterized by two or more, or three XRPD signals selected from the group consisting of 17.1 °2θ, 6.8 °2θ, and 10.2 °2θ (±0.2 °2θ; ±0.1 °2θ; or ±0.0 °2θ; Cu Kα1 radiation). In some embodiments, the solid form of Freeform Type L is crystalline Freeform Type L characterized by XRPD signals at 17.1 °2θ, 6.8 °2θ, and 10.2 °2θ (±0.2 °2θ; ±0.1 °2θ; or ±0.0 °2θ; Cu Kα1 radiation).

In some embodiments, the solid form of Freeform Type L is crystalline Freeform Type L characterized by two or more, or three or more XRPD signals selected from the group consisting of 17.1 °2θ, 6.8 °2θ, 10.2 °2θ, and 13.5 °2θ (±0.2 °2θ; ±0.1 °2θ; or ±0.0 °2θ; Cu Kα1 radiation).

In some embodiments, the solid form of Freeform Type L is crystalline Freeform Type L characterized by XRPD signals at 17.1 °2θ, 6.8 °2θ, 10.2 °2θ, and 13.5 °2θ (±0.2 °2θ; ±0.1 °2θ; or ±0.0 °2θ; Cu Kα1 radiation).

In some embodiments, the crystalline Freeform Type L is characterized by one, two, three, or four XRPD signals selected from those set forth in Table 38.

TABLE 38
Freeform Type L XRPD Signals

| Signal | Pos. | d-spacing | Rel. Int. |
|---|---|---|---|
| No. | [°2θ] | [Å] | [%] |
| 1 | 6.78 | 13.05 | 25.74 |
| 2 | 10.23 | 8.65 | 19.47 |
| 3 | 13.51 | 6.55 | 5.96 |
| 4 | 17.07 | 5.2 | 100 |

### Freeform Type M

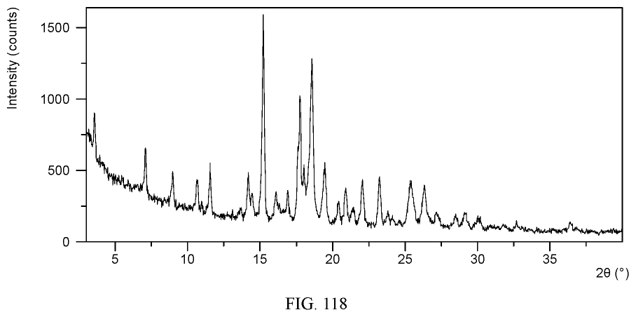

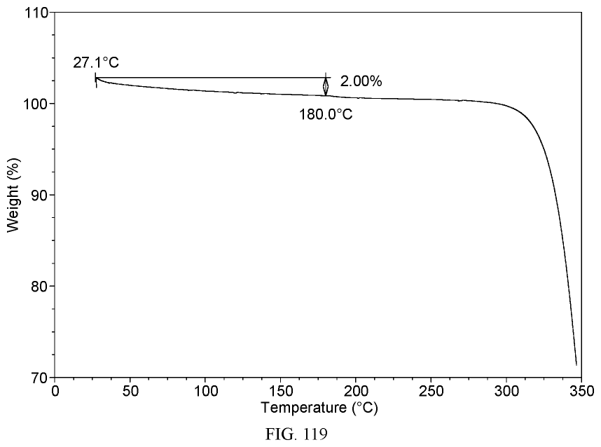

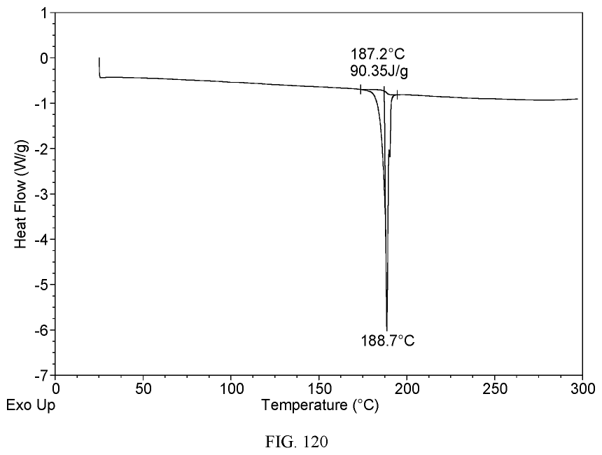

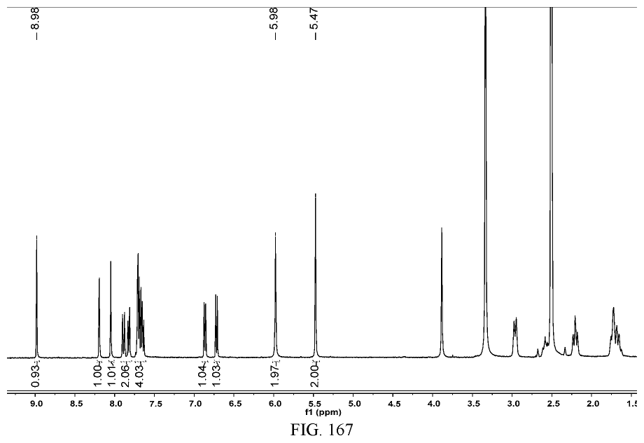

In some embodiments, the present disclosure provides solid forms of Freeform Type M, e.g., crystalline forms of Freeform Type M. In some embodiments, the Freeform Type M XRPD profile is substantially similar to that shown in FIG. 118. In some embodiments, the Freeform Type M TGA profile is substantially similar to that shown in FIG. 119. In some embodiments, the Freeform Type M DSC profile is substantially similar to that shown in FIG. 120. In some embodiments, the Freeform Type M ¹H NMR profile is substantially similar to that shown in FIG. 167. In some embodiments, the Freeform Type M is characterized by a DSC curve having an endotherm at about 188.7° C.

In some embodiments, the solid form of Freeform Type M is crystalline Freeform Type M characterized by two or more, or three XRPD signals selected from the group consisting of 15.2 °2θ, 18.6 °2θ, and 17.8 °2θ (±0.2 °2θ; ±0.1 °2θ; or ±0.0 °2θ; Cu Kα1 radiation). In some embodiments, the solid form of Freeform Type M is crystalline Freeform Type M characterized by XRPD signals at 15.2 °2θ, 18.6 °2θ, and 17.8 °2θ (±0.2 °2θ; ±0.1 °2θ; or ±0.0 °2θ; Cu Kα1 radiation).

In some embodiments, the solid form of Freeform Type M is crystalline Freeform Type M characterized by two or more, or three or more XRPD signals selected from the group consisting of 15.2 °2θ, 18.6 °2θ, 17.8 °2θ, 19.5 °2θ, and 23.2 °2θ (±0.2 °2θ; ±0.1 °2θ; or ±0.0 °2θ; Cu Kα1 radiation). In some embodiments, the solid form of Freeform Type M is crystalline Freeform Type M characterized by XRPD signals at 15.2 °2θ, 18.6 °2θ, 17.8 °2θ, 19.5 °2θ, and 23.2 °2θ (±0.2 °2θ; ±0.1 °2θ; or ±0.0 °2θ; Cu Kα1 radiation).

In some embodiments, the solid form of Freeform Type M is crystalline Freeform Type M characterized by two or more, or three or more XRPD signals selected from the group consisting of 15.2 °2θ, 18.6 °2θ, 17.8 °2θ, 19.5 °2θ, 23.2 °2θ, 7.1 °2θ, and 11.6 °2θ (±0.2 °2θ; ±0.1 °2θ; or ±0.0 °2θ; Cu Kα1 radiation). In some embodiments, the solid form of Freeform Type M is crystalline Freeform Type M characterized by XRPD signals at 15.2 °2θ, 18.6 °2θ, 17.8 °2θ, 19.5 °2θ, 23.2 °2θ, 7.1 °2θ, and 11.6 °2θ (±0.2 °2θ; ±0.1 °2θ; or ±0.0 °2θ; Cu Kα1 radiation).

In some embodiments, the solid form of Freeform Type M is crystalline Freeform Type M characterized by two or more, or three or more XRPD signals selected from the group consisting of 15.2 °2θ, 18.6 °2θ, 17.8 °2θ, 19.5 °2θ, 23.2 °2θ, 7.1 °2θ, 11.6 °2θ, 14.2 °2θ, 3.6 °2θ, and 26.4 °2θ (±0.2 °2θ; ±0.1 °2θ; or ±0.0 °2θ; Cu Kα1 radiation). In some embodiments, the solid form of Freeform Type M is crystalline Freeform Type M characterized by XRPD signals at 15.2 °2θ, 18.6 °2θ, 17.8 °2θ, 19.5 °2θ, 23.2 °2θ, 7.1 °2θ, 11.6 °2θ, 14.2 °2θ, 3.6 °2θ, and 26.4 °2θ (±0.2 °2θ; ±0.1 °2θ; or ±0.0 °2θ; Cu Kα1 radiation).

In some embodiments, the crystalline Freeform Type M is characterized by one, two, three, four, five, six, seven, eight, nine, ten, eleven, twelve, thirteen, fourteen, fifteen, sixteen, seventeen, eighteen, nineteen, twenty, twenty-one, twenty-two, or twenty-three XRPD signals selected from those set forth in Table 39.

TABLE 39
Freeform Type M XRPD Signals

| Signal | Pos. | d-spacing | Rel. Int. |
|---|---|---|---|
| No. | [°2θ] | [Å] | [%] |
| 1 | 3.57 | 24.74 | 20.68 |
| 2 | 7.09 | 12.46 | 23.32 |
| 3 | 8.98 | 9.85 | 16.27 |
| 4 | 10.7 | 8.27 | 15.69 |
| 5 | 11.57 | 7.65 | 22.22 |
| 6 | 14.18 | 6.24 | 21.18 |
| 7 | 15.23 | 5.82 | 100 |
| 8 | 16.08 | 5.51 | 12.88 |
| 9 | 16.91 | 5.24 | 13.24 |
| 10 | 17.76 | 4.99 | 61.12 |
| 11 | 18.59 | 4.77 | 80.32 |
| 12 | 19.45 | 4.56 | 28.17 |
| 13 | 20.41 | 4.35 | 9.96 |
| 14 | 20.94 | 4.24 | 16.01 |
| 15 | 21.44 | 4.15 | 7.12 |
| 16 | 22.03 | 4.03 | 20.13 |
| 17 | 23.23 | 3.83 | 23.92 |
| 18 | 25.33 | 3.52 | 18.56 |
| 19 | 26.35 | 3.38 | 20.41 |
| 20 | 28.5 | 3.13 | 5.93 |
| 21 | 29.13 | 3.07 | 7.87 |
| 22 | 30.09 | 2.97 | 5.07 |
| 23 | 36.43 | 2.47 | 4.79 |

### Freeform Type N

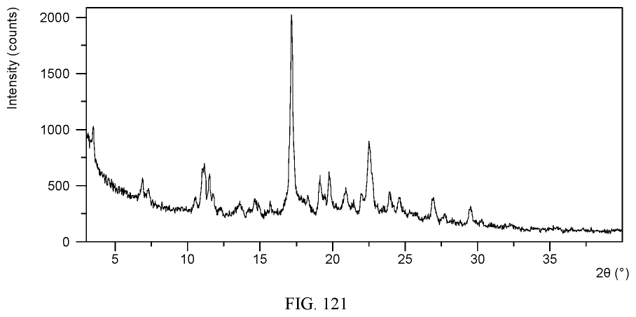

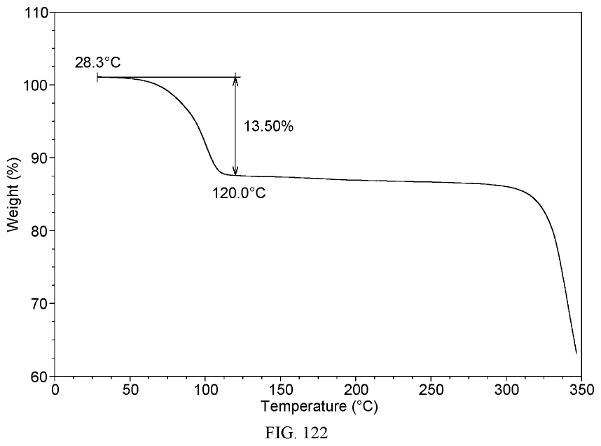

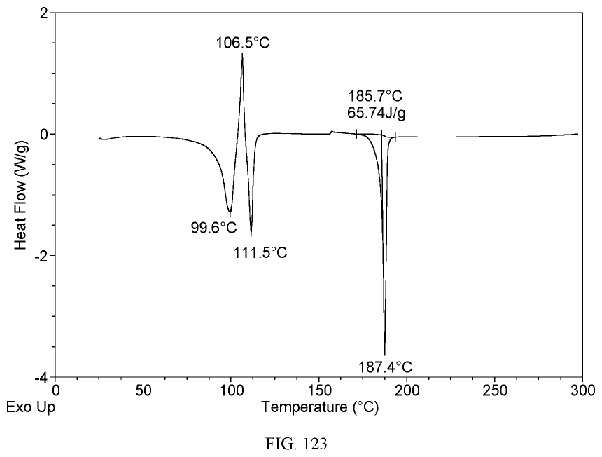

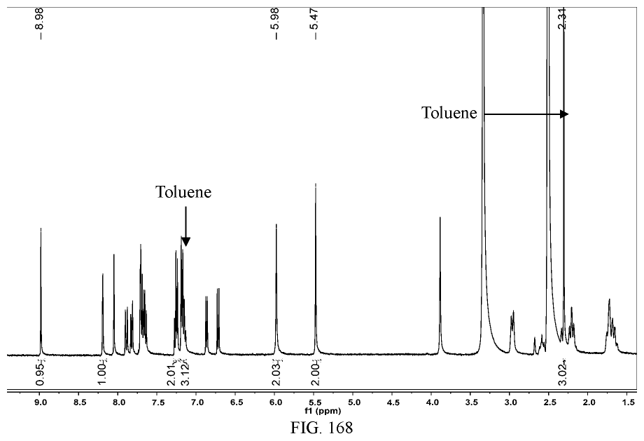

In some embodiments, the present disclosure provides solid forms of Freeform Type N, e.g., crystalline forms of Freeform Type N. In some embodiments, the Freeform Type N XRPD profile is substantially similar to that shown in FIG. 121. In some embodiments, the Freeform Type N TGA profile is substantially similar to that shown in FIG. 122. In some embodiments, the Freeform Type N DSC profile is substantially similar to that shown in FIG. 123. In some embodiments, the Freeform Type N ¹H NMR profile is substantially similar to that shown in FIG. 168. In some embodiments, the Freeform Type N is characterized by a DSC curve having endotherms at about 99.6° C., about 111.5° C., and about 187.4° C.

In some embodiments, the solid form of Freeform Type N is crystalline Freeform Type N characterized by two or more, or three XRPD signals selected from the group consisting of 17.2 °2θ, 22.5 °2θ, and 11.1 °2θ (±0.2 °2θ; ±0.1 °2θ; or ±0.0 °2θ; Cu Kα1 radiation). In some embodiments, the solid form of Freeform Type N is crystalline Freeform Type N characterized by XRPD signals at 17.2 °2θ, 22.5 °2θ, and 11.1 °2θ (±0.2 °2θ; ±0.1 °2θ; or ±0.0 °2θ; Cu Kα1 radiation).

In some embodiments, the solid form of Freeform Type N is crystalline Freeform Type N characterized by two or more, or three or more XRPD signals selected from the group consisting of 17.2 °2θ, 22.5 °2θ, 11.1 °2θ, 19.8 °2θ, and 11.5 °2θ (±0.2 °2θ; ±0.1 °2θ; or ±0.0 °2θ; Cu Kα1 radiation). In some embodiments, the solid form of Freeform Type N is crystalline Freeform Type N characterized by XRPD signals at 17.2 °2θ, 22.5 °2θ, 11.1 °2θ, 19.8 °2θ, and 11.5 °2θ (±0.2 °2θ; ±0.1 °2θ; or ±0.0 °2θ; Cu Kα1 radiation).

In some embodiments, the solid form of Freeform Type N is crystalline Freeform Type N characterized by two or more, or three or more XRPD signals selected from the group consisting of 17.2 °2θ, 22.5 °2θ, 11.1 °2θ, 19.8 °2θ, 11.5 °2θ, 19.1 °2θ, and 3.5 °2θ (±0.2 °2θ; ±0.1 °2θ; or ±0.0 °2θ; Cu Kα1 radiation). In some embodiments, the solid form of Freeform Type N is crystalline Freeform Type N characterized by XRPD signals at 17.2 °2θ, 22.5 °2θ, 11.1 °2θ, 19.8 °2θ, 11.5 °2θ, 19.1 °2θ, and 3.5 °2θ (±0.2 °2θ; ±0.1 °2θ; or ±0.0 °2θ; Cu Kα1 radiation).

In some embodiments, the solid form of Freeform Type N is crystalline Freeform Type N characterized by two or more, or three or more XRPD signals selected from the group consisting of 17.2 °2θ, 22.5 °2θ, 11.1 °2θ, 19.8 °2θ, 11.5 °2θ, 19.1 °2θ, 3.5 °2θ, 23.9 °2θ, 20.9 °2θ, and 26.9 °2θ (±0.2 °2θ; ±0.1 °2θ; or ±0.0 °2θ; Cu Kα1 radiation). In some embodiments, the solid form of Freeform Type N is crystalline Freeform Type N characterized by XRPD signals at 17.2 20, 22.5 °2θ, 11.1 °2θ, 19.8 °2θ, 11.5 °2θ, 19.1 °2θ, 3.5 °2θ, 23.9 °2θ, 20.9 °2θ, and 26.9 °2θ (±0.2 °2θ; ±0.1 °2θ; or ±0.0 °2θ; Cu Kα1 radiation).

In some embodiments, the crystalline Freeform Type N is characterized by one, two, three, four, five, six, seven, eight, nine, ten, eleven, twelve, thirteen, fourteen, fifteen, sixteen, or seventeen XRPD signals selected from those set forth in Table 40.

TABLE 40
Freeform Type N XRPD Signals

| Signal | Pos. | d-spacing | Rel. Int. |
|---|---|---|---|
| No. | [°2θ] | [Å] | [%] |
| 1 | 3.48 | 25.37 | 14.28 |
| 2 | 6.87 | 12.87 | 10.92 |
| 3 | 11.12 | 7.95 | 23.19 |
| 4 | 11.5 | 7.7 | 19.25 |
| 5 | 13.61 | 6.51 | 5.78 |
| 6 | 14.75 | 6.01 | 5.44 |
| 7 | 15.69 | 5.65 | 4.17 |
| 8 | 17.17 | 5.16 | 100 |
| 9 | 19.13 | 4.64 | 18.52 |
| 10 | 19.75 | 4.49 | 19.83 |
| 11 | 20.91 | 4.25 | 12.19 |
| 12 | 21.99 | 4.04 | 10.47 |
| 13 | 22.5 | 3.95 | 35.15 |
| 14 | 23.93 | 3.72 | 12.83 |
| 15 | 24.61 | 3.62 | 10.03 |
| 16 | 26.93 | 3.31 | 11.12 |
| 17 | 29.51 | 3.03 | 8.71 |

### Freeform Type O

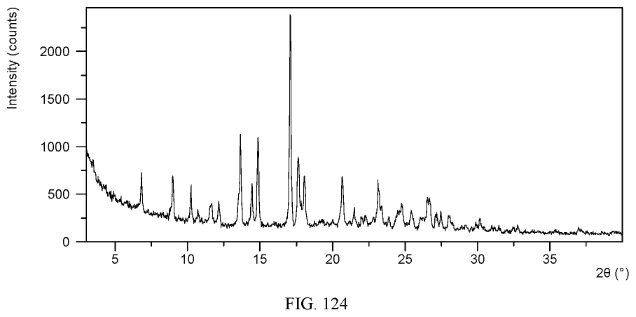

In some embodiments, the present disclosure provides solid forms of Freeform Type O, e.g., crystalline forms of Freeform Type O. In some embodiments, the Freeform Type O XRPD profile is substantially similar to that shown in FIG. 124.

In some embodiments, the solid form of Freeform Type O is crystalline Freeform Type O characterized by two or more, or three XRPD signals selected from the group consisting of 17.1 °2θ, 13.7 °2θ, and 14.9 °2θ (±0.2 °2θ; ±0.1 °2θ; or ±0.0 °2θ; Cu Kα1 radiation). In some embodiments, the solid form of Freeform Type O is crystalline Freeform Type O characterized by XRPD signals at 17.1 °2θ, 13.7 °2θ, and 14.9 °2θ (±0.2 °2θ; ±0.1 °2θ; or ±0.0 °2θ; Cu Kα1 radiation).

In some embodiments, the solid form of Freeform Type O is crystalline Freeform Type O characterized by two or more, or three or more XRPD signals selected from the group consisting of 17.1 °2θ, 13.7 °2θ, 14.9 °2θ, 17.6 °2θ, and 18.1 °2θ (±0.2 °2θ; ±0.1 °2θ; or ±0.0 °2θ; Cu Kα1 radiation). In some embodiments, the solid form of Freeform Type O is crystalline Freeform Type O characterized by XRPD signals at 17.1 °2θ, 13.7 °2θ, 14.9 °2θ, 17.6 °2θ, and 18.1 °2θ (±0.2 °2θ; ±0.1 °2θ; or ±0.0 °2θ; Cu Kα1 radiation).

In some embodiments, the solid form of Freeform Type O is crystalline Freeform Type O characterized by two or more, or three or more XRPD signals selected from the group consisting of 17.1 °2θ, 13.7 °2θ, 14.9 °2θ, 17.6 °2θ, 18.1 °2θ, 20.7 °2θ, and 23.1 °2θ (±0.2 °2θ; ±0.1 °2θ; or ±0.0 °2θ; Cu Kα1 radiation). In some embodiments, the solid form of Freeform Type O is crystalline Freeform Type O characterized by XRPD signals at 17.1 °2θ, 13.7 °2θ, 14.9 °2θ, 17.6 °2θ, 18.1 °2θ, 20.7 °2θ, and 23.1 °2θ (±0.2 °2θ; ±0.1 °2θ; or ±0.0 °2θ; Cu Kα1 radiation).

In some embodiments, the solid form of Freeform Type O is crystalline Freeform Type O characterized by two or more, or three or more XRPD signals selected from the group consisting of 17.1 °2θ, 13.7 °2θ, 14.9 °2θ, 17.6 °2θ, 18.1 °2θ, 20.7 °2θ, 23.1 °2θ, 9.0 °2θ, 14.4 °2θ, and 6.8 °2θ (±0.2 °2θ; ±0.1 °2θ; or ±0.0 °2θ; Cu Kα1 radiation). In some embodiments, the solid form of Freeform Type O is crystalline Freeform Type O characterized by XRPD signals at 17.1 °2θ, 13.7 °2θ, 14.9 °2θ, 17.6 °2θ, 18.1 °2θ, 20.7 °2θ, 23.1 °2θ, 9.0 °2θ, 14.4 °2θ, and 6.8 °2θ (±0.2 °2θ; ±0.1 °2θ; or ±0.0 °2θ; Cu Kα1 radiation).

In some embodiments, the crystalline Freeform Type O is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 41.

TABLE 41
Freeform Type O XRPD Signals

| Signal | Pos. | d-spacing | Rel. Int. |
|---|---|---|---|
| No. | [°2θ] | [Å] | [%] |
| 1 | 6.82 | 12.97 | 16.5 |
| 2 | 8.98 | 9.85 | 19.95 |
| 3 | 10.23 | 8.65 | 16.36 |
| 4 | 11.6 | 7.63 | 8.75 |
| 5 | 12.15 | 7.28 | 10.62 |
| 6 | 13.65 | 6.49 | 42.6 |
| 7 | 14.44 | 6.13 | 19.03 |
| 8 | 14.86 | 5.96 | 40.16 |
| 9 | 17.08 | 5.19 | 100 |
| 10 | 17.63 | 5.03 | 32.08 |
| 11 | 18.06 | 4.91 | 23.24 |
| 12 | 20.68 | 4.3 | 22.9 |
| 13 | 21.49 | 4.13 | 8.92 |
| 14 | 22.16 | 4.01 | 3.11 |
| 15 | 23.14 | 3.84 | 21.6 |
| 16 | 23.9 | 3.72 | 4.62 |
| 17 | 24.79 | 3.59 | 11 |
| 18 | 25.46 | 3.5 | 7.64 |
| 19 | 26.72 | 3.34 | 12.9 |
| 20 | 27.48 | 3.25 | 7.77 |
| 21 | 28.04 | 3.18 | 6.73 |
| 22 | 30.18 | 2.96 | 5.93 |
| 23 | 32.59 | 2.75 | 1.98 |
| 24 | 37.07 | 2.43 | 2.17 |

### Freeform Type P

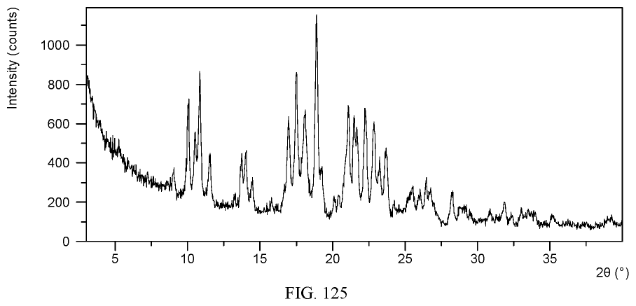

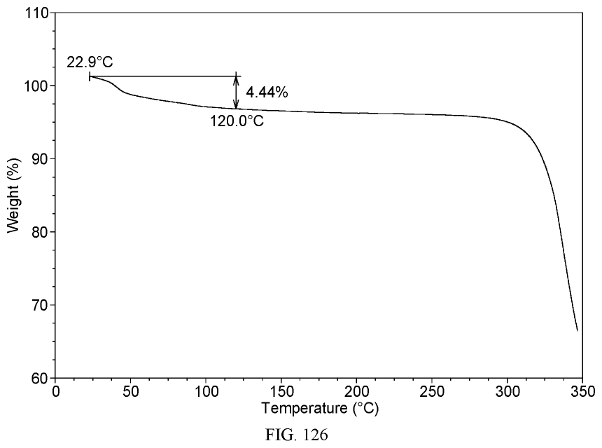

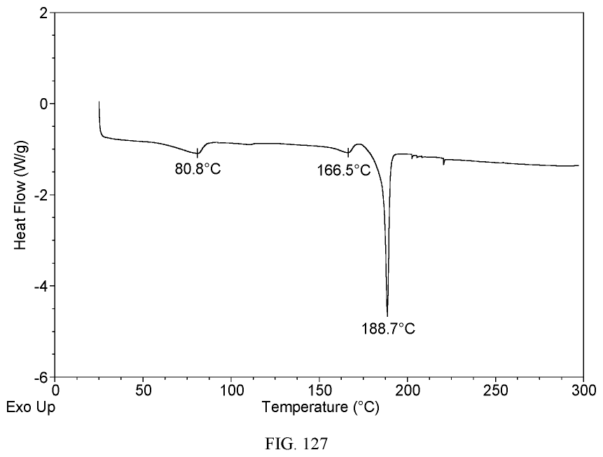

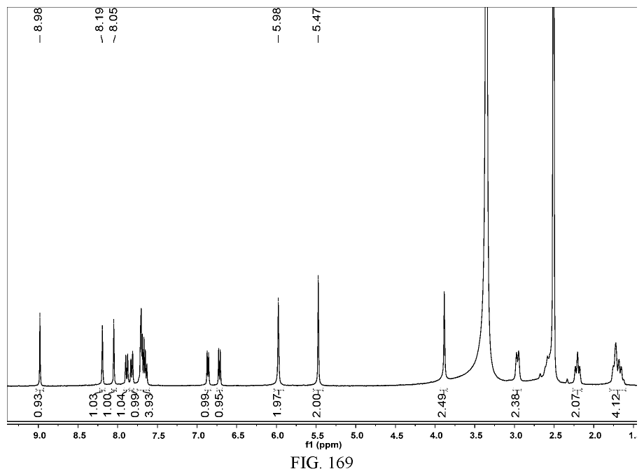

In some embodiments, the present disclosure provides solid forms of Freeform Type P, e.g., crystalline forms of Freeform Type P. In some embodiments, the Freeform Type P XRPD profile is substantially similar to that shown in FIG. 125. In some embodiments, the Freeform Type P TGA profile is substantially similar to that shown in FIG. 126. In some embodiments, the Freeform Type P DSC profile is substantially similar to that shown in FIG. 127. In some embodiments, the Freeform Type P ¹H NMR profile is substantially similar to that shown in FIG. 169. In some embodiments, the Freeform Type P is characterized by a DSC curve having endotherms at about 80.8° C., about 166.5° C., and about 188.7° C.

In some embodiments, the solid form of Freeform Type P is crystalline Freeform Type P characterized by two or more, or three XRPD signals selected from the group consisting of 18.9 °2θ, 17.5 °2θ, and 10.9 °2θ (±0.2 °2θ; ±0.1 °2θ; or ±0.0 °2θ; Cu Kα1 radiation). In some embodiments, the solid form of Freeform Type P is crystalline Freeform Type P characterized by XRPD signals at 18.9 °2θ, 17.5 °2θ, and 10.9 °2θ (±0.2 °2θ; ±0.1 °2θ; or ±0.0 °2θ; Cu Kα1 radiation).

In some embodiments, the solid form of Freeform Type P is crystalline Freeform Type P characterized by two or more, or three or more XRPD signals selected from the group consisting of 18.9 °2θ, 17.5 °2θ, 10.9 °2θ, 21.1 °2θ, and 22.3 °2θ (±0.2 °2θ; ±0.1 °2θ; or ±0.0 °2θ; Cu Kα1 radiation). In some embodiments, the solid form of Freeform Type P is crystalline Freeform Type O characterized by XRPD signals at 18.9 °2θ, 17.5 °2θ, 10.9 °2θ, 21.1 °2θ, and 22.3 °2θ (±0.2 °2θ; ±0.1 °2θ; or ±0.0 °2θ; Cu Kα1 radiation).

In some embodiments, the solid form of Freeform Type P is crystalline Freeform Type P characterized by two or more, or three or more XRPD signals selected from the group consisting of 18.9 °2θ, 17.5 °2θ, 10.9 °2θ, 21.1 °2θ, 22.3 °2θ, 18.1 °2θ, and 21.5 °2θ (±0.2 °2θ; ±0.1 °2θ; or ±0.0 °2θ; Cu Kα1 radiation). In some embodiments, the solid form of Freeform Type P is crystalline Freeform Type P characterized by XRPD signals at 18.9 °2θ, 17.5 °2θ, 10.9 °2θ, 21.1 °2θ, 22.3 °2θ, 18.1 °2θ, and 21.5 °2θ (±0.2 °2θ; ±0.1 °2θ; or ±0.0 °2θ; Cu Kα1 radiation).

In some embodiments, the solid form of Freeform Type O is crystalline Freeform Type P characterized by two or more, or three or more XRPD signals selected from the group consisting of 18.9 °2θ, 17.5 °2θ, 10.9 °2θ, 21.1 °2θ, 22.3 °2θ, 18.1 °2θ, 21.5 °2θ, 17.0 °2θ, 10.1 °2θ, and 22.9 °2θ (±0.2 °2θ; ±0.1 °2θ; or ±0.0 °2θ; Cu Kα1 radiation). In some embodiments, the solid form of Freeform Type O is crystalline Freeform Type P characterized by XRPD signals at 18.9 °2θ, 17.5 °2θ, 10.9 °2θ, 21.1 °2θ, 22.3 °2θ, 18.1 °2θ, 21.5 °2θ, 17.0 °2θ, 10.1 °2θ, and 22.9 °2θ (0.2 °2θ; ±0.1 °2θ; or ±0.0 °2θ; Cu Kα1 radiation).

In some embodiments, the crystalline Freeform Type P is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five XRPD signals selected from those set forth in Table 42.

TABLE 42
Freeform Type P XRPD Signals

| Signal | Pos. | d-spacing | Rel. Int. |
|---|---|---|---|
| No. | [°2θ] | [Å] | [%] |
| 1 | 9.04 | 9.78 | 10.55 |
| 2 | 10.05 | 8.8 | 46.74 |
| 3 | 10.52 | 8.41 | 33.47 |
| 4 | 10.85 | 8.15 | 64.13 |
| 5 | 11.55 | 7.66 | 21.48 |
| 6 | 13.75 | 6.44 | 24.17 |
| 7 | 14.03 | 6.31 | 30.05 |
| 8 | 14.46 | 6.13 | 15.05 |
| 9 | 16.96 | 5.23 | 47.92 |
| 10 | 17.5 | 5.07 | 70.78 |
| 11 | 18.08 | 4.91 | 50.22 |
| 12 | 18.9 | 4.69 | 100 |
| 13 | 21.1 | 4.21 | 55.41 |
| 14 | 21.45 | 4.14 | 48.28 |
| 15 | 21.69 | 4.1 | 44.08 |
| 16 | 22.25 | 4 | 54.43 |
| 17 | 22.89 | 3.89 | 44.59 |
| 18 | 23.73 | 3.75 | 31.49 |
| 19 | 25.42 | 3.5 | 13.66 |
| 20 | 26.44 | 3.37 | 20.53 |
| 21 | 28.18 | 3.17 | 14.54 |
| 22 | 28.9 | 3.09 | 7.58 |
| 23 | 31.87 | 2.81 | 11.66 |
| 24 | 33.46 | 2.68 | 5.62 |
| 25 | 35.19 | 2.55 | 5.14 |

### IV. Pharmaceutical Compositions and Formulations

In some aspects, the present disclosure provides a pharmaceutical composition comprising a form of Compound 1 disclosed herein as an active ingredient.

As used herein, the term “composition” is intended to encompass a product comprising the specified ingredients in the specified amounts, as well as any product which results, directly or indirectly, from combination of the specified ingredients in the specified amounts.

The forms of Compound 1 of the present disclosure can be formulated for oral administration in forms such as tablets, capsules (each of which includes immediate release, sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent a GLP-1R related condition referred to herein, slow its progression and/or reduce the symptoms associated with the condition.

An effective amount of a compound of the present disclosure for use in therapy is an amount sufficient to treat a GLP-1R related condition referred to herein, slow its progression and/or reduce the symptoms associated with the condition.

The size of the dose for therapeutic or prophylactic purposes of a form of Compound 1 disclosed herein will naturally vary according to the nature and severity of the conditions, the age and sex of the animal or patient and the route of administration, according to well-known principles of medicine.

### V. Administration

The forms of Compound 1 disclosed herein, or pharmaceutical compositions comprising the same, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the form of Compound 1 with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which GLP-1R activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, the forms of compound 1 disclosed herein may also be useful as pharmacological tools in the development and standardization of in vitro and in vivo test systems for the evaluation of the effects of modulators of GLP-1R activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The forms of Compound 1 of the disclosure or pharmaceutical compositions comprising these forms may be administered to a subject by any route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### VI. Methods of Treatment

In some aspects, the present disclosure provides a method of modulating GLP-1R activity (e.g., in vitro or in vivo), comprising contacting a cell with an effective amount of a form of Compound 1 disclosed herein.

In some aspects, the present disclosure provides a method of modulating GLP-1R activity (e.g., in vitro or in vivo), comprising contacting a cell with a form of Compound 1 disclosed herein.

In some aspects, the present disclosure provides a method of modulating GLP-1R activity (e.g., in vitro or in vivo), comprising contacting a cell with a form of Compound 1 disclosed herein.

In some aspects, the present disclosure provides a method of treating or preventing a disease or disorder disclosed herein in a subject in need thereof, comprising administering to the subject a therapeutically effective amount of a form of Compound 1 disclosed herein.

In some aspects, the present disclosure provides a method of treating a disease or disorder disclosed herein in a subject in need thereof, comprising administering to the subject a therapeutically effective amount of a form of Compound 1 disclosed herein, or a pharmaceutical composition of the present disclosure.

In some aspects, the present disclosure provides a method of treating or preventing a disease or disorder disclosed herein in a subject in need thereof, comprising administering to the subject a form of Compound 1 disclosed herein.

In some aspects, the present disclosure provides a method of treating a disease or disorder disclosed herein in a subject in need thereof, comprising administering to the subject a form of Compound 1 disclosed herein, or a pharmaceutical composition of the present disclosure.

In some embodiments, the disease or disorder is associated with an implicated GLP-1R activity. In some embodiments, the disease or disorder is a disease or disorder in which GLP-1R activity is implicated.

In some embodiments, the disease or disorder is diabetes, NASH, insulinoma, obesity, and/or hyperglycemia.

In some aspects, the present disclosure provides a method of treating or preventing diabetes, NASH, insulinoma, obesity, and/or hyperglycemia in a subject in need thereof, comprising administering to the subject a therapeutically effective amount of a form of Compound 1 disclosed herein, or a pharmaceutical composition of the present disclosure.

In some aspects, the present disclosure provides a method of treating diabetes, NASH, insulinoma, obesity, and/or hyperglycemia in a subject in need thereof, comprising administering to the subject a therapeutically effective amount of a form of Compound 1 disclosed herein, or a pharmaceutical composition of the present disclosure.

In some aspects, the present disclosure provides a method of treating or preventing diabetes, NASH, insulinoma, obesity, and/or hyperglycemia in a subject in need thereof, comprising administering to the subject a form of Compound 1 disclosed herein, or a pharmaceutical composition of the present disclosure.

In some aspects, the present disclosure provides a method of treating diabetes, NASH, insulinoma, obesity, and/or hyperglycemia in a subject in need thereof, comprising administering to the subject a form of Compound 1 disclosed herein, or a pharmaceutical composition of the present disclosure.

In some aspects, the present disclosure provides a form of Compound 1 disclosed herein for use in modulating GLP-1R activity (e.g., in vitro or in vivo).

In some aspects, the present disclosure provides a form of Compound 1 disclosed herein for use in modulating GLP-1R activity (e.g., in vitro or in vivo).

In some aspects, the present disclosure provides a form of Compound 1 disclosed herein for use in treating or preventing a disease or disorder disclosed herein.

In some aspects, the present disclosure provides a form of Compound 1 disclosed herein for use in treating a disease or disorder disclosed herein.

In some aspects, the present disclosure provides a form of Compound 1 disclosed herein for use in treating or preventing diabetes, NASH, insulinoma, obesity, and/or hyperglycemia in a subject in need thereof.

In some aspects, the present disclosure provides a form of Compound 1 disclosed herein for use in treating diabetes, NASH, insulinoma, obesity, and/or hyperglycemia in a subject in need thereof.

In some aspects, the present disclosure provides use of a form of Compound 1 disclosed herein in the manufacture of a medicament for modulating GLP-1R activity (e.g., in vitro or in vivo).

In some aspects, the present disclosure provides use of a form of Compound 1 disclosed herein in the manufacture of a medicament for treating or preventing a disease or disorder disclosed herein.

In some aspects, the present disclosure provides use of a form of Compound 1 disclosed herein in the manufacture of a medicament for treating a disease or disorder disclosed herein.

In some aspects, the present disclosure provides use of a form of Compound 1 disclosed herein in the manufacture of a medicament for treating or preventing diabetes, NASH, insulinoma, obesity, and/or hyperglycemia in a subject in need thereof.

In some aspects, the present disclosure provides use of a form of Compound 1 disclosed herein in the manufacture of a medicament for treating cancer in a subject in need thereof.

The present disclosure provides compounds that function as modulators of GLP-1R activity.

In some embodiments, the compounds of the present disclosure are agonists of the GLP-1 receptor.

In some embodiments, the modulation of the GLP-1R receptor is activation of the GLP-1R receptor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which GLP-1R activity is implicated in a patient in need of such treatment, said method comprising administering to said patient a therapeutically effective amount of a compound, or a pharmaceutically acceptable salt thereof, or a pharmaceutical composition as defined herein.

In accordance with the present application, a disease or condition to be treated and/or prevented is selected from the group consisting of cardiometabolic and associated diseases including diabetes (T1 D and/or T2DM, including pre-diabetes), idiopathic T1 D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), hyperinsulinemia, liver diseases such as NAFLD, steatosis, NASH, fibrosis, cirrhosis, and hepatocellular carcinoma, cardiovascular disease, atherosclerosis (including coronary artery disease), peripheral vascular disease, hypertension, endothelial dysfunction, impaired vascular compliance, congestive heart failure, myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome and addiction (e.g., alcohol and/or drug abuse), prevention or treatment of Polycystic Ovary Syndrome and treatment of addiction (e.g., alcohol and/or drug abuse).

In some embodiments, provided herein is a method of treating a cardiometabolic disease in a subject (e.g., a human patient) in need thereof, comprising administering to the subject a therapeutically effective amount of a compound described herein, or a pharmaceutically acceptable salt thereof.

In some embodiments, provided herein is a method of treating diabetes in a subject (e.g., a human patient) in need thereof, comprising administering to the subject a therapeutically effective amount of a form of Compound 1 described herein. Exemplary diabetes include, but are not limited to, T1 D, T2DM, pre-diabetes, idiopathic T1 D, LADA, EOD, YOAD, MODY, malnutrition-related diabetes, and gestational diabetes.

In some embodiments, provided herein is a method of treating a liver disorder in a subject (e.g., a human patient) in need thereof, comprising administering to the subject a therapeutically effective amount of a compound described herein, or a pharmaceutically acceptable salt thereof. Exemplary liver disorders include, without limitation, liver inflammation, fibrosis, and steatohepatitis. In some embodiments, the liver disorder is selected from the list consisting of primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, and oti-antitrypsin deficiency. In some embodiments, the liver disorder is selected from the list consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is selected from the group consisting of liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In accordance with the present application, a compound described herein, or a pharmaceutically acceptable salt thereof, can be administered by any suitable route in the form of a pharmaceutical composition adapted to such a route, and in a dose effective for the treatment intended. In some embodiments, it is a compound of any embodiment of Formula (I) or a sub-gormula thereof, or selected from the compounds of Table 1, or a pharmaceutically acceptable salt thereof. The compounds and/or compositions described herein may be administered orally, rectally, vaginally, parenterally, or topically.

In some embodiments, the compounds and/or compositions may be administered orally. Oral administration may involve swallowing, so that the compound enters the gastrointestinal tract, or buccal or sublingual administration may be employed by which the compound enters the bloodstream directly from the mouth.

In some embodiments, the compounds and/or compositions may be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In some embodiments, the compounds and/or compositions may be administered topically to the skin or mucosa, that is, dermally or transdermally. In some embodiments, the compounds and/or compositions may be administered intranasally or by inhalation. In some embodiments, the compounds and/or compositions may be administered rectally or vaginally. In some embodiments, the compounds and/or compositions may be administered directly to the eye or ear.

The compounds and/or compositions described herein can be used alone, or in combination with other therapeutic agents. The administration of two or more agents “in combination” means that all of the agents are administered closely enough in time that each may generate a biological effect in the same time frame. The presence of one agent may alter the biological effects of the other agent(s). The two or more agents may be administered simultaneously, concurrently or sequentially. Additionally, simultaneous administration may be carried out by mixing the agents prior to administration or by administering the compounds at the same point in time but as separate dosage forms at the same or different site of administration.

In some embodiments, the one or more other therapeutic agent is an anti-diabetic agent including but not limited to a biguanide (e.g., metformin), a sulfonylurea (e.g., tolbutamide, glibenclamide, gliclazide, chlorpropamide, tolazamide, acetohexamide, glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL 1 inhibitor, a GPR40 agonist (FFAR 1/FFA 1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In some embodiments, the one or more other therapeutic agent is an antiobesity agent including but not limited to peptide YY or an analogue thereof, a neuropeptide Y receptor type 2 (NPYR2) agonist, a NPYR1 or NPYR5 antagonist, a cannabinoid receptor type 1 (CB1 R) antagonist, a lipase inhibitor (e.g., orlistat), a human proislet peptide (HIP), a melanocortin receptor 4 agonist (e.g., setmelanotide), a melanin concentrating hormone receptor 1 antagonist, a farnesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues thereof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL 1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1 R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In some embodiments, the one or more other therapeutic agent is an agent to treat NASH including but not limited to PF-05221304, an FXR agonist (e.g., obeticholic acid), a PPAR a/d agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor21 (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976), a ketohexokinase (KHK) inhibitor, a diacylglyceryl acyltransferase 2 (DGAT2) inhibitor, a CB1 receptor antagonist, an anti-CB1 R antibody, or an apoptosis signal-regulating kinase 1 (ASK1) inhibitor, including the pharmaceutically acceptable salts of the specifically named agents and the pharmaceutically acceptable solvates of said agents and salts.

### EXAMPLES

### Example 1. Salt and Polymorph Screening and Evaluation of Compound 1

### Summary

Starting from Freeform Type A, a total of 60 salt screening experiments were performed using 20 acids or bases in 3 solvent systems. The obtained solids were characterized by X-ray powder diffraction (XRPD). Based on the results, a total of 15 salts (24 crystal forms) and two freeform forms (Freeform Type B and C) were obtained. The representative samples of all the salt hits and freeform crystalline hits were characterized by thermogravimetric analysis (TGA), differential scanning calorimetry (DSC), nuclear magnetic resonance (1H NMR) and high-performance liquid chromatography (HPLC). The stoichiometry was determined by 1H NMR or HPLC combined with ion chromatography (IC).

Based on the characterization results of salt hits and freeform crystalline forms, Fumarate Type A, Potassium salt Type A, Meglumine salt Type A, Tris salt Type C and Freeform Type C showed desirable solid state properties, therefore, the four salt leads and Freeform Type C were re-prepared at 300 mg scale for kinetic solubility, hygroscopicity and solid state stability evaluation. The results are summarized in Table 43.

As a result:

1) The solubility of the four salt leads and Freeform Type C samples was tested in H₂O and bio-relevant media (simulated gastric fluid (SGF), fasted state simulated intestinal fluid (FaSSIF) and fed state simulated intestinal fluid (FeSSIF)) at time points of 1/2/4/24 hours. The results showed that Meglumine salt Type A exhibited higher solubility than other salts and Freeform Type C in H₂O, SGF and FaSSIF. Moreover, Meglumine salt Type A maintained good to excellent solubility in H₂O throughout the 24-hour process (≥7.0 mg/mL), much higher than in SGF and FaSSIF. In FeSSIF, the 24-hour solubility of Tris salt Type C was higher (0.14 mg/mL). XRPD results showed that no form change was observed for Fumarate Type A and Freeform Type C in all the media, while form change was observed for Meglumine salt Type A, Potassium salt Type A and Tris salt Type C after 24 hours.

2) Solubility test was performed on Meglumine salt Type A and Freeform Type C in pH 1.0/2.0/3.5/4.5/5.5/6.8/8.0 buffers for 2 and 24 hours at RT. As a result: Meglumine salt Type A showed relatively higher solubility (0.15˜1.3 mg/mL) in pH 1.0 and 8.0 buffers. The solubility in pH 2.0 and 6.8 buffers was 0.016˜0.2 mg/mL, and that in pH 3.5, 4.5 and 5.5 buffers was ≤0.021 mg/mL. Freeform Type C showed relatively higher solubility (≥0.13 mg/mL) in pH 1.0 buffer, while the solubility in pH 2.0˜8.0 buffers was low (≤0.034 mg/mL).

3) Dynamic vapor sorption (DVS) results showed that Fumarate Type A and Freeform Type C were non-hygroscopic, while Potassium salt Type A, Meglumine salt Type A and Tris salt Type C were slightly hygroscopic. No form change was observed after DVS test.

4) Solid-state stability results showed that no form change or significant purity decrease was observed for Fumarate Type A, Potassium salt Type A, Meglumine salt Type A, Tris salt Type C or Freeform Type C after storage at 25° C./60% RH/open and 40° C./75% RH/open for 1 week, indicating good physical and chemical stability under the evaluated conditions.

According to the salt lead evaluation results, Meglumine salt Type A showed desirable solid state stability, lower hygroscopicity, and higher solubility in H₂O, SGF and FaSSIF. Combined with the results of PK experiments, Meglumine salt was selected as the leading salt form for further study and a polymorph screening study was conducted on Meglumine salt. Meglumine salt Type A was scaled-up on 3 g scale. Starting with the re-prepared Meglumine salt Type A, 50 polymorph screening experiments were set up using methods of anti-solvent addition, slurry at RT/50° C., slow evaporation, solid-vapor diffusion, liquid-vapor diffusion, slow cooling and grinding. The solids obtained were characterized by XRPD.

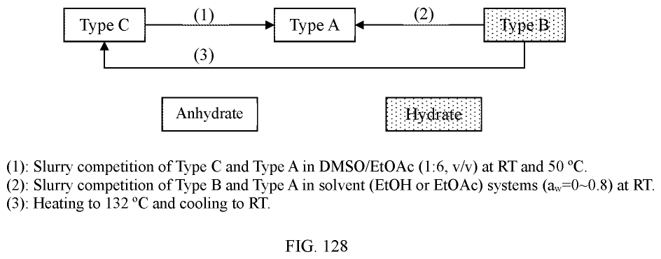

A total of two new Meglumine salt crystal forms were discovered from follow-up scale-up preparation and form identification, named as Meglumine salt Type B/C. The new forms of Meglumine salt Type B/C were characterized by TGA, DSC and 1H NMR. Based on the characterization results, it was speculated that Meglumine salt Type A and C were anhydrates and Meglumine salt Type B was a hydrate. To investigate the thermodynamic stability relationship between Meglumine salt anhydrates Type A and C, slurry competition experiments were set up between Meglumine salt Type A and C in MeOH/MTBE (8:13, v/v) and DMSO/EtOAc (1:6, v/v) at RT and 50° C. The results showed that the physical mixture of Meglumine salt Type A and C transformed to Meglumine salt Type A after slurry competition in DMSO/EtOAc (1:6, v/v) at RT and 50° C., indicating that Meglumine salt Type A was a thermodynamically more stable anhydrate from RT to 50° C.; a crystal form similar to Meglumine salt Type B was obtained in MeOH/MTBE (8:13, v/v) at RT and 50° C., and it turned to Meglumine salt Type B after drying under ambient conditions. The results suggested that there might be an intermediate solvate state in MeOH/MTBE (8:13, v/v), which turned to Meglumine salt Type B after desolvation under ambient conditions. In order to further investigate the transformation relationship between Meglumine salt Type A and hydrous Meglumine salt Type B, slurry competition experiments were set up at RT under different water activities (aw). The results showed that the mixtures of Meglumine salt Type A and B transformed to Meglumine salt Type A in aw˜0, 0.2, 0.4, 0.6, 0.8 systems after slurry competition. The form conversion diagram of meglumine salt forms was shown in FIG. 128.

In the meanwhile, to assess the polymorph landscape of the freeform, starting from a new batch of freeform sample (freeform D), a total of 50 polymorph screening experiments were set up using methods of anti-solvent addition, slurry at RT/50° C., slow evaporation, solid-vapor diffusion, liquid-vapor diffusion and slow cooling. The solids obtained were characterized by XRPD. A total of 13 new crystal forms were discovered, named as Freeform Type D˜P. The Freeform Type D/E/G˜I/K˜P were characterized by TGA, DSC and 1H NMR. Based on the characterization results, Freeform Type C/M were speculated to be anhydrates, Freeform Type I/UP were speculated to be hydrates, Freeform Type A/E were speculated to be anhydrates or hydrates, Freeform Type B/D/F/G/H/I/J/K/N/O were speculated to be solvates. To investigate the thermodynamic stability relationship between anhydrates, slurry competition experiments were set up for Freeform Type A/C/M in EtOAc and ACN at RT and 50° C. The results showed that the physical mixture of Freeform Type A/C/M transformed to Freeform Type C after slurry competition in EtOAc and ACN at RT and 50° C., indicating that Freeform Type C was a thermodynamically more stable anhydrate from RT to 50° C. In order to further investigate the transformation relationship between Freeform Type C and hydrates, slurry competition experiments were set up among Freeform Type C/IP/A/E/I at RT under different water activities in Acetone/H₂O (aw=0˜1). The results showed that the mixtures transformed to Freeform Type C in aw=0˜0.8 systems, and transformed to Freeform Type P in H₂O, indicating the critical water activity at RT between Freeform Type C and Type P was between 0.8 and 1.0.

According to the results of salt evaluation and polymorph screening results, Meglumine salt Type A is a more thermodynamically stable form between RT and 50° C.

TABLE 43
Characterization and evaluation results of selected salts and Freeform Type C

| | FreeForm | Fumarate | Potassium salt | Meglumine salt | Tris salt |
|---|---|---|---|---|---|
| Crystal Form | Type C | Type A | Type A | Type A | Type C |
| Safety Class of | — | 1 | 1 | 1 | 2 |
| Acid/Base | | | | | |
| TGAWeight | 1.05 | 1.23 | 4.20 | 2.66 | 1.07 |
| Loss | (150° C.) | (150° C.) | (150° C.) | (150° C.) | (150° C.) |
| (%, T) | | | | | |
| DSC | 188.7 | 210.9 | 241.9 | 163.9 | 192.6 |
| endotherms | | | | | |
| (° C., peak) | | | | | |
| Solvent Residue | 0.6 | 1.5 | 0.9 | Not | Not |
| by NMR (wt %) | (EtOAc) | (Acetone) | (EtOH) | detected | detected |
| HPLC Purity | 96.59 | 96.37 | 96.41 | 96.73 | 96.55 |
| (Area %) | | | | | |
| Stoichiometry | — | 0.5 | 0.4 | 1.0 | 1.0 |
| (Acid or | | | | | |
| Base/Freeform) | | | | | |
| Hygroscopicity | Non- | Non- | Slightly | Slightly | Slightly |
| | hygroscopic | hygroscopic | hygroscopic | hygroscopic | hygroscopic |

| | |
|---|---|
| Solid state | No form change or significant purity decrease was observed after storage at |
| Stability | 25° C./60% RH/open and 40° C./75% RH/open for 7 days. |
| Kinetic | 1) In H₂O, SGF and FaSSIF, Meglumine salt Type A exhibited higher solubility than other salts and Freeform Type C; |
| Solubility | 2) In FeSSIF, the 24-h solubility of Tris salt Type C was higher. |

### Characterization of Starting Material

The starting material of Compound 1 freeform was characterized by XRPD, TGA, DSC and 1H NMR. XRPD result showed that the starting material was crystalline, named as Freeform Type A. TGA/DSC results in revealed a weight loss of 3.60% up to 150° C. and three endotherms at 103.6° C., 161.5° C. and 185.6° C. (peak temperature). 1H NMR data was collected using DMSO-d6 as solvent. The result showed that negligible organic solvent was detected. This batch of freeform was used in salt screening experiments. After heating Freeform Type A to 110° C., cooling to RT and exposure to ambient conditions, an amorphous form was obtained. After heating to 161° C., cooling to RT and exposure to ambient conditions, Freeform Type M was obtained (Freeform Type M was an anhydrate, the characterization and form identification could refer to FIGS. 118-120). Based on the results of heating experiments, the endotherm at 103.6° C. and 161.5° C. might be the signal of form change to amorphous and recrystallization to Freeform Type M, respectively. Based on the characterization, Freeform Type A was speculated to be an anhydrate or hydrate.

pKa and Log D value of the compound was measured using starting material (Freeform Type A. The results were summarized in Table 44 and Table 45. pKa value was measured using Sirius T3Dt instrument and the result was 4.40 (pKa1) and 5.41 (pKa2). Log D value was measured by shake flask method. The result was Log D_{7.2}=1.96. Log P was calculated as 4.78 by pKa and Log D values.

The second batch of freeform starting material was characterized by XRPD, TGA, DSC and 1H NMR. XRPD result revealed that it was a new form, named as Freeform Type D. TGA/DSC results in showed a stepwise weight loss of 10.67% up to 180° C. and two endotherms at 145.6° C. and 148.6° C. (peak temperature). 1H NMR result showed that the molar ratio of solvent THF/API was 0.9 (10.1 wt %, close to the TGA weight loss). After heating Freeform Type D to 180° C. and cooling to RT, amorphous sample was obtained (sample melted). Based on the characterization results, this batch of Freeform Type D was speculated to be a THF solvate. This batch of freeform was used for scale up of Meglumine salt Type A and polymorph screening of freeform.

TABLE 44
pKa result of starting material

pKa value

| Mw | # | pH metric | UV metric | Average |
|---|---|---|---|---|
| 582.65 | 1 | 4.43 | 4.36 | 4.40 |
| | 2 | 5.52 | 5.29 | 5.41 |

TABLE 45
LogD and LogP results of starting material

| | Conc. (mg/mL) | | | | Average | |
|---|---|---|---|---|---|---|
| # | Octanol | Water | pH | LogD_{7.2} | LogD_{7.2} | LogP |
| 1 | 0.52 | 0.0062 | 7.19 | 1.93 | 1.96 | 4.78 |
| 2 | 0.55 | 0.0058 | 7.22 | 1.98 | | |
| 3 | 0.50 | 0.0051 | 7.21 | 1.98 | | |

LogD = Log[Conc (octanol)/Conc (aqueous)]
LogP = LogD + Lg[1 + 10^{(pka (base)-pH)} + 10^{(pH-pka (acid))} + 10^{(pka (base)-pka (acid))}]

### Summary of Salt Screening

According to the approximate solubility of Freeform Type A at room temperature (RT), a total of 60 salt screening experiments were performed using 20 acids or bases in 3 solvent systems via solvent-assisted reaction crystallization. In details, weigh about 20 mg of freeform and equal molar counterions into an HPLC vial. Add 0.5 mL solvent and stir (˜1000 rpm) the mixture at RT for about 3 days. Solids were isolated for XRPD analysis. As summarized in Table 46, 15 salts (24 crystal forms) and two freeform forms (Freeform Type B and C) were obtained based on the XRPD comparison. All salt hits and freeform forms were characterized by TGA, DSC, 1H NMR and HPLC purity. The stoichiometric ratio was determined by 1H NMR or HPLC/IC. Characterization results of salt hits were listed in Table 47.

TABLE 46
Summary of salt screening results

| | | Safety | Solvent | | |
|---|---|---|---|---|---|
| Counterion | | class | A: EtOH | B: Acetone | C: EtOAc |
| 0 | Blank | — | Freeform Type B | Freeform Type C | Freeform Type C |
| 1 | HCl | 1 | Freeform Type B | Freeform Type C | HCl salt Type A |
| 2 | H₂SO₄ | 1 | Sulfate Type A | Sulfate Type B | Sulfate Type C |
| 3 | H₃PO₄ | 1 | Phosphate Type A | Phosphate Type A + | Freeform Type C |
| | | | | Freeform Type B | |
| 4 | Maleic acid | 1 | Freeform Type B | Gel | Amorphous |
| 5 | L-Tartaric acid | 1 | Freeform Type B | Tartrate Type A | Tartrate Type A |
| 6 | Glycolic acid | 1 | Freeform Type B | Freeform Type C | Freeform Type C |
| 7 | Fumaric acid | 1 | Fumarate Type A | Fumarate Type A | Fumarate Type A + |
| | | | | | acid |
| 8 | Oxalic acid | 2 | Freeform Type B | Gel | Amorphous |
| 9 | Mg(OH)₂ | 1 | Freeform Type B + | Freeform Type C + | Freeform Type C + |
| | | | base | base | base |
| 10 | Methanesulfonic | 2 | Gel | Mesylate Type A | Amorphous |
| | acid | | | | |
| 11 | p- | 2 | Tosylate Type B | Tosylate Type B | Tosylate Type A |
| | Toluenesulfonic | | | | |
| | acid | | | | |
| 12 | L-Arginine | 1 | Arginine salt Type B | Arginine salt Type A | Freeform Type C |
| 13 | L-Lysine | 1 | Lysine salt Type A | Lysine salt Type B | Freeform Type C + |
| | | | | | acid |
| 14 | NaOH | 1 | Na salt Type A | Freeform Type C | Freeform Type C |
| 15 | KOH | 1 | K salt Type A | K salt Type B | K salt Type B |
| 16 | Ca(OH)₂ | 1 | Freeform Type B + | Freeform Type C + | Freeform Type C |
| | | | base | base | |
| 17 | Choline | 1 | Freeform Type B | Choline salt Type A | Choline salt Type A |
| 18 | Ammonium | 1 | Ammonium salt | Ammonium salt | Ammonium salt |
| | | | Type A | Type A + B | Type B |
| 19 | Meglumine | 1 | Meglumine salt | Meglumine salt | Meglumine salt |
| | | | Type A + base | Type A | Type A + base |
| 20 | Tromethamine | 2 | Tris salt Type A | Tris salt Type A | Tris salt Type A |

TABLE 47
Characterization results of salt hits

| Salt hit | Safety | TGA weight | DSC endotherm | Molar ratio | Purity |
|---|---|---|---|---|---|
| (Sample ID) | class | loss (%) | (° C., peak) | (Acid/API)^{#} | (Area %) |
| Fumarate Type A | 1 | 1.83 (150° C.) | 209.9 | 0.6 | 96.23 |
| K salt Type A | 1 | 1.35 (150° C.) | 242.4 | 0.5 | 97.31 |
| Meglumine salt Type A | 2 | 1.42 (150° C.) | 163.2 | 1.0 | 96.61 |
| Tris salt Type C | 2 | 5.81 (150° C.) | 192.1 | 1.0 | — |
| HCl salt Type A | 1 | 4.18 (150° C.) | 116.9, 154.7, 172.5 | 0.7 | 96.24 |
| Sulfate Type A | 1 | 7.10 (150° C.) | 70.1, 116.6, 150.7 | 1.0 | 92.91 |
| Sulfate Type B | 1 | 3.81 (150° C.) | 131.5, 169.8 | 1.1 | 95.33 |
| Sulfate Type C | 1 | 6.72 (150° C.) | 93.2 | 1.2 | 95.70 |
| Phosphate Type A | 1 | 2.40 (150° C.) | 133.9, 159.3 | 0.7 | 95.62 |
| Tartrate Type A | 1 | 4.62 (150° C.) | 122.8, 189.0 | 1.1 | 96.28 |
| Mesylate Type A | 2 | 5.91 (150° C.) | 131.3 | 0.9 | 96.59 |
| Tosylate Type A | 2 | 5.23 (150° C.) | 76.6, 117.8 | 0.8 | 94.30 |
| Tosylate Type B | 2 | 5.86 (150° C.) | 125.8, 128.2 | 0.9 | 95.87 |
| Arginine salt Type A | 1 | 6.56 (150° C.) | 92.4 | 0.5 | 95.29 |
| Arginine salt Type B | 1 | 4.34 (150° C.) | 140.8 | 1.0 | 96.44 |
| Lysine salt Type A | 1 | 5.72 (150° C.) | 74.2, 174.0 | 0.9 | 97.25 |
| Lysine salt Type B | 1 | 7.06 (150° C.) | 207.7 | 1.2 | 92.87 |
| Na salt Type A | 1 | 7.00 (150° C.) | 92.2, 135.5 | 1.0 | 96.99 |
| K salt Type B | 1 | 3.90 (150° C.) | 150.6, 192.9*, | 1.0 | 96.93 |
| | | | 224.0, 232.8 | | |
| Choline salt Type A | 1 | 3.38 (150° C.) | 184.5, 210.4 | 0.3 | 96.71 |
| Ammonium salt Type A | 1 | 4.02 (150° C.) | 132.8, 156.2, 192.2 | 1.1 | 96.65 |
| Ammonium salt Type B | 1 | 4.10 (150° C.) | 140.7, 152.2*, 189.0 | 0.7 | 96.90 |
| Tris salt Type A | 2 | 5.39 (150° C.) | 133.1, 182.0, 191.2 | 1.0 | 96.65 |
| Tris salt Type B | 2 | 5.51 (150° C.) | 104.8, 118.1*, 191.6 | 0.9 | 97.46 |

*Exotherm, peak temperature.
^{#}Determined by ¹H NMR or HPLC/IC.

### Re-Preparation and Characterization of Selected Salts

Based on the characterization results, Fumarate Type A, Potassium salt Type A, Meglumine salt Type A and Tris salt Type C showed relatively good solid state properties (high crystallinity, negligible TGA weight loss, neat DSC signal, and low safety risk of counterion). Meanwhile, Freeform Type C exhibited better solid state properties than Freeform Type A and B (anhydrate with lower TGA weight loss and neat DSC signal), therefore, Freeform Type C was also re-prepared. The re-prepared samples were characterized by XRPD, TGA, DSC, H NMR and HPLC, and the stoichiometry was determined by 1H NMR or HPLC/IC. Detailed procedures for re-preparation are displayed in Table 48.

TABLE 48
Preparation procedures of selected salts

| Salt lead | | |
|---|---|---|
| (Sample ID) | Procedure | |
| Fumarate | 1. | Weigh 299.7 mg of freeform and 60.0 mg of fumaric acid into a 20 mL |
| Type A | | vial. Add 6 mL acetone. |
| | 2. | Magnetically stir at RT for 2 days. Isolate solids by centrifugation. |
| | 3. | Dry solid at RT under vacuum for 3 days. |
| Potassium salt | Article I. Weigh 300.4 mg of freeform) and 15.8 mg KOH into a | |
| Type A | 20 mL vial. Add 3 mL EtOH. | |
| | Article II. Magnetically stir at RT for 1 day. Isolate solids by | |
| | centrifugation. | |
| | Article III. Dry solid at RT under vacuum for 1 day. A total of 281 | |
| | mg of solid was obtained (Yield: 88%). | |
| Meglumine salt | 1. | Weigh 300.2 mg of freeform and 100.8 mg meglumine into a 20 mL |
| Type A | | vial. Add 6 mL acetone. |
| | 2. | Magnetically stir at RT for 2 days. Isolate solids by centrifugation. |
| | 3. | Dry solid at RT under vacuum for 3 days. A total of 326 mg of solid |
| | | was obtained (Yield: 81%). |
| Tris salt | 1. | Weigh 300.4 mg of freeform and 64.2 mg tromethamine into a 20 mL |
| Type C | | vial. Add 7 mL acetone. |
| | 2. | Magnetically stir at RT for 2 hrs and then stir with temperature cycling |
| | | between 5~50° C. (3 cycles). |
| | 3. | Isolate solids by centrifugation. Dry solid at RT under vacuum for 1 |
| | | day. |
| | 4. | Heat the sample to 177° C., keep it at 177° C. for 3 min and col to RT. |
| | | A total of 230 mg of solid was obtained (Yield: 76%). |
| Freeform | 1. | Weigh 299.6 mg of freeform into a 20 mL vial. Add 6 mL EtOAc. |
| Type C | 2. | Magnetically stir at RT for 2 days. Isolate solids by centrifugation. |
| | 3. | Dry solid at RT under vacuum for 3 days. A total of 268 mg of solid |
| | | was obtained (Yield: 89%). |

### Fumarate Type A

XRPD pattern of Fumarate Type A was shown in FIG. 5. TGA/DSC results were displayed in FIGS. 6 and 7, respectively. TGA result showed a weight loss of 1.23% up to 150° C. DSC result showed an endotherm at 210.9° C. (peak temperature). The 1H NMR result in FIG. 131 showed that the molar ratio of acid/freeform was 0.5 and solvent acetone/API was 0.2 (1.5 wt %). HPLC purity of Fumarate Type A was 96.37 area %.

### Potassium Type A

XRPD pattern of Potassium salt Type A was shown in FIG. 9. HPLC/IC result showed that the molar ratio of K+/freeform was 0.4. TGA/DSC results were displayed in FIGS. 10 and 11, respectively.. TGA result showed a weight loss of 0.42% up to 150° C. DSC result showed one endotherm at 241.9° C. (peak temperature). The 1H NMR result in FIG. 132 showed that the molar ratio of EtOH/API was 0.1 (0.9 wt %). HPLC purity of Potassium salt Type A was 96.41 area %.

### Meglumine Type A

XRPD pattern of Meglumine salt Type A was shown in FIG. 1. TGA/DSC results were displayed in FIGS. 2 and 3, respectively. TGA result showed a weight loss 2.66% up to 150° C. DSC result showed an endotherm at 163.9° C. (peak temperature). 1H NMR result in FIG. 130 showed that the molar ratio of meglumine/freeform was 1.0 and negligible solvent acetone was detected. HPLC purity of Meglumine salt Type A was 96.73 area %.

### Tris Type C

XRPD pattern of Tris salt Type C was shown in FIG. 13. TGA/DSC results were displayed in FIGS. 14 and 15. TGA result showed a weight loss 1.07% up to 150° C. DSC result showed an endotherm at 192.6° C. (peak temperature). 1H NMR result in FIG. 133 showed that the molar ratio of tromethamine/freeform was 1.0 and negligible solvent acetone was detected. HPLC purity of Tris salt Type C was 96.55 area %.

### Freeform Type C

XRPD pattern of Freeform Type C was shown in FIG. 89. TGA/DSC results were displayed in FIGS. 90 and 91, respectively. TGA result showed a weight loss 1.05% up to 150° C. DSC result showed an endotherm at 188.7° C. (peak temperature). 1H NMR result in FIG. 158 showed that the molar ratio of residual solvent EtOAc/API was 0.04 (0.6 wt %). HPLC purity of Freeform Type C was 96.59 area %.

### Evaluation of Selected Salts and Freeform Type C

To assess the solid state properties and developability, evaluation including solubility, hygroscopicity and solid state stability was performed for Fumarate Type A, Potassium salt Type A, Meglumine salt Type A and Tris salt Type C. Freeform Type C was also evaluated to compare properties with selected salts.

### Kinetic Solubility

Solubility of Fumarate Type A, Potassium salt Type A, Meglumine salt Type A, Tris salt Type C and Freeform Type C was measured in water and bio-relevant media (SGF, FaSSIF and FeSSIF) at 37° C. for 1, 2, 4 and 24 hrs. Detailed procedures are as follows:
Added ˜40 mg of Fumarate Type A, Potassium salt Type A, Meglumine salt Type A, Tris salt Type C and Freeform Type C respectively into a 5-mL glass vial. Add 4 mL of medium (H₂O, SGF, FaSSIF and FeSSIF).
Rolled at 37° C. (˜25 rpm). Sampling at 1, 2, 4 and 24 hrs.
At each time-point, sampled 0.8 mL of solution or suspension into a centrifuge tube prior to centrifugation (12000 rpm, 37° C., 5 min) and filtration through 0.45 μm PTFE membrane.
Tested pH and concentration for the filtrate. Analyze residual solids by XRPD.

Solubility results are summarized in Table 49. The results showed that:
In H₂O, Meglumine salt Type A showed higher solubility than other salts and Freeform Type C. For Meglumine salt Type A, the solubility at 1, 2 and 4 hrs was >8.3 mg/mL and decreased to 7.0 mg/mL at 24 hrs.
In SGF and FaSSIF, Meglumine salt Type A also showed higher solubility than other salts and Freeform Type C. For Meglumine salt Type A, the solubility in SGF and FaSSIF was lower than in H₂O. The highest solubility was observed at 2 hrs (0.72 mg/mL and 1.3 mg/mL respectively).
In FeSSIF, Tris salt Type C showed higher solubility (0.14 mg/mL) at 24 hrs than other salts and Freeform Type C.
Fumarate Type A and Freeform Type C showed the lowest solubility in all the media.
No form change was observed for Fumarate Type A and Freeform Type C in all the media while form change was observed for Meglumine salt Type A, Potassium salt Type A and Tris salt Type C in all the media after 24 hrs.

TABLE 49
Summary of solubility results

| | | 1 hr | | | 2 hrs | | | 4 hrs | | | 24 hrs | | |
|---|---|---|---|---|---|---|---|---|---|---|---|---|---|
| Material | Medium | S | pH | FC | S | pH | FC | S | pH | FC | S | pH | FC |
| Fumarate | H₂O | 0.003 | 3.8 | No | 0.003 | 3.9 | No | 0.002 | 3.8 | No | 0.003 | 3.7 | No |
| Type A | SGF | 0.046 | 1.7 | No | 0.044 | 2.0 | No | 0.043 | 1.8 | No | 0.048 | 1.8 | No |
| | FaSSIF | 0.004 | 6.3 | No | 0.004 | 6.4 | No | 0.007 | 6.4 | No | 0.003 | 6.2 | No |
| | FeSSIF | 0.010 | 5.1 | No | 0.009 | 5.3 | No | 0.009 | 5.1 | No | 0.020 | 5.0 | No |
| Potassium | H₂O | 0.92 | 9.3 | No | 1.7 | 9.4 | Yes | 3.8 | 9.3 | Yes | 4.9 | 9.2 | Yes |
| salt Type A | SGF | 0.28 | 1.9 | Yes | 0.069 | 2.4 | Yes | 0.039 | 2.5 | Yes | 0.050 | 2.5 | Yes |
| | FaSSIF | 0.024 | 6.6 | No | 0.021 | 6.5 | No | 0.022 | 6.5 | No | 0.029 | 7.6 | Yes |
| | FeSSIF | 0.038 | 5.2 | No | 0.035 | 5.2 | No | 0.034 | 5.2 | No | 0.021 | 5.5 | Yes |
| Meglumine | H₂O | >8.6 | 9.3 | — | >8.9 | 9.5 | — | >8.3 | 9.3 | — | 7.0 | 9.4 | Yes |
| salt Type A | SGF | 0.38 | 7.0 | Yes | 0.72 | 7.2 | Yes | 0.68 | 7.0 | Yes | 0.29 | 7.6 | Yes |
| | FaSSIF | 0.90 | 7.3 | Yes | 1.3 | 7.3 | Yes | 0.72 | 7.3 | Yes | 0.07 | 7.6 | Yes |
| | FeSSIF | 0.17 | 5.4 | Yes | 0.17 | 5.6 | Yes | 0.086 | 5.5 | Yes | 0.027 | 5.6 | Yes |
| Tris salt | H₂O | 0.46 | 8.7 | No | 0.59 | 9.0 | Yes | 2.5 | 8.9 | Yes | 0.99 | 9.2 | Yes |
| Type C | SGF | 0.15 | 3.6 | No | 0.03* | 3.8 | No | 0.04* | 3.6 | Yes | 0.17 | 7.9 | Yes |
| | FaSSIF | 0.16 | 7.0 | Yes | 0.29 | 7.2 | Yes | 0.28 | 7.2 | Yes | 0.04 | 7.7 | Yes |
| | FeSSIF | 0.17 | 5.2 | No | 0.13 | 5.3 | No | 0.02* | 5.2 | No | 0.14 | 6.5 | Yes |
| Freeform | H₂O | 0.020 | 8.8 | No | 0.027 | 8.9 | No | 0.020 | 8.8 | No | 0.008 | 8.9 | No |
| Type C | SGF | 0.083 | 1.9 | No | 0.072 | 2.0 | No | 0.075 | 1.9 | No | 0.078 | 1.8 | No |
| | FaSSIF | 0.002 | 6.7 | No | 0.007 | 6.7 | No | 0.005 | 6.7 | No | 0.003 | 6.5 | No |
| | FeSSIF | 0.006 | 5.2 | No | 0.006 | 5.2 | No | 0.006 | 5.0 | No | 0.008 | 5.2 | No |

S: Solubility (mg/mL), calculated by freeform.
FC: Form change.
—: Clear solution was obtained, no solid for XRPD analysis.
*Precipitation was observed after filtration (0.22 μm PTFE), so the result might be lower than the actual value.

### Hygroscopicity

To investigate the solid form stability as a function of humidity, DVS isotherm plot of Fumarate Type A, Potassium salt Type A, Meglumine salt Type A, Tris salt Type C and Freeform Type C were collected at 25° C. between 0 and 95% RH. The results were summarized in Table 50.

The water uptake of Fumarate Type A and Freeform Type C at 25° C./80% RH was 0.12%, indicating they were non-hygroscopic. XRPD results revealed that no form change was observed after DVS test.

The water uptake of Potassium salt Type A, Meglumine salt Type A and Tris salt Type C at 25° C./80% RH was 0.62%, 1.43% and 1.44%, respectively, indicating they were slightly hygroscopic. XRPD results revealed that no form change was observed after DVS test.

TABLE 50
Hygroscopicity results of selected salts and Freeform Type C

| | Water uptake at | For change |
|---|---|---|
| Sample ID | 25° C./80% RH(wt %) | after DVS |
| Fumarate Type A | 0.12 | No |
| Potassium salt Type A | 0.62 | No |
| Meglumine salt Type A | 1.43 | No |
| Tris salt Type C | 1.44 | No |
| Freeform Type C | 0.12 | No |

### Solid State Stability

To evaluate the solid state stability of Fumarate Type A, Potassium salt Type A, Meglumine salt Type A, Tris salt Type C and Freeform Type C, samples were stored at 25° C./60% RH and 40° C./75% RH. Each sample was added into a glass vial, sealed by parafilm with several holes, and kept under tested conditions. After one week, samples were taken for XRPD and HPLC purity test. All the characterization data were summarized in Table 51.

As a result: after storage at 25° C./60% RH and 40° C./75% RH for 1 week, no form change or obvious purity decrease was observed for all the samples, indicating good physical and chemical stability at the evaluated conditions.

TABLE 51
Summary of solid state stability evaluation

| | | Purity | Form |
|---|---|---|---|
| Sample ID | Condition | (Area %) | change |
| Fumarate | Initial | 96.37 | — |
| Type A | 25° C./60% RH/Open/1 week | 96.13 | No |
| | 40° C./75% RH/Open/1 week | 96.37 | No |
| Potassium salt | Initial | 96.41 | — |
| Type A | 25° C./60% RH/Open/1 week | 96.47 | No |
| | 40° C./75% RH/Open/1 week | 96.58 | No |
| Meglumine salt | Initial | 96.73 | — |
| Type A | 25° C./60% RH/Open/1 week | 96.98 | No |
| | 40° C./75% RH/Open/1 week | 96.88 | No |
| Tris salt | Initial | 96.55 | — |
| Type C | 25° C./60% RH/Open/1 week | 96.76 | No |
| | 40° C./75% RH/Open/1 week | 96.82 | No |
| Freeform | Initial | 96.59 | — |
| Type C | 25° C./60% RH/Open/1 week | 96.58 | No |
| | 40° C./75% RH/Open/1 week | 96.69 | No |

TABLE 52
HPLC results of Fumarate Type A after stability evaluation

| | | Area (%) | | |
|---|---|---|---|---|
| #Peak | RRT | Initial | 25° C./60% RH | 40° C./75% RH |
| 1 | 0.73 | 0.13 | 0.14 | 0.13 |
| 2 | 0.80 | <0.05 | 0.05 | <0.05 |
| 3 | 0.83 | 1.91 | 1.98 | 1.89 |
| 4 | 0.90 | 0.09 | 0.10 | 0.09 |
| 5 | 0.99 | <0.05 | 0.06 | <0.05 |
| 6 | 1.00 | 96.37 | 96.13 | 96.37 |
| 7 | 1.05 | 0.27 | 0.28 | 0.29 |
| 8 | 1.10 | 0.87 | 0.89 | 0.87 |
| 9 | 1.12 | 0.05 | 0.06 | 0.05 |
| 10 | 1.15 | 0.24 | 0.25 | 0.26 |
| 11 | 1.34 | 0.06 | 0.07 | 0.06 |

TABLE 53
HPLC results of Potassium salt Type A after stability evaluation

| | | Area (%) | | |
|---|---|---|---|---|
| #Peak | RRT | Initial | 25° C./60% RH | 40° C./75% RH |
| 1 | 0.70 | 0.05 | 0.06 | 0.05 |
| 2 | 0.73 | 0.16 | 0.15 | 0.14 |
| 3 | 0.83 | 1.84 | 1.80 | 1.78 |
| 4 | 0.90 | 0.09 | 0.09 | 0.09 |
| 5 | 0.99 | 0.05 | 0.06 | <0.05 |
| 6 | 1.00 | 96.41 | 96.47 | 96.58 |
| 7 | 1.05 | 0.27 | 0.27 | 0.28 |
| 8 | 1.10 | 0.70 | 0.67 | 0.66 |
| 9 | 1.11 | 0.05 | 0.05 | 0.06 |
| 10 | 1.12 | 0.06 | 0.06 | 0.06 |
| 11 | 1.15 | 0.22 | 0.21 | 0.22 |
| 12 | 1.34 | 0.10 | 0.10 | 0.07 |

TABLE 54
HPLC results of Meglumine salt Type A after stability evaluation

| | | Area (%) | | |
|---|---|---|---|---|
| #Peak | RRT | Initial | 25° C./60% RH | 40° C./75% RH |
| 1 | 0.70 | 0.07 | 0.07 | 0.06 |
| 2 | 0.73 | 0.13 | 0.14 | 0.15 |
| 3 | 0.80 | 0.05 | 0.05 | 0.05 |
| 4 | 0.83 | 1.95 | 1.93 | 1.96 |
| 5 | 0.90 | 0.12 | 0.12 | 0.11 |
| 6 | 0.99 | 0.11 | <0.05 | <0.05 |
| 7 | 1.00 | 96.73 | 96.98 | 96.88 |
| 8 | 1.05 | 0.28 | 0.28 | 0.28 |
| 9 | 1.10 | 0.24 | 0.23 | 0.34 |
| 10 | 1.12 | 0.05 | <0.05 | <0.05 |
| 11 | 1.15 | 0.11 | 0.11 | 0.13 |
| 12 | 1.34 | 0.16 | 0.08 | 0.05 |

TABLE 55
HPLC results of Tris salt Type C after stability evaluation

| | | Area (%) | | |
|---|---|---|---|---|
| #Peak | RRT | Initial | 25° C./60% RH | 40° C./75% RH |
| 1 | 0.73 | 0.14 | 0.14 | 0.14 |
| 2 | 0.79 | 0.21 | 0.23 | 0.25 |
| 3 | 0.83 | 2.02 | 1.99 | 1.98 |
| 4 | 0.85 | 0.08 | 0.07 | <0.05 |
| 5 | 0.90 | 0.06 | 0.06 | 0.06 |
| 6 | 0.93 | 0.09 | 0.09 | 0.09 |
| 7 | 0.99 | 0.07 | <0.05 | <0.05 |
| 8 | 1.00 | 96.55 | 96.76 | 96.82 |
| 9 | 1.05 | 0.24 | 0.24 | 0.24 |
| 10 | 1.10 | 0.24 | 0.15 | 0.19 |
| 11 | 1.12 | 0.06 | 0.08 | 0.05 |
| 12 | 1.15 | 0.13 | 0.11 | 0.12 |
| 13 | 1.34 | 0.11 | 0.07 | 0.06 |

TABLE 56
HPLC results of Tris salt Type C after stability evaluation

| | | Area (%) | | |
|---|---|---|---|---|
| #Peak | RRT | Initial | 25° C./60% RH | 40° C./75% RH |
| 1 | 0.70 | 0.05 | 0.06 | 0.05 |
| 2 | 0.73 | 0.13 | 0.13 | 0.13 |
| 3 | 0.80 | 0.05 | 0.05 | 0.05 |
| 4 | 0.83 | 1.89 | 1.91 | 1.92 |
| 5 | 0.90 | 0.10 | 0.10 | 0.10 |
| 6 | 0.99 | 0.09 | 0.08 | <0.05 |
| 7 | 1.00 | 96.59 | 96.58 | 96.69 |
| 8 | 1.05 | 0.27 | 0.27 | 0.28 |
| 9 | 1.10 | 0.49 | 0.51 | 0.50 |
| 10 | 1.12 | 0.06 | 0.05 | 0.05 |
| 11 | 1.15 | 0.16 | 0.16 | 0.16 |
| 12 | 1.34 | 0.12 | 0.11 | 0.07 |

### pH Solubility

Since Meglumine salt Type A was chosen for further study based on, e.g., the higher solubility in H₂O, SGF and FaSSIF, solubility test in pH buffers was then performed on Meglumine salt Type A for 2 and 24 hours at RT. Solubility of Freeform Type C in pH buffers was also measured for comparison. The procedures were listed below:

1. Weighed ˜6 mg of Meglumine salt Type A or Freeform Type C samples into a 3-ml, glass vial. Add 2 ml, of the corresponding medium.

2. Mixed well and stirred at RT (1000 rpm). Check the pH and adjust it back to the target pH after dosing.

3. Sampled after 2 hours and 24 hours. The supernatant was isolated by centrifugation (12000 rpm, 5 min) and filtration (0.22 μm, PTFE) for pH and HPLC test.

The results were summarized in Table 57.

TABLE 57
pH solubility results of Meglumine
salt Type A and Freeform Type C

| | | 2 Hours | | 24 Hours | |
|---|---|---|---|---|---|
| Starting | | Solubility | | Solubility | |
| material | Medium | (mg/mL) | pH | (mg/mL) | pH |
| Meglumine salt | pH 1.0 buffer | 1.3 | 1.1 | 0.15 | 1.1 |
| Type A | pH 2.0 buffer | 0.20 | 2.1 | 0.016 | 2.1 |
| | pH 3.5 buffer | 0.021 | 3.5 | 0.0075 | 3.5 |
| | pH 4.5 buffer | 0.0037 | 4.6 | 0.0001 | 4.5 |
| | pH 5.5 buffer | 0.0028 | 5.6 | <LOQ* | 5.6 |
| | pH 6.8 buffer | 0.095 | 6.9 | 0.19 | 6.8 |
| | pH 8.0 buffer | 0.64 | 8.0 | 1.3 | 8.0 |
| Freeform Type | pH 1.0 buffer | 0.18 | 1.0 | 0.13 | 1.0 |
| C | pH 2.0 buffer | 0.034 | 2.1 | 0.017 | 2.1 |
| | pH 3.5 buffer | 0.016 | 3.5 | 0.0003 | 3.8 |
| | pH 4.5 buffer | ND | 4.5 | <LOQ | 4.5 |
| | pH 5.5 buffer | <LOQ | 5.5 | <LOQ | 5.4 |
| | pH 6.8 buffer | 0.0001 | 6.8 | 0.0003 | 6.7 |
| | pH 8.0 buffer | 0.0066 | 8.0 | 0.0067 | 7.9 |

*LOQ = 0.0001 mg/mL

### Characterization of Meglumine Salt Forms

Based on salt evaluation results, Meglumine salt Type A showed relatively good solid state stability and low hygroscopicity, and exhibited higher solubility in H₂O, SGF and FaSSIF and was selected as the salt candidate for following polymorph study. Starting from Compound 1 Meglumine salt Type A, polymorph screening experiments were performed under 50 conditions using different solvent-mediated crystallization or solid conversion methods. XRPD comparison result showed only Meglumine salt Type A was obtained. A new meglumine salt form (Meglumine salt Type B) was observed during scale up in MeOH/MTBE. Meglumine salt Type C was obtained during form identification of Meglumine salt Type B.

### Meglumine Type A

Meglumine salt Type A was obtained via procedures as follows. Weigh 3 g freeform Type D and 879.8 mg of meglumine into a 60 mL glass vial. Add 50 mL acetone and stir the mixture at RT for 1 day. Isolate the suspension by suction filtration and dry solid at RT under vacuum for 1 day. XRPD and TGA/DSC results were shown in FIG. 5 2 and FIG. 5 3. TGA result showed a weight loss of 1.51% up to 150° C. DSC result showed an endotherm at 165.3° C. (peak temperature). 1H NMR result in FIG. 5 4 showed that the molar ratio of meglumine/freeform was 1.0 and negligible solvent acetone was detected.

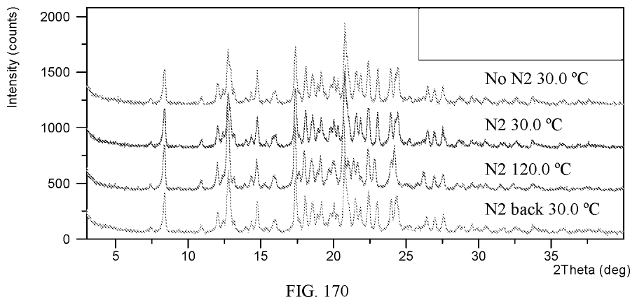

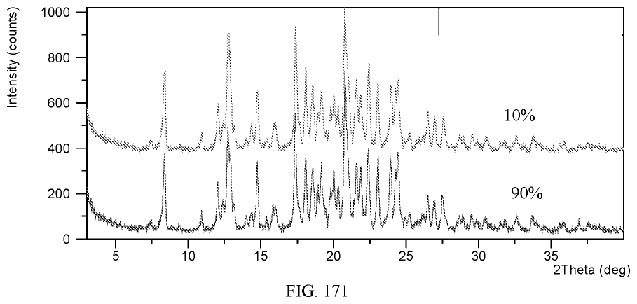

Varied temperature XRPD (VT-XRPD) results were shown in FIG. 170. Slight form change (as highlighted in the figure) was observed after heating Meglumine salt Type A to 120° C. under N2 protection. Meglumine salt Type A was re-obtained after cooling to 30° C. under N2 protection. Varied humidity XRPD (VH-XRPD) results were shown in FIG. 171. No obvious difference was observed for Meglumine salt Type A at 25° C./90% RH and 25° C./10% RH. Based on VT-XRPD and VH-XRPD results, Meglumine salt Type A was speculated to be an anhydrate. The single crystal structure determination result revealed that there was a small cavity in Meglumine salt Type A. In Example 3, the single crystal sample may contain 0.15 water of crystallization (i.e. the molar ratio of API to water is 1:0.15 in this single crystal structure), suggesting the possibility of water or organic solvent molecule in the crystal structure.

The 1/2/4/24 hours solubility of Meglumine salt Type A in SGF and FaSSIF was measured at 37° C. with adjusting pH to that of the blank medium. Detailed procedures are as follows.

1) Added ˜40 mg of sample into a 5-mL glass vial. Add 4 mL of medium. Adjust pH to 1.8 or 6.5 using HCl (1 M) or NaOH (1 M).

2) Rolled at 37° C. (˜25 rpm). Sample at 1, 2, 4 and 24 hrs.

3) At each time-point, sample 0.8 mL of solution or suspension into a centrifuge tube prior to centrifugation (12000 rpm, 37° C., 5 min) and filtration through 0.45 μm PTFE membrane.

4) Adjusted pH of the rest of suspension to 1.8 or 6.5 using HCl (1 M) or NaOH (1 M).

The solubility results were summarized in Table 57a. In SGF, the solubility at 1 hr was 0.73 mg/mL and decreased to 0.14 mg/ml at 24 hrs. In FaSSIF, the solubility at 1 hr was 0.029 mg/ml and decreased to 0.006 mg/mL at 24 hrs.

TABLE 57a
Solubility result of Meglumine salt Type A in SGF and FaSSIF

| | Initial | 1 hr | | 2 hrs | | 4 hrs | | 24 hrs | |
|---|---|---|---|---|---|---|---|---|---|
| Medium | pH | S | pH* | S | pH* | S | pH* | S | pH* |
| SGF | 2.4 | 0.73 | 1.7 | 0.16 | 1.8 | 0.12 | 1.8 | 0.14 | 1.9 |
| FaSSIF | 6.7 | 0.029 | 6.8 | 0.014 | 6.5 | 0.011 | 6.5 | 0.006 | 6.6 |

### Meglumine Type B

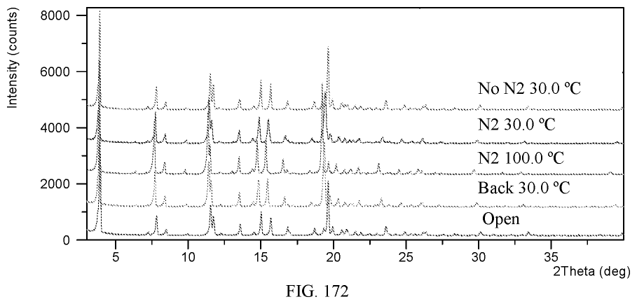

Meglumine salt Type B (was characterized by XRPD, TGA, DSC and 1H NMR. XRPD and TGA/DSC result were shown in FIGS. 77, 78, and 79, respectively. TGA result showed a weight loss of 5.69% to 150° C. DSC results showed three endotherms at 80.9° C., 132.2° C. and 167.1° C. (peak temperature) and an exotherm at 148.5° C. (peak temperature). 1H NMR result was shown in FIG. 154 showed the molar ration of meglumine/freeform was 1.0 and residual solvent MeOH/API and MTBE/API was 0.2 (0.8 wt %) and 0.02 (0.2 wt %). VT-XRPD was performed for Meglumine salt Type B and the result was shown in FIG. 172. Form change was observed after N2 purging for 20 min at 30° C. No further form change was observed after heating to 100° C. and cooling to 30° C. with N2 protection. After exposure to ambient conditions, Meglumine salt Type B was re-obtained. Based on VT-XRPD results, Meglumine salt Type B was speculated to be a hydrate. XRPD results showed that after heating Meglumine salt Type B to 132° C. or 150° C., and cooling to RT with exposure to ambient conditions, a new form was obtained, which was named as Meglumine salt Type C.

### Meglumine Type C

Meglumine salt Type C was obtained via heating Meglumine salt Type B to 132° C. and cooling to RT. XRPD and TGA/DSC results were shown in FIGS. 80, 81, and 82, respectively. TGA result showed a weight loss of 2.35% to 150° C. DSC result showed an exotherm at 131.4° C. (peak temperature) and an endotherm at 166.7° C. (peak temperature). 1H NMR result was shown in FIG. 155. The molar ratio of meglumine/freeform was 1.0 and residual solvent MTBE/API was 0.01 (0.2 wt %), which might be surface-adsorbed or occluded solvent. Based on the characterization results, Meglumine salt Type C was speculated to be an anhydrate due to the gradual TGA weight loss and neat DSC signal before the exotherm.

### Inter-Conversion Relationship Among Meglumine Salt Forms

### Inter-Conversion Relationship Between Anhydrates

To determine the inter-conversion relationship among anhydrates of Meglumine salt, slurry competition experiments were set up for anhydrates Meglumine salt Type A and Type C in MeOH/MTBE (8:13, v/v) and DMSO/EtOAc (1:6, v/v). The detailed procedures are as following:

1) Weigh around 10 mg Meglumine salt Type A into an HPLC vial. Add 1 mL corresponding solvent.

2) Magnetically stir at RT or 50° C. for 1 day. Filter through 0.45 μm PTFE filter to obtain clear solution.

3) Add ˜5 mg of Meglumine salt Type A and Type B to the clear solution. Magnetically stir at RT or 50° C. for 8 days, and then add ˜5 mg Meglumine salt Type C.

4) Magnetically stir at RT or 50° C. for 1 day. Test XRPD of the wet cake.

The slurry competition results are summarized in Table 58. The mixture of Meglumine salt Type A/B/C converted to a new form after slurry in MeOH/MTBE (8:13, v/v) at RT and 50° C. Compared with Meglumine salt Type B, the new form showed differences at ˜13.5° and 18˜22°, and it turned to Meglumine salt Type B after drying under ambient conditions. It is speculated that an intermediate solvate was formed in MeOH/MTBE (8:13, v/v), which could turn to Meglumine salt Type B after desolvation under ambient conditions. The mixture of Meglumine salt Type A/B/C converted to Meglumine salt Type A after slurry in DMSO/EtOAc (1:6, v/v) at RT and 50° C., indicating that Meglumine salt Type A was the thermodynamically more stable form between RT and 50° C.

TABLE 58
Summary of slurry competition
among Meglumine salt Type A/B/C

| Starting Form | Solvent (v/v) | Temperature | Result |
|---|---|---|---|
| Meglumine salt | MeOH/MTBE (8:13) | RT | New form* |
| Type A/B/C | MeOH/MTBE (8:13) | 50° C. | |
| | DMSO/EtOAc (1:6) | RT | Meglumine salt |
| | DMSO/EtOAc (1:6) | 50° C. | Type A |

*Turned to Meglumine salt Type B under ambient conditions.

### Inter-Conversion Relationship Between Hydrate and Anhydrate

To determine the inter-conversion relationship between hydrate Meglumine salt Type B and the thermodynamically more stable anhydrate Meglumine salt Type A, slurry competition experiments were set up for anhydrate Meglumine salt Type A and hydrate Meglumine salt Type B under different water activities (aw˜0, 0.2, 0.4, 0.6, 0.8) at RT. The detailed procedures are as following:

1) Weigh around 10 mg Meglumine salt Type A into an HPLC vial. Add 1 mL corresponding solvent.

2) Magnetically stir at RT for 1 day. Filter through 0.45 μm PTFE filter to obtain clear solution.

3) Add ˜5 mg of Meglumine salt Type A and Type B to the clear solution.

4) Magnetically stir at RT. Test XRPD of the wet cake.

The slurry competition results were summarized in Table 59. The mixture of Meglumine salt Type A/B converted to Meglumine salt Type A after slurry in aw=0˜0.8 systems at RT.

TABLE 59
Summary of slurry competition between
Meglumine salt Type A and B

| Starting Form | Solvent (v/v) | a_{w} | Result |
|---|---|---|---|
| Meglumine | EtOH | ~0.0 | Meglumine salt Type A |
| salt Type A/B | EtOH/H₂O (984:16) | ~0.2 | Meglumine salt Type A |
| | EtOH/H₂O (950:50) | ~0.4 | Meglumine salt Type A |
| | EtOH/H₂O (857:143) | ~0.6 | Meglumine salt Type A |
| | EtOAc/H₂O (976:24) | ~0.8 | Meglumine salt Type A |

### Characterization and Identification of Freeform Forms

Starting from Compound 1 Freeform Type D, polymorph screening experiments were performed under 50 conditions using different solvent-mediated crystallization or solid transition methods. The obtained solids were characterized by XRPD. According to XRPD comparison results, a total of 16 crystal forms were discovered, named as Type A˜P. The characterization results of all the forms were listed in Table 60.

TABLE 60
Characterization results of Compound 1 freeform forms

| Form | TGA weight | | DSC endotherm | Residual solvent | Speculated |
|---|---|---|---|---|---|
| (Sample ID) | loss (%) | | (° C., peak) | by NMR (wt %) | form |
| Freeform Type C | 0.72 | (150° C.) | 187.2* | 0.2 (Acetone) | Anhydrate |
| Freeform Type M | 2.00 | (150° C.) | 187.2* | Not detected | Anhydrate |
| Freeform Type I | 11.68 | (130° C.) | 113.7*, 186.7* | Not detected | Hydrate |
| Freeform Type L | 3.25 | (80° C.) | 64.7, 89.0, | 0.8 (Acetone) | Hydrate |
| | 3.25 | (150° C.) | 120.9^{#}, 188.3 | | |
| Freeform Type P | 4.44 | (120° C.) | 80.8, 166.5, 188.7 | Not detected | Hydrate |
| Freeform Type A | 3.60 | (150° C.) | 103.6, 161.5, 185.6 | Not detected | Anhydrate or |
| | | | | | hydrate |
| Freeform Type E | 3.27 | (150° C.) | 127.3 | 0.3 (MeOH) | Anhydrate or |
| | | | | | hydrate |
| Freeform Type B | 3.63 | (170° C.) | 132.7, 147.9, 189.0 | 3.1 (EtOH) | EtOH solvate |
| Freeform Type D | 10.67 | (180° C.) | 145.6, 148.6 | 10.1 (THF) | Isomorphic |
| | | | | | solvate |
| Freeform Type G | 1.25 | (90° C.) | 61.1, 106.0 | 8.3 (DMSO) | Isomorphic |
| | 11.72 | (250° C.) | | | solvate |
| Freeform Type H | 0.96 | (90° C.) | 107.6, 178.6 | 8.3 (Anisole) | Anisole |
| | 11.25 | (240° C.) | | | solvate |
| Freeform Type J | 1.50 | (100° C.) | 132.0*, 144.4^{#}, 188.0* | 9.9 (DCM) | DCM solvate |
| | 8.03 | (190° C.) | | | |
| Freeform Type K | 12.61 | (120° C.) | 99.6*, 184.6* | 12.1 (DMSO) | DMSO solvate |
| Freeform Type N | 13.50 | (120° C.) | 99.6, 106.5^{#}, | 13.7 (Toluene) | Toluene |

| | |
|---|---|
| 111.5, 187.4 | solvate |

| | | |
|---|---|---|
| Freeform Type F | Change to Freeform Type J after storage at RT. | DCM solvate |
| Freeform Type O | Change to Freeform Type K after drying at RT under vacuum. | DMSO solvate |

*Onset temperature.
^{#}Exotherm, peak temperature.

### Anhydrate

### Freeform Type C

Freeform Type C was obtained via slurry of Freeform Type A in acetone at RT for 3 days and drying at RT under vacuum. XRPD and TGA/DSC results were shown in FIGS. 89, 90, and 91, respectively. TGA result showed a weight loss of 0.72% to 150° C. DSC result showed a sharp endothermic peak at 187.2° C. (onset temperature). 1H NMR result in FIG. 158 showed the molar ratio of residual solvent Acetone/freeform was 0.02 (0.2 wt %), which might be adsorbed on the surface of sample. Based on the characterization results, Freeform Type C was speculated to be an anhydrate due to the low TGA weight loss (lower than the theoretical water content of hemi-hydrate (1.5%)) and neat DSC signal (only one endotherm).

### Freeform Type M

Freeform Type was obtained via heating hydrate Freeform Type I to 130° C. and cooling to RT. XRPD and TGA/DSC results were shown in FIGS. 118, 119, and 120, respectively. TGA result showed a weight loss of 2.00% to 180° C. DSC result showed a sharp endothermic peak at 187.2° C. (onset temperature). 1H NMR result in FIG. 167 revealed negligible solvent was detected. Based on the characterization results, Freeform Type M was speculated to be an anhydrate due to the low and gradual TGA weight loss and neat DSC signal (only one melting signal was observed).

### Hydrate

### Freeform Type I

Freeform Type I was obtained via slurry of starting material Freeform Type D in toluene at 50° C. for 11 days and drying at RT under vacuum for 1 day. XRPD and TGA/DSC results were shown in FIGS. 106, 107, and 108. TGA result showed a stepwise weight of 11.68% up to 130° C. DSC result showed two endotherms at 113.7° C. and 186.7° C. (onset temperature). 1H NMR result in FIG. 163 revealed that negligible solvent toluene was detected, suggesting the TAG weight loss was caused by loss of water. After heating Freeform Type I to 130° C. and cooling to RT, anhydrate Freeform Type M was obtained. Based on the results, Freeform Type I exhibited stepwise TGA weight loss and corresponding endotherm and converted to anhydrate after loss of water over 130° C. Therefore, Freeform Type I was speculated to be a hydrate.

### Freeform Type L

Freeform Type L was obtained via slow evaporation of acetone solution of starting material Freeform Type D at RT. XRPD and TGA/DSC results were shown in FIGS. 115, 116, and 117. TGA result showed a weight loss of 3.25% to 80° C. and 3.25% between 80° C. and 150° C. DSC result showed three endotherms at 64.7° C., 89.0° C. and 188.3° C. (peak temperature) and an exotherm at 120.9° C. (peak temperature). 1H NMR result in FIG. 166 revealed the molar ratio of Acetone/API was 0.18 (1.8 wt %), which might be surface-adsorbed. Heating experiments were performed for identification of Freeform Type L. XRPD comparison results showed no form change was observed after heating to 65° C. and cooling to RT; Amorphous with several weak peaks (might be attributed to Freeform Type M) was observed after heating to 90° C. and cooling to RT with exposure to ambient conditions. Freeform Type M was obtained after heating to 150° C. and cooling to RT. 1H NMR result showed that the molar ratio of residual acetone/API decreased to 0.1 (1.0 wt %). Based on the results, Freeform Type L was speculated to be a hydrate. The endotherm at 89° C. might be the signal of loss of water and form conversion to amorphous and the exotherm at 120.9° C. might be the signal of recrystallization to Freeform Type M.

### Freeform Type P

Freeform Type P was obtained from slurry competition of Freeform Type A/C/E/G/L in H2O at RT and drying at RT under vacuum. XRPD and TGA/DSC results were shown in FIGS. 125, 126, and 127, respectively. TGA result showed a weight loss of 4.44% to 120° C. DSC result showed three endotherms at 80.8° C., 166.5° C. and 188.7° C. (peak temperature). 1H NMR result in FIG. 169 revealed that negligible organic solvent was detected. After heating Freeform Type P to 115° C. and cooling to RT, no form change was observed. After heating to 167° C. and cooling to RT, anhydrate Freeform Type C was obtained. TGA/DSC results of the sample after heating to 100° C. were consistent with Type P before heating. A weight loss of 3.01% to 120° C. and three endotherms at 66.4° C., 166.6° C. and 188.4° C. (peak temperature) were observed. Freeform Type P exhibited a stepwise TGA weight loss and corresponding DSC endotherm. Moreover, Freeform Type P was obtained after slurry competition in H2O. Therefore, Freeform Type P was speculated to be a hydrate. The endotherm at 80.8° C. might be the signal of loss of water and form conversion (form changed after loss of water over 100° C. and back to Type P after adsorption of water at ambient conditions). The endotherm at 164.5° C. might be the signal of form conversion to Freeform Type C.

### Anhydrate/Hydrate

### Freeform Type A

The characterization and identification of Freeform Type A were summarized above. Based on the results, Freeform Type A was speculated to be an anhydrate or hydrate.

### Freeform Type E

Freeform Type E was obtained via adding anti-solvent H2O into MeOH solution of Freeform Type D and drying at ambient conditions. XRPD result was shown in FIG. 96.

TGA/DSC results in FIGS. 97 and 98, respectively, showed a weight loss of 3.27% and one endotherm at 127.3° C. (peak temperature). 1H NMR result was shown in FIG. 160. The molar ratio of residual solvent MeOH/API was 0.06 (0.3 wt %), which might be surface-adsorbed. After heating Freeform Type E to 150° C. and cooling to RT, the sample melted. Based on the characterization results, Freeform Type E was speculated to be an anhydrate or hydrate.

### Solvate

### Freeform Type B

Freeform Type B was obtained via slurry of Freeform Type A in EtOH at RT for 3 days and drying at RT under vacuum. XRPD and TGA/DSC results were shown in FIGS. 86, 87, and 88, respectively. TGA result showed a stepwise weight loss of 3.63% up to 170° C. DSC result showed three endotherms of 132.7° C., 147.9° C. and 189.0° C. (peak temperature). 1H NMR result was shown in FIG. 157. The molar ratio of EtOH/API was 0.4 (3.1 wt %, close to TGA weight loss). After heating Freeform Type B to 130° C., cooling to RT and being exposed to ambient conditions, XRPD result showed that amorphous was obtained. After heating to 150° C. and cooling to RT, the sample melted. Based on the characterization results, Freeform Type B was speculated to be an EtOH solvate.

### Freeform Type D

Freeform Type D were obtained via slurry of Freeform Type D in 2-MeTHF and 1,4-Dioxane/H2O (1:1, v/v) at 50° C. for 25 days and drying at RT under vacuum overnight. XRPD results were shown in FIG. 93. 1H NMR results were shown in FIG. 159. For Freeform Type D, the molar ratio of 2-MeTHF/API was 0.7 (9.5 wt %). For Freeform Type D, the molar ratio of 1,4-Dioxane/API was 0.8 (10.9 wt %). It is speculated that these two batches of Freeform Type D were 2-MeTHF solvate and 1,4-Dioxane solvate, respectively. Since Freeform Type D was THF solvate, Type D was potentially an isomorphic form with similar crystal structure to allow participation of various guest molecules.

### Freeform Type G

Freeform Type G was obtained via slurry of Freeform Type D in DMSO/H2O (1:4, v/v) at RT. XRPD and TGA/DSC results were shown in FIGS. 100, 101, and 102, respectively. TGA result showed a weight of 0.96% up to 90° C. and 11.25% between 90° C. and 240° C. DSC result showed two endotherms at 107.6° C. and 178.6° C. (peak temperature). 1H NMR result in FIG. 161 showed that the molar ratio of DMSO/freeform was 0.9 (8.3 wt %). After heating Freeform Type G to 120° C. and cooling to RT, XRPD result showed anhydrate Type C was obtained. Based on the results, Freeform Type G was speculated to be a DMSO solvate.

Another batch of Freeform Type G was evaluated. TGA/DSC results in FIG. 7 39 showed a stepwise weight loss of 3.88% to 150° C. and one endotherm at 132.6° C. (peak temperature). 1H NMR result showed negligible organic solvent detected. After heating Freeform Type G to 150° C. and cooling to RT, amorphous sample was obtained (sample melted). Based on the characterization results, this batch of Freeform Type G showed a stepwise TGA weight loss without residual organic solvate, therefore, it was speculated to be a hydrate. According to the characterization results of two batches of Freeform Type G, it was potentially an isomorphic form with similar crystal structure to allow participation of various guest molecules

### Freeform Type H

Freeform Type H was obtained via slurry of Freeform Type D in anisole at 50° C. for 11 days and drying at RT under vacuum overnight. XRPD and TGA/DSC results were shown in FIGS. 103, 104, and 105, respectively. TGA result showed a weight of 1.50% up to 100° C. and 8.03% between 100° C. and 190° C. DSC result showed two endotherms at 132.0° C. and 188.0° C. (onset temperature) and an exotherm at 144.4° C. (peak temperature). 1H NMR result was shown in FIG. 162, the molar ratio of solvent anisole/API was ˜0.5 (8.3 wt %). After heating Freeform Type H to 135° C. or 150° C. and then cooling to RT, anhydrate Type M was obtained. Based on the result, Freeform Type H was speculated to be an Anisole solvate. The endotherm and exotherm at 132.0° C. and 144.4° C. might be the signal of form conversion to Freeform Type M.

### Freeform Type J

Freeform Type J was obtained via solid vapor diffusion of starting material Freeform Type D in DCM atmosphere at RT for 7 days. XRPD and TGA/DSC results were shown in FIGS. 109, 110, and 111, respectively. TGA result showed a stepwise weight of 12.61% up to 120° C. DSC result showed two endotherms at 99.6° C. and 184.6° C. (onset temperature). 1H NMR result was shown in FIG. 164, the molar ratio of solvent DCM/API was ˜0.8 (9.9 wt %). After heating Freeform Type J to 120° C. and cooling to RT, anhydrate Type M was obtained. Based on the characterization results, Freeform Type J was speculated to be a DCM solvate. The endotherm at 99.6° C. was the signal of loss of solvent and form conversion to Freeform Type M.

### Freeform Type K

Freeform Type K was obtained via solid vapor diffusion of starting material Freeform Type D in DMSO atmosphere at RT for 7 days. XRPD and TGA/DSC results were shown in FIGS. 112, 113, and 114, respectively. TGA result showed a weight of 1.25% up to 90° C. and 11.72% between 90° C. and 250° C. DSC result showed two endotherms at 61.1° C. and 106.0° C. (peak temperature). 1H NMR result was shown in FIG. 165, the molar ratio of solvent DMSO/API was ˜1.0 (12.1 wt %, close to the second stage weight loss). For Freeform Type K after heating to 70° C., no form change was observed. After heating to 150° C. and cooling to RT, sample melted. The sample after heating to 70° C. was characterized by TGA/DSC and 1H NMR. TGA result showed a weight of 12.14% up to 90° C. and 12.85% between 90° C. and 250° C. DSC result showed an endotherm at 104.8° C. (onset temperature). 1H NMR result showed the molar ratio of solvent DMSO/API was still 1.0 (12.1 wt %). Based on the characterization results, Freeform Type K was a DMSO solvate.

### Freeform Type N

Freeform Type N was obtained via slurry of starting material in toluene at 50° C. for 7 days. XRPD and TGA/DSC results were shown in FIGS. 121, 122, and 123, respectively. TGA result showed a weight of 13.50% up to 120° C. DSC result showed three endotherms at 99.6° C., 111.5° C. and 187.4° C. (peak) and an exotherm at 106.5° C. (peak). 1H NMR result was shown in FIG. 168, the molar ratio of residual API was 1.0 (13.7 wt %, close to the TGA weight loss). After heating to 95° C. and cooling to RT, amorphous with several weak diffractions were obtained. After heating to 105° C. and 130° C., anhydrate Freeform Type M was obtained. Based on the results, Freeform Type N was speculated to be a Toluene solvate. The signal before 187.4° C. might be caused by loss of solvent and form conversion to Freeform Type M.

### Freeform Type F

Freeform Type F was obtained via slurry of Freeform Type D in DCM at RT. After storage at ambient conditions overnight, Freeform Type F converted to Type J. XRPD results were shown in FIG. 99. As Freeform Type J was a DCM solvate, it is speculated that Freeform Type F was also a DCM solvate.

### Freeform Type O

Freeform Type O was obtained after adding anti-solvent toluene into DMSO solution of Freeform Type D and drying at ambient conditions. After drying at RT under vacuum for 1 day, diffraction peaks of Freeform Type K were observed. XRPD results were shown in FIG. 124. As Freeform Type K was a DMSO solvate, it is speculated that Freeform Type O was also a DMSO solvate.

### Inter-Conversion Relationship Among Freeform Forms

To determine the inter-conversion relationship among anhydrates of freeform, slurry competition experiments were set up for anhydrates Freeform Type C/M and anhydrate or hydrate Freeform Type A in EtOAc and ACN. The detailed procedures are as following:

1. Weighed around 9 mg Freeform Type C into an HPLC vial. Add 1 mL corresponding solvent.

2. Magnetically stirred at RT or 50° C. for 4 hours. Filter through 0.45 μm PTFE filter to obtain clear solution.

3. Added equal amount (˜5 mg) of Freeform Type A, Freeform Type C, Freeform Type M and Freeform Type G to the clear solution.

4. Magnetically stirred at RT or 50° C. for 4 days. Tested XRPD of the wet cake.

The slurry competition results are summarized in Table 61. The mixture of Freeform Type A/C/G/M converted to Freeform Type C after slurry in EtOAc and ACN at RT and 50° C., indicating that Freeform Type C was the thermodynamically more stable form between RT and 50° C.

TABLE 61
Summary of slurry competition among Freeform Type A/C/M

| Starting Form | Solvent | Temperature | Result |
|---|---|---|---|
| Freeform Type | EtOAc | RT | Freeform Type C |
| A/C/G/M | EtOAc | 50° C. | Freeform Type C |
| | ACN | RT | Freeform Type C |
| | ACN | 50° C. | Freeform Type C |

### Inter-Conversion Relationship Among Hydrates and Freeform Type C

To determine the inter-conversion relationship between hydrates of freeform and the thermodynamically more stable anhydrate Freeform Type C, slurry competition experiments were set up for anhydrate Freeform Type C, hydrates Freeform Type UG and anhydrates or hydrates Freeform Type A/E under different water activities (aw) in Acetone/H2O at RT. The detailed procedures are as following:

1. Weighed around 7 mg Freeform Type C into an HPLC vial. Add 1 mL corresponding solvent.

2. Magnetically stirred at RT for 16 hours. Filter through 0.45 μm PTFE filter to obtain clear solution.

3. Added equal amount (˜5 mg) of Freeform Type A, Freeform Type C, Freeform Type, Freeform Type L and Freeform Type ET=G to the clear solution.

4. Magnetically stirred at RT. Tested XRPD of the wet cake.

The slurry competition results are summarized in Table 8 2. XRPD comparison results are shown from FIG. 8 3 to FIG. 8 5. The mixture of Freeform Type A/C/E/G/L converted to a new form of Freeform Type P in H₂O (aw=1) after 76 days. Freeform Type P was added to aw=0˜0.8 systems. As a result, Freeform Type C was obtained. The slurry competition results indicated that the critical water activity at RT between anhydrate Freeform Type C and hydrate Freeform Type P was between 0.8 and 1.

TABLE 63
Summary of slurry competition among Freeform Type A/C/E/G/L

| Starting Form | Solvent (v/v) | Aw | Result |
|---|---|---|---|
| Freeform Type | Acetone | ~0.0 | Freeform Type C |
| A/C/E/G/L/P | Acetone/H₂O (984:16) | ~0.2 | Freeform Type C |
| | Acetone/H₂O (950:50) | ~0.4 | Freeform Type C |
| | Acetone/H₂O (857:143) | ~0.6 | Freeform Type C |
| | Acetone/H₂O (604:396) | ~0.8 | Freeform Type C |
| Freeform Type | H₂O | 1.0 | Freeform Type P |
| A/C/E/G/L | | | |

### Approximate Solubility

Approximate solubility of Compound 1 freeform Type A, freeform Type D and meglumine salt Type A was measured in different solvents at RT. For each experiment, approximately 2 mg of sample was added into a 3-mL glass vial. Corresponding solvents were then added stepwise (50/50/200/700/1000 μL) into the vials until the solids were dissolved visually or a total volume of 2 mL was reached. Solubility results summarized in Table 64 to Table 66 were used to guide the solvent selection in polymorph screening design.

TABLE 64
Approximate solubility of Freeform Type A

| Solvent | Solubility (mg/mL) | Solvent | Solubility (mg/mL) |
|---|---|---|---|
| MeOH | 6.7 < S < 20.0 | CPME | 1.1 < S < 2.1 |
| EtOH | 2.1 < S < 7.0 | 1,4-Dioxane | 1.9 < S < 6.3 |
| IPA | 1.1 < S < 2.2 | Anisole | 1.1 < S < 2.1 |
| Acetone | 6.7 < S < 20.0 | ACN | 1.0 < S < 1.9 |
| MIBK | 1.0 < S < 2.0 | DCM | 7.3 < S < 22.0 |
| EtOAc | 2.0 < S < 6.7 | CHCl₃ | 6.3 < S < 19.0 |
| IPAc | 1.0 < S < 1.9 | n-Heptane | S < 1.0 |
| MTBE | S < 1.0 | Toluene | S < 1.1 |
| THF | S > 38.0 | DMSO | S > 40.0 |
| 2-MeTHF | 1.9 < S < 6.3 | H₂O | S < 1.1 |

TABLE 65
Approximate solubility of Freeform Type D

| Solvent | Solubility (mg/mL) | Solvent | Solubility (mg/mL) |
|---|---|---|---|
| MeOH | 6.0 < S < 18.0 | CPME | S < 0.9 |
| EtOH | 2.7 < S < 6.3 | 1,4-Dioxane | S < 1.1 |
| IPA | S < 1.1 | Anisole | S < 1.0 |
| Acetone | 7.0 < S < 21.0 | ACN | 1.1 < S < 2.2 |
| MIBK | 2.0 < S < 6.7 | DCM | 2.1 < S < 7.0 |
| EtOAc | 2.1 < S < 7.0 | CHCl₃ | S < 1.1 |
| IPAc | 0.9 < S < 1.8 | n-Heptane | S < 1.0 |
| MTBE | S < 1.1 | Toluene | S < 1.0 |
| THF | 2.2 < S < 7.3 | DMSO | S > 44.0 |
| 2-MeTHF | S < 1.0 | H₂O | S < 1.1 |

TABLE 66
Approximate solubility of Meglumine salt Type A

| Solvent | Solubility (mg/mL) | Solvent | Solubility (mg/mL) |
|---|---|---|---|
| MeOH | 6.7 < S < 20.0 | CPME | S < 1.0 |
| EtOH | 1.1 < S < 2.1 | 1,4-Dioxane | 1.0 < S < 1.9 |
| IPA | S < 1.1 | Anisole | S < 1.1 |
| Acetone | S < 1.1 | ACN | S < 1.0 |
| MIBK | S < 1.0 | DCM | S < 1.1 |
| EtOAc | S < 1.1 | CHCl₃ | S < 1.0 |
| IPAc | S < 1.0 | n-Heptane | S < 1.0 |
| MTBE | S < 1.0 | Toluene | S < 1.2 |
| THF | S < 1.1 | DMSO | 22.0 < S < 44.0 |
| 2-MeTHF | S < 1.0 | H₂O | S > 44.0 |

### Log D Test Method

### Preparation of Phosphate pH 7.5 Buffer

0.1 M KH₂PO₄ solution: added 1.36 g of solid KH₂PO₄ into a 100-mL volumetric flask, dissolve with ultrapure water and dilute to volume. 0.1 M K₂HPO₄ solution: added 1.74 g of solid K₂HPO₄ into a 100-mL volumetric flask, dissolve with ultrapure water and dilute to volume.

Transferred 100 mL of 0.1 M K₂HPO₄ solution and 30 mL of 0.1 M KH₂PO₄ to a 250-mL beaker and mix well. Adjusted to pH 7.4 with the KH₂PO₄ solution to obtain the aqueous pH 7.4 buffer. Pre-equilibrated n-octanol and aqueous buffer (phosphate pH 7.4 buffer) by adding 10 mL n-octanol and 10 mL aqueous buffer into a glass vial and keep it rolling for 24 hrs.

### Setting Up Experiment:

Weighed approximately 1.0 mg solids into a 3-mL glass vial which contained 2.0 mL of saturated n-octanol and accelerate dissolution by ultrasound sonication. Added 1.0 mL of complementary saturated aqueous buffer into the vial. Prepare samples in triplicate. Sealed the glass vial and mix on a rotary mixer at 25° C. for 24 hrs.

### Data Analysis:

The phases were separated. The pH of the aqueous phase was measured by pH meter, and the concentrations of compounds in each phase were determined by HPLC. Samples in n-octanol phase were diluted 10 times by ACN. Distribution coefficient, referred to as Dow, was calculated as the concentration of the test compound in the n-octanol phase divided by the corresponding concentration in the aqueous phase.

### Characterization of the Salts

### HCl Salt

HCl salt Type A was obtained via slurry freeform and HCl (molar ratio of 1:1, acid/freeform) in EtOAc at RT for 3 days. The XRPD pattern was displayed in FIG. 17. The TGA/DSC curves of HCl salt Type A were displayed in FIGS. 18 and 19, respectively, which showed a weight loss of 4.18% up to 150° C. and 3 endotherms at 116.9, 154.7 and 172.5° C. (peak). The 1H NMR result in FIG. 134 showed molar ratio of EtOAc/API was 0.05 (0.7 wt %). HPLC/IC results showed the purity was 96.24 area % and the molar ratio was 0.7 (Cl—/freeform).

### Sulfate

Sulfate Type A/B/C were obtained via slurry freeform and H₂SO₄ (molar ratio of 1:1, acid/freeform) in EtOH, Acetone and EtOAc, respectively, at RT for 3 days. The XRPD patterns were displayed in FIGS. 20, 23, and 26 for A, B, and C, respectively.

The TGA/DSC curves of Sulfate Type A were displayed in FIGS. 21 and 22, which showed a weight loss of 7.10% up to 150° C. and three endotherms at 70.1, 116.6 and 150.7° C. (peak). The 1H NMR result in FIG. 135 showed molar ratio of EtOH/API was 0.09 (0.6 wt %). HPLC/IC results showed the purity was 92.91 area % and the molar ratio was 1.0 (SO₄²⁻/freeform).

The TGA/DSC curves of Sulfate Type B were displayed in FIGS. 24 and 25, which showed a weight loss of 3.81% up to 150° C. and two endotherms at 131.5 and 169.8° C. (peak). The signal of acetone was observed in the 1H NMR (FIG. 136, since the signal of acetone was overlapped with the API peak, the exact content of residual solvent was not determined).

HPLC/IC results showed the purity was 95.33 area % and the molar ratio was 1.1 (SO₄²⁻/freeform).

The TGA/DSC curves of Sulfate Type C were displayed in FIGS. 27 and 28, which showed a weight loss of 6.72% up to 150° C. and one endotherm at 93.2° C. (peak). The 1H NMR result in FIG. 137 showed molar ratio of EtOAc/API was 0.2 (3.0 wt %). HPLC/IC results showed the purity was 95.70 area % and the molar ratio was 1.2 (SO₄²⁻/freeform).

### Phosphate

Phosphate Type A was obtained via slurry Freeform Type A and H₃PO₄ (molar ratio of 1:1, acid/freeform) in EtOH at RT for 3 days. The XRPD pattern was displayed in FIG. 29. The TGA/DSC curves were displayed in FIGS. 30 and 31, which showed a weight loss of 2.40% up to 150° C. and two endotherms at 133.9 and 159.3° C. (peak). The ¹H NMR result in FIG. 138 showed molar ratio of EtOH/API was 0.2 (1.3 wt %). HPLC/IC results showed the purity was 95.62 area % and the molar ratio was 0.7 (PO₄³⁻/Freeform).

### Tartrate

Tartrate Type A was obtained via slurry freeform and tartaric acid (molar ratio of 1:1, acid/FB) in Acetone at RT for 3 days. The XRPD pattern was displayed in FIG. 32. The TGA/DSC curves of Tartrate Type A were displayed in FIGS. 33 and 34, respectively, which showed a weight loss of 4.62% up to 150° C. and two endotherms at 122.8 and 189.0° C. (peak). The 1H NMR result in FIG. 139 showed the molar ratio of acid/freeform was 1.1 and the molar ratio of Acetone/API was 0.1 (0.8 wt %). HPLC results showed the purity was 96.28 area %.

### Fumarate

Fumarate Type A was obtained via slurry Freeform Type A and fumaric acid (molar ratio of 1:1, acid/freeform) in Acetone at RT for 3 days. The XRPD pattern was displayed in FIG. 5. The TGA/DSC curves of Fumarate Type A were displayed in FIGS. 6 and 7, respectively, which showed a weight loss of 1.83% up to 150° C. and one endotherm at 209.9° C. (peak). The 1H NMR result in FIG. 131 showed the molar ratio of acid/freeform was 0.6 and the molar ratio of Acetone/API was 0.2 (1.4 wt %). HPLC results showed the purity was 96.23 area %.

### Mesylate

Procedure of preparation of Mesylate Type A was listed below: Clear solution was obtained via slurry Freeform Type A and methanesulfonic acid (molar ratio of 1:1, acid/freeform) in Acetone at RT for 3 days and slurry at 5/−20° C. Solid was obtained via anti-solvent addition using n-Heptane. The XRPD pattern was displayed in FIG. 35. The TGA/DSC curves of Mesylate Type A were displayed in FIGS. 36 and 37, which showed a weight loss of 5.91% up to 150° C. and one endotherm at 131.3° C. (peak). The 1H NMR result in FIG. 140 showed the molar ratio of acid/freeform was 0.9 and the molar ratio of Acetone/API was 0.06 (0.5 wt %). HPLC results showed the purity was 96.59 area %.

### Tosylate

Tosylate Type A was obtained via slurry Freeform Type A and p-toluenesulfonic acid (molar ratio of 1:1, acid/freeform) in EtOAc at RT for 3 days. Tosylate Type B was obtained via slurry Freeform Type A and p-toluenesulfonic acid (molar ratio of 1:1, acid/freeform) in EtOH at RT for 3 days, slurry at 5° C. for 3 days and slurry at −20° C. for 4 days. The XRPD patterns were displayed in FIGS. 38 and 41, for A and B respectively.

The TGA/DSC curves of Tosylate Type A (were displayed in FIGS. 39 and 40, respectively, which showed a weight loss of 5.23% up to 150° C. and four endotherms at 76.6 and 117.8° C. (peak). The 1H NMR result in FIG. 141 showed the molar ratio of acid/freeform was 0.8 and no obvious solvent signal was observed. HPLC results showed the purity was 94.30 area %.

The TGA/DSC curves of Tosylate Type B were displayed in FIGS. 42 and 43, which showed a weight loss of 5.86% up to 150° C. and two endotherms at 125.8 and 128.2° C. (peak). The 1H NMR result in FIG. 142 showed the molar ratio of acid/freeform was 0.9 and the molar ratio of EtOH/API was 0.8 (4.9 wt %). HPLC results showed the purity was 95.87 area %.

### Arginine

Arginine salt Type A were obtained via slurry Freeform Type A and arginine (molar ratio of 1:1, acid/freeform) in Acetone at RT for 3 days. Arginine Type B was obtained via slurry freeform Type A and arginine (molar ratio of 1:1, acid/FB) in EtOH at RT for 3 days and temperature cycling from 50-5° C. The XRPD patterns were displayed in FIGS. 44 and 47, for A and B respectively.

The TGA/DSC curves of Arginine salt Type A were displayed in FIGS. 45 and 46, which showed a weight loss of 6.56% up to 150° C. and one endotherm at 92.4° C. (peak). The 1H NMR result in FIG. 143 showed the molar ratio of acid/freeform was 0.5 and the molar ratio of Acetone/API was 0.1:1 (1.2 wt %). HPLC results showed the purity was 95.29 area %.

The TGA/DSC curves of Arginine salt Type B were displayed in FIGS. 48 and 49, which showed a weight loss of 4.34% up to 150° C. and one endotherm at 140.8° C. (peak). The 1H NMR result in FIG. 144 showed the molar ratio of acid/freeform was 1.0 and the molar ratio of EtOH/API was 0.8 (5.1 wt %). HPLC results showed the purity was 96.44 area %.

### Lysine

Lysine salt Type A/B were obtained via slurry Freeform Type A and lysine (1:1, acid/freeform) in EtOH and Acetone, respectively, at RT for 3 days. The XRPD patterns were displayed in FIGS. 50 and 53 for A and B respectively.

The TGA/DSC curves of Lysine salt Type A were displayed in FIGS. 51 and 52, which showed a weight loss of 5.72% up to 150° C. and two endotherms at 74.2 and 174.0° C. (peak). The 1H NMR result in FIG. 145 showed the molar ratio of acid/freeform was 0.9 and the signal of EtOH was detected.

The TGA/DSC curves of Lysine salt Type B were displayed in FIGS. 54 and 55, which showed a weight loss of 7.06% up to 150° C. and one endotherm at 207.7° C. (peak). The 1H NMR result in FIG. 146 showed the molar ratio of acid/freeform was 1.2 and the molar ratio of Acetone/API was 0.4 (3.3 wt %). HPLC results showed the purity was 92.87 area %.

### Sodium

Na salt Type A was obtained via slurry Freeform Type A and NaOH (molar ratio of 1:1, freeform/base) in EtOH at RT for 3 days. The XRPD pattern was displayed in FIG. 56. The TGA/DSC curves of Na salt Type A was displayed in FIGS. 57 and 58, respectively, which showed a weight loss of 7.00% up to 150° C. and two endotherms at 92.2 and 135.5° C. (peak). The 1H NMR result in FIG. showed molar ratio of EtOH/API was 0.3 (2.6 wt %). HPLC/IC results showed the purity was 96.99 area % and the molar ratio was 1.0 (freeform/Na⁺).

### Potassium

K salt A/B were obtained via slurry Freeform Type A and KOH (1:1, base/freeform) in EtOH and Acetone, respectively, at RT for 3 days. The XRPD patterns were displayed in FIGS. 9 and 59, A and B respectively.

The TGA/DSC curves of K salt A were displayed in FIGS. 10 and 11, which showed a weight loss of 1.35% up to 150° C. and one endotherm at 242.4° C. (peak). The 1H NMR result in FIG. 132 showed molar ratio of EtOH/API was 0.09 (0.7 wt %). HPLC/IC results showed the purity was 97.31 area % and the molar ratio was 0.5 (K⁺/freeform).

The TGA/DSC curves of K salt B were displayed in FIGS. 60 and 61, which showed a weight loss of 3.90% up to 150° C. and three endotherms at 150.6, 224.0 and 232.8° C. (peak). The 1H NMR result in FIG. 148 showed molar ratio of Acetone/API was 0.03 (0.2 wt %). HPLC/IC results showed the purity was 96.93 area % and the molar ratio was 1.0 (K⁺/freeform).

### Choline

Choline salt Type A was obtained via slurry Freeform Type A and choline (molar ratio of 1:1, freeform/base) in Acetone at RT for 3 days. The XRPD pattern was displayed in FIG. 62.

The TGA/DSC curves of Choline salt Type A was displayed in FIGS. 63 and 64, which showed a weight loss of 3.38% up to 150° C. and two endotherms at 184.5 and 210.4° C. (peak). The 1H NMR result in FIG. 149 showed the molar ratio of Base/freeform was 0.3 and the molar ratio of Acetone/API was 0.07 (0.7 wt %). HPLC results showed the purity was 96.71 area %.

### Ammonium

Ammonium salt A/B) were obtained via slurry Freeform Type A and ammonia (1:1, base/freeform) in EtOH and EtOAc, respectively, at RT for 3 days. The XRPD patterns were displayed in FIGS. 65 and 68, A and B respectively.

The TGA/DSC curves of Ammonium salt A were displayed in FIGS. 66 and 67, which showed a weight loss of 4.02% up to 150° C. and three endotherms at 132.8, 156.2 and 192.2° C. (peak). The 1H NMR result in FIG. 150 showed molar ratio of EtOH/API was 0.25 (1.9 wt %). HPLC/IC results showed the purity was 96.99 area % and the molar ratio was 1.1 (NH4⁺/freeform).

The TGA/DSC curves of Ammonium salt B were displayed in FIGS. 69 and 70, which showed a weight loss of 4.10% up to 150° C. and two endotherms at 140.7 and 189.0° C. (peak). The 1H NMR result in FIG. 151 showed molar ratio of EtOAc/API was 0.05 (0.7 wt %). HPLC/IC results showed the purity was 96.90 area % and the molar ratio was 0.7 (NH4⁺/freeform).

### Meglumine

Meglumine salt Type A was obtained via slurry Freeform Type A and meglumine (molar ratio of 1:1, freeform/base) in Acetone at RT for 3 days. The XRPD pattern was displayed in FIG. 1. The TGA/DSC curves of Meglumine salt Type A was displayed in FIGS. 2 and 3, which showed a weight loss of 1.42% up to 150° C. and one endotherm at 163.2° C. (peak). The 1H NMR result in FIG. 130 showed the molar ratio of base/freeform was 1.0 and no signal of Acetone was observed. HPLC results showed the purity was 96.61 area %.

### Tris

Tris salt A were obtained via slurry Freeform Type A and trometamol (molar ratio of 1:1, base/freeform) in EtOAc at RT for 3 days and temperature from 50-5° C. Tris salt B were obtained via slurry Freeform Type A and trometamol (molar ratio of 1:1, base/freeform) in Acetone at RT for 3 days and temperature from 50-5° C. Tris salt C were obtained via heating Tris salt B to 120° C. and cooling down to RT. The XRPD patterns were displayed in FIGS. 71, 74, and 13, A, B, and C respectively.

The TGA/DSC curves of Tris salt A were displayed in FIGS. 72 and 73, which showed a weight loss of 5.39% up to 150° C. and three endotherms at 133.1, 182.0 and 191.2° C. (peak). The 1H NMR result in FIG. 152 showed the molar ratio of base/freeform was 1.0 and the molar ratio of EtOAc/API was 0.06 (0.7 wt %). HPLC results showed the purity was 96.65 area %. PLM result showed that Tris salt A was irregular particle.

The TGA/DSC curves of Tris salt B were displayed in FIGS. 75 and 76, which showed a weight loss of 5.51% up to 150° C. and two endotherms at 104.8 and 191.6° C. (peak). The 1H NMR result in FIG. 153 showed the molar ratio of base/freeform was 0.9 and the molar ratio of Acetone/API was 0.06 (0.5 wt %). HPLC results showed the purity was 97.46 area %. PLM result showed that Tris salt B was irregular lamellar crystal.

The TGA/DSC curves of Tris salt C were displayed in FIGS. 14 and 15, which showed a weight loss of 5.81% up to 150° C. and one endotherm at 192.1° C. (peak). The 1H NMR result in FIG. 133 showed the molar ratio of base/freeform was 1.0 and no obvious solvent signal was observed.

### Preparation of Bio-Relevant Media and pH Buffers

### Preparation of Bio-Relevant Media

### Simulated Gastric Fluid (SGF)

0.1 g of NaCl and 0.05 g of Triton X-100 were weighed into a 50-mL volumetric flask. Purified water was added to dissolve the solid. 67.5 μL HCl (38%, 12M) was added after the solid was dissolved completely. pH was then adjusted to 1.8 using HCl (1 M) or NaOH (1 M). Purified water was added to the volume.

### Fasted-State Simulated Intestinal Fluid (FaSSIF)

0.17 g of anhydrous NaH2PO4, 0.021 g of NaOH and 0.31 g of NaCl were weighed into a 50-mL volumetric flask. ˜48 mL purified water was added to dissolve the solid. pH was adjusted to 6.5 using 1M HCl or 1M NaOH. Purified water was then added to the volume. 0.11 g of SIF powder was added into the volumetric flask.

### Fed-State Simulated Intestinal Fluid (FeSSIF)

0.41 mL of acetic acid, 0.20 g of NaOH and 0.59 g of NaCl were weighed into a 50-mL volumetric flask. ˜48 mL purified water was added to dissolve the solid. pH was adjusted to 5.0 using 1M HCl or 1M NaOH. Purified water was then added to the volume. 0.56 g of SIF powder was added into the volumetric flask.

### Preparation of pH Buffers

### pH 1.0 Buffer (KCl—HCl)

Transferred 2.5 mL of 0.2 M KCl into a 10-mL volumetric flask, and adjusted the pH to 1.0 with 0.2 M HCl. Diluted to the target volume with purified water and mixed well.

### pH 2.0 Buffer (KCl—HCl)

Transferred 2.5 mL of 0.2 M KCl into a 10-mL volumetric flask, and adjusted the pH to 2.0 with 0.2 M HCl. Diluted to the target volume with purified water and mixed well.

### pH 3.5 Buffer (KCl—HCl)

Transferred 2.5 mL of 0.2 M KCl into a 10-mL volumetric flask, and adjusted the pH to 3.5 with 0.2 M HCl. Diluted to the target volume with purified water and mixed well.

### pH 4.5 Buffer (NaAc—HAc)

Weighed 18.02 mg of NaAc into a 10-mL volumetric flask, and added appropriate volume of purified water to dissolve the solids. Adjusted the pH to 4.5 with 2 M HAc, and added purified water to the target volume and mix well.

### pH 5.5 Buffer (NaAc—HAc)

Weighed 36.07 mg of NaAc·3H2O into a 10-mL volumetric flask, and added appropriate volume of purified water to dissolve the solids. Adjusted the pH to 5.5 with 2 M HAc, and added purified water to the target volume and mix well.

pH 6.8 Buffer (KH₂PO₄—NaOH)

Transferred 2.5 mL of 0.2 M KH₂PO₄ into a 10-mL volumetric flask, adjusted the pH to 6.8 with 0.2 M NaOH. Added purified water to the target volume and mixed well.

pH 8.0 Buffer (KH₂PO₄—NaOH)

Transferred 2.5 mL of 0.2 M KH₂PO₄ into a 10-mL volumetric flask, adjusted the pH to 8.0 with 0.2 M NaOH. Added purified water to the target volume and mix well.

### Polymorph Screening for Meglumine Salt

A total of 50 polymorph screening experiments were performed for meglumine salt Type A using different crystallization or solid transformation methods. The methods utilized and results were summarized in Table 67 and Meglumine salt Type A was observed.

TABLE 67
Summary of polymorph screening experiments of Meglumine salt

| | No. of | |
|---|---|---|
| Method | Experiment | Result |
| Anti-solvent | 7 | Meglumine salt Type A |
| addition | | |
| Slurry at RT | 15 | Meglumine salt Type A, gel |
| Slurry at 50° C. | 10 | Meglumine salt Type A, amorphous |
| Slow evaporation | 2 | Amorphous |
| Vapor-solid | 6 | meglumine salt Type A |
| diffusion | | |
| Vapor-solution | 5 | Amorphous, gel |
| diffusion | | |
| Slow cooling | 2 | Meglumine salt Type A, amorphous |
| Grinding | 3 | Meglumine salt Type A |
| Total | 50 | Meglumine salt Type A, amorphous, |
| | | gel |

### Anti-Solvent Addition

About 20 mg of meglumine salt Type A was dissolved in 0.5˜1.0 mL solvent to obtain a clear solution and the solution was magnetically stirred followed by addition of anti-solvent until precipitant appeared. The samples were isolated via centrifugation. The solids were isolated for XRPD analysis. Results in Table 68 showed that Meglumine salt Type A was obtained.

TABLE 68
Summary of anti-solvent addition experiments

| Solvent | Anti-solvent | Result |
|---|---|---|
| MeOH | MIBK | Meglumine |
| | IPAc | Meglumine salt Type A* |
| | CPME | Meglumine salt Type A* |
| DMSO | MTBE | Meglumine salt Type A* |
| | ACN | Meglumine salt Type A |
| | DCM | Meglumine salt Type A |
| H₂O | IPA | Meglumine salt Type A^{#} |

### Slurry a RT

About 20 mg of meglumine salt Type A was suspended in 0.5 ml, of solvent in an HPLC glass vial. After the suspension was stirred magnetically (˜1000 rpm) for ˜7 days at RT, the remaining solids were centrifuged (10000 rpm, 2 min) for XRPD analysis. Results summarized in Table 69 indicated that Meglumine salt Type A and gel-like samples were obtained.

TABLE 69
Summary of slurry conversion experiments at RT

| Solvent (v/v) | Result |
|---|---|
| MeOH | Meglumine salt Type A* |
| EtOH | Meglumine salt Type A |
| Acetone | Meglumine salt Type A |
| MIBK | Meglumine salt Type A |
| EtOAc | Meglumine salt Type A |
| MTBE | Meglumine salt Type A |
| THF | Meglumine salt Type A |
| 1,4-Dioxane | Meglumine salt Type A |
| ACN | Meglumine salt Type A |
| DCM | Meglumine salt Type A |
| EtOH/n-heptane (1:1) | Meglumine salt Type A |
| IPA/IPAc (1:1) | Meglumine salt Type A |
| DMSO/CPME (1:1) | Gel* |
| MeOH/Anisole (1:1) | Meglumine salt Type A* |
| ACN/H₂O (92:8, a_{w}~0.8) | Meglumine salt Type A |

*Clear solution was obtained after slurry at RT for 4 days, slurry at 5° C. for 2 days, slurry at −20° C. for 1 day and evaporation in the air.

### Slurry at 50° C.

About 20 mg of meglumine salt Type A was suspended in 0.5 ml, of solvent in an HPLC glass vial. After the suspension was stirred (1000 rpm) for about 7 days at 50° C., the remaining solids were centrifuged (10000 rpm, 2 min) for XRPD analysis. Results summarized in Table 70 indicated that Meglumine salt Type A was generated.

TABLE 70
Summary of slurry conversion experiments at 50° C.

| Solvent (v/v) | Result |
|---|---|
| IPA | Meglumine salt Type A |
| MEK | Meglumine salt Type A |
| IPAc | Meglumine salt Type A |
| 2-MeTHF | Meglumine salt Type A |
| Anisole | Meglumine salt Type A |
| CHCl₃ | Meglumine salt Type A |
| Toluene | Meglumine salt Type A |
| EtOH/Acetone (1:1) | Meglumine salt Type A |
| THF/H₂O (4:1) | Amorphous* |
| DMAc/EtOAc (1:2) | Meglumine salt Type A |

### Slow Evaporation

Slow evaporation experiments were performed under 2 conditions. Briefly, ˜20 mg of meglumine salt Type A was dissolved in 2.0 ml of solvent in a 3-ml glass vial. The resulting solutions were subjected to slow evaporation at RT with vials sealed by Parafilm® and poked with 4 pinholes. The solids were isolated for XRPD analysis and the results summarized in Table 71 indicated that amorphous was obtained.

TABLE 71
Summary of slow evaporation experiments

| Solvent | Result |
|---|---|
| MeOH | Amorphous |
| EtOH | Amorphous |

### Solid Vapor Diffusion

Solid vapor diffusion experiments were conducted using 6 different solvents. Approximately 20 mg of meglumine salt Type A was weighed into a 3-ml, vial, which was placed into a 20-ml, vial with 4 ml, of volatile solvent. The 20-ml, vial was sealed with a cap and kept at RT for 7 days allowing solvent vapor to interact with sample. The solids were tested by XRPD and the results summarized in Table 71a showed that Meglumine salt Type A was obtained.

TABLE 71a
Summary of slow evaporation experiments

| Solvent | Result |
|---|---|
| EtOH | Meglumine salt Type A |
| Acetone | Meglumine salt Type A |
| EtOAc | Meglumine salt Type A |
| THF | Meglumine salt Type A |
| DCM | Meglumine salt Type A |
| DMSO | Meglumine salt Type A |

### Liquid Vapor Diffusion

Approximate 20 mg of meglumine salt Type A was dissolved in 0.6-1.0 ml of appropriate solvent to obtain a clear solution in a 3-ml, vial (0.45 μm PTFE filter was used for filtration if not completely dissolved). This solution was then placed into a 20-ml, vial with 4 ml, of volatile solvent. The 20-ml, vial was sealed with a cap and kept at RT allowing sufficient time for organic vapor to interact with the solution. The precipitants were isolated for XRPD analysis. The results summarized in Table 72 showed that Meglumine salt Type A were observed.

TABLE 72
Summary of liquid vapor diffusion experiments

| Solvent | Anti-solvent | Result |
|---|---|---|
| Acetone | MTBE | Amorphous* |
| | H₂O | Amorphous* |
| 1,4-Dioxane | IPAc | Gel* |
| | Toluene | Gel* |
| | IPA | Gel* |

### Slow Cooling

Slow cooling experiments were conducted in 2 solvent systems. About 20 mg of meglumine salt Type A was suspended in 1.0 ml, of solvent in a 3-ml, glass vial at RT. The suspension was then heated to 50° C., equilibrated for about 2 hrs and filtered to a new vial. Filtrates were slowly cooled down to 5° C. at a rate of 0.1° C./min. The obtained solids were kept isothermal at 5° C. and then solids were tested by XRPD. Results summarized in Table 73 indicated that Meglumine salt Type A were obtained.

TABLE 73
Summary of slow cooling experiments

| Solvent | Result |
|---|---|
| IPA | Amorphous* |
| Acetone | Meglumine salt Type A* |

*No solid was obtained after cooling to 5 and storage at −20° C. for 3 days. The sample was transferred to evaporation in the air.

### Grinding

Grinding experiments were performed with or without solvent addition. Approximate 20 mg of meglumine salt Type A was weighed into the mortar. 10 sL solvent was added into the mortar. The solids were grinded for 3 min. The solids were isolated for XRPD analysis. Results summarized in Table 74 showed that only Meglumine salt Type A was obtained.

TABLE 74
Summary of Grinding Experiments

| Solvent | Result |
|---|---|
| NA | Meglumine salt Type A |
| H₂O | Meglumine salt Type A |
| EtOH | Meglumine salt Type A |

### Polymorph Screening for Freeform

A total of 50 polymorph screening experiments were performed on Compound 1 freeform Type D using different solvent mediated crystallization or solid phase transition methods. The methods utilized and crystal forms identified are summarized in Table 75.

TABLE 75
Summary of polymorph screening experiments
for Compound 1 Freeform

| | No. of | |
|---|---|---|
| Method | Experiments | Results |
| Anti-solvent Addition | 8 | Freeform Type C/E/O/I + J, |
| | | amorphous, gel |
| Slurry at RT | 10 | Freeform Type B/C/D/E/F/G |
| Slurry at 50° C. | 10 | Freeform Type B/C/D/H/I |
| Slow Evaporation | 6 | Freeform Type B/C/E/J/L |
| Solid Vapor Diffusion | 6 | Freeform Type A/D/J/K |
| Liquid Vapor Diffusion | 6 | Freeform Type B/C/E/J/I/K |
| Slow Cooling | 4 | Freeform Type B/C/D, |
| | | amorphous |
| Total | 50 | Freeform Type B~L, |
| | | amorphous, gel |

### Anti-Solvent Addition

For each experiment, about 20 mg of starting material was weighed into a 20-mL glass vial, followed by the addition of 0.5-1.0 mL corresponding solvent to dissolve the compound. The samples were filtered using a PTFE membrane (pore size of 0.45 μm) into a new glass vial. A clear solution was obtained and magnetically stirred at the speed of 1000 rpm. Subsequently, the relative anti-solvent was added to the solution to induce precipitation. The solids were isolated for XRPD analysis. Results summarized in Table 76 showed that freeform Type C/E/O/I+J, amorphous and gel were obtained.

TABLE 76
Summary of anti-solvent addition experiments

| Solvent | Anti-solvent | Results |
|---|---|---|
| DMSO | H₂O | Amorphous |
| | Toluene | Freeform Type O* |
| | Anisole | Gel^{#} |
| MeOH | CHCl₃ | Freeform Type I + J* |
| | H₂O | Freeform Type E |
| Acetone | MTBE | Freeform Type E |
| | n-Heptane | Freeform Type C |
| | CPME | Freeform Type C* |

*No solid was obtained after anti-solvent addition and slurry at 5/−20° C. for one day. The samples were transferred to evaporation in the air;
^{#}No solid was obtained after anti-solvent addition and slurry at 5/−20° C. for one day. The samples were transferred to evaporation in the air and vacuum-evaporation at 50° C.

### Slurry at RT

Slurry conversion experiments were conducted at RT in different solvent systems. For each experiment, about 20 mg of starting material was suspended in 0.5 mL corresponding solvent in an HPLC glass vial. After the suspension was magnetically stirred at RT (˜1000 rpm) for ˜7 days, the remaining solids were isolated for XRPD analysis. Results summarized in Table 77 showed that freeform Type B/C/D/E/F/G were obtained.

TABLE 77
Summary of slurry conversion experiments at RT

| Solvent (v/v) | Results |
|---|---|
| Acetone | Freeform Type C |
| 1,4-Dioxane | Freeform Type D |
| DCM | Freeform Type F |
| ACN | Freeform Type C |
| EtOAc | Freeform Type C |
| IPA/Acetone (1:1) | Freeform Type C |
| DMSO/H₂O (1:4) | Freeform Type G |
| MeOH/MTBE (1:1) | Freeform Type E |
| EtOH/H₂O (a_{w}~0.3) | Freeform Type B |
| EtOH/H₂O (a_{w}~0.7) | Freeform Type B |

### Slurry at 50° C.

Slurry conversion experiments were conducted at 50° C. in different solvent systems. For each experiment, about 20 mg of starting material was suspended in 0.5 mL corresponding solvent in an HPLC glass vial. After the suspension was magnetically stirred at 50° C. (˜1000 rpm) for ˜7 days, the remaining solids were isolated for XRPD analysis. Results summarized in Table 78 indicated that freeform Type B/C/D/H/I were produced.

TABLE 78
Summary of slurry conversion experiments at 50° C.

| Solvent (v/v) | Results |
|---|---|
| IPA | Freeform Type C |
| IPAc | Freeform Type C |
| Anisole | Freeform Type H |
| 2-MeTHF | Freeform Type D |
| Toluene | Freeform Type I |
| MEK | Freeform Type C |
| EtOAc/ACN (1:1) | Freeform Type C |
| 1,4-Dioxane/H₂O (1:1) | Freeform Type D |
| THF/CHCl₃ (1:1) | Freeform Type D |
| EtOH/CPME (1:1) | Freeform Type B |

### Slow Evaporation

Slow evaporation experiments were performed under six conditions. For each experiment, around 20 mg of starting material was weighed into a 3-ml, glass vial. Corresponding solvent was added, and a solution was obtained. The samples were filtered using a PTFE membrane (pore size of 0.45 μm) into a new 3-ml, glass vial. The vial was covered with Parafilm with 4 pinholes to let the solvent evaporate slowly at RT to induce precipitation. The isolated solids were tested by XRPD. As summarized in Table 79, freeform Type B/C/E/J/L were generated.

TABLE 79
Summary of slow evaporation experiments

| Solvent (v/v) | Solid Form |
|---|---|
| EtOH | Freeform Type B |
| Acetone | Freeform Type L |
| EtOAc | Freeform Type C |
| DCM | Freeform Type J |
| ACN/H₂O (1:1) | Freeform Type C |
| MeOH/CHCl₃ (1:1) | Freeform Type E |

Solid vapor diffusion experiments were conducted using six solvents. For each experiment, about 20 mg of starting material was weighed into a 3-ml, vial, which was placed into a 20-ml, vial with 3 ml, of corresponding solvent. The 20-ml, vial was sealed with a cap and kept at RT to allow the solvent vapor to interact with the solid sample. The isolated solids were tested by XRPD. The results summarized in Table 80 indicated that freeform Type A/D/J/K were obtained.

TABLE 80
Summary of solid vapor diffusion experiments

| Solvent | Solid Form |
|---|---|
| H₂O | Freeform Type D (peak shift) |
| IPA | Freeform Type A + peak |
| Acetone | Freeform Type D |
| DCM | Freeform Type J |
| 1,4-Dioxane | Freeform Type D |
| DMSO | Freeform Type K |

### Liquid Vapor Diffusion

For each experiment, about 20 mg of starting material was weighed into a 3-ml, vial. 0.6˜1.0 ml, solvent in the table was added to get a clear solution. The vial was sealed into the 20-ml, glass vial with 4 ml, of relative anti-solvent and the system was kept at RT to allow the solvent vapor to interact with the solution. The isolated solids were tested by XRPD. The results summarized in Table 81 indicated that freeform Type B/C/I/I/J/K were obtained.

TABLE 81
Summary of liquid vapor diffusion experiments

| Solvent | Anti-solvent | Results |
|---|---|---|
| DCM | n-Pentane | Freeform Type J |
| EtOH | n-Hexane | Freeform Type B |
| Acetone | | Freeform Type I |
| EtOAc | MTBE | Freeform Type C* |
| MeOH | | Freeform Type E* |
| DMSO | | Freeform Type K + peak* |

*No solid was observed after liquid vapor diffusion for 12 days and the sample was transferred to evaporation in the air.

### Slow Cooling

For each experiment, about 20 mg of starting material was suspended in 1.0 mL of corresponding solvent in a 3-mL glass vial at RT. The suspension was transferred to slurry at 50° C. on a magnetic stir plate with the speed of 1000 rpm. The sample was equilibrated at 50° C. for 2 hrs and filtered using a 0.45 μm PTFE membrane. Subsequently, the filtrate was slowly cooled down from 50° C. to 5° C. at a rate of 0.1° C./min. The sample was stored at 5° C. before XRPD analysis. The results summarized in Table 82 indicated that freeform Type B/C/D and amorphous were obtained.

TABLE 82
Summary of slow cooling experiments

| Solvent | Results |
|---|---|
| ACN | Amorphous* |
| EtOH | Freeform Type B* |
| 2-MeTHF | Freeform Type D (peak shift)* |
| EtOAc | Freeform Type C* |

*Clear solution obtained from slow cooling was transferred to storage at −20° C.

### Instruments and Methods

### XRPD

For XRPD analysis, PANalytical Empyrean and X′ Pert3 X-ray powder diffract meters were used. The XRPD parameters used are listed in Table 83.

TABLE 83
Parameters for XRPD test

| | | |
|---|---|---|
| Parameters | Reflection XRPD | VT/VH XRPD |

| | |
|---|---|
| X-Ray wavelength | Cu, Kα; |
| | Kα1 (Å): 1.540598 |
| | Kα2 (Å): 1.544426 |
| | intensity ratio Kα2/Kα1: 0.50 |

| | | |
|---|---|---|
| X-Ray tube setting | 45 kV, 40 mA | 45 kV, 40 mA |
| Divergence slit | ⅛° | Automatic |
| Scan mode | Continuous | Continuous |
| Scan range (2θ/°) | 3°~40° | 3°~40° |
| Step size (2θ/°) | 0.0263 | 0.0167 |
| Scan step time (s) | 46.67 | 33.02 |
| Test time | 5 min 04 s | 10 min 12 s |

### TGA and DSC

TGA data were collected using a TA Discovery 5500 TGA from TA Instruments. DSC was performed using a TA Discovery 2500 DSC from TA Instruments. Detailed parameters used are listed in Table 84.

TABLE 84
Parameters for TGA and DSC test

| Parameters | TGA | DSC |
|---|---|---|
| Method | Ramp | Ramp |
| Sample pan | Aluminum, open | Aluminum, crimped/open |
| Temperature | RT-300° C. | 25° C.-250° C. |
| Heating rate | 10° C./min | 10° C./min |
| Purge gas | N₂ | N₂ |

### DVS

DVS was measured via a SMS (Surface Measurement Systems) DVS Intrinsic. The relative humidity at 25° C. were calibrated against deliquescence point of LiCl, Mg(NO₃)₂ and KCl. Parameters for DVS test are listed in Table 85.

TABLE 85
Parameters for TGA and DSC test

| Parameters | Values | |
|---|---|---|
| Temperature | 25° | C. |
| Sample size | 10-20 | mg |
| Gas and flow rate | N2, 200 mL/min | |
| dm/dt | 0.002%/min | |
| Min. dm/dt stability duration | 10 | min |
| Max. equilibrium time | 180 | min |

| | |
|---|---|
| RH range | RT~95% RH~0% RH~95% |
| | RH (freeform Type A) |
| | 0% RH~95% RH~0% RH |
| | (other samples) |
| RH step size | 10% (90% RH-0% RH-90% RH) |
| | 5% (95% RH-90% RH and |
| | 90% RH-95% RH) |

### PLM

PLM picture was captured on Axio Lab. A1 upright microscope, purchased from Carl Zeiss German.

### Solution NMR

Solution NMR was collected on Bruker 400M NMR Spectrometer using DMSO-d6.

### HPLC

Agilent 1260 HPLC were utilized and detailed chromatographic conditions are listed in Table 86 and Table 87.

TABLE 86
HPLC methods for purity test

| Parameters | Agilent 1260 (DAD) | |
|---|---|---|
| Column | Genimi C18, 150 × 4.6 mm, 3.4 μm | |
| | A: 0.037% TFA in H₂O | |
| | B: 0.018% TFA in ACN | |
| Mobile phase | Time (min) | % B |
| | 0.0 | 10 |
| | 16.00 | 80 |
| | 19.00 | 100 |
| | 21.00 | 100 |
| | 21.01 | 10 |
| | 23.00 | 10 |

| | |
|---|---|
| Run time | 23.0 min |
| Flow rate | 1.0 mL/min |
| Injection volume | 3 μL |
| Detector wavelength | 220 nm |
| Column temperature | 40° C. |
| Sampler temperature | RT |
| Diluent | ACN/H₂O (1:1, v/v) |

TABLE 87
HPLC methods for solubility test

| UPLC | Agilent 1260 (DAD) | |
|---|---|---|
| Column | Genimi C18, 150 × 4.6 mm, 3.4 μm | |
| | A: 0.037% TFA in H₂O | |
| | B: 0.018% TFA in ACN | |
| Mobile phase | Time (min) | % B |
| | 0.00 | 20 |
| | 5.00 | 6 |
| | 6.00 | 60 |
| | 6.01 | 20 |
| | 8.00 | 210 |

| | |
|---|---|
| Run time | 8.0 min |
| Flow rate | 1.0 mL/min |
| Injection volume | 2 μL |
| Detector wavelength | 220 nm |
| Column temperature | 40° C. |
| Sampler temperature | RT |
| Diluent | ACN/H₂O (1:1, v/v) |

### IC

ThermoFisher ICS-1100 ion chromatography was utilized and detailed IC parameters were listed in Table 88 and Table 89.

TABLE 88
IC parameters for stoichiometric test (anion)

| IC | ThermoFisher ICS-1100 | |
|---|---|---|
| Column | Dionex IonPac ™ AS18 RFIC ™ (4 × 250 mm) | |
| Mobile phase | 25 | mM NaOH |
| Injection volume | 25 | μL |
| Flow rate | 1.0 | mL/min |
| Cell temperature | 35° | C. |
| Column temperature | 35° | C. |
| Current | 80 | mA |
| Run time | 25 | mins |

TABLE 89
IC parameters for stoichiometric test (cation)

| IC | ThermoFisher ICS-1100 | |
|---|---|---|
| Column | Dionex IonPac ™ CS12A RFIC ™ (4 × 250 mm) | |
| Mobile phase | 25 mM methanesulfonic acid | |
| Injection volume | 25 | μL |
| Flow rate | 1.0 | mL/min |
| Cell temperature | 35° | C. |
| Column temperature | 35° | C. |
| Current | 80 | mA |
| Run time | 7.0 | min |

### Example 2. Manufacturing Process of Compound 1 Meglumine Salt Type A

A manufacturing process was developed successfully to prepare Compound 1 meglumine salt Type A. The key conclusions during development are shown below:
Type A was selected as the target form for manufacturing process development.
Solubility studies indicated that Compound 1 meglumine salt had high solubility in in dimethylsulfoxide (DMSO) at 50° C. and solubility decreased with further addition of ethyl acetate (EA). Therefore, anti-solvent crystallization (DMSO as solvent and EA as anti-solvent) was promising.
Chemical stability studies indicated that Compound 1 meglumine salt was generally stable at high temperatures in both the solution and solid states.
Type A was thermodynamically more stable in different ratio of DMSO/EA system at different temperature, while Type B was thermodynamically more stable in MeOH/MTBE system (see Table 97).
Type B could be converted to Type A by slurrying in EA or other organic solvents at 25° C. or 50° C.
The crystallization process was finalized in DMSO/EA=4V/24V after detailed studies. Stress tests indicated that the equivalent of meglumine was suggested to be 1.0-1.5 to ensure the yield of crystallization. Other stress tests indicated that crystallization in DMSO/EA (4V/24V) system was robust.
Jet-milling with different condition could deliver solids of varying PSD (D₉₀ 5-20 um).

TABLE 97
Competitive experiments of Type A and Type B in
different solvent system at different temperature

| | Input forms | | | |
|---|---|---|---|---|
| Experiment ID | (1:1 ratio) | Solvent system | T (° C.) | XRPD (1 d) |
| PS10226-18-FPA-1 | Form A/B | EtOH | 50 | Form A |
| PS10226-18-FPA-2 | | EA | | |
| PS10226-18-FPA-3 | | MeCN | | |
| PS10226-18-FPA-4 | | Acetone | | |
| PS10226-18-FPA-5 | | THF | | |
| PS10226-18-FPA-6 | | MeOH/MTBE (6v/2v) | | Form B |
| PS10226-18-FPA-7 | | MeOH/MTBE (6v/6v) | | |
| PS10226-18-FPA-8 | | MeOH/MTBE (6v/15v) | | |
| PS10226-18-FPA-9 | | EtOH | 25 | Form A |
| PS10226-18-FPA-10 | | EA | | |
| PS10226-18-FPA-11 | | MeCN | | |
| PS10226-18-FPA-12 | | Acetone | | |
| PS10226-18-FPA-13 | | THF | | |
| PS10226-18-FPA-14 | | MeOH/MTBE (6v/2v) | | Form B |
| PS10226-18-FPA-15 | | MeOH/MTBE (6v/6v) | | |
| PS10226-18-FPA-16 | | MeOH/MTBE (6v/15v) | | |

The manufacturing process of Compound 1 meglumine salt Type A is shown below:
1. Dissolve Compound 1 (1×) and meglumine (1.0-1.1 eq.) in 7V DMSO/EA (1 v/1 v) at 30° C.
2. Polish filter
3. Rinse with 1V DMSO/EA (1 v/1 v)
4. Adjust to 50° C.
5. Charge 10V EA
6. Add 0.5% seed (Compound 1 meglumine salt Type A)
7. Stir for 2 h
8. Add 10V EA for 5 h
9. Stir at 50° C. for 2 h
10. Cool to 5° C. for 9 h11. Stir for 6-12 h
12. Filter the suspension
13. Wash wet cake with 4*4V EA
14. Dry under vacuum at 50° C. for 6-24 h

Scale up production batch at 5.5 kg was carried out. After work up, 6.3 kg of Compound 1 meglumine salt Type A as off-white powder was obtained with 99.4% purity in 85.8% isolated yield.

### Example 3. Single Crystal Structure Determination of Compound 1 Meglumine Type A

### Summary

The needle-like single crystal sample of Compound 1 meglumine salt Type A was crystallized from MeOH by slow evaporation method using the meglumine salt Type A sample as starting material. SCXRD characterization indicated the single crystal belonged to monoclinic crystal system and P21 space group. The unit cell dimensions were determined as {a=9.83990(10) Å, b=11.0578(2) Å, c=35.6500(4) Å, α=90°, β=92.3940(10°), γ=90°, V=3875.61(9) Å3}. Single crystal structure determination showed the asymmetric unit of the crystal structure was comprised of two Compound 1 anions (−1 charge), two meglumine cations (+1 charge) and a water molecule with occupancy of 0.3 (i.e. the total number of water molecule is 0.3), which indicated the acid/base molar ratio of the Compound 1 meglumine salt Type A is 1:1, and the compound 1 meglumine salt Type A single crystal structure contained 0.15 crystalline water.

The single crystal sample of Compound 1 meglumine salt Type A used for SCXRD characterization was obtained from slow evaporation crystallization method. The experiment details are elaborated below.

To a 3 mL glass vial were added 48.5 mg Compound 1 meglumine salt Type A starting material and 0.5 mL MeOH. The sample suspension was magnetically stirred for ˜20 hrs, after which it was filtered by syringe and syringe filter (0.45 μm PTFE filter membrane). The filtrate was transferred to a clear 4 mL shell vial (44.6 mm×14.65 mm). The shell vial was covered by PE-Plug with one pinhole on it and enclosed into a 20 mL glass vial with 3 mL PEG-400 (absorbent). The 20 mL glass vial was sealed by cap and placed in fume hood to perform liquid vapor diffusion in a confined space. After 1 day, a cluster of needle-like crystal was obtained. XRPD characterization indicated the obtained needle-like single crystal was Compound 1 meglumine salt Type A.

### Single Crystal Structure Determination

A suitable single crystal with suitable size and good diffraction quality was cut out and selected from the needle-like crystal sample and characterized by single-crystal X-ray diffraction. The structure of the single crystal was determined successfully. The crystal system of the single crystal was monoclinic and the space group was P21. The unit cell dimensions were determined as {a=9.83990(10) Å, b=11.0578(2) Å, c=35.6500(4) Å, α=90°, β=92.3940(10) °, γ=90°, V=3875.61(9) Å3}. Other crystallographic data and the refinement parameters are listed in Table 118.

The asymmetric unit of the single crystal structure was comprised of two Compound 1 (−1 charge), two meglumine cations (+1 charge) and a water molecule with occupancy of 0.3 (i.e. the total number of water molecule is 0.3), which indicated the acid/base molar ratio of the Compound 1 meglumine salt Type A is 1:1, and the Compound 1 meglumine salt Type A single crystal structure contained 0.15 crystalline water. It is worth noting that the identification of water molecules in this crystal structure is mainly based on: 1) In the process of single crystal structure solving and refinement, it was found that after modeling all the atoms of the Compound 1 anions and meglumine cations, there was still an obvious residual electron density peak (Q1=1.0 e Å-3) in the difference fourier map. This largest residual electron peak usually suggested that a potential solvent molecule (water molecule) may exist in the crystal structure. 2) Further Solvent Mask analysis showed that the largest residual electron density peak Q1 was located in a small lattice void (void volume=20 Å-3) of the single crystal structure and the number of electron in this void was about 3 e−, which was equivalent to the electron number of 0.3 water molecules (every water molecule contains 10 electrons). Therefore, the Q1 peak was assigned as the oxygen atom (O1W) of water molecule and the atomic occupancy was refined as 0.3. After modeling the water molecule in the single crystal structure model, the residual factor (R1) of final structure model is significantly improved (from 4.51% to 3.96%), and other refinement parameters are all reasonable. These indicated the rationality of the existence of the water molecule in the structure.

The atomic thermal ellipsoid plots (ORTEP drawing) of the two Compound 1 anions (crystallographically independent) in the above asymmetric unit diagram are shown in FIG. 3 4 and FIG. 3 5, respectively. The single crystal structure data confirmed the stereo-chemical structure of the Compound 1 meglumine salt. The absolute configurations of chiral atoms in the meglumine cation were determined as {C2(R), C3(S), C4(S), C5(S)})¹. ¹The Flack parameter x(μ) was refined to 0.008(8). The standard uncertainty (μ)<0.04. The |x| was closed to 0 and |x<3μ (in general, |x|<0.1). This Flack parameter showed the validity of the absolute structure determination. In addition, Bayesian statistics on Bijvoet differences (Hooft y=0.004(7), P2(true)=1.000, P3(true)=1.000, P3(rac-twin)=0.0E+00, P3(false)=0.0E+00) also indicate the validity of the absolute structure determination. (Reference: Flack, H. D. Acta Cryst. A39(1983), 876-881; Flack H. D., Bernardinelli, G. CHIRALITY. 20(2008), 681-690; Hooft, R. W. W., Straver, L. H. and Spek, A. L. J. Appl. Cryst. 41(2008), 96-103).

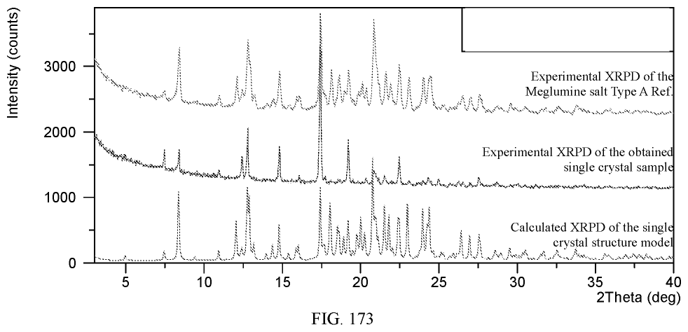

The unit cell diagram of the Compound 1 meglumine salt Type A single crystal structure shows that adjacent Compound 1 anions and meglumine cations are connected with each other by hydrogen bonds. The corresponding hydrogen bond information are listed in Table 119. FIG. 173 shows the calculated XRPD pattern of the Compound 1 meglumine salt Type A single crystal structure model and the experimental XRPD pattern of the single crystal sample and the Compound 1 meglumine salt Type A reference. The comparison result showed that they were in good agreement with each other.

Tables of non-hydrogen atomic positional parameters, anisotropic displacement factor coefficients, bond distances, bond angles, torsion angles, hydrogen atom coordinates and occupancy information are provided below.

TABLE 118
Crystallographic data and refinement parameters of
the Compound 1 meglumine salt Type A single crystal

| | |
|---|---|
| Empirical formula | C₃₈H₄₄FN₇O₈S•0.15H₂O |
| Formula weight | 777.86 |
| Temperature | 298.74(10) K |
| Wavelength | Cu/K_{α} (λ = 1.54184 Å) |
| Crystal system, space group | Monoclinic, P2₁ |
| Unit cell dimensions | a = 9.83990(10) Å |
| | b = 11.0578(2) Å |
| | c = 35.6500(4) Å |
| | α = 90° |
| | β = 92.3940(10)° |
| | γ = 90° |
| Volume | 3875.61(9) Å³ |
| Z, Calculated density | 4, 1.333 g/cm |
| Absorption coefficient | 1.296 mm⁻¹ |
| F(000) | 1640.0 |
| Crystal size | 0.10 × 0.03 × 0.01 mm³ |
| 2 Theta range for data collection | 4.962 to 144.248 |
| Limiting indices | −12 ≤ h ≤ 12 |
| | −12 ≤ k ≤ 13 |
| | −43 ≤ 1 ≤ 43 |
| Reflections collected/Independent reflections | 83696/14708 [Rᵢₙₜ = 0.0381, R_{sigma} = 0.0301] |
| Refinement method | Full-matrix least-squares on F² |
| Completeness | 96.68% |
| Data / restraints / parameters | 14708/1/1015 |
| Goodness-of-fit on F² | 1.050 |
| Final R indices [I ≥ 2sigma(I)] | R₁ = 0.0396, wR₂ = 0.1064 |
| Final R indices [all data] | R₁ = 0.0420 wR₂ = 0.1080 |
| Largest diff. peak and hole | 0.31/−0.38Å⁻³ |
| Flack parameter | 0.008(8) |
| Bayesian statistics on Bijvoet differences² | Hooft y = 0.004(7), P2(true) = 1.000, |
| | P3(true) = 1.000, P3(rac-twin) = 0.0E+00, |
| | P3(false) = 0.0E+00 |

²Calculated by PLATON (version: 140621). Reference: Hooft, R. W. W., Straver, L. H. and Spek, A. L.. J. Appl. Cryst. 41(2008), 96-103.

TABLE 119
H-bond table of the Compound 1 meglumine salt Type A single crystal structure model

| Type | D-H . . . A | d(D-H)/Å | d(H . . . A)/Å | d(D . . . A)/Å | (D-H . . . A)/° |
|---|---|---|---|---|---|
| Intramolecular | N1D-H1DA . . . O5D | 0.89 | 2.39 | 2.806(3) | 109 |
| Intermolecular | N1D-H1DA . . . N5A^{#1} | 0.89 | 2.17 | 2.977(3) | 150 |
| Intermolecular | O1C-H1C . . . O2D | 0.82 | 1.94 | 2.734(3) | 163 |
| Intermolecular | O1D-H1D . . . O3C | 0.82 | 1.93 | 2.682(3) | 152 |
| Intramolecular | N1D-H1B . . . O2A^{#2} | 0.89 | 2.57 | 3.054(3) | 115 |
| Intermolecular | N1D-H1B . . . O3A^{#2} | 0.89 | 1.92 | 2.792(3) | 167 |
| Intermolecular | O2C-H2C . . . O5D^{#3} | 0.82 | 2.00 | 2.801(3) | 165 |
| Intermolecular | O2D-H2D . . . O2C^{#4} | 0.82 | 1.98 | 2.801(3) | 177 |
| Intermolecular | N1C-H1CA . . . O3B^{#5} | 0.89 | 1.85 | 2.720(3) | 166 |
| Intermolecular | O3C-H3C . . . O3D^{#6} | 0.82 | 1.96 | 2.775(3) | 171 |
| Intermolecular | O3D-H3D . . . O5C^{#7} | 0.82 | 2.18 | 2.992(3) | 172 |
| Intramolecular | N1C-H1CB . . . O5C | 0.89 | 2.41 | 2.822(3) | 108 |
| Intermolecular | N1C-H1CB . . . N5B^{#8} | 0.89 | 2.14 | 2.949(3) | 150 |
| Intermolecular | O4C-H4C . . . O1D^{#8} | 0.82 | 2.04 | 2.790(3) | 152 |
| Intermolecular | O4D-H4D . . . O1C^{#4} | 0.82 | 2.01 | 2.711(3) | 144 |
| Intermolecular | O5C-H5C . . . O2A | 0.82 | 1.84 | 2.640(3) | 164 |
| Intermolecular | O5D-H5D . . . O2B | 0.82 | 1.83 | 2.638(3) | 167 |

Calculated by PLATON program (version: 140621), (Analysis of Potential Hydrogen Bonds with d(D . . . A) < R(D) + R(A) + 0.50 Ang., d(H . . . A) < R(H) + R(A) − 0.12 Ang., D-H . . . A > 100.0 Deg).
Symmetric operation code:
^{#1}(2 − x, 3/2 + y, 1 − z);
^{#2}(2 − x, 1/2 + y, 1 − z);
^{#3}(x, −1 + y, z);
^{#4}(1 − x, 1/2 + y, 1 − z);
^{#5}(1 − x, −1/2 + y, 1 − z);
^{#6}(2 − x, −1/2 + y, 1 − z);
^{#7}(x, 1 + y, z);
^{#8}(1 − x, −3/2 + y, 1 − z);

### Single Crystal Diffraction Data Collection

A suitable single crystal with suitable size and good diffraction quality was cut out and selected from the needle-like single crystal sample of the Compound 1 meglumine salt Type A and wrapped with Paratone-N (an oil based cryoprotectant). The selected single crystal was mounted on a Cryoloop and fixed on the goniometer head with a random orientation. Preliminary examination and data collection were performed on a Rigaku XtaLAB Synergy R (Cu/Kα X-ray radiation, λ=1.54184 Å) diffractometer at room temperature.

Cell parameters and orientation matrixes for data collection were retrieved and refined (T-vector algorithm) by CrysAlisPro (version: 1.171.42.51a) software using the setting angles of 26530 reflections in the range 2.481°<θ<72.124°. The data were collected to a minimum diffraction angle (θ) of 2.447° and a maximum diffraction angle (θ) of 72.009°. The completeness of data collection is 96.68%. The mean 1/a of the collected data is 33.2 and the highest resolution is truncated at 0.81 Å.

### Single Crystal Diffraction Data Reduction

Frames were integrated with CrysAlisPro (version: 1.171.42.51a). A total of 83696 reflections in the range 2.481°<θ<72.124° were collected, of which 14708 were unique. Lorentz and polarization corrections were applied to the data. An empirical absorption correction was performed using CrysAlisPro (version: 1.171.42.51a) using spherical harmonicsas implemented in SCALE3 ABSPACK. The absorption coefficient μ of this material is 1.296 mm−1 at this wavelength (λ=1.54184 Å) and the minimum and maximum transmissions are 0.71658 and 1.00000, respectively. Intensities of equivalent reflections were averaged. The average agreement factor of all equivalent reflections (Rint) was 3.81% based on intensity.

### Methods of Single Crystal Structure Solving and Refinement

The structure was solved in the space group P21 with the ShelXT (version: 2018/2) structure solution program using Intrinsic Phasing method and refined with ShelXL (Version 2018/3) refinement package using full-matrix least-squares on F2 contained in Olex2 (version: 1.5). All non-hydrogen atoms were refined anisotropically. The nitrogen-bonding and oxygen-bonding hydrogen atoms were found from Fourier map, and refined isotropically and positionally. Other hydrogen atoms were calculated geometrically and refined using the riding model.

### Software for Calculation of XRPD Pattern

The calculated XRPD pattern was generated for Cu radiation using Mercury (version: 4.3.1) program and the atomic coordinates, space group, and unit cell parameters from the single crystal structure.

### Software for Single Crystal Structure Visualization

The crystal structure representations were generated by Olex2 (version: 1.5) and Diamond (version: 3.2k). The thermal ellipsoids drawing was generated by ORTEP-III (version: 2014.1) software.

### Instruments and Parameters

The PLM image of the Compound 1 meglumine salt Type A single crystal sample was captured using OLYMPUS SZX-7 stereoscopic microscope. The XRPD data were collected by PANalytical Empyrean X-ray diffractometer at room temperature. The single crystal X-ray diffraction data was collected by Rigaku XtaLAB Synergy R diffractometer at room temperature. The SCXRD instrument and the XRPD instrument parameters are shown in Table 120 and Table 121, respectively.

TABLE 120
SCXRD instrument parameters

| Instrument | Rigaku XtaLAB Synergy R |
|---|---|
| X-ray sources generator | PhotonJet R (Cu) X-ray Source |
| | (Cu/Kα: 1.54184 Å) |
| Detector | Hypix 6000C detector |
| Goniometer | Four-circle Kappa Goniometer |
| Low Temperature Devices | Cryostream-800 Plus |
| | (80~500 K) |
| Software package | CrysAlisPro |

TABLE 121
XRPD instrument parameters

| Instrument | PANalytical Empyrean |
|---|---|
| Model | Reflection mode |
| | CuKα, |
| X-Ray wavelength | Kα₁(Å): 1.540598 |
| | Kα₂(Å): 1.544426 |
| | Kα₂/Kα₁ intensity ratio: 0.50 |
| X-Ray tube setting | 45 kV, 40 mA |
| Divergence slit (°) | 1/8 |
| Scan mode | Continuous |
| Scan range (°2Theta) | 3~40 |
| Counting time (s) | 46.665 |
| Step size (°2Theta) | 0.0263 |
| Test time (h:m:s) | About 5 min |

TABLE 122
Fractional atomic coordinates (×104) and equivalent isotropic
displacement parameters (Å2 × 103) for the Compound
1 meglumine salt Type A single crystal structure model

| Atom | x | y | z | U(eq) |
|---|---|---|---|---|
| S1B | 2833.4(13) | 12638.3(11) | 7916.8(3) | 79.6(3) |
| S1A | 12005.3(14) | −2730.7(12) | 2062.5(3) | 89.5(4) |
| O5C | 8168.0(19) | 334.9(17) | 4221.7(6) | 41.8(4) |
| O3D | 8965.9(17) | 8747.4(18) | 4874.0(5) | 42.0(4) |
| O2C | 6373.2(18) | 2235.2(19) | 5417.5(6) | 45.4(4) |
| O3A | 11608(2) | 1422.9(19) | 3351.4(6) | 48.9(5) |
| O5D | 7004(2) | 10037.8(18) | 5775.0(6) | 48.2(5) |
| O3C | 8496.6(19) | 3845.0(19) | 4757.2(6) | 48.6(5) |
| O1C | 6150(2) | 4828(2) | 5328.6(7) | 52.6(5) |
| O1D | 9139.6(18) | 6079.6(18) | 4531.7(6) | 45.0(4) |
| O4C | 9382(2) | 1552(2) | 4799.4(6) | 54.3(5) |
| O2D | 6293.7(19) | 6483(2) | 4761.8(6) | 53.7(5) |
| O2B | 5033(2) | 9049(2) | 6149.8(7) | 61.2(6) |
| N3A | 13071(2) | −5360(2) | 2491.1(6) | 43.3(5) |
| O4D | 6270.3(19) | 9567.3(19) | 5054.7(7) | 51.9(5) |
| O3B | 3344(2) | 8614.5(19) | 6521.2(6) | 51.7(5) |
| N5B | 3062(2) | 14294(2) | 6654.9(6) | 41.1(5) |
| N1D | 8674(2) | 8536(2) | 6230.4(6) | 42.8(5) |
| O2A | 10122(3) | 991(2) | 3778.8(7) | 64.5(6) |
| N4A | 13130(2) | −2968(2) | 2963.7(6) | 39.3(5) |
| N4B | 1955(2) | 12917(2) | 6988.2(6) | 40.4(5) |
| N1C | 6069(2) | 1417(2) | 3774.5(6) | 37.7(4) |
| N5A | 12149(2) | −4285(2) | 3344.3(6) | 41.5(5) |
| N3B | 1982(2) | 15267(2) | 7493.5(7) | 45.3(5) |
| C22B | 3483(3) | 13137(2) | 6554.6(7) | 36.3(5) |
| C21B | 2787(2) | 12266(2) | 6756.6(7) | 35.7(5) |
| C28B | 4657(3) | 11560(2) | 6254.3(7) | 38.4(5) |
| C5C | 7681(2) | 1509(2) | 4317.0(7) | 33.2(5) |
| C20A | 12972(3) | −4153(3) | 3065.2(7) | 40.1(6) |
| C29A | 11258(2) | −645(2) | 3498.0(7) | 36.0(5) |
| C21A | 12351(2) | −2277(2) | 3197.8(7) | 35.0(5) |
| C24B | 1868(3) | 11830(3) | 7592.0(8) | 49.9(7) |
| C27B | 4448(3) | 12782(3) | 6300.7(7) | 39.4(5) |
| C6C | 6191(2) | 1548(3) | 4191.0(7) | 39.2(5) |
| F1B | 5748(4) | 11663(4) | 8842.3(11) | 136.6(14) |
| C4C | 7939(2) | 1714(2) | 4737.9(7) | 37.0(5) |
| C30A | 12135(3) | −1040(2) | 3227.4(7) | 37.1(5) |
| C31B | 4117(3) | 9351(3) | 6365.4(8) | 39.4(5) |
| N2A | 10676(3) | −6064(3) | 1325.5(8) | 61.7(7) |
| C3C | 7551(2) | 2966(2) | 4880.8(7) | 35.3(5) |
| O1A | 9400(4) | −5277(3) | 826.2(8) | 99.2(11) |
| C29B | 3914(3) | 10682(2) | 6445.8(7) | 36.3(5) |
| C31A | 10975(3) | 690(3) | 3546.0(7) | 40.6(6) |
| C23B | 1078(3) | 12393(3) | 7267.5(8) | 48.8(7) |
| C5D | 7734(2) | 9126(2) | 5590.1(7) | 37.6(5) |
| C19A | 13655(3) | −5174(3) | 2873.8(8) | 46.8(6) |
| C4D | 6939(2) | 8601(2) | 5243.9(8) | 38.4(5) |
| O1B | 5556(4) | 15195(4) | 9175.1(9) | 97.9(11) |
| C14A | 11967(4) | −6841(3) | 1869.9(9) | 55.1(7) |
| C22A | 11748(3) | −3124(3) | 3433.3(7) | 37.7(5) |
| C2C | 7492(2) | 2976(2) | 5311.5(7) | 38.2(5) |
| C30B | 2968(3) | 11028(2) | 6704.1(7) | 37.2(5) |
| C3D | 7906(2) | 7956(2) | 4984.6(7) | 34.8(5) |
| C20B | 2156(3) | 14120(2) | 6912.1(7) | 40.5(6) |
| C2D | 7176(2) | 7405(3) | 4639.5(7) | 41.4(6) |
| N2B | 4285(3) | 15978(3) | 8674.1(8) | 65.9(8) |
| C1C | 7327(3) | 4228(3) | 5474.0(8) | 47.9(6) |
| C13A | 11382(4) | −6995(3) | 1476.0(9) | 58.7(8) |
| N6B | 2760(5) | 10349(4) | 7991.1(12) | 89.1(11) |
| C28A | 10630(3) | −1496(3) | 3728.7(8) | 43.5(6) |
| C6D | 8124(3) | 8102(3) | 5858.4(8) | 45.2(6) |
| C15A | 11076(3) | −6073(3) | 2115.6(9) | 53.2(7) |
| C17A | 13965(3) | −6124(4) | 2277.1(10) | 62.4(9) |
| C27A | 10874(3) | −2718(3) | 3706.1(8) | 45.7(6) |
| C17B | 3330(3) | 15847(3) | 7491.4(8) | 52.7(7) |
| F1A | 9201(4) | −1739(4) | 1152.8(11) | 139.5(16) |
| C23A | 13956(3) | −2491(3) | 2665.5(8) | 46.2(6) |
| C7C | 4660(3) | 1212(3) | 3625.9(10) | 55.6(8) |
| C1D | 8171(3) | 6888(3) | 4366.0(8) | 49.0(7) |
| C18B | 3929(3) | 16010(3) | 7886.8(9) | 55.3(7) |
| C19B | 1440(3) | 15111(3) | 7108.1(9) | 49.1(7) |
| C16A | 11723(3) | −5910(3) | 2505.5(9) | 52.9(7) |
| C24A | 13113(3) | −1942(3) | 2350.6(8) | 48.4(7) |
| C15B | 1589(4) | 16135(4) | 8112.9(10) | 67.0(10) |
| C8A | 9213(5) | −4225(5) | 1045.1(11) | 80.0(12) |
| C25B | 1980(4) | 10647(4) | 7678.6(11) | 71.5(10) |
| C7D | 10093(3) | 8969(4) | 6232.2(11) | 62.6(9) |
| C18A | 13388(3) | −6282(4) | 1876.3(9) | 64.2(9) |
| C13B | 3559(4) | 16907(4) | 8521.7(10) | 63.8(9) |
| C16B | 1048(3) | 15989(4) | 7709.9(10) | 65.3(10) |
| C14B | 2991(4) | 16741(3) | 8127.1(9) | 56.8(8) |
| N6A | 12198(5) | −468(4) | 1951.6(12) | 93.2(12) |
| C26B | 3264(5) | 11306(6) | 8137.5(12) | 88.2(14) |
| C2B | 8160(4) | 11670(5) | 9624.4(12) | 79.7(12) |
| C25A | 13052(4) | −768(4) | 2249.1(11) | 73.5(10) |
| C5A | 8349(4) | −3375(4) | 805.3(11) | 70.9(10) |
| C4B | 6583(5) | 12081(5) | 9126.2(13) | 82.3(12) |
| C9A | 10163(5) | −6190(5) | 982.2(11) | 83.6(13) |
| C9B | 4790(5) | 16102(5) | 9017.9(11) | 83.8(13) |
| C5B | 6632(4) | 13303(5) | 9186.7(11) | 74.0(11) |
| C1A | 6033(5) | −944(5) | 121.6(13) | 87.5(13) |
| C3B | 7314(5) | 11239(5) | 9337.5(14) | 92.0(14) |
| C4A | 8383(5) | −2165(5) | 867.5(12) | 81.7(12) |
| C8B | 5764(5) | 14159(5) | 8951.7(12) | 80.3(12) |
| C12B | 3362(6) | 17957(5) | 8717.1(13) | 102.6(18) |
| N1B | 9556(6) | 10232(6) | 10060.2(16) | 124.0(18) |
| C12A | 11580(6) | −8048(5) | 1280.8(12) | 91.8(16) |
| C3A | 7642(5) | −1329(5) | 653.2(14) | 90.2(13) |
| C2A | 6808(4) | −1767(5) | 363.7(12) | 79.1(12) |
| N1A | 5415(6) | −349(5) | −77.4(14) | 119.5(17) |
| C1B | 8946(6) | 10844(6) | 9861.5(14) | 95.7(15) |
| C6A | 7499(6) | −3771(5) | 512.4(16) | 103.0(18) |
| C7B | 8270(6) | 12890(6) | 9691.5(15) | 99.2(16) |
| C6B | 7492(5) | 13691(5) | 9475.4(15) | 98.8(16) |
| C11A | 11015(8) | −8137(6) | 918.1(16) | 139(3) |
| C10A | 10299(8) | −7216(7) | 765.7(15) | 138(3) |
| C26A | 11592(5) | −1417(6) | 1831.2(13) | 93.6(15) |
| C7A | 6723(6) | −2971(5) | 300.3(16) | 101.9(17) |
| C10B | 4631(8) | 17112(7) | 9234.4(15) | 136(3) |
| C11B | 3910(9) | 18049(7) | 9080.1(17) | 151(4) |
| O1W | 11112(15) | 8297(13) | 7023(4) | 105(4) |

U_{eq} is defined as 1/3 of the trace of the orthogonalised Uij tensor.

TABLE 123
Anisotropic displacement parameters (Å2 × 103) for the Compound
1 meglumine salt Type A single crystal structure model

| Atom | U₁₁ | U₂₂ | U₃₃ | U₂₃ | U₁₃ | U₁₂ |
|---|---|---|---|---|---|---|
| S1B | 102.5(7) | 73.7(7) | 60.6(5) | 0.5(5) | −22.6(5) | −5.5(6) |
| S1A | 116.5(9) | 74.7(7) | 73.8(6) | 0.6(5) | −38.6(6) | −16.0(6) |
| O5C | 43.7(10) | 31.6(10) | 50.3(10) | −3.2(8) | 5.1(8) | 5.1(7) |
| O3D | 32.2(8) | 41.0(11) | 53.1(10) | 6.2(8) | 4.0(7) | −1.3(7) |
| O2C | 39.2(9) | 42.6(11) | 54.9(11) | 4.8(8) | 7.2(8) | 2.8(8) |
| O3A | 59.9(11) | 32.9(10) | 54.4(11) | −2.1(8) | 7.9(9) | −6.0(9) |
| O5D | 56.9(11) | 31.5(11) | 57.2(12) | −2.1(8) | 13.5(9) | 2.9(8) |
| O3C | 40.2(9) | 40.0(11) | 65.3(12) | 10.3(9) | −1.4(8) | −2.3(8) |
| O1C | 47.0(10) | 44.3(12) | 66.4(13) | −5.3(9) | 0.5(9) | 8.9(9) |
| O1D | 40.2(9) | 37.9(11) | 56.3(11) | −1.4(8) | −4.8(8) | 1.2(8) |
| O4C | 45.4(10) | 66.5(15) | 49.8(10) | −7.2(10) | −11.3(8) | 20.2(10) |
| O2D | 38.3(9) | 50.8(13) | 70.9(13) | 0.3(10) | −13.3(9) | −6.5(9) |
| O2B | 69.8(13) | 34.6(12) | 81.4(15) | −6.1(10) | 28.8(12) | −5.1(10) |
| N3A | 44.3(11) | 43.5(14) | 41.8(12) | −7.6(10) | −1.7(9) | 4.4(10) |
| O4D | 38.9(9) | 37.5(11) | 78.2(14) | 4.5(10) | −9.6(9) | 4.9(8) |
| O3B | 57.2(11) | 33.2(11) | 65.6(12) | −1.4(9) | 13.4(9) | −3.1(9) |
| N5B | 51.1(12) | 32.5(12) | 39.0(11) | 0.7(9) | −4.8(9) | 1.2(10) |
| N1D | 51.1(12) | 30.8(12) | 46.7(12) | −2.3(9) | 3.6(9) | 2.5(10) |
| O2A | 74.6(15) | 37.9(12) | 83.9(16) | −9.4(11) | 36.7(13) | −3.4(11) |
| N4A | 43.6(11) | 38.0(13) | 36.0(10) | −1.9(9) | 0.3(8) | 2.7(9) |
| N4B | 41.8(11) | 40.0(13) | 39.2(11) | 0.8(9) | −0.5(9) | 3.6(9) |
| N1C | 35.2(10) | 28.7(11) | 48.6(12) | −4.6(9) | −6.1(8) | 3.9(8) |
| N5A | 52.7(13) | 32.0(12) | 39.5(11) | −1.2(9) | −2.8(9) | 1.6(9) |
| N3B | 42.1(11) | 44.9(14) | 48.7(13) | −8.3(10) | −0.9(9) | 7.6(10) |
| C22B | 42.6(12) | 30.2(13) | 35.5(12) | 2.1(9) | −7.6(9) | −2.3(10) |
| C21B | 37.2(11) | 34.8(14) | 34.5(11) | −0.2(9) | −6.1(9) | 2.4(10) |
| C28B | 42.5(12) | 33.8(14) | 38.8(12) | −0.1(10) | 1.3(10) | −2.5(10) |
| C5C | 34.7(11) | 24.3(12) | 40.8(12) | 1.8(9) | 3.3(9) | 0.8(9) |
| C20A | 45.6(13) | 35.9(14) | 38.1(12) | −2.6(11) | −8.1(10) | 3.1(11) |
| C29A | 37.1(11) | 33.2(14) | 37.3(12) | −2.5(10) | −3.8(9) | −2.0(10) |
| C21A | 36.8(11) | 34.3(13) | 33.3(11) | −2.0(10) | −4.4(9) | −0.6(10) |
| C24B | 48.5(15) | 55.8(19) | 45.9(15) | 6.5(13) | 7.2(12) | −3.0(13) |
| C27B | 48.4(13) | 31.0(13) | 38.7(12) | 2.0(10) | 0.1(10) | −5.3(10) |
| C6C | 31.9(11) | 39.8(15) | 45.8(13) | −0.5(11) | 1.4(9) | 0.5(10) |
| F1B | 167(3) | 111(3) | 125(2) | −24(2) | −78(2) | 27(2) |
| C4C | 37.4(12) | 31.5(14) | 41.9(13) | 4.8(10) | 0.3(9) | 6.2(10) |
| C30A | 40.1(12) | 35.6(14) | 35.3(12) | 0.9(10) | −3.5(9) | −4.0(10) |
| C31B | 40.8(12) | 31.7(14) | 45.6(13) | 1.6(10) | −0.9(10) | −2.3(10) |
| N2A | 79.9(18) | 55.3(18) | 48.9(14) | −2.5(12) | −8.9(12) | 8.2(14) |
| C3C | 31.2(10) | 30.5(13) | 43.8(12) | 1.3(9) | −3.1(9) | 1.4(9) |
| O1A | 141(3) | 92(2) | 61.5(16) | −5.5(15) | −34.6(17) | 40(2) |
| C29B | 41.2(12) | 30.2(14) | 36.8(12) | −0.5(10) | −7.6(9) | 0.0(10) |
| C31A | 42.4(13) | 36.3(15) | 42.8(13) | −3.9(11) | −2.4(11) | −4.9(11) |
| C23B | 41.4(13) | 54.7(19) | 50.5(15) | 2.9(13) | 3.4(11) | −0.1(12) |
| C5D | 37.1(12) | 29.4(13) | 46.9(13) | 1.3(10) | 6.9(10) | 2.6(10) |
| C19A | 51.5(15) | 42.4(17) | 45.6(15) | −5.7(12) | −8.1(12) | 11.0(12) |
| C4D | 32.2(11) | 29.5(13) | 53.7(14) | 3.8(11) | 4.1(10) | −0.4(10) |
| O1B | 134(3) | 96(2) | 60.6(16) | −2.5(16) | −28.5(17) | 31(2) |
| C14A | 79(2) | 41.3(17) | 44.3(15) | −6.2(12) | −6.6(14) | 7.1(15) |
| C22A | 44.0(13) | 32.9(14) | 35.7(12) | −0.9(10) | −4.3(10) | −2.6(10) |
| C2C | 30.6(11) | 41.2(15) | 42.2(13) | −2.3(10) | −5.8(9) | 3.7(10) |
| C30B | 39.3(12) | 31.4(14) | 40.4(12) | 4.9(10) | −4.0(10) | −4.5(10) |
| C3D | 29.7(10) | 31.9(13) | 42.6(12) | 3.2(10) | 0.6(9) | −1.2(9) |
| C20B | 46.6(13) | 32.5(14) | 41.4(13) | −1.4(10) | −10.4(10) | 4.0(11) |
| C2D | 37.3(12) | 40.7(15) | 45.3(13) | 4.1(11) | −7.5(10) | 4.0(11) |
| N2B | 85(2) | 63.2(19) | 48.8(14) | −3.6(13) | −9.2(13) | 6.4(16) |
| C1C | 45.4(14) | 46.6(17) | 50.9(15) | −11.4(12) | −8.7(11) | 4.0(12) |
| C13A | 79(2) | 49(2) | 47.1(16) | −4.7(13) | −8.7(14) | 4.3(16) |
| N6B | 105(3) | 80(3) | 81(2) | 27(2) | −11(2) | 0(2) |
| C28A | 49.1(14) | 37.7(15) | 44.3(14) | −2.1(11) | 8.1(11) | −2.1(11) |
| C6D | 55.6(15) | 32.5(15) | 47.4(14) | −2.3(11) | −1.1(12) | 0.6(11) |
| C15A | 54.6(16) | 52.9(19) | 52.1(16) | −10.0(14) | 1.1(13) | −7.1(14) |
| C17A | 54.5(17) | 76(3) | 56.0(17) | −18.4(17) | −0.2(13) | 17.5(17) |
| C27A | 55.6(15) | 37.0(15) | 45.2(14) | 3.5(11) | 9.5(12) | −4.4(12) |
| C17B | 52.5(16) | 56(2) | 49.4(15) | −7.6(14) | 1.1(12) | −3.8(14) |
| F1A | 178(3) | 105(2) | 127(3) | −32(2) | −91(3) | 37(2) |
| C23A | 39.7(12) | 53.4(18) | 45.6(14) | 2.8(12) | 3.8(10) | 0.9(12) |
| C7C | 42.3(14) | 49.0(19) | 74(2) | −15.1(15) | −15.9(13) | −0.4(13) |
| C1D | 54.6(16) | 46.8(17) | 44.8(14) | −1.6(12) | −6.4(12) | 8.4(13) |
| C18B | 51.0(16) | 60(2) | 54.9(17) | −7.5(15) | −3.7(13) | −5.2(14) |
| C19B | 51.0(15) | 47.8(18) | 47.8(16) | −4.9(12) | −7.8(12) | 10.0(13) |
| C16A | 52.9(15) | 57(2) | 48.5(15) | −9.2(14) | 3.2(12) | −7.2(14) |
| C24A | 50.1(14) | 52.9(19) | 42.8(14) | 4.5(12) | 8.8(11) | −2.1(13) |
| C15B | 64(2) | 83(3) | 54.2(18) | −15.8(17) | 7.1(15) | 16.6(18) |
| C8A | 93(3) | 83(3) | 62(2) | 7(2) | −15.3(19) | 19(2) |
| C25B | 85(3) | 65(3) | 64(2) | 16.0(18) | −4.1(18) | −9(2) |
| C7D | 51.6(16) | 54(2) | 82(2) | −17.4(17) | 1.6(15) | −2.3(14) |
| C18A | 61.2(18) | 82(3) | 49.9(17) | −14.8(17) | 4.9(14) | 13.8(18) |
| C13B | 78(2) | 58(2) | 54.1(18) | −9.4(16) | −7.4(16) | 11.4(17) |
| C16B | 50.9(17) | 83(3) | 62.1(19) | −21.1(18) | 0.4(14) | 16.6(17) |
| C14B | 76(2) | 45.3(18) | 48.7(16) | −4.8(13) | −6.9(14) | 6.3(15) |
| N6A | 108(3) | 85(3) | 85(2) | 34(2) | −15(2) | 2(2) |
| C26B | 97(3) | 105(4) | 61(2) | 21(2) | −17(2) | 4(3) |
| C2B | 77(2) | 87(3) | 73(2) | 17(2) | −11.5(19) | 10(2) |
| C25A | 87(3) | 62(2) | 71(2) | 18.2(18) | −7.2(19) | −14(2) |
| C5A | 73(2) | 76(3) | 63(2) | 14.2(19) | −8.9(17) | 8(2) |
| C4B | 84(3) | 89(4) | 72(2) | 0(2) | −21(2) | 10(2) |
| C9A | 115(3) | 83(3) | 51.3(19) | −5.0(19) | −22(2) | 22(3) |
| C9B | 108(3) | 84(3) | 58(2) | −4(2) | −19(2) | 20(3) |
| C5B | 75(2) | 80(3) | 66(2) | 13(2) | −6.5(18) | 5(2) |
| C1A | 92(3) | 89(4) | 80(3) | 13(2) | −15(2) | 15(3) |
| C3B | 103(3) | 79(3) | 93(3) | 1(3) | −20(3) | 14(3) |
| C4A | 87(3) | 89(3) | 67(2) | −1(2) | −20(2) | 12(2) |
| C8B | 94(3) | 83(3) | 63(2) | 5(2) | −10.9(19) | 13(2) |
| C12B | 148(5) | 83(3) | 74(3) | −30(2) | −34(3) | 44(3) |
| N1B | 133(4) | 129(4) | 108(3) | 29(3) | −23(3) | 36(3) |
| C12A | 137(4) | 70(3) | 66(2) | −25(2) | −24(2) | 37(3) |
| C3A | 108(3) | 75(3) | 85(3) | 2(2) | −25(2) | 15(3) |
| C2A | 75(2) | 88(3) | 73(2) | 15(2) | −12.7(19) | 8(2) |
| N1A | 137(4) | 119(4) | 100(3) | 17(3) | −28(3) | 44(3) |
| C1B | 98(3) | 104(4) | 84(3) | 15(3) | −14(2) | 13(3) |
| C6A | 123(4) | 74(3) | 107(4) | 18(3) | −50(3) | −10(3) |
| C7B | 99(3) | 96(4) | 99(3) | 19(3) | −44(3) | −11(3) |
| C6B | 112(4) | 79(3) | 101(3) | 19(3) | −41(3) | −10(3) |
| C11A | 225(8) | 105(5) | 82(3) | −51(3) | −55(4) | 72(5) |
| C10A | 213(7) | 127(5) | 69(3) | −39(3) | −65(4) | 72(5) |
| C26A | 103(3) | 107(4) | 68(2) | 22(3) | −29(2) | −1(3) |
| C7A | 112(4) | 81(4) | 107(4) | 18(3) | −52(3) | −12(3) |
| C10B | 200(7) | 134(6) | 68(3) | −44(3) | −57(4) | 69(5) |
| C11B | 236(9) | 120(6) | 92(4) | −63(4) | −66(5) | 76(6) |
| O1W | 122(10) | 104(9) | 92(8) | 17(7) | 18(7) | 13(8) |

The anisotropic displacement factor exponent takes the form: −2π²[h²a*²U₁₁ + 2hka*b*U₁₂ + . . .].

TABLE 124
Bond lengths for the Compound 1 meglumine
salt Type A single crystal structure model

| Atom | Atom | Length/Å | Atom | Atom | Length/Å |
|---|---|---|---|---|---|
| S1B | C24B | 1.717(3) | O1A | C8A | 1.417(6) |
| S1B | C26B | 1.715(5) | O1A | C9A | 1.363(5) |
| S1A | C24A | 1.706(3) | C29B | C30B | 1.390(4) |
| S1A | C26A | 1.711(6) | C5D | C4D | 1.546(4) |
| O5C | C5C | 1.430(3) | C5D | C6D | 1.521(4) |
| O3D | C3D | 1.429(3) | C4D | C3D | 1.531(4) |
| O2C | C2C | 1.435(3) | O1B | C9B | 1.361(6) |
| O3A | C31A | 1.250(3) | O1B | C8B | 1.415(6) |
| O5D | C5D | 1.416(3) | C14A | C13A | 1.505(4) |
| O3C | C3C | 1.428(3) | C14A | C15A | 1.524(5) |
| O1C | C1C | 1.414(3) | C14A | C18A | 1.528(5) |
| O1D | C1D | 1.418(3) | C22A | C27A | 1.399(4) |
| O4C | C4C | 1.439(3) | C2C | C1C | 1.513(4) |
| O2D | C2D | 1.419(4) | C3D | C2D | 1.526(4) |
| O2B | C31B | 1.254(4) | C20B | C19B | 1.492(4) |
| N3A | C19A | 1.472(4) | C2D | C1D | 1.522(4) |
| N3A | C17A | 1.457(4) | N2B | C13B | 1.352(5) |
| N3A | C16A | 1.462(4) | N2B | C9B | 1.311(5) |
| O4D | C4D | 1.411(3) | C13A | C12A | 1.374(5) |
| O3B | C31B | 1.259(3) | N6B | C25B | 1.367(5) |
| N5B | C22B | 1.396(4) | N6B | C26B | 1.271(7) |
| N5B | C20B | 1.320(4) | C28A | C27A | 1.375(4) |
| N1D | C6D | 1.491(4) | C15A | C16A | 1.515(4) |
| N1D | C7D | 1.476(4) | C17A | C18A | 1.525(5) |
| O2A | C31A | 1.250(4) | C17B | C18B | 1.516(4) |
| N4A | C20A | 1.370(4) | F1A | C4A | 1.354(5) |
| N4A | C21A | 1.386(3) | C23A | C24A | 1.496(4) |
| N4A | C23A | 1.464(4) | C18B | C14B | 1.518(5) |
| N4B | C21B | 1.388(3) | C24A | C25A | 1.348(5) |
| N4B | C23B | 1.464(4) | C15B | C16B | 1.520(5) |
| N4B | C20B | 1.374(4) | C15B | C14B | 1.533(5) |
| N1C | C6C | 1.492(3) | C8A | C5A | 1.509(5) |
| N1C | C7C | 1.481(3) | C13B | C14B | 1.503(5) |
| N5A | C20A | 1.317(4) | C13B | C12B | 1.372(6) |
| N5A | C22A | 1.384(4) | N6A | C25A | 1.366(5) |
| N3B | C17B | 1.474(4) | N6A | C26A | 1.273(7) |
| N3B | C19B | 1.463(4) | C2B | C3B | 1.377(7) |
| N3B | C16B | 1.462(4) | C2B | C1B | 1.446(6) |
| C22B | C21B | 1.399(4) | C2B | C7B | 1.373(8) |
| C22B | C27B | 1.395(4) | C5A | C4A | 1.357(7) |
| C21B | C30B | 1.393(4) | C5A | C6A | 1.382(6) |
| C28B | C27B | 1.378(4) | C4B | C5B | 1.369(7) |
| C28B | C29B | 1.409(4) | C4B | C3B | 1.380(7) |
| C5C | C6C | 1.516(3) | C9A | C10A | 1.382(7) |
| C5C | C4C | 1.528(4) | C9B | C10B | 1.370(8) |
| C20A | C19A | 1.494(4) | C5B | C8B | 1.506(6) |
| C29A | C30A | 1.392(4) | C5B | C6B | 1.374(6) |
| C29A | C31A | 1.513(4) | C1A | C2A | 1.448(6) |
| C29A | C28A | 1.410(4) | C1A | N1A | 1.127(6) |
| C21A | C30A | 1.389(4) | C4A | C3A | 1.387(6) |
| C21A | C22A | 1.405(4) | C12B | C11B | 1.385(7) |
| C24B | C23B | 1.502(4) | N1B | C1B | 1.134(7) |
| C24B | C25B | 1.348(6) | C12A | C11A | 1.390(6) |
| F1B | C4B | 1.358(5) | C3A | C2A | 1.379(7) |
| C4C | C3C | 1.529(4) | C2A | C7A | 1.353(8) |
| C31B | C29B | 1.514(4) | C6A | C7A | 1.374(7) |
| N2A | C13A | 1.341(5) | C7B | C6B | 1.384(7) |
| N2A | C9A | 1.312(5) | C11A | C10A | 1.340(8) |
| C3C | C2C | 1.539(4) | C10B | C11B | 1.359(9) |

TABLE 125
Bond angles for the Compound 1 meglumine salt
Type A single crystal structure model

| Atom | Atom | Atom | Angle/° | Atom | Atom | Atom | Angle/° |
|---|---|---|---|---|---|---|---|
| C26B | S1B | C24B | 88.9(2) | C2D | C3D | C4D | 112.9(2) |
| C24A | S1A | C26A | 89.4(2) | N5B | C20B | N4B | 112.7(2) |
| C17A | N3A | C19A | 110.2(2) | N5B | C20B | C19B | 124.3(3) |
| C17A | N3A | C16A | 110.2(3) | N4B | C20B | C19B | 123.0(3) |
| C16A | N3A | C19A | 110.2(2) | O2D | C2D | C3D | 108.2(2) |
| C20B | N5B | C22B | 105.1(2) | O2D | C2D | C1D | 110.2(2) |
| C7D | N1D | C6D | 114.6(2) | C1D | C2D | C3D | 111.9(2) |
| C20A | N4A | C21A | 107.3(2) | C9B | N2B | C13B | 118.0(4) |
| C20A | N4A | C23A | 127.5(2) | O1C | C1C | C2C | 112.8(2) |
| C21A | N4A | C23A | 125.2(2) | N2A | C13A | C14A | 117.3(3) |
| C21B | N4B | C23B | 125.3(2) | N2A | C13A | C12A | 121.9(3) |
| C20B | N4B | C21B | 107.0(2) | C12A | C13A | C14A | 120.8(3) |
| C20B | N4B | C23B | 127.7(2) | C26B | N6B | C25B | 109.3(4) |
| C7C | N1C | C6C | 114.0(2) | C27A | C28A | C29A | 122.7(3) |
| C20A | N5A | C22A | 105.1(2) | N1D | C6D | C5D | 113.1(2) |
| C19B | N3B | C17B | 109.8(2) | C16A | C15A | C14A | 111.4(3) |
| C16B | N3B | C17B | 110.5(3) | N3A | C17A | C18A | 110.4(3) |
| C16B | N3B | C19B | 110.3(2) | C28A | C27A | C22A | 118.0(3) |
| N5B | C22B | C21B | 110.0(2) | N3B | C17B | C18B | 111.2(3) |
| C27B | C22B | N5B | 129.9(2) | N4A | C23A | C24A | 112.6(2) |
| C27B | C22B | C21B | 120.1(2) | O1D | C1D | C2D | 114.1(2) |
| N4B | C21B | C22B | 105.2(2) | C17B | C18B | C14B | 111.5(3) |
| N4B | C21B | C30B | 132.2(3) | N3B | C19B | C20B | 111.4(2) |
| C30B | C21B | C22B | 122.6(2) | N3A | C16A | C15A | 111.4(2) |
| C27B | C28B | C29B | 122.3(2) | C23A | C24A | S1A | 124.4(2) |
| O5C | C5C | C6C | 106.6(2) | C25A | C24A | S1A | 108.1(3) |
| O5C | C5C | C4C | 108.9(2) | C25A | C24A | C23A | 127.5(3) |
| C6C | C5C | C4C | 113.9(2) | C16B | C15B | C14B | 110.9(3) |
| N4A | C20A | C19A | 123.0(3) | O1A | C8A | C5A | 106.4(3) |
| N5A | C20A | N4A | 112.7(2) | C24B | C25B | N6B | 117.3(4) |
| N5A | C20A | C19A | 124.3(3) | C17A | C18A | C14A | 111.4(3) |
| C30A | C29A | C31A | 120.5(2) | N2B | C13B | C14B | 116.9(3) |
| C30A | C29A | C28A | 119.6(3) | N2B | C13B | C12B | 121.5(3) |
| C28A | C29A | C31A | 119.9(2) | C12B | C13B | C14B | 121.6(4) |
| N4A | C21A | C30A | 132.8(2) | N3B | C16B | C15B | 110.7(3) |
| N4A | C21A | C22A | 104.5(2) | C18B | C14B | C15B | 108.4(3) |
| C30A | C21A | C22A | 122.7(2) | C13B | C14B | C18B | 112.5(3) |
| C23B | C24B | S1B | 123.9(3) | C13B | C14B | C15B | 112.5(3) |
| C25B | C24B | S1B | 108.3(3) | C26A | N6A | C25A | 109.1(4) |
| C25B | C24B | C23B | 127.8(3) | N6B | C26B | S1B | 116.2(3) |
| C28B | C27B | C22B | 117.6(2) | C3B | C2B | C1B | 120.5(5) |
| N1C | C6C | C5C | 109.18(19) | C7B | C2B | C3B | 120.7(4) |
| O4C | C4C | C5C | 104.70(19) | C7B | C2B | C1B | 118.8(5) |
| O4C | C4C | C3C | 108.7(2) | C24A | C25A | N6A | 117.5(4) |
| C5C | C4C | C3C | 115.3(2) | C4A | C5A | C8A | 120.8(4) |
| C21A | C30A | C29A | 117.6(2) | C4A | C5A | C6A | 116.5(4) |
| O2B | C31B | O3B | 124.1(3) | C6A | C5A | C8A | 122.7(5) |
| O2B | C31B | C29B | 118.6(2) | F1B | C4B | C5B | 118.0(4) |
| O3B | C31B | C29B | 117.3(2) | F1B | C4B | C3B | 117.6(5) |
| C9A | N2A | C13A | 117.7(3) | C5B | C4B | C3B | 124.4(4) |
| O3C | C3C | C4C | 109.8(2) | N2A | C9A | O1A | 119.1(4) |
| O3C | C3C | C2C | 110.8(2) | N2A | C9A | C10A | 124.4(4) |
| C4C | C3C | C2C | 111.1(2) | O1A | C9A | C10A | 116.4(4) |
| C9A | O1A | C8A | 117.7(3) | O1B | C9B | C10B | 116.3(4) |
| C28B | C29B | C31B | 120.2(2) | N2B | C9B | O1B | 119.3(4) |
| C30B | C29B | C28B | 120.4(2) | N2B | C9B | C10B | 124.4(5) |
| C30B | C29B | C31B | 119.4(2) | C4B | C5B | C8B | 121.2(4) |
| O3A | C31A | C29A | 118.1(2) | C4B | C5B | C6B | 116.3(4) |
| O2A | C31A | O3A | 124.0(3) | C6B | C5B | C8B | 122.6(5) |
| O2A | C31A | C29A | 117.9(2) | N1A | C1A | C2A | 176.7(6) |
| N4B | C23B | C24B | 112.8(2) | C2B | C3B | C4B | 117.2(5) |
| O5D | C5D | C4D | 112.9(2) | F1A | C4A | C5A | 118.4(4) |
| O5D | C5D | C6D | 110.9(2) | F1A | C4A | C3A | 117.7(5) |
| C6D | C5D | C4D | 109.2(2) | C5A | C4A | C3A | 123.9(4) |
| N3A | C19A | C20A | 111.3(2) | O1B | C8B | C5B | 106.7(3) |
| O4D | C4D | C5D | 107.9(2) | C13B | C12B | C11B | 118.6(5) |
| O4D | C4D | C3D | 110.7(2) | C13A | C12A | C11A | 118.2(4) |
| C3D | C4D | C5D | 110.48(19) | C2A | C3A | C4A | 117.5(5) |
| C9B | O1B | C8B | 117.1(3) | C3A | C2A | C1A | 120.5(5) |
| C13A | C14A | C15A | 113.1(3) | C7A | C2A | C1A | 119.4(5) |
| C13A | C14A | C18A | 111.9(3) | C7A | C2A | C3A | 120.1(4) |
| C15A | C14A | C18A | 108.3(3) | N1B | C1B | C2B | 176.9(7) |
| N5A | C22A | C21A | 110.5(2) | C7A | C6A | C5A | 121.1(5) |
| N5A | C22A | C27A | 130.3(3) | C2B | C7B | C6B | 119.6(5) |
| C27A | C22A | C21A | 119.3(3) | C5B | C6B | C7B | 121.8(5) |
| O2C | C2C | C3C | 108.6(2) | C10A | C11A | C12A | 120.4(5) |
| O2C | C2C | C1C | 109.0(2) | C11A | C10A | C9A | 117.3(4) |
| C1C | C2C | C3C | 113.5(2) | N6A | C26A | S1A | 116.0(3) |
| C29B | C30B | C21B | 116.9(2) | C2A | C7A | C6A | 120.8(5) |
| O3D | C3D | C4D | 111.2(2) | C11B | C10B | C9B | 117.6(4) |
| O3D | C3D | C2D | 110.3(2) | C10B | C11B | C12B | 119.9(5) |

TABLE 126
Torsion angles for the Compound 1 meglumine
salt Type A single crystal structure model

| Atom | Atom | Atom | Atom | Angle/° |
|---|---|---|---|---|
| S1B | C24B | C23B | N4B | 67.9(3) |
| S1B | C24B | C25B | N6B | 1.4(5) |
| S1A | C24A | C25A | N6A | −1.3(5) |
| O5C | C5C | C6C | N1C | −63.5(3) |
| O5C | C5C | C4C | O4C | 57.1(3) |
| O5C | C5C | C4C | C3C | 176.48(19) |
| O3D | C3D | C2D | O2D | 170.5(2) |
| O3D | C3D | C2D | C1D | 48.8(3) |
| O2C | C2C | C1C | O1C | −63.6(3) |
| O5D | C5D | C4D | O4D | 40.0(3) |
| O5D | C5D | C4D | C3D | 161.2(2) |
| O5D | C5D | C6D | N1D | −44.7(3) |
| O3C | C3C | C2C | O2C | 170.25(19) |
| O3C | C3C | C2C | C1C | 48.8(3) |
| O4C | C4C | C3C | O3C | 45.1(3) |
| O4C | C4C | C3C | C2C | −77.7(2) |
| O2D | C2D | C1D | O1D | −68.6(3) |
| O2B | C31B | C29B | C28B | 5.8(4) |
| O2B | C31B | C29B | C30B | −175.6(3) |
| N3A | C17A | C18A | C14A | −58.5(5) |
| O4D | C4D | C3D | O3D | 63.3(3) |
| O4D | C4D | C3D | C2D | −61.2(3) |
| O3B | C31B | C29B | C28B | −174.1(2) |
| O3B | C31B | C29B | C30B | 4.5(4) |
| N5B | C22B | C21B | N4B | −1.3(3) |
| N5B | C22B | C21B | C30B | 177.7(2) |
| N5B | C22B | C27B | C28B | −179.6(3) |
| N5B | C20B | C19B | N3B | −105.0(3) |
| N4A | C20A | C19A | N3A | −70.2(3) |
| N4A | C21A | C30A | C29A | 179.3(2) |
| N4A | C21A | C22A | N5A | 0.0(3) |
| N4A | C21A | C22A | C27A | −179.5(2) |
| N4A | C23A | C24A | S1A | −64.8(3) |
| N4A | C23A | C24A | C25A | 113.2(4) |
| N4B | C21B | C30B | C29B | −179.5(2) |
| N4B | C20B | C19B | N3B | 73.6(4) |
| N5A | C20A | C19A | N3A | 109.0(3) |
| N5A | C22A | C27A | C28A | −178.6(3) |
| N3B | C17B | C18B | C14B | −57.1(4) |
| C22B | N5B | C20B | N4B | 0.1(3) |
| C22B | N5B | C20B | C19B | 178.8(2) |
| C22B | C21B | C30B | C29B | 1.7(4) |
| C21B | N4B | C23B | C24B | 68.2(4) |
| C21B | N4B | C20B | N5B | −0.9(3) |
| C21B | N4B | C20B | C19B | −179.7(2) |
| C21B | C22B | C27B | C28B | 1.2(4) |
| C28B | C29B | C30B | C21B | 1.0(4) |
| C5C | C4C | C3C | O3C | −72.0(3) |
| C5C | C4C | C3C | C2C | 165.1(2) |
| C20A | N4A | C21A | C30A | 179.1(3) |
| C20A | N4A | C21A | C22A | −0.2(3) |
| C20A | N4A | C23A | C24A | 113.1(3) |
| C20A | N5A | C22A | C21A | 0.3(3) |
| C20A | N5A | C22A | C27A | 179.6(3) |
| C29A | C28A | C27A | C22A | −2.2(4) |
| C21A | N4A | C20A | N5A | 0.4(3) |
| C21A | N4A | C20A | C19A | 179.7(2) |
| C21A | N4A | C23A | C24A | −66.9(3) |
| C21A | C22A | C27A | C28A | 0.7(4) |
| C24B | S1B | C26B | N6B | 0.1(4) |
| C27B | C22B | C21B | N4B | 178.0(2) |
| C27B | C22B | C21B | C30B | −2.9(4) |
| C27B | C28B | C29B | C31B | 175.9(2) |
| C27B | C28B | C29B | C30B | −2.7(4) |
| C6C | C5C | C4C | O4C | 175.9(2) |
| C6C | C5C | C4C | C3C | −64.7(3) |
| F1B | C4B | C5B | C8B | 1.4(7) |
| F1B | C4B | C5B | C6B | −179.8(5) |
| F1B | C4B | C3B | C2B | 179.9(5) |
| C4C | C5C | C6C | N1C | 176.4(2) |
| C4C | C3C | C2C | O2C | −67.5(2) |
| C4C | C3C | C2C | C1C | 171.1(2) |
| C30A | C29A | C31A | O3A | −4.0(4) |
| C30A | C29A | C31A | O2A | 176.2(3) |
| C30A | C29A | C28A | C27A | 1.8(4) |
| C30A | C21A | C22A | N5A | −179.4(2) |
| C30A | C21A | C22A | C27A | 1.1(4) |
| C31B | C29B | C30B | C21B | −177.6(2) |
| N2A | C13A | C12A | C11A | −0.5(9) |
| N2A | C9A | C1OA | C11A | −0.6(12) |
| C3C | C2C | C1C | O1C | 57.6(3) |
| O1A | C8A | C5A | C4A | −155.9(5) |
| O1A | C8A | C5A | C6A | 23.5(7) |
| O1A | C9A | C10A | C11A | −178.2(7) |
| C29B | C28B | C27B | C22B | 1.5(4) |
| C31A | C29A | C30A | C21A | −180.0(2) |
| C31A | C29A | C28A | C27A | −178.2(3) |
| C23B | N4B | C21B | C22B | −176.7(2) |
| C23B | N4B | C21B | C30B | 4.4(4) |
| C23B | N4B | C20B | N5B | 177.0(2) |
| C23B | N4B | C20B | C19B | −1.7(4) |
| C23B | C24B | C25B | N6B | −179.9(3) |
| C5D | C4D | C3D | O3D | −56.2(3) |
| C5D | C4D | C3D | C2D | 179.3(2) |
| C19A | N3A | C17A | C18A | −178.0(3) |
| C19A | N3A | C16A | C15A | 178.1(3) |
| C4D | C5D | C6D | N1D | −169.7(2) |
| C4D | C3D | C2D | O2D | −64.5(3) |
| C4D | C3D | C2D | C1D | 173.9(2) |
| O1B | C9B | C10B | C11B | 177.9(8) |
| C14A | C13A | C12A | C11A | 180.0(6) |
| C14A | C15A | C16A | N3A | 57.5(4) |
| C22A | N5A | C20A | N4A | −0.4(3) |
| C22A | N5A | C20A | C19A | −179.7(2) |
| C22A | C21A | C30A | C29A | −1.5(4) |
| C3D | C2D | C1D | O1D | 51.9(3) |
| C20B | N5B | C22B | C21B | 0.8(3) |
| C20B | N5B | C22B | C27B | −178.5(3) |
| C20B | N4B | C21B | C22B | 1.3(3) |
| C20B | N4B | C21B | C30B | −177.6(3) |
| C20B | N4B | C23B | C24B | −109.4(3) |
| N2B | C13B | C14B | C18B | −35.1(5) |
| N2B | C13B | C14B | C15B | 87.7(4) |
| N2B | C13B | C12B | C11B | −0.8(10) |
| N2B | C9B | C10B | C11B | −0.6(13) |
| C13A | N2A | C9A | O1A | 177.7(4) |
| C13A | N2A | C9A | C10A | 0.1(9) |
| C13A | C14A | C15A | C16A | −178.3(3) |
| C13A | C14A | C18A | C17A | 179.7(3) |
| C13A | C12A | C11A | C10A | 0.0(12) |
| C28A | C29A | C30A | C21A | 0.0(4) |
| C28A | C29A | C31A | O3A | 176.0(2) |
| C28A | C29A | C31A | O2A | −3.8(4) |
| C6D | C5D | C4D | O4D | 163.9(2) |
| C6D | C5D | C4D | C3D | −75.0(3) |
| C15A | C14A | C13A | N2A | 33.9(5) |
| C15A | C14A | C13A | C12A | −146.6(4) |
| C15A | C14A | C18A | C17A | 54.3(4) |
| C17A | N3A | C19A | C20A | 165.0(3) |
| C17A | N3A | C16A | C15A | −60.1(4) |
| C17B | N3B | C19B | C20B | 73.5(3) |
| C17B | N3B | C16B | C15B | −59.4(4) |
| C17B | C18B | C14B | C15B | 54.5(4) |
| C17B | C18B | C14B | C13B | 179.6(3) |
| F1A | C4A | C3A | C2A | −179.9(5) |
| C23A | N4A | C20A | N5A | −179.6(2) |
| C23A | N4A | C20A | C19A | −0.3(4) |
| C23A | N4A | C21A | C30A | −1.0(4) |
| C23A | N4A | C21A | C22A | 179.7(2) |
| C23A | C24A | C25A | N6A | −179.6(4) |
| C7C | N1C | C6C | C5C | 169.2(2) |
| C19B | N3B | C17B | C18B | −179.4(3) |
| C19B | N3B | C16B | C15B | 178.9(3) |
| C16A | N3A | C19A | C20A | −73.2(3) |
| C16A | N3A | C17A | C18A | 60.2(4) |
| C24A | S1A | C26A | N6A | −0.3(5) |
| C8A | O1A | C9A | N2A | 0.6(7) |
| C8A | O1A | C9A | C10A | 178.4(6) |
| C8A | C5A | C4A | F1A | −0.7(7) |
| C8A | C5A | C4A | C3A | 178.4(5) |
| C8A | C5A | C6A | C7A | 179.8(6) |
| C25B | C24B | C23B | N4B | −110.7(4) |
| C25B | N6B | C26B | S1B | 0.6(6) |
| C7D | N1D | C6D | C5D | −76.9(3) |
| C18A | C14A | C13A | N2A | −88.8(4) |
| C18A | C14A | C13A | C12A | 90.8(5) |
| C18A | C14A | C15A | C16A | −53.6(4) |
| C13B | N2B | C9B | O1B | −178.3(4) |
| C13B | N2B | C9B | C10B | 0.1(9) |
| C13B | C12B | C11B | C10B | 0.3(13) |
| C16B | N3B | C17B | C18B | 58.7(4) |
| C16B | N3B | C19B | C20B | −164.4(3) |
| C16B | C15B | C14B | C18B | −55.2(4) |
| C16B | C15B | C14B | C13B | 179.7(3) |
| C14B | C15B | C16B | N3B | 58.6(5) |
| C14B | C13B | C12B | C11B | 179.8(6) |
| C26B | S1B | C24B | C23B | −179.6(3) |
| C26B | S1B | C24B | C25B | −0.8(3) |
| C26B | N6B | C25B | C24B | −1.3(6) |
| C2B | C7B | C6B | C5B | −1.8(9) |
| C25A | N6A | C26A | S1A | −0.4(6) |
| C5A | C4A | C3A | C2A | 1.0(8) |
| C5A | C6A | C7A | C2A | 2.5(10) |
| C4B | C5B | C8B | O1B | 153.6(5) |
| C4B | C5B | C6B | C7B | 0.3(8) |
| C9A | N2A | C13A | C14A | 180.0(4) |
| C9A | N2A | C13A | C12A | 0.4(7) |
| C9A | O1A | C8A | C5A | 179.7(4) |
| C9B | O1B | C8B | C5B | 179.9(4) |
| C9B | N2B | C13B | C14B | 180.0(4) |
| C9B | N2B | C13B | C12B | 0.6(7) |
| C9B | C10B | C11B | C12B | 0.3(14) |
| C5B | C4B | C3B | C2B | −1.0(8) |
| C1A | C2A | C7A | C6A | 176.6(6) |
| C3B | C2B | C7B | C6B | 1.9(9) |
| C3B | C4B | C5B | C8B | −177.8(5) |
| C3B | C4B | C5B | C6B | 1.1(8) |
| C4A | C5A | C6A | C7A | −0.7(9) |
| C4A | C3A | C2A | C1A | −178.3(5) |
| C4A | C3A | C2A | C7A | 0.8(8) |
| C8B | O1B | C9B | N2B | 0.3(8) |
| C8B | O1B | C9B | C10B | −178.2(6) |
| C8B | C5B | C6B | C7B | 179.2(5) |
| C12B | C13B | C14B | C18B | 144.3(5) |
| C12B | C13B | C14B | C15B | −92.9(6) |
| C12A | C11A | C10A | C9A | 0.5(13) |
| C3A | C2A | C7A | C6A | −2.5(10) |
| C1B | C2B | C3B | C4B | 179.2(5) |
| C1B | C2B | C7B | C6B | −177.9(5) |
| C6A | C5A | C4A | F1A | 179.8(5) |
| C6A | C5A | C4A | C3A | −1.0(8) |
| C7B | C2B | C3B | C4B | −0.6(8) |
| C6B | C5B | C8B | O1B | −25.2(6) |
| C26A | S1A | C24A | C23A | 179.2(3) |
| C26A | S1A | C24A | C25A | 0.8(3) |
| C26A | N6A | C25A | C24A | 1.1(6) |

TABLE 127
Hydrogen atom coordinates (Å × 104) and
isotropic displacement parameters (Å2 × 103)
for the Compound 1meglumine salt Type A single crystal structure model

| Atom | x | y | z | U(eq) |
|---|---|---|---|---|
| H5C | 8826.07 | 403.25 | 4089.53 | 63 |
| H3D | 8673.32 | 9195.42 | 4706.1 | 63 |
| H2C | 6660.03 | 1576.36 | 5490.74 | 68 |
| H5D | 6317.37 | 9744.48 | 5860.58 | 72 |
| H3C | 9239.39 | 3733.31 | 4864.52 | 73 |
| H1C | 6352.48 | 5262.51 | 5152.97 | 79 |
| H1D | 8743.8 | 5527.39 | 4635.14 | 68 |
| H4C | 9560.55 | 1464.46 | 5024.46 | 81 |
| H2D | 5501.83 | 6681.56 | 4715.01 | 81 |
| H4D | 5575.77 | 9316.83 | 4945.73 | 78 |
| H1DA | 8148.29 | 9133.67 | 6307.34 | 51 |
| H1DB | 8623.03 | 7934.59 | 6395.29 | 51 |
| H1CA | 6391.98 | 2083.07 | 3669.91 | 45 |
| H1CB | 6584.95 | 799.86 | 3706.73 | 45 |
| H28B | 5311.37 | 11305.97 | 6090.83 | 46 |
| H5CA | 8177.55 | 2120.31 | 4178.23 | 40 |
| H27B | 4932.79 | 13349.47 | 6167.7 | 47 |
| H6CA | 5704.36 | 897.43 | 4309.12 | 47 |
| H6CB | 5794.05 | 2309.63 | 4265.34 | 47 |
| H4CA | 7457.97 | 1094.47 | 4877.38 | 44 |
| H30A | 12562.01 | −495.73 | 3072.06 | 45 |
| H3CA | 6646.25 | 3170.63 | 4773.73 | 42 |
| H23A | 500.95 | 11782.55 | 7147.43 | 59 |
| H23B | 493.86 | 13020.92 | 7361.35 | 59 |
| H5DA | 8574.62 | 9487.29 | 5503.56 | 45 |
| H19C | 14619.3 | −5002.95 | 2861.99 | 56 |
| H19D | 13553.94 | −5908.87 | 3018.64 | 56 |
| H4DA | 6259.99 | 8023.44 | 5327.86 | 46 |
| H14A | 12045.52 | −7645.68 | 1983.97 | 66 |
| H2CA | 8336.2 | 2623.53 | 5418.2 | 46 |
| H30B | 2479.55 | 10460.73 | 6835.87 | 45 |
| H3DA | 8335.41 | 7292.61 | 5128.03 | 42 |
| H2DA | 6630.19 | 8033.7 | 4511.42 | 50 |
| H1CC | 7280.76 | 4165.77 | 5744.6 | 58 |
| H1CD | 8121.79 | 4708.23 | 5420.8 | 58 |
| H28A | 10025.41 | −1220.65 | 3903.3 | 52 |
| H6DA | 7328.7 | 7604.76 | 5895.85 | 54 |
| H6DB | 8801.87 | 7598.66 | 5744.96 | 54 |
| H15C | 10933.96 | −5286.53 | 1999.79 | 64 |
| H15D | 10195.91 | −6457.63 | 2134.17 | 64 |
| H17C | 14056.95 | −6908.69 | 2397.11 | 75 |
| H17D | 14860 | −5758.8 | 2272.75 | 75 |
| H27A | 10469.18 | −3258.48 | 3867.24 | 55 |
| H17A | 3246.67 | 16630.18 | 7369.82 | 63 |
| H17B | 3937.12 | 15353.96 | 7348.18 | 63 |
| H23C | 14499.99 | −3140.1 | 2567.28 | 55 |
| H23D | 14572.43 | −1883.04 | 2770.52 | 55 |
| H7CA | 4674.92 | 979.39 | 3366.67 | 83 |
| H7CB | 4143.24 | 1943.89 | 3647.55 | 83 |
| H7CC | 4247.38 | 581.56 | 3767.01 | 83 |
| H1DC | 7660.69 | 6468.27 | 4167.1 | 59 |
| H1DD | 8650.34 | 7551.9 | 4252.36 | 59 |
| H18A | 4799.4 | 16418.32 | 7876.92 | 66 |
| H18B | 4085.05 | 15222.68 | 8000.64 | 66 |
| H19A | 1541.97 | 15860.98 | 6970.96 | 59 |
| H19B | 476.9 | 14925.66 | 7111.45 | 59 |
| H16C | 11144.16 | −5400.41 | 2652.48 | 63 |
| H16D | 11800.15 | −6690.32 | 2628.62 | 63 |
| H15A | 1657.01 | 15346.8 | 8231.94 | 80 |
| H15B | 960.22 | 16621.27 | 8251.09 | 80 |
| H8AA | 10083.16 | −3855.75 | 1112.88 | 96 |
| H8AB | 8759.47 | −4425.69 | 1273.38 | 96 |
| H25B | 1543.74 | 10056.83 | 7531.74 | 86 |
| H7DA | 10191.87 | 9532.53 | 6030.32 | 94 |
| H7DB | 10692.39 | 8295.38 | 6200.6 | 94 |
| H7DC | 10316.6 | 9361.6 | 6466.93 | 94 |
| H18C | 13989.25 | −6797.72 | 1738.44 | 77 |
| H18D | 13345.75 | −5500.79 | 1752.58 | 77 |
| H16A | 165.7 | 15596.91 | 7707.56 | 78 |
| H16B | 933.18 | 16779.04 | 7594.95 | 78 |
| H14B | 2887.48 | 17544.29 | 8013.45 | 68 |
| H26B | 3834.3 | 11284.87 | 8352.17 | 106 |
| H25A | 13570.85 | −184.02 | 2376.71 | 88 |
| H3B | 7238.03 | 10415.81 | 9288.18 | 110 |
| H8BA | 6219.53 | 14377.83 | 8725.48 | 96 |
| H8BB | 4900.03 | 13783.55 | 8880.27 | 96 |
| H12B | 2869.99 | 18593.46 | 8608.12 | 123 |
| H12A | 12077.5 | −8683.25 | 1388.41 | 110 |
| H3A | 7704.24 | −505.04 | 702.82 | 108 |
| H6A | 7450.62 | −4592.68 | 457.8 | 124 |
| H7B | 8863.83 | 13176.22 | 9881.05 | 119 |
| H6B | 7553 | 14514.07 | 9527.09 | 119 |
| H11A | 11134.07 | −8839.99 | 780.11 | 167 |
| H10A | 9908.64 | −7265.7 | 524.08 | 166 |
| H26A | 10961.07 | −1396.87 | 1629.53 | 112 |
| H7A | 6131.8 | −3261.63 | 110.89 | 122 |
| H10B | 5003.69 | 17154.91 | 9478.09 | 163 |
| H11B | 3785.09 | 18750.52 | 9218.09 | 182 |
| H1WA | 11535.25 | 8926.21 | 7099.53 | 158 |
| H1WB | 10512.81 | 8491.47 | 6855.58 | 158 |

TABLE 128
Atomic occupancy of the water molecule in the Compound
1 meglumine salt Type A single crystal structure model

| Atom | Occupancy | Atom | Occupancy | Atom | Occupancy |
|---|---|---|---|---|---|
| O1W | 0.3 | H1WA | 0.3 | H1WB | 0.3 |

### Example 4: Crystallization Process Development of Compound 1 Freeform C

The manufacturing process of Compound 1 freeform C is shown below:
1. Dissolved Compound 1 in DMSO/IPA (2.5V/2.5V) at 60° C.;
2. Charge 3.65V IPA;
3. Charge a suspension of 2% milled seed (Type C) in 0.1V IPA;
4. Stir for 2 h;
5. Charge 18.75 V IPA for 10.5 h;
6. Stir at for 1 h;
7. Cool to 5° C. for 5.5 h;
8. Stir at 5° C. for 11 h;
9. Filter and wash with 4V IPA for 4 times and dry.

The process was validated at 25 g and 800 g scale. Compound 1 freeform C was obtained with 99.3% purity in 82% isolated yield.

### NUMBERED EMBODIMENTS

1. A solid form of Compound 1.

2. The solid form of any one of the previous embodiments, wherein the solid form of Compound 1 is crystalline.

3. The solid form of any one of the previous embodiments, wherein the solid form of Compound 1 is a salt.

### Solid Forms of Compound 1 Meglumine Type A

4. The solid form of any one of the previous embodiments, wherein the solid form of solid form 1 is a crystalline meglumine type A salt.

4A. The solid form of any one of the previous embodiments, wherein the solid form of Compound 1 Meglumine Type A is a crystalline polymorph of Compound 1 Meglumine Type A characterized by two or more, or three XRPD signals selected from the group consisting of 20.8 °2θ, 17.4 °2θ, and 12.8 °2θ (±0.2 °2θ; ±0.1 °2θ; or ±0.0 °2θ; Cu Kα1 radiation).

4B. The solid form of any one of the previous embodiments, wherein the solid form of Compound 1 Meglumine Type A is a crystalline polymorph of Compound 1 Meglumine Type A characterized by signals at 20.8 °2θ, 17.4 °2θ, and 12.8 °2θ (±0.2 °2θ; ±0.1 °2θ; or ±0.0 °2θ; Cu Kα1 radiation).

4C. The solid form of any one of the previous embodiments, wherein the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, and 13.0 °2θ (±0.2 °2θ; ±0.1 °2θ; or ±0.0 °2θ; Cu Kα1 radiation).

4D. The solid form of any one of the previous embodiments, wherein the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, and 22.5 °2θ (±0.2 °2θ; ±0.1 °2θ; or ±0.0 °2θ; Cu Kα1 radiation).

4E. The solid form of any one of the previous embodiments, wherein the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, 22.5 °2θ, 19.2 °2θ, 18.1 °2θ, and 24.0 °2θ (±0.2 °2θ; ±0.1 °2θ; or ±0.0 °2θ; Cu Kα1 radiation).

4F. The solid form of any one of the previous embodiments, wherein the solid form of Compound 1 Meglumine Type A is a crystalline polymorph of Compound 1 Meglumine Type A characterized by a XRPD diffractogram substantially similar to that shown in FIG. 1.

4G. The solid form of any one of the previous embodiments, wherein the solid form of Compound 1 Meglumine Type A is a crystalline polymorph of Compound 1 Meglumine Type A characterized by any combination of the XRPD peaks set forth in Table 24.

4H. The solid form of any one of the previous embodiments, wherein the solid form of Compound 1 Meglumine Type A is a crystalline polymorph of Compound 1 Meglumine Type A characterized by a 1H NMR spectrum substantially similar to that shown in FIG. 130.

4I. The solid form of any one of the previous embodiments, wherein the solid form of Compound 1 Meglumine Type A is a crystalline polymorph of Compound 1 Meglumine Type A characterized by a DSC curve having an endotherm at about 163.9° C.

### Solid Forms of Compound 1 Fumarate Type A

5. The solid form of any one of the previous embodiments, wherein the solid form of solid form 1 is a crystalline fumarate type A salt.

5A. The solid form of any one of the previous embodiments, wherein the solid form of Compound 1 Fumarate Type A is a crystalline polymorph of Compound 1 Fumarate Type A characterized by two or more, or three XRPD signals selected from the group consisting of 8.7 °2θ, 20.7 °2θ, and 19.5 °2θ (±0.2 °2θ; ±0.1 °2θ; or ±0.0 °2θ; Cu Kα1 radiation).

5B. The solid form of any one of the previous embodiments, wherein the solid form of Compound 1 Fumarate Type A is a crystalline polymorph of Compound 1 Fumarate Type A characterized by signals at 8.7 °2θ, 20.7 °2θ, and 19.5 °2θ (±0.2 °2θ; ±0.1 °2θ; or ±0.0 °2θ; Cu Kα1 radiation).

5C. The solid form of any one of the previous embodiments, wherein the solid form of Compound 1 Fumarate Type A is a crystalline polymorph of Compound 1 Fumarate Type A characterized by two or more, or three or more XRPD signals selected from the group consisting of 8.7 °2θ, 20.7 °2θ, 19.5 °2θ, 17.5 °2θ, and 21.9 °2θ (±0.2 °2θ; ±0.1 °2θ; or ±0.0 °2θ; Cu Kα1 radiation).

5D. The solid form of any one of the previous embodiments, wherein the solid form of Compound 1 Fumarate Type A is a crystalline polymorph of Compound 1 Fumarate Type A characterized by two or more, or three or more XRPD signals selected from the group consisting of 8.7 °2θ, 20.7 °2θ, 19.5 °2θ, 17.5 °2θ, 21.9 °2θ, 20.5 °2θ, and 15.6 °2θ (±0.2 °2θ; ±0.1 °2θ; or ±0.0 °2θ; Cu Kα1 radiation).

5E. The solid form of any one of the previous embodiments, wherein the solid form of Compound 1 Fumarate Type A is a crystalline polymorph of Compound 1 Fumarate Type A characterized by two or more, or three or more XRPD signals selected from the group consisting of 8.7 °2θ, 20.7 °2θ, 19.5 °2θ, 17.5 °2θ, 21.9 °2θ, 20.5 °2θ, 15.6 °2θ, 14.5 °2θ, 13.1 °2θ, and 18.6 °2θ (±0.2 °2θ; ±0.1 °2θ; or ±0.0 °2θ; Cu Kα1 radiation).

5F. The solid form of any one of the previous embodiments, wherein the solid form of Compound 1 Fumarate Type A is a crystalline polymorph of Compound 1 Fumarate Type A characterized by a XRPD diffractogram substantially similar to that shown in FIG. 5.

5G. The solid form of any one of the previous embodiments, wherein the solid form of Compound 1 Fumarate Type A is a crystalline polymorph of Compound 1 Fumarate Type A characterized by any combination of the XRPD peaks set forth in Table 1.

5H. The solid form of any one of the previous embodiments, wherein the solid form of Compound 1 Fumarate Type A is a crystalline polymorph of Compound 1 Fumarate Type A characterized by a 1H NMR spectrum substantially similar to that shown in FIG. 131.

5I. The solid form of any one of the previous embodiments, wherein the solid form of Compound 1 Fumarate Type A is a crystalline polymorph of Compound 1 Fumarate Type A characterized by a DSC curve having an endotherm at 210.9° C.

### Solid Forms of Compound 1 Potassium Type A

6. The solid form of any one of the previous embodiments, wherein the solid form of solid form 1 is a crystalline potassium type A salt.

6A. The solid form of any one of the previous embodiments, wherein the solid form of Compound 1 Potassium Type A is a crystalline polymorph of Compound 1 Potassium Type A characterized by two or more, or three XRPD signals selected from the group consisting of 16.1 °2θ, 19.3 °2θ, and 17.7 °2θ (±0.2 °2θ; ±0.1 °2θ; or ±0.0 °2θ; Cu Kα1 radiation).

6B. The solid form of any one of the previous embodiments, wherein the solid form of Compound 1 Potassium Type A is a crystalline polymorph of Compound 1 Potassium Type A characterized by signals at 16.1 °2θ, 19.3 °2θ, and 17.7 °2θ (±0.2 °2θ; ±0.1 °2θ; or ±0.0 °2θ; Cu Kα1 radiation).

6C. The solid form of any one of the previous embodiments, wherein the solid form of Compound 1 Potassium Type A is a crystalline polymorph of Compound 1 Potassium Type A characterized by two or more, or three or more XRPD signals selected from the group consisting of 16.1 °2θ, 19.3 °2θ, 17.7 °2θ, 26.9 °2θ, and 18.9 °2θ (±0.2 °2θ; ±0.1 °2θ; or ±0.0 °2θ; Cu Kα1 radiation).

6D. The solid form of any one of the previous embodiments, wherein the solid form of Compound 1 Potassium Type A is a crystalline polymorph of Compound 1 Potassium Type A characterized by two or more, or three or more XRPD signals selected from the group consisting of 16.1 °2θ, 19.3 °2θ, 17.7 °2θ, 26.9 °2θ, 18.9 °2θ, 26.5 °2θ, and 14.5 °2θ (±0.2 °2θ; ±0.1 °2θ; or ±0.0 °2θ; Cu Kα1 radiation).

6E. The solid form of any one of the previous embodiments, wherein the solid form of Compound 1 Potassium Type A is a crystalline polymorph of Compound 1 Potassium Type A characterized by two or more, or three or more XRPD signals selected from the group consisting of 16.1 °2θ, 19.3 °2θ, 17.7 °2θ, 26.9 °2θ, 18.9 °2θ, 26.5 °2θ, 14.5 °2θ, 21.1 °2θ, 25.1 °2θ, and 21.7 °2θ (±0.2 °2θ; ±0.1 °2θ; or ±0.0 °2θ; Cu Kα1 radiation).

6F. The solid form of any one of the previous embodiments, wherein the solid form of Compound 1 Potassium Type A is a crystalline polymorph of Compound 1 Potassium Type A characterized by a XRPD diffractogram substantially similar to that shown in FIG. 9.

6G. The solid form of any one of the previous embodiments, wherein the solid form of Compound 1 Potassium Type A is a crystalline polymorph of Compound 1 Potassium Type A characterized by any combination of the XRPD peaks set forth in Table 2.

6H. The solid form of any one of the previous embodiments, wherein the solid form of Compound 1 Potassium Type A is a crystalline polymorph of Compound 1 Potassium Type A characterized by a 1H NMR spectrum substantially similar to that shown in FIG. 132.

6I. The solid form of any one of the previous embodiments, wherein the solid form of Compound 1 Potassium Type A is a crystalline polymorph of Compound 1 Potassium Type A characterized by a DSC curve having an endotherm at 241.9° C.

### Solid Forms of Compound 1 Tris Type C

7. The solid form of any one of the previous embodiments, wherein the solid form of solid form 1 is a crystalline Tris type C salt.

7A. The solid form of any one of the previous embodiments, wherein the solid form of Compound 1 Tris Type C is a crystalline polymorph of Compound 1 Tris Type C characterized by two or more, or three XRPD signals selected from the group consisting of 7.7 °2θ, 19.3 °2θ, and 11.6 °2θ (±0.2 °2θ; ±0.1 °2θ; or ±0.0 °2θ; Cu Kα1 radiation).

7B. The solid form of any one of the previous embodiments, wherein the solid form of Compound 1 Tris Type C is a crystalline polymorph of Compound 1 Tris Type C characterized by signals at 7.7 °2θ, 19.3 °2θ, and 11.6 °2θ (±0.2 °2θ; ±0.1 °2θ; or ±0.0 °2θ; Cu Kα1 radiation).

7C. The solid form of any one of the previous embodiments, wherein the solid form of Compound 1 Tris Type C is a crystalline polymorph of Compound 1 Tris Type C characterized by two or more, or three or more XRPD signals selected from the group consisting of 7.7 °2θ, 19.3 °2θ, 11.6 °2θ, 19.5 °2θ, and 15.4 °2θ (±0.2 °2θ; ±0.1 °2θ; or ±0.0 °2θ; Cu Kα1 radiation).

7D. The solid form of any one of the previous embodiments, wherein the solid form of Compound 1 Tris Type C is a crystalline polymorph of Compound 1 Tris Type C characterized by two or more, or three or more XRPD signals selected from the group consisting of 7.7 °2θ, 19.3 °2θ, 11.6 °2θ, 19.5 °2θ, 15.4 °2θ, 3.9 °2θ, and 18.0 °2θ (±0.2 °2θ; ±0.1 °2θ; or ±0.0 °2θ; Cu Kα1 radiation).

7E. The solid form of any one of the previous embodiments, wherein the solid form of Compound 1 Tris Type C is a crystalline polymorph of Compound 1 Tris Type C characterized by two or more, or three or more XRPD signals selected from the group consisting of 7.7 °2θ, 19.3 °2θ, 11.6 °2θ, 19.5 °2θ, 15.4 °2θ, 3.9 °2θ, 18.0 °2θ, 8.9 °2θ, 21.9 °2θ, and 23.1 °2θ (±0.2 °2θ; ±0.1 °2θ; or ±0.0 °2θ; Cu Kα1 radiation).

7F. The solid form of any one of the previous embodiments, wherein the solid form of Compound 1 Tris Type C is a crystalline polymorph of Compound 1 Tris Type C characterized by a XRPD diffractogram substantially similar to that shown in FIG. 13.

7G. The solid form of any one of the previous embodiments, wherein the solid form of Compound 1 Tris Type C is a crystalline polymorph of Compound 1 Tris Type C characterized by any combination of the XRPD peaks set forth in Table 23.

7H. The solid form of any one of the previous embodiments, wherein the solid form of Compound 1 Tris Type C is a crystalline polymorph of Compound 1 Tris Type C characterized by a 1H NMR spectrum substantially similar to that shown in FIG. 133.

7I. The solid form of any one of the previous embodiments, wherein the solid form of Compound 1 Tris Type C is a crystalline polymorph of Compound 1 Tris Type C characterized by a DSC curve having an endotherm at about 192.6° C.

### Solid Forms of Compound 1 HCl Type A

8. The solid form of any one of the previous embodiments, wherein the solid form of solid form 1 is a crystalline HCl type A salt.

8A. The solid form of any one of the previous embodiments, wherein the solid form of Compound 1 HCl Type A is a crystalline polymorph of Compound 1 HCl Type A characterized by two or more, or three XRPD signals selected from the group consisting of 17.7 °2θ, 10.8 °2θ, and 22.1 °2θ (±0.2 °2θ; ±0.1 °2θ; or ±0.0 °2θ; Cu Kα1 radiation).

8B. The solid form of any one of the previous embodiments, wherein the solid form of Compound 1 HCl Type A is a crystalline polymorph of Compound 1 HCl Type A characterized by signals at 17.7 °2θ, 10.8 °2θ, and 22.1 °2θ (±0.2 °2θ; ±0.1 °2θ; or ±0.0 °2θ; Cu Kα1 radiation).

8C. The solid form of any one of the previous embodiments, wherein the solid form of Compound 1 HCl Type A is a crystalline polymorph of Compound 1 HCl Type A characterized by two or more, or three or more XRPD signals selected from the group consisting of 17.7 °2θ, 10.8 °2θ, 22.1 °2θ, 18.1 °2θ, and 23.9 °2θ (±0.2 °2θ; ±0.1 °2θ; or ±0.0 °2θ; Cu Kα1 radiation).

8D. The solid form of any one of the previous embodiments, wherein the solid form of Compound 1 HCl Type A is a crystalline polymorph of Compound 1 HCl Type A characterized by two or more, or three or more XRPD signals selected from the group consisting of 17.7 °2θ, 10.8 °2θ, 22.1 °2θ, 18.1 °2θ, 23.9 °2θ, 21.5 °2θ, and 13.2 °2θ (±0.2 °2θ; ±0.1 °2θ; or ±0.0 °2θ; Cu Kα1 radiation).

8E. The solid form of any one of the previous embodiments, wherein the solid form of Compound 1 HCl Type A is a crystalline polymorph of Compound 1 HCl Type A characterized by two or more, or three or more XRPD signals selected from the group consisting of 17.7 °2θ, 10.8 °2θ, 22.1 °2θ, 18.1 °2θ, 23.9 °2θ, 21.5 °2θ, 13.2 °2θ, 27.2 °2θ, 26.7 °2θ, and 30.3 °2θ (±0.2 °2θ; ±0.1 °2θ; or ±0.0 °2θ; Cu Kα1 radiation).

8F. The solid form of any one of the previous embodiments, wherein the solid form of Compound 1 HCl Type A is a crystalline polymorph of Compound 1 HCl Type A characterized by a XRPD diffractogram substantially similar to that shown in FIG. 17.

8G. The solid form of any one of the previous embodiments, wherein the solid form of Compound 1 HCl Type A is a crystalline polymorph of Compound 1 HCl Type A characterized by any combination of the XRPD peaks set forth in Table 3.

8H. The solid form of any one of the previous embodiments, wherein the solid form of Compound 1 HCl Type A is a crystalline polymorph of Compound 1 HCl Type A characterized by a 1H NMR spectrum substantially similar to that shown in FIG. 134.

8I. The solid form of any one of the previous embodiments, wherein the solid form of Compound 1 HCl Type A is a crystalline polymorph of Compound 1 HCl Type A characterized by a DSC curve having endotherms at about 116.9° C., about 154.7, and about 172.5° C.

### Solid Forms of Compound 1 Sulfate Type A

9. The solid form of any one of the previous embodiments, wherein the solid form of solid form 1 is a crystalline sulfate type A salt. 9A. The solid form of any one of the previous embodiments, wherein the solid form of Compound 1 Sulfate Type A is a crystalline polymorph of Compound 1 Sulfate Type A characterized by two or more, or three XRPD signals selected from the group consisting of 3.4 °2θ, 17.1 °2θ, and 17.8 °2θ (±0.2 °2θ; ±0.1 °2θ; or ±0.0 °2θ; Cu Kα1 radiation).

9B. The solid form of any one of the previous embodiments, wherein the solid form of Compound 1 Sulfate Type A is a crystalline polymorph of Compound 1 Sulfate Type A characterized by signals at 3.4 °2θ, 17.1 °2θ, and 17.8 °2θ (±0.2 °2θ; ±0.1 °2θ; or ±0.0 °2θ; Cu Kα1 radiation).

9C. The solid form of any one of the previous embodiments, wherein the solid form of Compound 1 Sulfate Type A is a crystalline polymorph of Compound 1 Sulfate Type A characterized by two or more, or three or more XRPD signals selected from the group consisting of 3.4 °2θ, 17.1 °2θ, 17.8 °2θ, 5.5 °2θ, and 22.0 °2θ (±0.2 °2θ; ±0.1 °2θ; or ±0.0 °2θ; Cu Kα1 radiation).

9D. The solid form of any one of the previous embodiments, wherein the solid form of Compound 1 Sulfate Type A is a crystalline polymorph of Compound 1 Sulfate Type A characterized by two or more, or three or more XRPD signals selected from the group consisting of 3.4 °2θ, 17.1 °2θ, 17.8 °2θ, 5.5 °2θ, 22.0 °2θ, 10.5 °2θ, and 15.3 °2θ (±0.2 °2θ; ±0.1 °2θ; or ±0.0 °2θ; Cu Kα1 radiation).

9E. The solid form of any one of the previous embodiments, wherein the solid form of Compound 1 Sulfate Type A is a crystalline polymorph of Compound 1 Sulfate Type A characterized by two or more, or three or more XRPD signals selected from the group consisting of 3.4 °2θ, 17.1 °2θ, 17.8 °2θ, 5.5 °2θ, 22.0 °2θ, 10.5 °2θ, 15.3 °2θ, 21.0 °2θ, 25.1 °2θ, and 13.6 °2θ (±0.2 °2θ; ±0.1 °2θ; or ±0.0 °2θ; Cu Kα1 radiation).

9F. The solid form of any one of the previous embodiments, wherein the solid form of Compound 1 Sulfate Type A is a crystalline polymorph of Compound 1 Sulfate Type A characterized by a XRPD diffractogram substantially similar to that shown in FIG. 20.

9G. The solid form of any one of the previous embodiments, wherein the solid form of Compound 1 Sulfate Type A is a crystalline polymorph of Compound 1 Sulfate Type A characterized by any combination of the XRPD peaks set forth in Table 4.

9H. The solid form of any one of the previous embodiments, wherein the solid form of Compound 1 Sulfate Type A is a crystalline polymorph of Compound 1 Sulfate Type A characterized by a 1H NMR spectrum substantially similar to that shown in FIG. 135.

9I. The solid form of any one of the previous embodiments, wherein the solid form of Compound 1 Sulfate Type A is a crystalline polymorph of Compound 1 Sulfate Type A characterized by a DSC curve having endotherms at about 70.1° C., about 116.6° C. and about 150.7° C.

### Solid Forms of Compound 1 Sulfate Type B

10. The solid form of any one of the previous embodiments, wherein the solid form of solid form 1 is a crystalline sulfate type B salt.

10A. The solid form of any one of the previous embodiments, wherein the solid form of Compound 1 Sulfate Type B is a crystalline polymorph of Compound 1 Sulfate Type B characterized by two or more, or three XRPD signals selected from the group consisting of 3.7 °2θ, 17.7 °2θ, and 5.5 °2θ (±0.2 °2θ; ±0.1 °2θ; or ±0.0 °2θ; Cu Kα1 radiation).

10B. The solid form of any one of the previous embodiments, wherein the solid form of Compound 1 Sulfate Type B is a crystalline polymorph of Compound 1 Sulfate Type B characterized by signals at 3.7 °2θ, 17.7 °2θ, and 5.5 °2θ (±0.2 °2θ; ±0.1 °2θ; or ±0.0 °2θ; Cu Kα1 radiation).

10C. The solid form of any one of the previous embodiments, wherein the solid form of Compound 1 Sulfate Type B is a crystalline polymorph of Compound 1 Sulfate Type B characterized by two or more, or three or more XRPD signals selected from the group consisting of 3.7 °2θ, 17.7 °2θ, 5.5 °2θ, 10.9 °2θ, and 18.4 °2θ (±0.2 °2θ; ±0.1 °2θ; or ±0.0 °2θ; Cu Kα1 radiation).

10D. The solid form of any one of the previous embodiments, wherein the solid form of Compound 1 Sulfate Type B is a crystalline polymorph of Compound 1 Sulfate Type B characterized by two or more, or three or more XRPD signals selected from the group consisting of 3.7 °2θ, 17.7 °2θ, 5.5 °2θ, 10.9 °2θ, 18.4 °2θ, 16.7 °2θ, and 7.3 °2θ (±0.2 °2θ; ±0.1 °2θ; or ±0.0 °2θ; Cu Kα1 radiation).

10E. The solid form of any one of the previous embodiments, wherein the solid form of Compound 1 Sulfate Type B is a crystalline polymorph of Compound 1 Sulfate Type B characterized by two or more, or three or more XRPD signals selected from the group consisting of 3.7 °2θ, 17.7 °2θ, 5.5 °2θ, 10.9 °2θ, 18.4 °2θ, 16.7 °2θ, 7.3 °2θ, 13.2 °2θ, 0.0 °2θ, and 0.0 °2θ (±0.2 °2θ; ±0.1 °2θ; or ±0.0 °2θ; Cu Kα1 radiation).

10F. The solid form of any one of the previous embodiments, wherein the solid form of Compound 1 Sulfate Type B is a crystalline polymorph of Compound 1 Sulfate Type B characterized by a XRPD diffractogram substantially similar to that shown in FIG. 23.

10G. The solid form of any one of the previous embodiments, wherein the solid form of Compound 1 Sulfate Type B is a crystalline polymorph of Compound 1 Sulfate Type B characterized by any combination of the XRPD peaks set forth in Table 5.

10H. The solid form of any one of the previous embodiments, wherein the solid form of Compound 1 Sulfate Type B is a crystalline polymorph of Compound 1 Sulfate Type B characterized by a 1H NMR spectrum substantially similar to that shown in FIG. 136.

10I. The solid form of any one of the previous embodiments, wherein the solid form of Compound 1 Sulfate Type B is a crystalline polymorph of Compound 1 Sulfate Type B characterized by a DSC curve having endotherms at about 131.5° C. and about 169.8° C.

### Solid Forms of Compound 1 Sulfate Type C

11. The solid form of any one of the previous embodiments, wherein the solid form of solid form 1 is a crystalline sulfate type C salt.

11A. The solid form of any one of the previous embodiments, wherein the solid form of Compound 1 Sulfate Type C is a crystalline polymorph of Compound 1 Sulfate Type C characterized by two or more, or three XRPD signals selected from the group consisting of 17.9 °2θ, 16.6 °2θ, and 22.5 °2θ (±0.2 °2θ; ±0.1 °2θ; or ±0.0 °2θ; Cu Kα1 radiation).

11B. The solid form of any one of the previous embodiments, wherein the solid form of Compound 1 Sulfate Type C is a crystalline polymorph of Compound 1 Sulfate Type C characterized by signals at 17.9 °2θ, 16.6 °2θ, and 22.5 °2θ (±0.2 °2θ; ±0.1 °2θ; or ±0.0 °2θ; Cu Kα1 radiation).

11C. The solid form of any one of the previous embodiments, wherein the solid form of Compound 1 Sulfate Type C is a crystalline polymorph of Compound 1 Sulfate Type C characterized by two or more, or three or more XRPD signals selected from the group consisting of 17.9 °2θ, 16.6 °2θ, 22.5 °2θ, 19.5 °2θ, and 9.5 °2θ (±0.2 °2θ; ±0.1 °2θ; or ±0.0 °2θ; Cu Kα1 radiation).

11D. The solid form of any one of the previous embodiments, wherein the solid form of Compound 1 Sulfate Type C is a crystalline polymorph of Compound 1 Sulfate Type C characterized by two or more, or three or more XRPD signals selected from the group consisting of 17.9 °2θ, 16.6 °2θ, 22.5 °2θ, 19.5 °2θ, 9.5 °2θ, 3.5 °2θ, and 7.2 °2θ (±0.2 °2θ; ±0.1 °2θ; or ±0.0 °2θ; Cu Kα1 radiation).

11E. The solid form of any one of the previous embodiments, wherein the solid form of Compound 1 Sulfate Type C is a crystalline polymorph of Compound 1 Sulfate Type C characterized by two or more, or three or more XRPD signals selected from the group consisting of 17.9 °2θ, 16.6 °2θ, 22.5 °2θ, 19.5 °2θ, 9.5 °2θ, 3.5 °2θ, 7.2 °2θ, 0.0 °2θ, 0.0 °2θ, and 0.0 °2θ (±0.2 °2θ; ±0.1 °2θ; or ±0.0 °2θ; Cu Kα1 radiation).

11F. The solid form of any one of the previous embodiments, wherein the solid form of Compound 1 Sulfate Type C is a crystalline polymorph of Compound 1 Sulfate Type C characterized by a XRPD diffractogram substantially similar to that shown in FIG. 26.

11G. The solid form of any one of the previous embodiments, wherein the solid form of Compound 1 Sulfate Type C is a crystalline polymorph of Compound 1 Sulfate Type C characterized by any combination of the XRPD peaks set forth in Table 6.

11H. The solid form of any one of the previous embodiments, wherein the solid form of Compound 1 Sulfate Type C is a crystalline polymorph of Compound 1 Sulfate Type C characterized by a 1H NMR spectrum substantially similar to that shown in FIG. 137.

11I. The solid form of any one of the previous embodiments, wherein the solid form of Compound 1 Sulfate Type C is a crystalline polymorph of Compound 1 Sulfate Type C characterized by a DSC curve having an endotherm at about 93.2° C.

### Solid Forms of Compound 1 Phosphate Type A

12. The solid form of any one of the previous embodiments, wherein the solid form of solid form 1 is a crystalline phosphate type A salt.

12A. The solid form of any one of the previous embodiments, wherein the solid form of Compound 1 Phosphate Type A is a crystalline polymorph of Compound 1 Phosphate Type A characterized by two or more, or three XRPD signals selected from the group consisting of 12.6 °2θ, 22.5 °2θ, and 12.0 °2θ (±0.2 °2θ; ±0.1 °2θ; or ±0.0 °2θ; Cu Kα1 radiation).

12B. The solid form of any one of the previous embodiments, wherein the solid form of Compound 1 Phosphate Type A is a crystalline polymorph of Compound 1 Phosphate Type A characterized by signals at 12.6 °2θ, 22.5 °2θ, and 12.0 °2θ (±0.2 °2θ; ±0.1 °2θ; or ±0.0 °2θ; Cu Kα1 radiation).

12C. The solid form of any one of the previous embodiments, wherein the solid form of Compound 1 Phosphate Type A is a crystalline polymorph of Compound 1 Phosphate Type A characterized by two or more, or three or more XRPD signals selected from the group consisting of 12.6 °2θ, 22.5 °2θ, 12.0 °2θ, 7.5 °2θ, and 22.1 °2θ (±0.2 °2θ; ±0.1 °2θ; or ±0.0 °2θ; Cu Kα1 radiation).

12D. The solid form of any one of the previous embodiments, wherein the solid form of Compound 1 Phosphate Type A is a crystalline polymorph of Compound 1 Phosphate Type A characterized by two or more, or three or more XRPD signals selected from the group consisting of 12.6 °2θ, 22.5 °2θ, 12.0 °2θ, 7.5 °2θ, 22.1 °2θ, 9.9 °2θ, and 21.7 °2θ (±0.2 °2θ; ±0.1 °2θ; or ±0.0 °2θ; Cu Kα1 radiation).

12E. The solid form of any one of the previous embodiments, wherein the solid form of Compound 1 Phosphate Type A is a crystalline polymorph of Compound 1 Phosphate Type A characterized by two or more, or three or more XRPD signals selected from the group consisting of 12.6 °2θ, 22.5 °2θ, 12.0 °2θ, 7.5 °2θ, 22.1 °2θ, 9.9 °2θ, 21.7 °2θ, 15.0 °2θ, 20.5 °2θ, and 24.0 °2θ (±0.2 °2θ; ±0.1 °2θ; or ±0.0 °2θ; Cu Kα1 radiation).

12F. The solid form of any one of the previous embodiments, wherein the solid form of Compound 1 Phosphate Type A is a crystalline polymorph of Compound 1 Phosphate Type A characterized by a XRPD diffractogram substantially similar to that shown in FIG. 29.

12G. The solid form of any one of the previous embodiments, wherein the solid form of Compound 1 Phosphate Type A is a crystalline polymorph of Compound 1 Phosphate Type A characterized by any combination of the XRPD peaks set forth in Table 7.

12H. The solid form of any one of the previous embodiments, wherein the solid form of Compound 1 Phosphate Type A is a crystalline polymorph of Compound 1 Phosphate Type A characterized by a 1H NMR spectrum substantially similar to that shown in FIG. 138.

12I. The solid form of any one of the previous embodiments, wherein the solid form of Compound 1 Phosphate Type A is a crystalline polymorph of Compound 1 Phosphate Type A characterized by a DSC curve having endotherms at about 133.9° C. and about 159.3° C.

### Solid Forms of Compound 1 Tartrate Type A

13. The solid form of any one of the previous embodiments, wherein the solid form of solid form 1 is a crystalline tartrate type A salt.

13A. The solid form of any one of the previous embodiments, wherein the solid form of Compound 1 Tartrate Type A is a crystalline polymorph of Compound 1 Tartrate Type A characterized by two or more, or three XRPD signals selected from the group consisting of 19.3 °2θ, 19.8 °2θ, and 21.5 °2θ (±0.2 °2θ; ±0.1 °2θ; or ±0.0 °2θ; Cu Kα1 radiation).

13B. The solid form of any one of the previous embodiments, wherein the solid form of Compound 1 Tartrate Type A is a crystalline polymorph of Compound 1 Tartrate Type A characterized by signals at 19.3 °2θ, 19.8 °2θ, and 21.5 °2θ (±0.2 °2θ; ±0.1 °2θ; or ±0.0 °2θ; Cu Kα1 radiation).

13C. The solid form of any one of the previous embodiments, wherein the solid form of Compound 1 Tartrate Type A is a crystalline polymorph of Compound 1 Tartrate Type A characterized by two or more, or three or more XRPD signals selected from the group consisting of 19.3 °2θ, 19.8 °2θ, 21.5 °2θ, 3.4 °2θ, and 14.2 °2θ (±0.2 °2θ; ±0.1 °2θ; or ±0.0 °2θ; Cu Kα1 radiation).

13D. The solid form of any one of the previous embodiments, wherein the solid form of Compound 1 Tartrate Type A is a crystalline polymorph of Compound 1 Tartrate Type A characterized by two or more, or three or more XRPD signals selected from the group consisting of 19.3 °2θ, 19.8 °2θ, 21.5 °2θ, 3.4 °2θ, 14.2 °2θ, 11.3 °2θ, and 23.1 °2θ (±0.2 °2θ; ±0.1 °2θ; or ±0.0 °2θ; Cu Kα1 radiation).

13E. The solid form of any one of the previous embodiments, wherein the solid form of Compound 1 Tartrate Type A is a crystalline polymorph of Compound 1 Tartrate Type A characterized by two or more, or three or more XRPD signals selected from the group consisting of 19.3 °2θ, 19.8 °2θ, 21.5 °2θ, 3.4 °2θ, 14.2 °2θ, 11.3 °2θ, 23.1 °2θ, 14.7 °2θ, 25.7 °2θ, and 24.8 °2θ (±0.2 °2θ; ±0.1 °2θ; or ±0.0 °2θ; Cu Kα1 radiation).

13F. The solid form of any one of the previous embodiments, wherein the solid form of Compound 1 Tartrate Type A is a crystalline polymorph of Compound 1 Tartrate Type A characterized by a XRPD diffractogram substantially similar to that shown in FIG. 32.

13G. The solid form of any one of the previous embodiments, wherein the solid form of Compound 1 Tartrate Type A is a crystalline polymorph of Compound 1 Tartrate Type A characterized by any combination of the XRPD peaks set forth in Table 8.

13H. The solid form of any one of the previous embodiments, wherein the solid form of Compound 1 Tartrate Type A is a crystalline polymorph of Compound 1 Tartrate Type A characterized by a 1H NMR spectrum substantially similar to that shown in FIG. 139.

13I. The solid form of any one of the previous embodiments, wherein the solid form of Compound 1 Tartrate Type A is a crystalline polymorph of Compound 1 Tartrate Type A characterized by a DSC curve having endotherms at about 122.8° C. and about 189.0° C.

### Solid Forms of Compound 1 Mesylate Type A

14. The solid form of any one of the previous embodiments, wherein the solid form of solid form 1 is a crystalline mesylate type A salt.

14A. The solid form of any one of the previous embodiments, wherein the solid form of Compound 1 Mesylate Type A is a crystalline polymorph of Compound 1 Mesylate Type A characterized by two or more, or three XRPD signals selected from the group consisting of 22.9 °2θ, 19.1 °2θ, and 18.5 °2θ (±0.2 °2θ; ±0.1 °2θ; or ±0.0 °2θ; Cu Kα1 radiation).

14B. The solid form of any one of the previous embodiments, wherein the solid form of Compound 1 Mesylate Type A is a crystalline polymorph of Compound 1 Mesylate Type A characterized by signals at 22.9 °2θ, 19.1 °2θ, and 18.5 °2θ (±0.2 °2θ; ±0.1 °2θ; or ±0.0 °2θ; Cu Kα1 radiation).

14C. The solid form of any one of the previous embodiments, wherein the solid form of Compound 1 Mesylate Type A is a crystalline polymorph of Compound 1 Mesylate Type A characterized by two or more, or three or more XRPD signals selected from the group consisting of 22.9 °2θ, 19.1 °2θ, 18.5 °2θ, 22.7 °2θ, and 26.7 °2θ (±0.2 °2θ; ±0.1 °2θ; or ±0.0 °2θ; Cu Kα1 radiation).

14D. The solid form of any one of the previous embodiments, wherein the solid form of Compound 1 Mesylate Type A is a crystalline polymorph of Compound 1 Mesylate Type A characterized by two or more, or three or more XRPD signals selected from the group consisting of 22.9 °2θ, 19.1 °2θ, 18.5 °2θ, 22.7 °2θ, 26.7 °2θ, 10.9 °2θ, and 20.1 °2θ (±0.2 °2θ; ±0.1 °2θ; or ±0.0 °2θ; Cu Kα1 radiation).

14E. The solid form of any one of the previous embodiments, wherein the solid form of Compound 1 Mesylate Type A is a crystalline polymorph of Compound 1 Mesylate Type A characterized by two or more, or three or more XRPD signals selected from the group consisting of 22.9 °2θ, 19.1 °2θ, 18.5 °2θ, 22.7 °2θ, 26.7 °2θ, 10.9 °2θ, 20.1 °2θ, 5.8 °2θ, 12.7 °2θ, and 25.7 °2θ (±0.2 °2θ; ±0.1 °2θ; or ±0.0 °2θ; Cu Kα1 radiation).

14F. The solid form of any one of the previous embodiments, wherein the solid form of Compound 1 Mesylate Type A is a crystalline polymorph of Compound 1 Mesylate Type A characterized by a XRPD diffractogram substantially similar to that shown in FIG. 35.

14G. The solid form of any one of the previous embodiments, wherein the solid form of Compound 1 Mesylate Type A is a crystalline polymorph of Compound 1 Mesylate Type A characterized by any combination of the XRPD peaks set forth in Table 9.

14H. The solid form of any one of the previous embodiments, wherein the solid form of Compound 1 Mesylate Type A is a crystalline polymorph of Compound 1 Mesylate Type A characterized by a 1H NMR spectrum substantially similar to that shown in FIG. 140.

14I. The solid form of any one of the previous embodiments, wherein the solid form of Compound 1 Mesylate Type A is a crystalline polymorph of Compound 1 Mesylate Type A characterized by a DSC curve having an endotherm at about 131.3° C.

### Solidforms of Compound 1 Tosylate Type A

15. The solid form of any one of the previous embodiments, wherein the solid form of solid form 1 is a crystalline tosylate type A salt.

15A. The solid form of any one of the previous embodiments, wherein the solid form of Compound 1 Tosylate Type A is a crystalline polymorph of Compound 1 Tosylate Type A characterized by two or more, or three XRPD signals selected from the group consisting of 5.6 °2θ, 21.5 °2θ, and 12.9 °2θ (±0.2 °2θ; ±0.1 °2θ; or ±0.0 °2θ; Cu Kα1 radiation).

15B. The solid form of any one of the previous embodiments, wherein the solid form of Compound 1 Tosylate Type A is a crystalline polymorph of Compound 1 Tosylate Type A characterized by signals at 5.6 °2θ, 21.5 °2θ, and 12.9 °2θ (±0.2 °2θ; ±0.1 °2θ; or ±0.0 °2θ; Cu Kα1 radiation).

15C. The solid form of any one of the previous embodiments, wherein the solid form of Compound 1 Tosylate Type A is a crystalline polymorph of Compound 1 Tosylate Type A characterized by two or more, or three or more XRPD signals selected from the group consisting of 5.6 °2θ, 21.5 °2θ, 12.9 °2θ, 17.7 °2θ, and 16.6 °2θ (±0.2 °2θ; ±0.1 °2θ; or ±0.0 °2θ; Cu Kα1 radiation).

15D. The solid form of any one of the previous embodiments, wherein the solid form of Compound 1 Tosylate Type A is a crystalline polymorph of Compound 1 Tosylate Type A characterized by two or more, or three or more XRPD signals selected from the group consisting of 5.6 °2θ, 21.5 °2θ, 12.9 °2θ, 17.7 °2θ, 16.6 °2θ, 19.9 °2θ, and 15.1 °2θ (±0.2 °2θ; ±0.1 °2θ; or ±0.0 °2θ; Cu Kα1 radiation).

15E. The solid form of any one of the previous embodiments, wherein the solid form of Compound 1 Tosylate Type A is a crystalline polymorph of Compound 1 Tosylate Type A characterized by two or more, or three or more XRPD signals selected from the group consisting of 5.6 °2θ, 21.5 °2θ, 12.9 °2θ, 17.7 °2θ, 16.6 °2θ, 19.9 °2θ, 15.1 °2θ, 0.0 °2θ, 0.0 °2θ, and 0.0 °2θ (±0.2 °2θ; ±0.1 °2θ; or ±0.0 °2θ; Cu Kα1 radiation).

15F. The solid form of any one of the previous embodiments, wherein the solid form of Compound 1 Tosylate Type A is a crystalline polymorph of Compound 1 Tosylate Type A characterized by a XRPD diffractogram substantially similar to that shown in FIG. 38.

15G. The solid form of any one of the previous embodiments, wherein the solid form of Compound 1 Tosylate Type A is a crystalline polymorph of Compound 1 Tosylate Type A characterized by any combination of the XRPD peaks set forth in Table 10.

15H. The solid form of any one of the previous embodiments, wherein the solid form of Compound 1 Tosylate Type A is a crystalline polymorph of Compound 1 Tosylate Type A characterized by a 1H NMR spectrum substantially similar to that shown in FIG. 141.

15I. The solid form of any one of the previous embodiments, wherein the solid form of Compound 1 Tosylate Type A is a crystalline polymorph of Compound 1 Tosylate Type A characterized by a DSC curve having endotherms at about 76.6° C. and about 117.8° C.

### Solid Forms of Compound 1 Tosylate Type B

16. The solid form of any one of the previous embodiments, wherein the solid form of solid form 1 is a crystalline tosylate type B salt.

16A. The solid form of any one of the previous embodiments, wherein the solid form of Compound 1 Tosylate Type B is a crystalline polymorph of Compound 1 Tosylate Type B characterized by two or more, or three XRPD signals selected from the group consisting of 6.5 °2θ, 5.1 °2θ, and 12.9 °2θ (±0.2 °2θ; ±0.1 °2θ; or ±0.0 °2θ; Cu Kα1 radiation).

16B. The solid form of any one of the previous embodiments, wherein the solid form of Compound 1 Tosylate Type B is a crystalline polymorph of Compound 1 Tosylate Type B characterized by signals at 6.5 °2θ, 5.1 °2θ, and 12.9 °2θ (±0.2 °2θ; ±0.1 °2θ; or ±0.0 °2θ; Cu Kα1 radiation).

16C. The solid form of any one of the previous embodiments, wherein the solid form of Compound 1 Tosylate Type B is a crystalline polymorph of Compound 1 Tosylate Type B characterized by two or more, or three or more XRPD signals selected from the group consisting of 6.5 °2θ, 5.1 °2θ, 12.9 °2θ, 11.3 °2θ, and 22.2 °2θ (±0.2 °2θ; ±0.1 °2θ; or ±0.0 °2θ; Cu Kα1 radiation).

16D. The solid form of any one of the previous embodiments, wherein the solid form of Compound 1 Tosylate Type B is a crystalline polymorph of Compound 1 Tosylate Type B characterized by two or more, or three or more XRPD signals selected from the group consisting of 6.5 °2θ, 5.1 °2θ, 12.9 °2θ, 11.3 °2θ, 22.2 °2θ, 20.7 °2θ, and 18.9 °2θ (±0.2 °2θ; ±0.1 °2θ; or ±0.0 °2θ; Cu Kα1 radiation).

16E. The solid form of any one of the previous embodiments, wherein the solid form of Compound 1 Tosylate Type B is a crystalline polymorph of Compound 1 Tosylate Type B characterized by two or more, or three or more XRPD signals selected from the group consisting of 6.5 °2θ, 5.1 °2θ, 12.9 °2θ, 11.3 °2θ, 22.2 °2θ, 20.7 °2θ, 18.9 °2θ, 13.3 °2θ, 7.7 °2θ, and 22.9 °2θ (±0.2 °2θ; ±0.1 °2θ; or ±0.0 °2θ; Cu Kα1 radiation).

16F. The solid form of any one of the previous embodiments, wherein the solid form of Compound 1 Tosylate Type B is a crystalline polymorph of Compound 1 Tosylate Type B characterized by a XRPD diffractogram substantially similar to that shown in FIG. 41.

16G. The solid form of any one of the previous embodiments, wherein the solid form of Compound 1 Tosylate Type B is a crystalline polymorph of Compound 1 Tosylate Type B characterized by any combination of the XRPD peaks set forth in Table 11.

16H. The solid form of any one of the previous embodiments, wherein the solid form of Compound 1 Tosylate Type B is a crystalline polymorph of Compound 1 Tosylate Type B characterized by a 1H NMR spectrum substantially similar to that shown in FIG. 142.

16I. The solid form of any one of the previous embodiments, wherein the solid form of Compound 1 Tosylate Type B is a crystalline polymorph of Compound 1 Tosylate Type B characterized by a DSC curve having endotherms at about 125.8° C. and about 128.2° C.

### Solid Forms of Compound 1 Arginine Type A

17. The solid form of any one of the previous embodiments, wherein the solid form of solid form 1 is a crystalline arginine type A salt.

17A. The solid form of any one of the previous embodiments, wherein the solid form of Compound 1 Arginine Type A is a crystalline polymorph of Compound 1 Arginine Type A characterized by two or more, or three XRPD signals selected from the group consisting of 17.1 °2θ, 18.4 20, and 19.5 °2θ (±0.2 °2θ; ±0.1 °2θ; or ±0.0 °2θ; Cu Kα1 radiation).

17B. The solid form of any one of the previous embodiments, wherein the solid form of Compound 1 Arginine Type A is a crystalline polymorph of Compound 1 Arginine Type A characterized by signals at 17.1 °2θ, 18.4 °2θ, and 19.5 °2θ (±0.2 °2θ; ±0.1 °2θ; or ±0.0 °2θ; Cu Kα1 radiation).

17C. The solid form of any one of the previous embodiments, wherein the solid form of Compound 1 Arginine Type A is a crystalline polymorph of Compound 1 Arginine Type A characterized by two or more, or three or more XRPD signals selected from the group consisting of 17.1 °2θ, 18.4 °2θ, 19.5 °2θ, 19.2 °2θ, and 23.1 °2θ (±0.2 °2θ; ±0.1 °2θ; or ±0.0 °2θ; Cu Kα1 radiation).

17D. The solid form of any one of the previous embodiments, wherein the solid form of Compound 1 Arginine Type A is a crystalline polymorph of Compound 1 Arginine Type A characterized by two or more, or three or more XRPD signals selected from the group consisting of 17.1 °2θ, 18.4 °2θ, 19.5 °2θ, 19.2 °2θ, 23.1 °2θ, 6.8 °2θ, and 27.5 °2θ (±0.2 °2θ; ±0.1 °2θ; or ±0.0 °2θ; Cu Kα1 radiation).

17E. The solid form of any one of the previous embodiments, wherein the solid form of Compound 1 Arginine Type A is a crystalline polymorph of Compound 1 Arginine Type A characterized by two or more, or three or more XRPD signals selected from the group consisting of 17.1 °2θ, 18.4 °2θ, 19.5 °2θ, 19.2 °2θ, 23.1 °2θ, 6.8 °2θ, 27.5 °2θ, 0.0 °2θ, 0.0 °2θ, and 0.0 °2θ (±0.2 °2θ; ±0.1 °2θ; or ±0.0 °2θ; Cu Kα1 radiation).

17F. The solid form of any one of the previous embodiments, wherein the solid form of Compound 1 Arginine Type A is a crystalline polymorph of Compound 1 Arginine Type A characterized by a XRPD diffractogram substantially similar to that shown in FIG. 44.

17G. The solid form of any one of the previous embodiments, wherein the solid form of Compound 1 Arginine Type A is a crystalline polymorph of Compound 1 Arginine Type A characterized by any combination of the XRPD peaks set forth in Table 12.

17H. The solid form of any one of the previous embodiments, wherein the solid form of Compound 1 Arginine Type A is a crystalline polymorph of Compound 1 Arginine Type A characterized by a 1H NMR spectrum substantially similar to that shown in FIG. 143.

17I. The solid form of any one of the previous embodiments, wherein the solid form of Compound 1 Arginine Type A is a crystalline polymorph of Compound 1 Arginine Type A characterized by a DSC curve having endotherms at about 92.4° C.

### Solid Forms of Compound 1 Arginine Type B

18. The solid form of any one of the previous embodiments, wherein the solid form of solid form 1 is a crystalline arginine type B salt.

18A. The solid form of any one of the previous embodiments, wherein the solid form of Compound 1 Arginine Type B is a crystalline polymorph of Compound 1 Arginine Type B characterized by two or more, or three XRPD signals selected from the group consisting of 10.3 °2θ, 18.2 °2θ, and 16.6 °2θ (±0.2 °2θ; ±0.1 °2θ; or ±0.0 °2θ; Cu Kα1 radiation).

18B. The solid form of any one of the previous embodiments, wherein the solid form of Compound 1 Arginine Type B is a crystalline polymorph of Compound 1 Arginine Type B characterized by signals at 10.3 °2θ, 18.2 °2θ, and 16.6 °2θ (±0.2 °2θ; ±0.1 °2θ; or ±0.0 °2θ; Cu Kα1 radiation).

18C. The solid form of any one of the previous embodiments, wherein the solid form of Compound 1 Arginine Type B is a crystalline polymorph of Compound 1 Arginine Type B characterized by two or more, or three or more XRPD signals selected from the group consisting of 10.3 °2θ, 18.2 °2θ, 16.6 °2θ, 20.2 °2θ, and 25.2 °2θ (±0.2 °2θ; ±0.1 °2θ; or ±0.0 °2θ; Cu Kα1 radiation).

18D. The solid form of any one of the previous embodiments, wherein the solid form of Compound 1 Arginine Type B is a crystalline polymorph of Compound 1 Arginine Type B characterized by two or more, or three or more XRPD signals selected from the group consisting of 10.3 °2θ, 18.2 °2θ, 16.6 °2θ, 20.2 °2θ, 25.2 °2θ, 15.6 °2θ, and 24.5 °2θ (±0.2 °2θ; ±0.1 °2θ; or ±0.0 °2θ; Cu Kα1 radiation).

18E. The solid form of any one of the previous embodiments, wherein the solid form of Compound 1 Arginine Type B is a crystalline polymorph of Compound 1 Arginine Type B characterized by two or more, or three or more XRPD signals selected from the group consisting of 10.3 °2θ, 18.2 °2θ, 16.6 °2θ, 20.2 °2θ, 25.2 °2θ, 15.6 °2θ, 24.5 °2θ, 26.0 °2θ, 13.3 °2θ, and 21.9 °2θ (±0.2 °2θ; ±0.1 °2θ; or ±0.0 °2θ; Cu Kα1 radiation).

18F. The solid form of any one of the previous embodiments, wherein the solid form of Compound 1 Arginine Type B is a crystalline polymorph of Compound 1 Arginine Type B characterized by a XRPD diffractogram substantially similar to that shown in FIG. 47.

18G. The solid form of any one of the previous embodiments, wherein the solid form of Compound 1 Arginine Type B is a crystalline polymorph of Compound 1 Arginine Type B characterized by any combination of the XRPD peaks set forth in Table 13.

18H. The solid form of any one of the previous embodiments, wherein the solid form of Compound 1 Arginine Type B is a crystalline polymorph of Compound 1 Arginine Type B characterized by a 1H NMR spectrum substantially similar to that shown in FIG. 144.

18I. The solid form of any one of the previous embodiments, wherein the solid form of Compound 1 Arginine Type B is a crystalline polymorph of Compound 1 Arginine Type B characterized by a DSC curve having an endotherm at about 140.8° C.

### Solid Forms of Compound 1 Lysine Type A

19. The solid form of any one of the previous embodiments, wherein the solid form of solid form 1 is a crystalline Lysine Type A salt.

19A. The solid form of any one of the previous embodiments, wherein the solid form of Compound 1 Lysine Type A is a crystalline polymorph of Compound 1 Lysine Type A characterized by two or more, or three XRPD signals selected from the group consisting of 7.3 °2θ, 22.0 °2θ, and 10.9 °2θ (±0.2 °2θ; ±0.1 °2θ; or ±0.0 °2θ; Cu Kα1 radiation).

19B. The solid form of any one of the previous embodiments, wherein the solid form of Compound 1 Lysine Type A is a crystalline polymorph of Compound 1 Lysine Type A characterized by signals at 7.3 °2θ, 22.0 °2θ, and 10.9 °2θ (±0.2 °2θ; ±0.1 °2θ; or ±0.0 °2θ; Cu Kα1 radiation).

19C. The solid form of any one of the previous embodiments, wherein the solid form of Compound 1 Lysine Type A is a crystalline polymorph of Compound 1 Lysine Type A characterized by two or more, or three or more XRPD signals selected from the group consisting of 7.3 °2θ, 22.0 °2θ, 10.9 °2θ, 21.4 °2θ, and 14.5 °2θ (±0.2 °2θ; ±0.1 °2θ; or ±0.0 °2θ; Cu Kα1 radiation).

19D. The solid form of any one of the previous embodiments, wherein the solid form of Compound 1 Lysine Type A is a crystalline polymorph of Compound 1 Lysine Type A characterized by two or more, or three or more XRPD signals selected from the group consisting of 7.3 °2θ, 22.0 °2θ, 10.9 °2θ, 21.4 °2θ, 14.5 °2θ, 24.2 °2θ, and 12.1 °2θ (±0.2 °2θ; ±0.1 °2θ; or ±0.0 °2θ; Cu Kα1 radiation).

19E. The solid form of any one of the previous embodiments, wherein the solid form of Compound 1 Lysine Type A is a crystalline polymorph of Compound 1 Lysine Type A characterized by two or more, or three or more XRPD signals selected from the group consisting of 7.3 °2θ, 22.0 °2θ, 10.9 °2θ, 21.4 °2θ, 14.5 °2θ, 24.2 °2θ, 12.1 °2θ, 24.9 °2θ, 26.6 °2θ, and 0.0 °2θ (±0.2 °2θ; ±0.1 °2θ; or ±0.0 °2θ; Cu Kα1 radiation).

19F. The solid form of any one of the previous embodiments, wherein the solid form of Compound 1 Lysine Type A is a crystalline polymorph of Compound 1 Lysine Type A characterized by a XRPD diffractogram substantially similar to that shown in FIG. 50. 19G. The solid form of any one of the previous embodiments, wherein the solid form of Compound 1 Lysine Type A is a crystalline polymorph of Compound 1 Lysine Type A characterized by any combination of the XRPD peaks set forth in Table 14.

19H. The solid form of any one of the previous embodiments, wherein the solid form of Compound 1 Lysine Type A is a crystalline polymorph of Compound 1 Lysine Type A characterized by a 1H NMR spectrum substantially similar to that shown in FIG. 145.

19I. The solid form of any one of the previous embodiments, wherein the solid form of Compound 1 Lysine Type A is a crystalline polymorph of Compound 1 Lysine Type A characterized by a DSC curve having endotherms at about 74.2° C. and about 174.0° C.

### Solid Forms of Compound 1 Lysine Type B

20. The solid form of any one of the previous embodiments, wherein the solid form of solid form 1 is a crystalline Lysine Type B salt.

20A. The solid form of any one of the previous embodiments, wherein the solid form of Compound 1 Lysine Type B is a crystalline polymorph of Compound 1 Lysine Type B characterized by two or more, or three XRPD signals selected from the group consisting of 3.9 °2θ, 11.6 °2θ, and 15.5 °2θ (±0.2 °2θ; ±0.1 °2θ; or ±0.0 °2θ; Cu Kα1 radiation).

20B. The solid form of any one of the previous embodiments, wherein the solid form of Compound 1 Lysine Type B is a crystalline polymorph of Compound 1 Lysine Type B characterized by signals at 3.9 °2θ, 11.6 °2θ, and 15.5 °2θ (±0.2 °2θ; ±0.1 °2θ; or ±0.0 °2θ; Cu Kα1 radiation).

20C. The solid form of any one of the previous embodiments, wherein the solid form of Compound 1 Lysine Type B is a crystalline polymorph of Compound 1 Lysine Type B characterized by two or more, or three or more XRPD signals selected from the group consisting of 3.9 °2θ, 11.6 °2θ, 15.5 °2θ, 23.3 °2θ, and 7.8 °2θ (±0.2 °2θ; ±0.1 °2θ; or ±0.0 °2θ; Cu Kα1 radiation).

20D. The solid form of any one of the previous embodiments, wherein the solid form of Compound 1 Lysine Type B is a crystalline polymorph of Compound 1 Lysine Type B characterized by two or more, or three or more XRPD signals selected from the group consisting of 3.9 °2θ, 11.6 °2θ, 15.5 °2θ, 23.3 °2θ, 7.8 °2θ, 27.2 °2θ, and 19.4 °2θ (±0.2 °2θ; ±0.1 °2θ; or ±0.0 °2θ; Cu Kα1 radiation).

20E. The solid form of any one of the previous embodiments, wherein the solid form of Compound 1 Lysine Type B is a crystalline polymorph of Compound 1 Lysine Type B characterized by two or more, or three or more XRPD signals selected from the group consisting of 3.9 °2θ, 11.6 °2θ, 15.5 °2θ, 23.3 °2θ, 7.8 °2θ, 27.2 °2θ, 19.4 °2θ, 31.2 °2θ, 35.2 °2θ, and 0.0 °2θ (±0.2 °2θ; ±0.1 °2θ; or ±0.0 °2θ; Cu Kα1 radiation).

20F. The solid form of any one of the previous embodiments, wherein the solid form of Compound 1 Lysine Type B is a crystalline polymorph of Compound 1 Lysine Type B characterized by a XRPD diffractogram substantially similar to that shown in FIG. 53.

20G. The solid form of any one of the previous embodiments, wherein the solid form of Compound 1 Lysine Type B is a crystalline polymorph of Compound 1 Lysine Type B characterized by any combination of the XRPD peaks set forth in Table 15.

20H. The solid form of any one of the previous embodiments, wherein the solid form of Compound 1 Lysine Type B is a crystalline polymorph of Compound 1 Lysine Type B characterized by a 1H NMR spectrum substantially similar to that shown in FIG. 146.

20I. The solid form of any one of the previous embodiments, wherein the solid form of Compound 1 Lysine Type B is a crystalline polymorph of Compound 1 Lysine Type B characterized by a DSC curve having an endotherm at about 207.7° C.

### Solid Forms of Compound 1 Sodium Type A

21. The solid form of any one of the previous embodiments, wherein the solid form of solid form 1 is a crystalline Sodium Type A salt.

21A. The solid form of any one of the previous embodiments, wherein the solid form of Compound 1 Sodium Type A is a crystalline polymorph of Compound 1 Sodium Type A characterized by two or more, or three XRPD signals selected from the group consisting of 10.0 °2θ, 16.5 °2θ, and 21.7 °2θ (±0.2 °2θ; ±0.1 °2θ; or ±0.0 °2θ; Cu Kα1 radiation).

21B. The solid form of any one of the previous embodiments, wherein the solid form of Compound 1 Sodium Type A is a crystalline polymorph of Compound 1 Sodium Type A characterized by signals at 10.0 °2θ, 16.5 °2θ, and 21.7 °2θ (±0.2 °2θ; ±0.1 °2θ; or ±0.0 °2θ; Cu Kα1 radiation).

21C. The solid form of any one of the previous embodiments, wherein the solid form of Compound 1 Sodium Type A is a crystalline polymorph of Compound 1 Sodium Type A characterized by two or more, or three or more XRPD signals selected from the group consisting of 10.0 °2θ, 16.5 °2θ, 21.7 °2θ, 9.4 °2θ, and 12.4 °2θ (±0.2 °2θ; ±0.1 °2θ; or ±0.0 °2θ; Cu Kα1 radiation).

21D. The solid form of any one of the previous embodiments, wherein the solid form of Compound 1 Sodium Type A is a crystalline polymorph of Compound 1 Sodium Type A characterized by two or more, or three or more XRPD signals selected from the group consisting of 10.0 °2θ, 16.5 °2θ, 21.7 °2θ, 9.4 °2θ, 12.4 °2θ, 8.4 °2θ, and 24.3 °2θ (±0.2 °2θ; ±0.1 °2θ; or ±0.0 °2θ; Cu Kα1 radiation).

21E. The solid form of any one of the previous embodiments, wherein the solid form of Compound 1 Sodium Type A is a crystalline polymorph of Compound 1 Sodium Type A characterized by two or more, or three or more XRPD signals selected from the group consisting of 10.0 °2θ, 16.5 °2θ, 21.7 °2θ, 9.4 °2θ, 12.4 °2θ, 8.4 °2θ, 24.3 °2θ, 19.4 °2θ, 8.9 °2θ, and 6.3 °2θ (±0.2 °2θ; ±0.1 °2θ; or ±0.0 °2θ; Cu Kα1 radiation).

21F. The solid form of any one of the previous embodiments, wherein the solid form of Compound 1 Sodium Type A is a crystalline polymorph of Compound 1 Sodium Type A characterized by a XRPD diffractogram substantially similar to that shown in FIG. 56.

21G. The solid form of any one of the previous embodiments, wherein the solid form of Compound 1 Sodium Type A is a crystalline polymorph of Compound 1 Sodium Type A characterized by any combination of the XRPD peaks set forth in Table 16.

21H. The solid form of any one of the previous embodiments, wherein the solid form of Compound 1 Sodium Type A is a crystalline polymorph of Compound 1 Sodium Type A characterized by a 1H NMR spectrum substantially similar to that shown in FIG. 147.

21I. The solid form of any one of the previous embodiments, wherein the solid form of Compound 1 Sodium Type A is a crystalline polymorph of Compound 1 Sodium Type A characterized by a DSC curve having endotherms at about 92.2° C. and about 135.5° C.

### Solid Forms of Compound 1 Potassium Type B

22. The solid form of any one of the previous embodiments, wherein the solid form of solid form 1 is a crystalline Potassium Type B salt.

22A. The solid form of any one of the previous embodiments, wherein the solid form of Compound 1 Potassium Type B is a crystalline polymorph of Compound 1 Potassium Type B characterized by two or more, or three XRPD signals selected from the group consisting of 14.3 °2θ, 17.2 °2θ, and 5.8 °2θ (±0.2 °2θ; ±0.1 °2θ; or ±0.0 °2θ; Cu Kα1 radiation).

22B. The solid form of any one of the previous embodiments, wherein the solid form of Compound 1 Potassium Type B is a crystalline polymorph of Compound 1 Potassium Type B characterized by signals at 14.3 °2θ, 17.2 °2θ, and 5.8 °2θ (±0.2 °2θ; ±0.1 °2θ; or ±0.0 °2θ; Cu Kα1 radiation).

22C. The solid form of any one of the previous embodiments, wherein the solid form of Compound 1 Potassium Type B is a crystalline polymorph of Compound 1 Potassium Type B characterized by two or more, or three or more XRPD signals selected from the group consisting of 14.3 °2θ, 17.2 °2θ, 5.8 °2θ, 12.5 °2θ, and 8.6 °2θ (±0.2 °2θ; ±0.1 °2θ; or ±0.0 °2θ; Cu Kα1 radiation).

22D. The solid form of any one of the previous embodiments, wherein the solid form of Compound 1 Potassium Type B is a crystalline polymorph of Compound 1 Potassium Type B characterized by two or more, or three or more XRPD signals selected from the group consisting of 14.3 °2θ, 17.2 °2θ, 5.8 °2θ, 12.5 °2θ, 8.6 °2θ, 21.1 °2θ, and 11.4 °2θ (±0.2 °2θ; ±0.1 °2θ; or ±0.0 °2θ; Cu Kα1 radiation).

22E. The solid form of any one of the previous embodiments, wherein the solid form of Compound 1 Potassium Type B is a crystalline polymorph of Compound 1 Potassium Type B characterized by two or more, or three or more XRPD signals selected from the group consisting of 14.3 °2θ, 17.2 °2θ, 5.8 °2θ, 12.5 °2θ, 8.6 °2θ, 21.1 °2θ, 11.4 °2θ, 22.3 °2θ, 20.5 °2θ, and 23.0 °2θ (±0.2 °2θ; ±0.1 °2θ; or ±0.0 °2θ; Cu Kα1 radiation).

22F. The solid form of any one of the previous embodiments, wherein the solid form of Compound 1 Potassium Type B is a crystalline polymorph of Compound 1 Potassium Type B characterized by a XRPD diffractogram substantially similar to that shown in FIG. 59.

22G. The solid form of any one of the previous embodiments, wherein the solid form of Compound 1 Potassium Type B is a crystalline polymorph of Compound 1 Potassium Type B characterized by any combination of the XRPD peaks set forth in Table 17.

22H. The solid form of any one of the previous embodiments, wherein the solid form of Compound 1 Potassium Type B is a crystalline polymorph of Compound 1 Potassium Type B characterized by a 1H NMR spectrum substantially similar to that shown in FIG. 148.

22I. The solid form of any one of the previous embodiments, wherein the solid form of Compound 1 Potassium Type B is a crystalline polymorph of Compound 1 Potassium Type B characterized by a DSC curve having endotherms at about 150.6° C., about 224.0° C., and about 232.8° C.

### Solid Forms of Compound 1 Choline Type A

23. The solid form of any one of the previous embodiments, wherein the solid form of solid form 1 is a crystalline Choline Type A salt.

23A. The solid form of any one of the previous embodiments, wherein the solid form of Compound 1 Choline Type A is a crystalline polymorph of Compound 1 Choline Type A characterized by two or more, or three XRPD signals selected from the group consisting of 10.8 °2θ, 17.6 °2θ, and 15.8 °2θ (±0.2 °2θ; ±0.1 °2θ; or ±0.0 °2θ; Cu Kα1 radiation).

23B. The solid form of any one of the previous embodiments, wherein the solid form of Compound 1 Choline Type A is a crystalline polymorph of Compound 1 Choline Type A characterized by signals at 10.8 °2θ, 17.6 °2θ, and 15.8 °2θ (±0.2 °2θ; ±0.1 °2θ; or ±0.0 °2θ; Cu Kα1 radiation).

23C. The solid form of any one of the previous embodiments, wherein the solid form of Compound 1 Choline Type A is a crystalline polymorph of Compound 1 Choline Type A characterized by two or more, or three or more XRPD signals selected from the group consisting of 10.8 °2θ, 17.6 °2θ, 15.8 °2θ, 22.3 °2θ, and 21.7 °2θ (±0.2 °2θ; ±0.1 °2θ; or ±0.0 °2θ; Cu Kα1 radiation).

23D. The solid form of any one of the previous embodiments, wherein the solid form of Compound 1 Choline Type A is a crystalline polymorph of Compound 1 Choline Type A characterized by two or more, or three or more XRPD signals selected from the group consisting of 10.8 °2θ, 17.6 °2θ, 15.8 °2θ, 22.3 °2θ, 21.7 °2θ, 17.9 °2θ, and 23.8 °2θ (±0.2 °2θ; ±0.1 °2θ; or ±0.0 °2θ; Cu Kα1 radiation).

23E. The solid form of any one of the previous embodiments, wherein the solid form of Compound 1 Choline Type A is a crystalline polymorph of Compound 1 Choline Type A characterized by two or more, or three or more XRPD signals selected from the group consisting of 10.8 °2θ, 17.6 °2θ, 15.8 °2θ, 22.3 °2θ, 21.7 °2θ, 17.9 °2θ, 23.8 °2θ, 19.8 °2θ, 20.5 °2θ, and 24.7 °2θ (±0.2 °2θ; ±0.1 °2θ; or ±0.0 °2θ; Cu Kα1 radiation).

23F. The solid form of any one of the previous embodiments, wherein the solid form of Compound 1 Choline Type A is a crystalline polymorph of Compound 1 Choline Type A characterized by a XRPD diffractogram substantially similar to that shown in FIG. 62.

23G. The solid form of any one of the previous embodiments, wherein the solid form of Compound 1 Choline Type A is a crystalline polymorph of Compound 1 Choline Type A characterized by any combination of the XRPD peaks set forth in Table 18.

23H. The solid form of any one of the previous embodiments, wherein the solid form of Compound 1 Choline Type A is a crystalline polymorph of Compound 1 Choline Type A characterized by a 1H NMR spectrum substantially similar to that shown in FIG. 149.

23I. The solid form of any one of the previous embodiments, wherein the solid form of Compound 1 Choline Type A is a crystalline polymorph of Compound 1 Choline Type A characterized by a DSC curve having endotherms at about 184.5° C. and about 210.4° C.

### Solid Forms of Compound 1 Ammonium Type A

24. The solid form of any one of the previous embodiments, wherein the solid form of solid form 1 is a crystalline Ammonium Type A salt.

24A. The solid form of any one of the previous embodiments, wherein the solid form of Compound 1 Ammonium Type A is a crystalline polymorph of Compound 1 Ammonium Type A characterized by two or more, or three XRPD signals selected from the group consisting of 11.7 °2θ, 22.0 °2θ, and 13.6 °2θ (±0.2 °2θ; ±0.1 °2θ; or ±0.0 °2θ; Cu Kα1 radiation).

24B. The solid form of any one of the previous embodiments, wherein the solid form of Compound 1 Ammonium Type A is a crystalline polymorph of Compound 1 Ammonium Type A characterized by signals at 11.7 °2θ, 22.0 °2θ, and 13.6 °2θ (±0.2 °2θ; ±0.1 °2θ; or ±0.0 °2θ; Cu Kα1 radiation).

24C. The solid form of any one of the previous embodiments, wherein the solid form of Compound 1 Ammonium Type A is a crystalline polymorph of Compound 1 Ammonium Type A characterized by two or more, or three or more XRPD signals selected from the group consisting of 11.7 °2θ, 22.0 °2θ, 13.6 °2θ, 25.3 °2θ, and 23.5 °2θ (±0.2 °2θ; ±0.1 °2θ; or ±0.0 °2θ; Cu Kα1 radiation).

24D. The solid form of any one of the previous embodiments, wherein the solid form of Compound 1 Ammonium Type A is a crystalline polymorph of Compound 1 Ammonium Type A characterized by two or more, or three or more XRPD signals selected from the group consisting of 11.7 °2θ, 22.0 °2θ, 13.6 °2θ, 25.3 °2θ, 23.5 °2θ, 16.0 °2θ, and 21.7 °2θ (±0.2 °2θ; ±0.1 °2θ; or ±0.0 °2θ; Cu Kα1 radiation).

24E. The solid form of any one of the previous embodiments, wherein the solid form of Compound 1 Ammonium Type A is a crystalline polymorph of Compound 1 Ammonium Type A characterized by two or more, or three or more XRPD signals selected from the group consisting of 11.7 °2θ, 22.0 °2θ, 13.6 °2θ, 25.3 °2θ, 23.5 °2θ, 16.0 °2θ, 21.7 °2θ, 26.4 °2θ, 24.3 °2θ, and 16.5 °2θ (±0.2 °2θ; ±0.1 °2θ; or ±0.0 °2θ; Cu Kα1 radiation).

24F. The solid form of any one of the previous embodiments, wherein the solid form of Compound 1 Ammonium Type A is a crystalline polymorph of Compound 1 Ammonium Type A characterized by a XRPD diffractogram substantially similar to that shown in FIG. 65.

24G. The solid form of any one of the previous embodiments, wherein the solid form of Compound 1 Ammonium Type A is a crystalline polymorph of Compound 1 Ammonium Type A characterized by any combination of the XRPD peaks set forth in Table 19.

24H. The solid form of any one of the previous embodiments, wherein the solid form of Compound 1 Ammonium Type A is a crystalline polymorph of Compound 1 Ammonium Type A characterized by a 1H NMR spectrum substantially similar to that shown in FIG. 150.

24I. The solid form of any one of the previous embodiments, wherein the solid form of Compound 1 Ammonium Type A is a crystalline polymorph of Compound 1 Ammonium Type A characterized by a DSC curve having endotherms at about 132.8° C., about 156.2° C., and about 192.2° C.

### Solid Forms of Compound 1 Ammonium Type B

25. The solid form of any one of the previous embodiments, wherein the solid form of solid form 1 is a crystalline Ammonium Type B salt.

25A. The solid form of any one of the previous embodiments, wherein the solid form of Compound 1 Ammonium Type B is a crystalline polymorph of Compound 1 Ammonium Type B characterized by two or more, or three XRPD signals selected from the group consisting of 10.7 °2θ, 17.6 °2θ, and 18.4 °2θ (±0.2 °2θ; ±0.1 °2θ; or ±0.0 °2θ; Cu Kα1 radiation).

25B. The solid form of any one of the previous embodiments, wherein the solid form of Compound 1 Ammonium Type B is a crystalline polymorph of Compound 1 Ammonium Type B characterized by signals at 10.7 °2θ, 17.6 °2θ, and 18.4 °2θ (±0.2 °2θ; ±0.1 °2θ; or ±0.0 °2θ; Cu Kα1 radiation).

25C. The solid form of any one of the previous embodiments, wherein the solid form of Compound 1 Ammonium Type B is a crystalline polymorph of Compound 1 Ammonium Type B characterized by two or more, or three or more XRPD signals selected from the group consisting of 10.7 °2θ, 17.6 °2θ, 18.4 °2θ, 19.3 °2θ, and 15.4 °2θ (±0.2 °2θ; ±0.1 °2θ; or ±0.0 °2θ; Cu Kα1 radiation).

25D. The solid form of any one of the previous embodiments, wherein the solid form of Compound 1 Ammonium Type B is a crystalline polymorph of Compound 1 Ammonium Type B characterized by two or more, or three or more XRPD signals selected from the group consisting of 10.7 °2θ, 17.6 °2θ, 18.4 °2θ, 19.3 °2θ, 15.4 °2θ, 21.5 °2θ, and 23.8 °2θ (±0.2 °2θ; ±0.1 °2θ; or ±0.0 °2θ; Cu Kα1 radiation).

25E. The solid form of any one of the previous embodiments, wherein the solid form of Compound 1 Ammonium Type B is a crystalline polymorph of Compound 1 Ammonium Type B characterized by two or more, or three or more XRPD signals selected from the group consisting of 10.7 °2θ, 17.6 °2θ, 18.4 °2θ, 19.3 °2θ, 15.4 °2θ, 21.5 °2θ, 23.8 °2θ, 18.0 °2θ, 9.6 °2θ, and 10.9 °2θ (±0.2 °2θ; ±0.1 °2θ; or ±0.0 °2θ; Cu Kα1 radiation).

25F. The solid form of any one of the previous embodiments, wherein the solid form of Compound 1 Ammonium Type B is a crystalline polymorph of Compound 1 Ammonium Type B characterized by a XRPD diffractogram substantially similar to that shown in FIG. 68.

25G. The solid form of any one of the previous embodiments, wherein the solid form of Compound 1 Ammonium Type B is a crystalline polymorph of Compound 1 Ammonium Type B characterized by any combination of the XRPD peaks set forth in Table 20.

25H. The solid form of any one of the previous embodiments, wherein the solid form of Compound 1 Ammonium Type B is a crystalline polymorph of Compound 1 Ammonium Type B characterized by a 1H NMR spectrum substantially similar to that shown in FIG. 151.

25I. The solid form of any one of the previous embodiments, wherein the solid form of Compound 1 Ammonium Type B is a crystalline polymorph of Compound 1 Ammonium Type B characterized by a DSC curve having endotherms at about 140.7° C. and about 189.0° C.

### Solid Forms of Compound 1 Tris Type A

26. The solid form of any one of the previous embodiments, wherein the solid form of solid form 1 is a crystalline Tris Type A salt.

26A. The solid form of any one of the previous embodiments, wherein the solid form of Compound 1 Tris Type A is a crystalline polymorph of Compound 1 Tris Type A characterized by two or more, or three XRPD signals selected from the group consisting of 3.9 °2θ, 15.5 °2θ, and 7.7 °2θ (±0.2 °2θ; ±0.1 °2θ; or ±0.0 °2θ; Cu Kα1 radiation).

26B. The solid form of any one of the previous embodiments, wherein the solid form of Compound 1 Tris Type A is a crystalline polymorph of Compound 1 Tris Type A characterized by signals at 3.9 °2θ, 15.5 °2θ, and 7.7 °2θ (±0.2 °2θ; ±0.1 °2θ; or ±0.0 °2θ; Cu Kα1 radiation).

26C. The solid form of any one of the previous embodiments, wherein the solid form of Compound 1 Tris Type A is a crystalline polymorph of Compound 1 Tris Type A characterized by two or more, or three or more XRPD signals selected from the group consisting of 3.9 °2θ, 15.5 °2θ, 7.7 °2θ, 19.2 °2θ, and 14.7 °2θ (±0.2 °2θ; ±0.1 °2θ; or ±0.0 °2θ; Cu Kα1 radiation).

26D. The solid form of any one of the previous embodiments, wherein the solid form of Compound 1 Tris Type A is a crystalline polymorph of Compound 1 Tris Type A characterized by two or more, or three or more XRPD signals selected from the group consisting of 3.9 °2θ, 15.5 °2θ, 7.7 °2θ, 19.2 °2θ, 14.7 °2θ, 22.0 °2θ, and 20.2 °2θ (±0.2 °2θ; ±0.1 °2θ; or ±0.0 °2θ; Cu Kα1 radiation).

26E. The solid form of any one of the previous embodiments, wherein the solid form of Compound 1 Tris Type A is a crystalline polymorph of Compound 1 Tris Type A characterized by two or more, or three or more XRPD signals selected from the group consisting of 3.9 °2θ, 15.5 °2θ, 7.7 °2θ, 19.2 °2θ, 14.7 °2θ, 22.0 °2θ, 20.2 °2θ, 17.9 °2θ, 22.9 °2θ, and 8.9 °2θ (±0.2 °2θ; ±0.1 °2θ; or ±0.0 °2θ; Cu Kα1 radiation).

26F. The solid form of any one of the previous embodiments, wherein the solid form of Compound 1 Tris Type A is a crystalline polymorph of Compound 1 Tris Type A characterized by a XRPD diffractogram substantially similar to that shown in FIG. 71.

26G. The solid form of any one of the previous embodiments, wherein the solid form of Compound 1 Tris Type A is a crystalline polymorph of Compound 1 Tris Type A characterized by any combination of the XRPD peaks set forth in Table 21.

26H. The solid form of any one of the previous embodiments, wherein the solid form of Compound 1 Tris Type A is a crystalline polymorph of Compound 1 Tris Type A characterized by a 1H NMR spectrum substantially similar to that shown in FIG. 152.

26I. The solid form of any one of the previous embodiments, wherein the solid form of Compound 1 Tris Type A is a crystalline polymorph of Compound 1 Tris Type characterized by a DSC curve having endotherms at about 133.1° C., about 182.0, and about 191.2° C.

### Solid Forms of Compound 1 Tris Type B

27. The solid form of any one of the previous embodiments, wherein the solid form of solid form 1 is a crystalline Tris Type B salt.

27A. The solid form of any one of the previous embodiments, wherein the solid form of Compound 1 Tris Type B is a crystalline polymorph of Compound 1 Tris Type B characterized by two or more, or three XRPD signals selected from the group consisting of 18.8 °2θ, 12.1 °2θ, and 17.9 °2θ (±0.2 °2θ; ±0.1 °2θ; or ±0.0 °2θ; Cu Kα1 radiation).

27B. The solid form of any one of the previous embodiments, wherein the solid form of Compound 1 Tris Type B is a crystalline polymorph of Compound 1 Tris Type B characterized by signals at 18.8 °2θ, 12.1 °2θ, and 17.9 °2θ (±0.2 °2θ; ±0.1 °2θ; or ±0.0 °2θ; Cu Kα1 radiation).

27C. The solid form of any one of the previous embodiments, wherein the solid form of Compound 1 Tris Type B is a crystalline polymorph of Compound 1 Tris Type B characterized by two or more, or three or more XRPD signals selected from the group consisting of 18.8 °2θ, 12.1 °2θ, 17.9 °2θ, 15.7 °2θ, and 21.4 °2θ (±0.2 °2θ; ±0.1 °2θ; or ±0.0 °2θ; Cu Kα1 radiation).

27D. The solid form of any one of the previous embodiments, wherein the solid form of Compound 1 Tris Type B is a crystalline polymorph of Compound 1 Tris Type B characterized by two or more, or three or more XRPD signals selected from the group consisting of 18.8 °2θ, 12.1 °2θ, 17.9 °2θ, 15.7 °2θ, 21.4 °2θ, 11.3 °2θ, and 6.5 °2θ (±0.2 °2θ; ±0.1 °2θ; or ±0.0 °2θ; Cu Kα1 radiation).

27E. The solid form of any one of the previous embodiments, wherein the solid form of Compound 1 Tris Type B is a crystalline polymorph of Compound 1 Tris Type B characterized by two or more, or three or more XRPD signals selected from the group consisting of 18.8 °2θ, 12.1 °2θ, 17.9 °2θ, 15.7 °2θ, 21.4 °2θ, 11.3 °2θ, 6.5 °2θ, 22.6 °2θ, 8.4 °2θ, and 19.9 °2θ (±0.2 °2θ; ±0.1 °2θ; or ±0.0 °2θ; Cu Kα1 radiation).

27F. The solid form of any one of the previous embodiments, wherein the solid form of Compound 1 Tris Type B is a crystalline polymorph of Compound 1 Tris Type B characterized by a XRPD diffractogram substantially similar to that shown in FIG. 74.

27G. The solid form of any one of the previous embodiments, wherein the solid form of Compound 1 Tris Type B is a crystalline polymorph of Compound 1 Tris Type B characterized by any combination of the XRPD peaks set forth in Table 22.

27H. The solid form of any one of the previous embodiments, wherein the solid form of Compound 1 Tris Type B is a crystalline polymorph of Compound 1 Tris Type B characterized by a 1H NMR spectrum substantially similar to that shown in FIG. 153.

27I. The solid form of any one of the previous embodiments, wherein the solid form of Compound 1 Tris Type B is a crystalline polymorph of Compound 1 Tris Type B characterized by a DSC curve having endotherms at about 104.8° C. and about 191.6° C.

### Solid Forms of Compound 1 Meglumine Type B

28. The solid form of any one of the previous embodiments, wherein the solid form of solid form 1 is a crystalline Meglumine Type B salt.

28A. The solid form of any one of the previous embodiments, wherein the solid form of Compound 1 Meglumine Type B is a crystalline polymorph of Compound 1 Meglumine Type B characterized by two or more, or three XRPD signals selected from the group consisting of 4.0 °2θ, 19.7 °2θ, and 11.6 °2θ (±0.2 °2θ; ±0.1 °2θ; or ±0.0 °2θ; Cu Kα1 radiation).

28B. The solid form of any one of the previous embodiments, wherein the solid form of Compound 1 Meglumine Type B is a crystalline polymorph of Compound 1 Meglumine Type B characterized by signals at 4.0 °2θ, 19.7 °2θ, and 11.6 °2θ (±0.2 °2θ; ±0.1 °2θ; or ±0.0 °2θ; Cu Kα1 radiation).

28C. The solid form of any one of the previous embodiments, wherein the solid form of Compound 1 Meglumine Type B is a crystalline polymorph of Compound 1 Meglumine Type B characterized by two or more, or three or more XRPD signals selected from the group consisting of 4.0 20, 19.7 20, 11.6 20, 15.1 °2θ, and 11.8 °2θ (±0.2 °2θ; ±0.1 °2θ; or ±0.0 °2θ; Cu Kα1 radiation).

28D. The solid form of any one of the previous embodiments, wherein the solid form of Compound 1 Meglumine Type B is a crystalline polymorph of Compound 1 Meglumine Type B characterized by two or more, or three or more XRPD signals selected from the group consisting of 4.0 °2θ, 19.7 °2θ, 11.6 °2θ, 15.1 °2θ, 11.8 °2θ, 15.8 °2θ, and 7.9 °2θ (±0.2 °2θ; ±0.1 °2θ; or ±0.0 °2θ; Cu Kα1 radiation).

28E. The solid form of any one of the previous embodiments, wherein the solid form of Compound 1 Meglumine Type B is a crystalline polymorph of Compound 1 Meglumine Type B characterized by two or more, or three or more XRPD signals selected from the group consisting of 4.0 °2θ, 19.7 °2θ, 11.6 °2θ, 15.1 °2θ, 11.8 °2θ, 15.8 °2θ, 7.9 °2θ, 13.6 °2θ, 20.0 °2θ, and 23.7 °2θ (±0.2 °2θ; ±0.1 °2θ; or ±0.0 °2θ; Cu Kα1 radiation).

28F. The solid form of any one of the previous embodiments, wherein the solid form of Compound 1 Meglumine Type B is a crystalline polymorph of Compound 1 Meglumine Type B characterized by a XRPD diffractogram substantially similar to that shown in FIG. 77.

28G. The solid form of any one of the previous embodiments, wherein the solid form of Compound 1 Meglumine Type B is a crystalline polymorph of Compound 1 Meglumine Type B characterized by any combination of the XRPD peaks set forth in Table 25.

28H. The solid form of any one of the previous embodiments, wherein the solid form of Compound 1 Meglumine Type B is a crystalline polymorph of Compound 1 Meglumine Type B characterized by a 1H NMR spectrum substantially similar to that shown in FIG. 154.

28I. The solid form of any one of the previous embodiments, wherein the solid form of Compound 1 Meglumine Type B is a crystalline polymorph of Compound 1 Meglumine Type B characterized by a DSC curve having endotherms at about 80.9° C., about 132.2° and about 167.1° C.

### Solid Forms of Compound 1 Meglumine Type C

29. The solid form of any one of the previous embodiments, wherein the solid form of solid form 1 is a crystalline Meglumine Type C salt.

29A. The solid form of any one of the previous embodiments, wherein the solid form of Compound 1 Meglumine Type C is a crystalline polymorph of Compound 1 Meglumine Type C characterized by two or more, or three XRPD signals selected from the group consisting of 12.7 °2θ, 20.2 °2θ, and 17.1 °2θ (±0.2 °2θ; ±0.1 °2θ; or ±0.0 °2θ; Cu Kα1 radiation).

29B. The solid form of any one of the previous embodiments, wherein the solid form of Compound 1 Meglumine Type C is a crystalline polymorph of Compound 1 Meglumine Type C characterized by signals at 12.7 °2θ, 20.2 °2θ, and 17.1 °2θ (±0.2 °2θ; ±0.1 °2θ; or ±0.0 °2θ; Cu Kα1 radiation).

29C. The solid form of any one of the previous embodiments, wherein the solid form of Compound 1 Meglumine Type C is a crystalline polymorph of Compound 1 Meglumine Type C characterized by two or more, or three or more XRPD signals selected from the group consisting of 12.7 °2θ, 20.2 °2θ, 17.1 °2θ, 21.4 °2θ, and 24.4 °2θ (±0.2 °2θ; ±0.1 °2θ; or ±0.0 °2θ; Cu Kα1 radiation).

29D. The solid form of any one of the previous embodiments, wherein the solid form of Compound 1 Meglumine Type C is a crystalline polymorph of Compound 1 Meglumine Type C characterized by two or more, or three or more XRPD signals selected from the group consisting of 12.7 °2θ, 20.2 °2θ, 17.1 °2θ, 21.4 °2θ, 24.4 °2θ, 8.4 °2θ, and 18.6 °2θ (±0.2 °2θ; ±0.1 °2θ; or ±0.0 °2θ; Cu Kα1 radiation).

29E. The solid form of any one of the previous embodiments, wherein the solid form of Compound 1 Meglumine Type C is a crystalline polymorph of Compound 1 Meglumine Type C characterized by two or more, or three or more XRPD signals selected from the group consisting of 12.7 °2θ, 20.2 °2θ, 17.1 °2θ, 21.4 °2θ, 24.4 °2θ, 8.4 °2θ, 18.6 °2θ, 13.4 °2θ, 22.9 °2θ, and 19.4 °2θ (±0.2 °2θ; ±0.1 °2θ; or ±0.0 °2θ; Cu Kα1 radiation).

29F. The solid form of any one of the previous embodiments, wherein the solid form of Compound 1 Meglumine Type C is a crystalline polymorph of Compound 1 Meglumine Type C characterized by a XRPD diffractogram substantially similar to that shown in FIG. 80.

29G. The solid form of any one of the previous embodiments, wherein the solid form of Compound 1 Meglumine Type C is a crystalline polymorph of Compound 1 Meglumine Type C characterized by any combination of the XRPD peaks set forth in Table 26.

29H. The solid form of any one of the previous embodiments, wherein the solid form of Compound 1 Meglumine Type C is a crystalline polymorph of Compound 1 Meglumine Type C characterized by a 1H NMR spectrum substantially similar to that shown in FIG. 155.

29I. The solid form of any one of the previous embodiments, wherein the solid form of Compound 1 Meglumine Type C is a crystalline polymorph of Compound 1 Meglumine Type C characterized by a DSC curve having an endotherm at about 166.7° C.

### Solid Forms of Compound 1 Freeform Type A

30. The solid form of any one of the previous embodiments, wherein the solid form of solid form 1 is crystalline Freeform Type A.

30A. The solid form of any one of the previous embodiments, wherein the solid form of Compound 1 Freeform Type A is a crystalline polymorph of Compound 1 Freeform Type A characterized by two or more, or three XRPD signals selected from the group consisting of 3.6 °2θ, 7.1 °2θ, and 17.6 °2θ (±0.2 °2θ; ±0.1 °2θ; or ±0.0 °2θ; Cu Kα1 radiation).

30B. The solid form of any one of the previous embodiments, wherein the solid form of Compound 1 Freeform Type A is a crystalline polymorph of Compound 1 Freeform Type A characterized by signals at 3.6 °2θ, 7.1 °2θ, and 17.6 °2θ (±0.2 °2θ; ±0.1 °2θ; or ±0.0 °2θ; Cu Kα1 radiation).

30C. The solid form of any one of the previous embodiments, wherein the solid form of Compound 1 Freeform Type A is a crystalline polymorph of Compound 1 Freeform Type A characterized by two or more, or three or more XRPD signals selected from the group consisting of 3.6 °2θ, 7.1 °2θ, 17.6 °2θ, 6.5 °2θ, and 12.6 °2θ (±0.2 °2θ; ±0.1 °2θ; or ±0.0 °2θ; Cu Kα1 radiation).

30D. The solid form of any one of the previous embodiments, wherein the solid form of Compound 1 Freeform Type A is a crystalline polymorph of Compound 1 Freeform Type A characterized by two or more, or three or more XRPD signals selected from the group consisting of 3.6 °2θ, 7.1 °2θ, 17.6 °2θ, 6.5 °2θ, 12.6 °2θ, 10.6 °2θ, and 14.1 °2θ (±0.2 °2θ; ±0.1 °2θ; or ±0.0 °2θ; Cu Kα1 radiation).

30E. The solid form of any one of the previous embodiments, wherein the solid form of Compound 1 Freeform Type A is a crystalline polymorph of Compound 1 Freeform Type A characterized by two or more, or three or more XRPD signals selected from the group consisting of 3.6 °2θ, 7.1 °2θ, 17.6 °2θ, 6.5 °2θ, 12.6 °2θ, 10.6 °2θ, 14.1 °2θ, 15.9 °2θ, 0.0 °2θ, and 0.0 °2θ (±0.2 °2θ; ±0.1 °2θ; or ±0.0 °2θ; Cu Kα1 radiation).

30F. The solid form of any one of the previous embodiments, wherein the solid form of Compound 1 Freeform Type A is a crystalline polymorph of Compound 1 Freeform Type A characterized by a XRPD diffractogram substantially similar to that shown in FIG. 83.

30G. The solid form of any one of the previous embodiments, wherein the solid form of Compound 1 Freeform Type A is a crystalline polymorph of Compound 1 Freeform Type A characterized by any combination of the XRPD peaks set forth in Table 27.

30H. The solid form of any one of the previous embodiments, wherein the solid form of Compound 1 Freeform Type A is a crystalline polymorph of Compound 1 Freeform Type A characterized by a 1H NMR spectrum substantially similar to that shown in FIG. 156.

30I. The solid form of any one of the previous embodiments, wherein the solid form of Compound 1 Freeform Type A is a crystalline polymorph of Compound 1 Freeform Type A characterized by a DSC curve having endotherms at about 103.6° C., about 161.5° C., and about 185.6° C.

### Solid Forms of Compound 1 Freeform Type B

31. The solid form of any one of the previous embodiments, wherein the solid form of solid form 1 is crystalline Freeform Type A.

31A. The solid form of any one of the previous embodiments, wherein the solid form of Compound 1 Freeform Type B is a crystalline polymorph of Compound 1 Freeform Type B characterized by two or more, or three XRPD signals selected from the group consisting of 16.5 °2θ, 21.7 °2θ, and 21.4 °2θ (±0.2 °2θ; ±0.1 °2θ; or ±0.0 °2θ; Cu Kα1 radiation).

31B. The solid form of any one of the previous embodiments, wherein the solid form of Compound 1 Freeform Type B is a crystalline polymorph of Compound 1 Freeform Type B characterized by signals at 16.5 °2θ, 21.7 °2θ, and 21.4 °2θ (±0.2 °2θ; ±0.1 °2θ; or ±0.0 °2θ; Cu Kα1 radiation).

31C. The solid form of any one of the previous embodiments, wherein the solid form of Compound 1 Freeform Type B is a crystalline polymorph of Compound 1 Freeform Type B characterized by two or more, or three or more XRPD signals selected from the group consisting of 16.5 °2θ, 21.7 °2θ, 21.4 °2θ, 8.9 °2θ, and 24.2 °2θ (±0.2 °2θ; ±0.1 °2θ; or ±0.0 °2θ; Cu Kα1 radiation).

31D. The solid form of any one of the previous embodiments, wherein the solid form of Compound 1 Freeform Type B is a crystalline polymorph of Compound 1 Freeform Type B characterized by two or more, or three or more XRPD signals selected from the group consisting of 16.5 °2θ, 21.7 °2θ, 21.4 °2θ, 8.9 °2θ, 24.2 °2θ, 22.1 °2θ, and 12.7 °2θ (±0.2 °2θ; ±0.1 °2θ; or ±0.0 °2θ; Cu Kα1 radiation).

31E. The solid form of any one of the previous embodiments, wherein the solid form of Compound 1 Freeform Type B is a crystalline polymorph of Compound 1 Freeform Type B characterized by two or more, or three or more XRPD signals selected from the group consisting of 16.5 °2θ, 21.7 °2θ, 21.4 °2θ, 8.9 °2θ, 24.2 °2θ, 22.1 °2θ, 12.7 °2θ, 19.4 °2θ, 8.4 °2θ, and 13.6 °2θ (±0.2 °2θ; ±0.1 °2θ; or ±0.0 °2θ; Cu Kα1 radiation).

31F. The solid form of any one of the previous embodiments, wherein the solid form of Compound 1 Freeform Type B is a crystalline polymorph of Compound 1 Freeform Type B characterized by a XRPD diffractogram substantially similar to that shown in FIG. 86.

31G. The solid form of any one of the previous embodiments, wherein the solid form of Compound 1 Freeform Type B is a crystalline polymorph of Compound 1 Freeform Type B characterized by any combination of the XRPD peaks set forth in Table 28.

31H. The solid form of any one of the previous embodiments, wherein the solid form of Compound 1 Freeform Type B is a crystalline polymorph of Compound 1 Freeform Type B characterized by a 1H NMR spectrum substantially similar to that shown in FIG. 157.

31I. The solid form of any one of the previous embodiments, wherein the solid form of Compound 1 Freeform Type B is a crystalline polymorph of Compound 1 Freeform Type B characterized by a DSC curve having endotherms at about 132.7° C., about 147.9° C., and about 189.0° C.

### Solid Forms of Compound 1 Freeform Type C

32. The solid form of any one of the previous embodiments, wherein the solid form of solid form 1 is crystalline Freeform Type C.

32A. The solid form of any one of the previous embodiments, wherein the solid form of Compound 1 Freeform Type C is a crystalline polymorph of Compound 1 Freeform Type C characterized by two or more, or three XRPD signals selected from the group consisting of 17.6 °2θ, 10.7 °2θ, and 18.0 °2θ (±0.2 °2θ; ±0.1 °2θ; or ±0.0 °2θ; Cu Kα1 radiation).

32B. The solid form of any one of the previous embodiments, wherein the solid form of Compound 1 Freeform Type C is a crystalline polymorph of Compound 1 Freeform Type C characterized by signals at 17.6 °2θ, 10.7 °2θ, and 18.0 °2θ (±0.2 °2θ; ±0.1 °2θ; or ±0.0 °2θ; Cu Kα1 radiation).

32C. The solid form of any one of the previous embodiments, wherein the solid form of Compound 1 Freeform Type C is a crystalline polymorph of Compound 1 Freeform Type C characterized by two or more, or three or more XRPD signals selected from the group consisting of 17.6 °2θ, 10.7 °2θ, 18.0 °2θ, 22.0 °2θ, and 21.5 °2θ (±0.2 °2θ; ±0.1 °2θ; or ±0.0 °2θ; Cu Kα1 radiation).

32D. The solid form of any one of the previous embodiments, wherein the solid form of Compound 1 Freeform Type C is a crystalline polymorph of Compound 1 Freeform Type C characterized by two or more, or three or more XRPD signals selected from the group consisting of 17.6 °2θ, 10.7 °2θ, 18.0 °2θ, 22.0 °2θ, 21.5 °2θ, 13.7 °2θ, and 23.8 °2θ (±0.2 °2θ; ±0.1 °2θ; or ±0.0 °2θ; Cu Kα1 radiation).

32E. The solid form of any one of the previous embodiments, wherein the solid form of Compound 1 Freeform Type C is a crystalline polymorph of Compound 1 Freeform Type C characterized by two or more, or three or more XRPD signals selected from the group consisting of 17.6 °2θ, 10.7 °2θ, 18.0 °2θ, 22.0 °2θ, 21.5 °2θ, 13.7 °2θ, 23.8 °2θ, 19.3 °2θ, 10.9 °2θ, and 27.1 °2θ (±0.2 °2θ; ±0.1 °2θ; or ±0.0 °2θ; Cu Kα1 radiation).

32F. The solid form of any one of the previous embodiments, wherein the solid form of Compound 1 Freeform Type C is a crystalline polymorph of Compound 1 Freeform Type C characterized by a XRPD diffractogram substantially similar to that shown in FIG. 89.

32G. The solid form of any one of the previous embodiments, wherein the solid form of Compound 1 Freeform Type C is a crystalline polymorph of Compound 1 Freeform Type C characterized by any combination of the XRPD peaks set forth in Table 29.

32H. The solid form of any one of the previous embodiments, wherein the solid form of Compound 1 Freeform Type C is a crystalline polymorph of Compound 1 Freeform Type C characterized by a 1H NMR spectrum substantially similar to that shown in FIG. 158.

32I. The solid form of any one of the previous embodiments, wherein the solid form of Compound 1 Freeform Type C is a crystalline polymorph of Compound 1 Freeform Type C characterized by a DSC curve having an endotherm at about 188.7° C.

Solid forms of Compound 1 Freeform Type D

33. The solid form of any one of the previous embodiments, wherein the solid form of solid form 1 is crystalline Freeform Type D.

33A. The solid form of any one of the previous embodiments, wherein the solid form of Compound 1 Freeform Type D is a crystalline polymorph of Compound 1 Freeform Type D characterized by two or more, or three XRPD signals selected from the group consisting of 8.7 °2θ, 17.5 °2θ, and 13.1 °2θ (±0.2 °2θ; ±0.1 °2θ; or ±0.0 °2θ; Cu Kα1 radiation).

33B. The solid form of any one of the previous embodiments, wherein the solid form of Compound 1 Freeform Type D is a crystalline polymorph of Compound 1 Freeform Type D characterized by signals at 8.7 °2θ, 17.5 °2θ, and 13.1 °2θ (±0.2 °2θ; ±0.1 °2θ; or ±0.0 °2θ; Cu Kα1 radiation).

33C. The solid form of any one of the previous embodiments, wherein the solid form of Compound 1 Freeform Type D is a crystalline polymorph of Compound 1 Freeform Type D characterized by two or more, or three or more XRPD signals selected from the group consisting of 8.7 °2θ, 17.5 °2θ, 13.1 °2θ, 21.9 °2θ, and 4.4 °2θ (±0.2 °2θ; ±0.1 °2θ; or ±0.0 °2θ; Cu Kα1 radiation).

33D. The solid form of any one of the previous embodiments, wherein the solid form of Compound 1 Freeform Type D is a crystalline polymorph of Compound 1 Freeform Type D characterized by two or more, or three or more XRPD signals selected from the group consisting of 8.7 °2θ, 17.5 °2θ, 13.1 °2θ, 21.9 °2θ, 4.4 °2θ, 10.5 °2θ, and 24.2 °2θ (±0.2 °2θ; ±0.1 °2θ; or ±0.0 °2θ; Cu Kα1 radiation).

33E. The solid form of any one of the previous embodiments, wherein the solid form of Compound 1 Freeform Type D is a crystalline polymorph of Compound 1 Freeform Type D characterized by two or more, or three or more XRPD signals selected from the group consisting of 8.7 °2θ, 17.5 °2θ, 13.1 °2θ, 21.9 °2θ, 4.4 °2θ, 10.5 °2θ, 24.2 °2θ, 16.9 °2θ, 20.4 °2θ, and 17.8 °2θ (±0.2 °2θ; ±0.1 °2θ; or ±0.0 °2θ; Cu Kα1 radiation).

33F. The solid form of any one of the previous embodiments, wherein the solid form of Compound 1 Freeform Type D is a crystalline polymorph of Compound 1 Freeform Type D characterized by a XRPD diffractogram substantially similar to that shown in FIG. 93.

33G. The solid form of any one of the previous embodiments, wherein the solid form of Compound 1 Freeform Type D is a crystalline polymorph of Compound 1 Freeform Type D characterized by any combination of the XRPD peaks set forth in Table 30.

33H. The solid form of any one of the previous embodiments, wherein the solid form of Compound 1 Freeform Type D is a crystalline polymorph of Compound 1 Freeform Type D characterized by a 1H NMR spectrum substantially similar to that shown in FIG. 159.

33I. The solid form of any one of the previous embodiments, wherein the solid form of Compound 1 Freeform Type D is a crystalline polymorph of Compound 1 Freeform Type D characterized by a DSC curve having endotherms at about 145.6° C. and about 148.6° C.

### Solid Forms of Compound 1 Freeform Type E

34. The solid form of any one of the previous embodiments, wherein the solid form of solid form 1 is crystalline Freeform Type E.

34A. The solid form of any one of the previous embodiments, wherein the solid form of Compound 1 Freeform Type E is a crystalline polymorph of Compound 1 Freeform Type E characterized by two or more, or three XRPD signals selected from the group consisting of 16.3 °2θ, 21.8 °2θ, and 24.1 °2θ (±0.2 °2θ; ±0.1 °2θ; or ±0.0 °2θ; Cu Kα1 radiation).

34B. The solid form of any one of the previous embodiments, wherein the solid form of Compound 1 Freeform Type E is a crystalline polymorph of Compound 1 Freeform Type E characterized by signals at 16.3 °2θ, 21.8 °2θ, and 24.1 °2θ (±0.2 °2θ; ±0.1 °2θ; or ±0.0 °2θ; Cu Kα1 radiation).

34C. The solid form of any one of the previous embodiments, wherein the solid form of Compound 1 Freeform Type E is a crystalline polymorph of Compound 1 Freeform Type E characterized by two or more, or three or more XRPD signals selected from the group consisting of 16.3 °2θ, 21.8 °2θ, 24.1 °2θ, 20.0 °2θ, and 22.2 °2θ (±0.2 °2θ; ±0.1 °2θ; or ±0.0 °2θ; Cu Kα1 radiation).

34D. The solid form of any one of the previous embodiments, wherein the solid form of Compound 1 Freeform Type E is a crystalline polymorph of Compound 1 Freeform Type E characterized by two or more, or three or more XRPD signals selected from the group consisting of 16.3 °2θ, 21.8 °2θ, 24.1 °2θ, 20.0 °2θ, 22.2 °2θ, 6.3 °2θ, and 12.7 °2θ (±0.2 °2θ; ±0.1 °2θ; or ±0.0 °2θ; Cu Kα1 radiation).

34E. The solid form of any one of the previous embodiments, wherein the solid form of Compound 1 Freeform Type E is a crystalline polymorph of Compound 1 Freeform Type E characterized by two or more, or three or more XRPD signals selected from the group consisting of 16.3 °2θ, 21.8 °2θ, 24.1 °2θ, 20.0 °2θ, 22.2 °2θ, 6.3 °2θ, 12.7 °2θ, 8.4 °2θ, 18.4 °2θ, and 9.1 °2θ (±0.2 °2θ; ±0.1 °2θ; or ±0.0 °2θ; Cu Kα1 radiation).

34F. The solid form of any one of the previous embodiments, wherein the solid form of Compound 1 Freeform Type E is a crystalline polymorph of Compound 1 Freeform Type E characterized by a XRPD diffractogram substantially similar to that shown in FIG. 96.

34G. The solid form of any one of the previous embodiments, wherein the solid form of Compound 1 Freeform Type E is a crystalline polymorph of Compound 1 Freeform Type E characterized by any combination of the XRPD peaks set forth in Table 31.

34H. The solid form of any one of the previous embodiments, wherein the solid form of Compound 1 Freeform Type E is a crystalline polymorph of Compound 1 Freeform Type E characterized by a 1H NMR spectrum substantially similar to that shown in FIG. 160.

34I. The solid form of any one of the previous embodiments, wherein the solid form of Compound 1 Freeform Type E is a crystalline polymorph of Compound 1 Freeform Type E characterized by a DSC curve having endotherms at about 127.3° C.

### Solid Forms of Compound 1 Freeform Type F

35. The solid form of any one of the previous embodiments, wherein the solid form of solid form 1 is crystalline Freeform Type F.

35A. The solid form of any one of the previous embodiments, wherein the solid form of Compound 1 Freeform Type F is a crystalline polymorph of Compound 1 Freeform Type F characterized by two or more, or three XRPD signals selected from the group consisting of 18.0 °2θ, 10.8 °2θ, and 14.4 °2θ (±0.2 °2θ; ±0.1 °2θ; or ±0.0 °2θ; Cu Kα1 radiation).

35B. The solid form of any one of the previous embodiments, wherein the solid form of Compound 1 Freeform Type F is a crystalline polymorph of Compound 1 Freeform Type F characterized by signals at 18.0 °2θ, 10.8 °2θ, and 14.4 °2θ (±0.2 °2θ; ±0.1 °2θ; or ±0.0 °2θ; Cu Kα1 radiation).

35C. The solid form of any one of the previous embodiments, wherein the solid form of Compound 1 Freeform Type F is a crystalline polymorph of Compound 1 Freeform Type F characterized by two or more, or three or more XRPD signals selected from the group consisting of 18.0 °2θ, 10.8 °2θ, 14.4 °2θ, 23.1 °2θ, and 8.9 °2θ (±0.2 °2θ; ±0.1 °2θ; or ±0.0 °2θ; Cu Kα1 radiation).

35D. The solid form of any one of the previous embodiments, wherein the solid form of Compound 1 Freeform Type F is a crystalline polymorph of Compound 1 Freeform Type F characterized by two or more, or three or more XRPD signals selected from the group consisting of 18.0 °2θ, 10.8 °2θ, 14.4 °2θ, 23.1 °2θ, 8.9 °2θ, 0.0 °2θ, and 0.0 °2θ (±0.2 °2θ; ±0.1 °2θ; or ±0.0 °2θ; Cu Kα1 radiation).

35E. The solid form of any one of the previous embodiments, wherein the solid form of Compound 1 Freeform Type F is a crystalline polymorph of Compound 1 Freeform Type F characterized by two or more, or three or more XRPD signals selected from the group consisting of 18.0 °2θ, 10.8 °2θ, 14.4 °2θ, 23.1 °2θ, 8.9 °2θ, 0.0 °2θ, 0.0 °2θ, 0.0 °2θ, 0.0 °2θ, and 0.0 °2θ (±0.2 °2θ; ±0.1 °2θ; or ±0.0 °2θ; Cu Kα1 radiation).

35F. The solid form of any one of the previous embodiments, wherein the solid form of Compound 1 Freeform Type F is a crystalline polymorph of Compound 1 Freeform Type F characterized by a XRPD diffractogram substantially similar to that shown in FIG. 99.

35G. The solid form of any one of the previous embodiments, wherein the solid form of Compound 1 Freeform Type F is a crystalline polymorph of Compound 1 Freeform Type F characterized by any combination of the XRPD peaks set forth in Table 32.

### Solid Forms of Compound 1 Freeform Type G

36. The solid form of any one of the previous embodiments, wherein the solid form of solid form 1 is crystalline Freeform Type G.

36A. The solid form of any one of the previous embodiments, wherein the solid form of Compound 1 Freeform Type G is a crystalline polymorph of Compound 1 Freeform Type G characterized by two or more, or three XRPD signals selected from the group consisting of 6.3 °2θ, 22.1 °2θ, and 9.2 °2θ (±0.2 °2θ; ±0.1 °2θ; or ±0.0 °2θ; Cu Kα1 radiation).

36B. The solid form of any one of the previous embodiments, wherein the solid form of Compound 1 Freeform Type G is a crystalline polymorph of Compound 1 Freeform Type G characterized by signals at 6.3 °2θ, 22.1 °2θ, and 9.2 °2θ (±0.2 °2θ; ±0.1 °2θ; or ±0.0 °2θ; Cu Kα1 radiation).

36C. The solid form of any one of the previous embodiments, wherein the solid form of Compound 1 Freeform Type G is a crystalline polymorph of Compound 1 Freeform Type G characterized by two or more, or three or more XRPD signals selected from the group consisting of 6.3 °2θ, 22.1 °2θ, 9.2 °2θ, 16.3 °2θ, and 19.9 °2θ (±0.2 °2θ; ±0.1 °2θ; or ±0.0 °2θ; Cu Kα1 radiation).

36D. The solid form of any one of the previous embodiments, wherein the solid form of Compound 1 Freeform Type G is a crystalline polymorph of Compound 1 Freeform Type G characterized by two or more, or three or more XRPD signals selected from the group consisting of 6.3 °2θ, 22.1 °2θ, 9.2 °2θ, 16.3 °2θ, 19.9 °2θ, 18.4 °2θ, and 12.7 °2θ (±0.2 °2θ; ±0.1 °2θ; or ±0.0 °2θ; Cu Kα1 radiation).

36E. The solid form of any one of the previous embodiments, wherein the solid form of Compound 1 Freeform Type G is a crystalline polymorph of Compound 1 Freeform Type G characterized by two or more, or three or more XRPD signals selected from the group consisting of 6.3 °2θ, 22.1 °2θ, 9.2 °2θ, 16.3 °2θ, 19.9 °2θ, 18.4 °2θ, 12.7 °2θ, 24.1 °2θ, 13.9 °2θ, and 19.0 °2θ (±0.2 °2θ; ±0.1 °2θ; or ±0.0 °2θ; Cu Kα1 radiation).

36F. The solid form of any one of the previous embodiments, wherein the solid form of Compound 1 Freeform Type G is a crystalline polymorph of Compound 1 Freeform Type G characterized by a XRPD diffractogram substantially similar to that shown in FIG. 100.

36G. The solid form of any one of the previous embodiments, wherein the solid form of Compound 1 Freeform Type G is a crystalline polymorph of Compound 1 Freeform Type G characterized by any combination of the XRPD peaks set forth in Table 33.

36H. The solid form of any one of the previous embodiments, wherein the solid form of Compound 1 Freeform Type G is a crystalline polymorph of Compound 1 Freeform Type G characterized by a 1H NMR spectrum substantially similar to that shown in FIG. 161.

36I. The solid form of any one of the previous embodiments, wherein the solid form of Compound 1 Freeform Type G is a crystalline polymorph of Compound 1 Freeform Type G characterized by a DSC curve having endotherms at about 107.6° C. and about 178.6° C.

### Solid Forms of Compound 1 Freeform Type H

37. The solid form of any one of the previous embodiments, wherein the solid form of solid form 1 is crystalline Freeform Type H.

37A. The solid form of any one of the previous embodiments, wherein the solid form of Compound 1 Freeform Type H is a crystalline polymorph of Compound 1 Freeform Type H characterized by two or more, or three XRPD signals selected from the group consisting of 17.4 °2θ, 12.5 °2θ, and 22.9 °2θ (±0.2 °2θ; ±0.1 °2θ; or ±0.0 °2θ; Cu Kα1 radiation).

37B. The solid form of any one of the previous embodiments, wherein the solid form of Compound 1 Freeform Type H is a crystalline polymorph of Compound 1 Freeform Type H characterized by signals at 17.4 °2θ, 12.5 °2θ, and 22.9 °2θ (±0.2 °2θ; ±0.1 °2θ; or ±0.0 °2θ; Cu Kα1 radiation).

37C. The solid form of any one of the previous embodiments, wherein the solid form of Compound 1 Freeform Type H is a crystalline polymorph of Compound 1 Freeform Type H characterized by two or more, or three or more XRPD signals selected from the group consisting of 17.4 °2θ, 12.5 °2θ, 22.9 °2θ, 12.2 °2θ, and 20.3 °2θ (±0.2 °2θ; ±0.1 °2θ; or ±0.0 °2θ; Cu Kα1 radiation).

37D. The solid form of any one of the previous embodiments, wherein the solid form of Compound 1 Freeform Type H is a crystalline polymorph of Compound 1 Freeform Type H characterized by two or more, or three or more XRPD signals selected from the group consisting of 17.4 °2θ, 12.5 °2θ, 22.9 °2θ, 12.2 °2θ, 20.3 °2θ, 7.0 °2θ, and 6.2 °2θ (±0.2 °2θ; ±0.1 °2θ; or ±0.0 °2θ; Cu Kα1 radiation).

37E. The solid form of any one of the previous embodiments, wherein the solid form of Compound 1 Freeform Type H is a crystalline polymorph of Compound 1 Freeform Type H characterized by two or more, or three or more XRPD signals selected from the group consisting of 17.4 °2θ, 12.5 °2θ, 22.9 °2θ, 12.2 °2θ, 20.3 °2θ, 7.0 °2θ, 6.2 °2θ, 14.3 °2θ, 10.4 °2θ, and 24.0 °2θ (±0.2 °2θ; ±0.1 °2θ; or ±0.0 °2θ; Cu Kα1 radiation).

37F. The solid form of any one of the previous embodiments, wherein the solid form of Compound 1 Freeform Type H is a crystalline polymorph of Compound 1 Freeform Type H characterized by a XRPD diffractogram substantially similar to that shown in FIG. 103.

37G. The solid form of any one of the previous embodiments, wherein the solid form of Compound 1 Freeform Type H is a crystalline polymorph of Compound 1 Freeform Type H characterized by any combination of the XRPD peaks set forth in Table 34. 37H. The solid form of any one of the previous embodiments, wherein the solid form of Compound 1 Freeform Type H is a crystalline polymorph of Compound 1 Freeform Type H characterized by a 1H NMR spectrum substantially similar to that shown in FIG. 162. 37I. The solid form of any one of the previous embodiments, wherein the solid form of Compound 1 Freeform Type H is a crystalline polymorph of Compound 1 Freeform Type H characterized by a DSC curve having endotherms at about 138.1° C. and about 190.6° C.

### Solid Forms of Compound 1 Freeform Type I

38. The solid form of any one of the previous embodiments, wherein the solid form of solid form 1 is crystalline Freeform Type I.

38A. The solid form of any one of the previous embodiments, wherein the solid form of Compound 1 Freeform Type I is a crystalline polymorph of Compound 1 Freeform Type I characterized by two or more, or three XRPD signals selected from the group consisting of 17.2 °2θ, 6.9 °2θ, and 10.4 °2θ (±0.2 °2θ; ±0.1 °2θ; or ±0.0 °2θ; Cu Kα1 radiation).

38B. The solid form of any one of the previous embodiments, wherein the solid form of Compound 1 Freeform Type I is a crystalline polymorph of Compound 1 Freeform Type I characterized by signals at 17.2 °2θ, 6.9 °2θ, and 10.4 °2θ (±0.2 °2θ; ±0.1 °2θ; or ±0.0 °2θ; Cu Kα1 radiation).

38C. The solid form of any one of the previous embodiments, wherein the solid form of Compound 1 Freeform Type I is a crystalline polymorph of Compound 1 Freeform Type I characterized by two or more, or three or more XRPD signals selected from the group consisting of 17.2 °2θ, 6.9 °2θ, 10.4 °2θ, 18.0 °2θ, and 3.5 °2θ (±0.2 °2θ; ±0.1 °2θ; or ±0.0 °2θ; Cu Kα1 radiation).

38D. The solid form of any one of the previous embodiments, wherein the solid form of Compound 1 Freeform Type I is a crystalline polymorph of Compound 1 Freeform Type I characterized by two or more, or three or more XRPD signals selected from the group consisting of 17.2 °2θ, 6.9 °2θ, 10.4 °2θ, 18.0 °2θ, 3.5 °2θ, 12.4 °2θ, and 23.0 °2θ (±0.2 °2θ; ±0.1 °2θ; or ±0.0 °2θ; Cu Kα1 radiation).

38E. The solid form of any one of the previous embodiments, wherein the solid form of Compound 1 Freeform Type I is a crystalline polymorph of Compound 1 Freeform Type I characterized by two or more, or three or more XRPD signals selected from the group consisting of 17.2 °2θ, 6.9 °2θ, 10.4 °2θ, 18.0 °2θ, 3.5 °2θ, 12.4 °2θ, 23.0 °2θ, 13.8 °2θ, 20.8 °2θ, and 0.0 °2θ (±0.2 °2θ; ±0.1 °2θ; or ±0.0 °2θ; Cu Kα1 radiation).

38F. The solid form of any one of the previous embodiments, wherein the solid form of Compound 1 Freeform Type I is a crystalline polymorph of Compound 1 Freeform Type I characterized by a XRPD diffractogram substantially similar to that shown in FIG. 106.

38G. The solid form of any one of the previous embodiments, wherein the solid form of Compound 1 Freeform Type I is a crystalline polymorph of Compound 1 Freeform Type I characterized by any combination of the XRPD peaks set forth in Table 35.

38H. The solid form of any one of the previous embodiments, wherein the solid form of Compound 1 Freeform Type I is a crystalline polymorph of Compound 1 Freeform Type I characterized by a 1H NMR spectrum substantially similar to that shown in FIG. 163.

38I. The solid form of any one of the previous embodiments, wherein the solid form of Compound 1 Freeform Type I is a crystalline polymorph of Compound 1 Freeform Type I characterized by a DSC curve having endotherms at about 115.5° C. and about 188.2° C.

### Solid Forms of Compound 1 Freeform Type J

39. The solid form of any one of the previous embodiments, wherein the solid form of solid form 1 is crystalline Freeform Type J.

39A. The solid form of any one of the previous embodiments, wherein the solid form of Compound 1 Freeform Type J is a crystalline polymorph of Compound 1 Freeform Type J characterized by two or more, or three XRPD signals selected from the group consisting of 18.9 °2θ, 7.5 °2θ, and 23.4 °2θ (±0.2 °2θ; ±0.1 °2θ; or ±0.0 °2θ; Cu Kα1 radiation).

39B. The solid form of any one of the previous embodiments, wherein the solid form of Compound 1 Freeform Type J is a crystalline polymorph of Compound 1 Freeform Type J characterized by signals at 18.9 °2θ, 7.5 °2θ, and 23.4 °2θ (±0.2 °2θ; ±0.1 °2θ; or ±0.0 °2θ; Cu Kα1 radiation).

39C. The solid form of any one of the previous embodiments, wherein the solid form of Compound 1 Freeform Type J is a crystalline polymorph of Compound 1 Freeform Type J characterized by two or more, or three or more XRPD signals selected from the group consisting of 18.9 °2θ, 7.5 °2θ, 23.4 °2θ, 22.7 °2θ, and 19.6 °2θ (±0.2 °2θ; ±0.1 °2θ; or ±0.0 °2θ; Cu Kα1 radiation).

39D. The solid form of any one of the previous embodiments, wherein the solid form of Compound 1 Freeform Type J is a crystalline polymorph of Compound 1 Freeform Type J characterized by two or more, or three or more XRPD signals selected from the group consisting of 18.9 °2θ, 7.5 °2θ, 23.4 °2θ, 22.7 °2θ, 19.6 °2θ, 11.8 °2θ, and 15.8 °2θ (±0.2 °2θ; ±0.1 °2θ; or ±0.0 °2θ; Cu Kα1 radiation).

39E. The solid form of any one of the previous embodiments, wherein the solid form of Compound 1 Freeform Type J is a crystalline polymorph of Compound 1 Freeform Type J characterized by two or more, or three or more XRPD signals selected from the group consisting of 18.9 °2θ, 7.5 °2θ, 23.4 °2θ, 22.7 °2θ, 19.6 °2θ, 11.8 °2θ, 15.8 °2θ, 15.1 °2θ, 3.8 °2θ, and 20.7 °2θ (±0.2 °2θ; ±0.1 °2θ; or ±0.0 °2θ; Cu Kα1 radiation).

39F. The solid form of any one of the previous embodiments, wherein the solid form of Compound 1 Freeform Type J is a crystalline polymorph of Compound 1 Freeform Type J characterized by a XRPD diffractogram substantially similar to that shown in FIG. 109.

39G. The solid form of any one of the previous embodiments, wherein the solid form of Compound 1 Freeform Type J is a crystalline polymorph of Compound 1 Freeform Type J characterized by any combination of the XRPD peaks set forth in Table 36.

39H. The solid form of any one of the previous embodiments, wherein the solid form of Compound 1 Freeform Type J is a crystalline polymorph of Compound 1 Freeform Type J characterized by a 1H NMR spectrum substantially similar to that shown in FIG. 164.

39I. The solid form of any one of the previous embodiments, wherein the solid form of Compound 1 Freeform Type J is a crystalline polymorph of Compound 1 Freeform Type J characterized by a DSC curve having endotherms at about 111.3° C. and about 187.7° C.

### Solid Forms of Compound 1 Freeform Type K

40. The solid form of any one of the previous embodiments, wherein the solid form of solid form 1 is crystalline Freeform Type K.

40A. The solid form of any one of the previous embodiments, wherein the solid form of Compound 1 Freeform Type K is a crystalline polymorph of Compound 1 Freeform Type K characterized by two or more, or three XRPD signals selected from the group consisting of 8.2 °2θ, 16.6 °2θ, and 11.6 °2θ (±0.2 °2θ; ±0.1 °2θ; or ±0.0 °2θ; Cu Kα1 radiation).

40B. The solid form of any one of the previous embodiments, wherein the solid form of Compound 1 Freeform Type K is a crystalline polymorph of Compound 1 Freeform Type K characterized by signals at 8.2 °2θ, 16.6 °2θ, and 11.6 °2θ (±0.2 °2θ; ±0.1 °2θ; or ±0.0 °2θ; Cu Kα1 radiation).

40C. The solid form of any one of the previous embodiments, wherein the solid form of Compound 1 Freeform Type K is a crystalline polymorph of Compound 1 Freeform Type K characterized by two or more, or three or more XRPD signals selected from the group consisting of 8.2 °2θ, 16.6 °2θ, 11.6 °2θ, 17.2 °2θ, and 16.2 °2θ (±0.2 °2θ; ±0.1 °2θ; or ±0.0 °2θ; Cu Kα1 radiation).

40D. The solid form of any one of the previous embodiments, wherein the solid form of Compound 1 Freeform Type K is a crystalline polymorph of Compound 1 Freeform Type K characterized by two or more, or three or more XRPD signals selected from the group consisting of 8.2 °2θ, 16.6 °2θ, 11.6 °2θ, 17.2 °2θ, 16.2 °2θ, 23.4 °2θ, and 11.3 °2θ (±0.2 °2θ; ±0.1 °2θ; or ±0.0 °2θ; Cu Kα1 radiation).

40E. The solid form of any one of the previous embodiments, wherein the solid form of Compound 1 Freeform Type K is a crystalline polymorph of Compound 1 Freeform Type K characterized by two or more, or three or more XRPD signals selected from the group consisting of 8.2 °2θ, 16.6 °2θ, 11.6 °2θ, 17.2 °2θ, 16.2 °2θ, 23.4 °2θ, 11.3 °2θ, 23.8 °2θ, 24.7 °2θ, and 21.9 °2θ (±0.2 °2θ; ±0.1 °2θ; or ±0.0 °2θ; Cu Kα1 radiation).

40F. The solid form of any one of the previous embodiments, wherein the solid form of Compound 1 Freeform Type K is a crystalline polymorph of Compound 1 Freeform Type K characterized by a XRPD diffractogram substantially similar to that shown in FIG. 112.

40G. The solid form of any one of the previous embodiments, wherein the solid form of Compound 1 Freeform Type K is a crystalline polymorph of Compound 1 Freeform Type K characterized by any combination of the XRPD peaks set forth in Table 37.

40H. The solid form of any one of the previous embodiments, wherein the solid form of Compound 1 Freeform Type K is a crystalline polymorph of Compound 1 Freeform Type K characterized by a 1H NMR spectrum substantially similar to that shown in FIG. 165.

40I. The solid form of any one of the previous embodiments, wherein the solid form of Compound 1 Freeform Type K is a crystalline polymorph of Compound 1 Freeform Type K characterized by a DSC curve having endotherms at about 61.1° C. and about 106.0° C.

### Solid Forms of Compound 1 Freeform Type L

41. The solid form of any one of the previous embodiments, wherein the solid form of solid form 1 is crystalline Freeform Type L.

41A. The solid form of any one of the previous embodiments, wherein the solid form of Compound 1 Freeform Type L is a crystalline polymorph of Compound 1 Freeform Type L characterized by two or more, or three XRPD signals selected from the group consisting of 17.1 °2θ, 6.8 °2θ, and 10.2 °2θ (±0.2 °2θ; ±0.1 °2θ; or ±0.0 °2θ; Cu Kα1 radiation).

41B. The solid form of any one of the previous embodiments, wherein the solid form of Compound 1 Freeform Type L is a crystalline polymorph of Compound 1 Freeform Type L characterized by signals at 17.1 °2θ, 6.8 °2θ, and 10.2 °2θ (±0.2 °2θ; ±0.1 °2θ; or ±0.0 °2θ; Cu Kα1 radiation).

41C. The solid form of any one of the previous embodiments, wherein the solid form of Compound 1 Freeform Type L is a crystalline polymorph of Compound 1 Freeform Type L characterized by two or more, or three or more XRPD signals selected from the group consisting of 17.1 °2θ, 6.8 °2θ, 10.2 °2θ, 13.5 °2θ, and 0.0 °2θ (±0.2 °2θ; ±0.1 °2θ; or ±0.0 °2θ; Cu Kα1 radiation).

41D. The solid form of any one of the previous embodiments, wherein the solid form of Compound 1 Freeform Type L is a crystalline polymorph of Compound 1 Freeform Type L characterized by two or more, or three or more XRPD signals selected from the group consisting of 17.1 °2θ, 6.8 °2θ, 10.2 °2θ, 13.5 °2θ, 0.0 °2θ, 0.0 °2θ, and 0.0 °2θ (±0.2 °2θ; ±0.1 °2θ; or ±0.0 °2θ; Cu Kα1 radiation).

41E. The solid form of any one of the previous embodiments, wherein the solid form of Compound 1 Freeform Type L is a crystalline polymorph of Compound 1 Freeform Type L characterized by two or more, or three or more XRPD signals selected from the group consisting of 17.1 °2θ, 6.8 °2θ, 10.2 °2θ, 13.5 °2θ, 0.0 °2θ, 0.0 °2θ, 0.0 °2θ, 0.0 °2θ, 0.0 °2θ, and 0.0 °2θ (±0.2 °2θ; ±0.1 °2θ; or ±0.0 °2θ; Cu Kα1 radiation).

41F. The solid form of any one of the previous embodiments, wherein the solid form of Compound 1 Freeform Type L is a crystalline polymorph of Compound 1 Freeform Type L characterized by a XRPD diffractogram substantially similar to that shown in FIG. 115.

41G. The solid form of any one of the previous embodiments, wherein the solid form of Compound 1 Freeform Type L is a crystalline polymorph of Compound 1 Freeform Type L characterized by any combination of the XRPD peaks set forth in Table 38.

41H. The solid form of any one of the previous embodiments, wherein the solid form of Compound 1 Freeform Type L is a crystalline polymorph of Compound 1 Freeform Type L characterized by a 1H NMR spectrum substantially similar to that shown in FIG. 166.

41I. The solid form of any one of the previous embodiments, wherein the solid form of Compound 1 Freeform Type L is a crystalline polymorph of Compound 1 Freeform Type L characterized by a DSC curve having endotherms at about 64.7° C., about 89.0° C. and about 188.3° C.

### Solid Forms of Compound 1 Freeform Type M

42. The solid form of any one of the previous embodiments, wherein the solid form of solid form 1 is crystalline Freeform Type M.

42A. The solid form of any one of the previous embodiments, wherein the solid form of Compound 1 Freeform Type M is a crystalline polymorph of Compound 1 Freeform Type M characterized by two or more, or three XRPD signals selected from the group consisting of 15.2 °2θ, 18.6 °2θ, and 17.8 °2θ (±0.2 °2θ; ±0.1 °2θ; or ±0.0 °2θ; Cu Kα1 radiation).

42B. The solid form of any one of the previous embodiments, wherein the solid form of Compound 1 Freeform Type M is a crystalline polymorph of Compound 1 Freeform Type M characterized by signals at 15.2 °2θ, 18.6 °2θ, and 17.8 °2θ (±0.2 °2θ; ±0.1 °2θ; or ±0.0 °2θ; Cu Kα1 radiation).

42C. The solid form of any one of the previous embodiments, wherein the solid form of Compound 1 Freeform Type M is a crystalline polymorph of Compound 1 Freeform Type M characterized by two or more, or three or more XRPD signals selected from the group consisting of 15.2 °2θ, 18.6 °2θ, 17.8 °2θ, 19.5 °2θ, and 23.2 °2θ (±0.2 °2θ; ±0.1 °2θ; or ±0.0 °2θ; Cu Kα1 radiation).

42D. The solid form of any one of the previous embodiments, wherein the solid form of Compound 1 Freeform Type M is a crystalline polymorph of Compound 1 Freeform Type M characterized by two or more, or three or more XRPD signals selected from the group consisting of 15.2 °2θ, 18.6 °2θ, 17.8 °2θ, 19.5 °2θ, 23.2 °2θ, 7.1 °2θ, and 11.6 °2θ (±0.2 °2θ; ±0.1 °2θ; or ±0.0 °2θ; Cu Kα1 radiation).

42E. The solid form of any one of the previous embodiments, wherein the solid form of Compound 1 Freeform Type M is a crystalline polymorph of Compound 1 Freeform Type M characterized by two or more, or three or more XRPD signals selected from the group consisting of 15.2 °2θ, 18.6 °2θ, 17.8 °2θ, 19.5 °2θ, 23.2 °2θ, 7.1 °2θ, 11.6 °2θ, 14.2 °2θ, 3.6 °2θ, and 26.4 °2θ (±0.2 °2θ; ±0.1 °2θ; or ±0.0 °2θ; Cu Kα1 radiation).

42F. The solid form of any one of the previous embodiments, wherein the solid form of Compound 1 Freeform Type M is a crystalline polymorph of Compound 1 Freeform Type M characterized by a XRPD diffractogram substantially similar to that shown in FIG. 118.

42G. The solid form of any one of the previous embodiments, wherein the solid form of Compound 1 Freeform Type M is a crystalline polymorph of Compound 1 Freeform Type M characterized by any combination of the XRPD peaks set forth in Table 39.

42H. The solid form of any one of the previous embodiments, wherein the solid form of Compound 1 Freeform Type M is a crystalline polymorph of Compound 1 Freeform Type M characterized by a 1H NMR spectrum substantially similar to that shown in FIG. 167.

42I. The solid form of any one of the previous embodiments, wherein the solid form of Compound 1 Freeform Type M is a crystalline polymorph of Compound 1 Freeform Type M characterized by a DSC curve having an endotherm at about 188.7° C.

### Solid Forms of Compound 1 Freeform Type N

43. The solid form of any one of the previous embodiments, wherein the solid form of solid form 1 is crystalline Freeform Type N.

43A. The solid form of any one of the previous embodiments, wherein the solid form of Compound 1 Freeform Type N is a crystalline polymorph of Compound 1 Freeform Type N characterized by two or more, or three XRPD signals selected from the group consisting of 17.2 °2θ, 22.5 °2θ, and 11.1 °2θ (±0.2 °2θ; ±0.1 °2θ; or ±0.0 °2θ; Cu Kα1 radiation).

43B. The solid form of any one of the previous embodiments, wherein the solid form of Compound 1 Freeform Type N is a crystalline polymorph of Compound 1 Freeform Type N characterized by signals at 17.2 °2θ, 22.5 °2θ, and 11.1 °2θ (±0.2 °2θ; ±0.1 °2θ; or ±0.0 °2θ; Cu Kα1 radiation).

43C. The solid form of any one of the previous embodiments, wherein the solid form of Compound 1 Freeform Type N is a crystalline polymorph of Compound 1 Freeform Type N characterized by two or more, or three or more XRPD signals selected from the group consisting of 17.2 °2θ, 22.5 °2θ, 11.1 °2θ, 19.8 °2θ, and 11.5 °2θ (±0.2 °2θ; ±0.1 °2θ; or ±0.0 °2θ; Cu Kα1 radiation).

43D. The solid form of any one of the previous embodiments, wherein the solid form of Compound 1 Freeform Type N is a crystalline polymorph of Compound 1 Freeform Type N characterized by two or more, or three or more XRPD signals selected from the group consisting of 17.2 °2θ, 22.5 °2θ, 11.1 °2θ, 19.8 °2θ, 11.5 °2θ, 19.1 °2θ, and 3.5 °2θ (±0.2 °2θ; ±0.1 °2θ; or ±0.0 °2θ; Cu Kα1 radiation).

43E. The solid form of any one of the previous embodiments, wherein the solid form of Compound 1 Freeform Type N is a crystalline polymorph of Compound 1 Freeform Type N characterized by two or more, or three or more XRPD signals selected from the group consisting of 17.2 °2θ, 22.5 °2θ, 11.1 °2θ, 19.8 °2θ, 11.5 °2θ, 19.1 °2θ, 3.5 °2θ, 23.9 °2θ, 20.9 °2θ, and 26.9 °2θ (±0.2 °2θ; ±0.1 °2θ; or ±0.0 °2θ; Cu Kα1 radiation).

43F. The solid form of any one of the previous embodiments, wherein the solid form of Compound 1 Freeform Type N is a crystalline polymorph of Compound 1 Freeform Type N characterized by a XRPD diffractogram substantially similar to that shown in FIG. 121.

43G. The solid form of any one of the previous embodiments, wherein the solid form of Compound 1 Freeform Type N is a crystalline polymorph of Compound 1 Freeform Type N characterized by any combination of the XRPD peaks set forth in Table 40.

43H. The solid form of any one of the previous embodiments, wherein the solid form of Compound 1 Freeform Type N is a crystalline polymorph of Compound 1 Freeform Type N characterized by a 1H NMR spectrum substantially similar to that shown in FIG. 168. 43I. The solid form of any one of the previous embodiments, wherein the solid form of Compound 1 Freeform Type N is a crystalline polymorph of Compound 1 Freeform Type N characterized by a DSC curve having endotherms at about 99.6° C., about 111.5° C., and about 187.4° C.

### Solid Forms of Compound 1 Freeform Type O

44. The solid form of any one of the previous embodiments, wherein the solid form of solid form 1 is crystalline Freeform Type O.

44A. The solid form of any one of the previous embodiments, wherein the solid form of Compound 1 Freeform Type O is a crystalline polymorph of Compound 1 Freeform Type O characterized by two or more, or three XRPD signals selected from the group consisting of 17.1 °2θ, 13.7 °2θ, and 14.9 °2θ (±0.2 °2θ; ±0.1 °2θ; or ±0.0 °2θ; Cu Kα1 radiation).

44B. The solid form of any one of the previous embodiments, wherein the solid form of Compound 1 Freeform Type O is a crystalline polymorph of Compound 1 Freeform Type O characterized by signals at 17.1 °2θ, 13.7 °2θ, and 14.9 °2θ (±0.2 °2θ; ±0.1 °2θ; or ±0.0 °2θ; Cu Kα1 radiation).

44C. The solid form of any one of the previous embodiments, wherein the solid form of Compound 1 Freeform Type O is a crystalline polymorph of Compound 1 Freeform Type O characterized by two or more, or three or more XRPD signals selected from the group consisting of 17.1 °2θ, 13.7 °2θ, 14.9 °2θ, 17.6 °2θ, and 18.1 °2θ (±0.2 °2θ; ±0.1 °2θ; or ±0.0 °2θ; Cu Kα1 radiation).

44D. The solid form of any one of the previous embodiments, wherein the solid form of Compound 1 Freeform Type O is a crystalline polymorph of Compound 1 Freeform Type O characterized by two or more, or three or more XRPD signals selected from the group consisting of 17.1 °2θ, 13.7 °2θ, 14.9 °2θ, 17.6 °2θ, 18.1 °2θ, 20.7 °2θ, and 23.1 °2θ (±0.2 °2θ; ±0.1 °2θ; or ±0.0 °2θ; Cu Kα1 radiation).

44E. The solid form of any one of the previous embodiments, wherein the solid form of Compound 1 Freeform Type O is a crystalline polymorph of Compound 1 Freeform Type O characterized by two or more, or three or more XRPD signals selected from the group consisting of 17.1 °2θ, 13.7 °2θ, 14.9 °2θ, 17.6 °2θ, 18.1 °2θ, 20.7 °2θ, 23.1 °2θ, 9.0 °2θ, 14.4 °2θ, and 6.8 °2θ (±0.2 °2θ; ±0.1 °2θ; or ±0.0 °2θ; Cu Kα1 radiation).

44F. The solid form of any one of the previous embodiments, wherein the solid form of Compound 1 Freeform Type O is a crystalline polymorph of Compound 1 Freeform Type O characterized by a XRPD diffractogram substantially similar to that shown in FIG. 124.

44G. The solid form of any one of the previous embodiments, wherein the solid form of Compound 1 Freeform Type O is a crystalline polymorph of Compound 1 Freeform Type O characterized by any combination of the XRPD peaks set forth in Table 41.

### Solid Forms of Compound 1 Freeform Type P

45. The solid form of any one of the previous embodiments, wherein the solid form of solid form 1 is crystalline Freeform Type P.

45A. The solid form of any one of the previous embodiments, wherein the solid form of Compound 1 Freeform Type P is a crystalline polymorph of Compound 1 Freeform Type P characterized by two or more, or three XRPD signals selected from the group consisting of 18.9 °2θ, 17.5 °2θ, and 10.9 °2θ (±0.2 °2θ; ±0.1 °2θ; or ±0.0 °2θ; Cu Kα1 radiation).

45B. The solid form of any one of the previous embodiments, wherein the solid form of Compound 1 Freeform Type P is a crystalline polymorph of Compound 1 Freeform Type P characterized by signals at 18.9 °2θ, 17.5 °2θ, and 10.9 °2θ (±0.2 °2θ; ±0.1 °2θ; or ±0.0 °2θ; Cu Kα1 radiation).

45C. The solid form of any one of the previous embodiments, wherein the solid form of Compound 1 Freeform Type P is a crystalline polymorph of Compound 1 Freeform Type P characterized by two or more, or three or more XRPD signals selected from the group consisting of 18.9 °2θ, 17.5 °2θ, 10.9 °2θ, 21.1 °2θ, and 22.3 °2θ (±0.2 °2θ; ±0.1 °2θ; or ±0.0 °2θ; Cu Kα1 radiation).

45D. The solid form of any one of the previous embodiments, wherein the solid form of Compound 1 Freeform Type P is a crystalline polymorph of Compound 1 Freeform Type P characterized by two or more, or three or more XRPD signals selected from the group consisting of 18.9 °2θ, 17.5 °2θ, 10.9 °2θ, 21.1 °2θ, 22.3 °2θ, 18.1 °2θ, and 21.5 °2θ (±0.2 °2θ; ±0.1 °2θ; or ±0.0 °2θ; Cu Kα1 radiation).

45E. The solid form of any one of the previous embodiments, wherein the solid form of Compound 1 Freeform Type P is a crystalline polymorph of Compound 1 Freeform Type P characterized by two or more, or three or more XRPD signals selected from the group consisting of 18.9 °2θ, 17.5 °2θ, 10.9 °2θ, 21.1 °2θ, 22.3 °2θ, 18.1 °2θ, 21.5 °2θ, 17.0 °2θ, 10.1 °2θ, and 22.9 °2θ (±0.2 °2θ; ±0.1 °2θ; or ±0.0 °2θ; Cu Kα1 radiation).

45F. The solid form of any one of the previous embodiments, wherein the solid form of Compound 1 Freeform Type P is a crystalline polymorph of Compound 1 Freeform Type P characterized by a XRPD diffractogram substantially similar to that shown in FIG. 125.

45G. The solid form of any one of the previous embodiments, wherein the solid form of Compound 1 Freeform Type P is a crystalline polymorph of Compound 1 Freeform Type P characterized by any combination of the XRPD peaks set forth in Table 42.

45H. The solid form of any one of the previous embodiments, wherein the solid form of Compound 1 Freeform Type P is a crystalline polymorph of Compound 1 Freeform Type P characterized by a 1H NMR spectrum substantially similar to that shown in FIG. 169.

45I. The solid form of any one of the previous embodiments, wherein the solid form of Compound 1 Freeform Type P is a crystalline polymorph of Compound 1 Freeform Type P characterized by a DSC curve having endotherms at about 80.8° C., about 166.5° C., and about 188.7° C.

46. A pharmaceutical composition comprising the solid form of any one of the previous embodiments.

47. A method of treating a disease mediated by glucagon-like peptide-1 receptor (GLP-1R) in an individual in need thereof, comprising administering to the individual a therapeutically effective amount of the solid form of any one of the previous embodiments.

48. The method of embodiment 47, wherein the disease is a liver disease.

49. The method of embodiment 47, wherein the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

50. The method of embodiment 47, wherein the disease is diabetes.

51. The method of embodiment 47, wherein the disease is a cardiometabolic disease.

52. The method of embodiment 47, wherein the disease is obesity.

53. Use of the solid form of any one of the previous embodiments, or a pharmaceutically acceptable salt thereof, in the manufacture of a medicament for treating a disease mediated by GLP-1R.

54. A method of decreasing food intake in an individual in need thereof, comprising administering to the individual a solid form of any one of the previous embodiments.

55. A method of increasing glucose tolerance in an individual in need thereof, comprising administering to the individual a solid form of any one of the previous embodiments.

1. A solid form of Compound 1.

2. The solid form of claim 1, wherein the solid form of Compound 1 is crystalline.

3. (canceled)

4. The solid form of claim 1, wherein the solid form of Compound 1 is a crystalline meglumine type A salt.

5. The solid form of claim 4, wherein the solid form of Compound 1 Meglumine Type A is a crystalline polymorph of Compound 1 Meglumine Type A characterized by two or more, or three XRPD signals selected from the group consisting of 20.8 °2θ, 17.4 °2θ, and 12.8 °2θ (±0.2 °2θ; ±0.1 °2θ; or ±0.0 °2θ; Cu Kα1 radiation).

6. The solid form of claim 4, wherein the solid form of Compound 1 Meglumine Type A is a crystalline polymorph of Compound 1 Meglumine Type A characterized by signals at 20.8 °2θ, 17.4 °2θ, and 12.8 °2θ (±0.2 °2θ; ±0.1 °2θ; or ±0.0 °2θ; Cu Kα1 radiation).

7. The solid form of claim 4, wherein the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, and 13.0 °2θ (±0.2 °2θ; ±0.1 °2θ; or ±0.0 °2θ; Cu Kα1 radiation).

8. The solid form of claim 4, wherein
the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, and 22.5 °2θ (±0.2 °2θ; ±0.1 °2θ; or ±0.0 °2θ; Cu Kα1 radiation).

9. The solid form of claim 4, wherein the solid form of Compound 1 Meglumine Type A is a crystalline polymorph of Compound 1 Meglumine Type A characterized by two or more, or three or more XRPD signals selected from the group consisting of 20.8 °2θ, 17.4 °2θ, 12.8 °2θ, 8.4 °2θ, 13.0 °2θ, 14.8 °2θ, 22.5 °2θ, 19.2 °2θ, 18.1 °2θ, and 24.0 °2θ (±0.2 °2θ; ±0.1 °2θ; or ±0.0 °2θ; Cu Kα1 radiation).

10. The solid form of claim 4, wherein the solid form of Compound 1 Meglumine Type A is a crystalline polymorph of Compound 1 Meglumine Type A characterized by a XRPD diffractogram substantially similar to that shown in FIG. 1.

11. The solid form of claim 4, wherein the solid form of Compound 1 Meglumine Type A is a crystalline polymorph of Compound 1 Meglumine Type A characterized by any combination of the XRPD peaks set forth in Table 24.

12. The solid form of claim 4, wherein the solid form of Compound 1 Meglumine Type A is a crystalline polymorph of Compound 1 Meglumine Type A characterized by a 1H NMR spectrum substantially similar to that shown in FIG. 130.

13. The solid form of claim 4, wherein the solid form of Compound 1 Meglumine Type A is a crystalline polymorph of Compound 1 Meglumine Type A characterized by a DSC curve having an endotherm at about 163.9° C.

14. The solid form of claim 1, wherein the solid form of solid form 1 is a crystalline fumarate type A salt.

15-23. (canceled)

24. The solid form of claim 1, wherein the solid form of solid form 1 is a crystalline potassium type A salt.

25-33. (canceled)

34. The solid form of claim 1, wherein the solid form of solid form 1 is a crystalline Tris type C salt.

35-43. (canceled)

44. The solid form of claim 1, wherein the solid form of solid form 1 is crystalline Freeform Type C.

45-53. (canceled)

54. A pharmaceutical composition comprising the solid form of claim 1.

55. A method of treating a disease mediated by glucagon-like peptide-1 receptor (GLP-1R) in an individual in need thereof, comprising administering to the individual a therapeutically effective amount of the solid form claim 1.

56-61. (canceled)

62. A method of decreasing food intake and/or increasing glucose tolerance in an individual in need thereof, comprising administering to the individual a solid form of claim 1.

63. (canceled)

64. A method of synthesizing the solid form of Compound 1 Meglumine Type A of claim 4 comprising the steps of:
(i) combining a sample of Compound 1 freeform and a sample of meglumine in acetone;
(ii) stirring the resultant mixture;
(iii) isolating the resulting solids; and
(iv) drying the resultant solids at room temperature.
